(19) United States
(12) Patent Application Publication
(10) Pub. No.: US 2024/0191176 A1
(43) Pub. Date: Jun. 13, 2024

(54) BACTERIA ENGINEERED TO SECRETE ACTIVE PROTEINS

(71) Applicant: Synlogic Operating Company, Inc., Cambridge, MA (US)

(72) Inventors: Aida Kalantari, Boston, MA (US); Douglas Kenny, Cambridge, MA (US); Analise Zaunbrecher Reeves, Cambridge, MA (US); Michael James, Melrose, MA (US); Mark Charbonneau, Cambridge, MA (US)

(21) Appl. No.: 18/286,661

(22) PCT Filed: Apr. 12, 2022
(86) PCT No.: PCT/US2022/024412

§ 371 Date: Oct. 12, 2023

Related U.S. Application Data
(60) Provisional application No. 63/174,349, filed on Apr. 13, 2021.
(60) Provisional application No. 63/210,903, filed on Jun. 15, 2021.

(51) Int. Cl. C12N 1/20; A61K 35/00; C07K 14/245; C07K 14/485; C12N 15/70; C12R 1/19

(52) U.S. Cl. CPC C12N 1/205; C07K 14/245; C07K 14/485; C12N 15/70; A61K 2035/11; C07K 2319/034; C12R 2001/19

(57) ABSTRACT

Recombinant bacteria capable of producing and secreting therapeutically active EGF, pharmaceutical compositions thereof, and methods of treating disorders are disclosed.

The present application claims the benefit of priority to U.S. Provisional Patent Application No. 63/174,349, filed Apr. 13, 2021, and U.S. Provisional Patent Application No. 63/210,903, filed Jun. 15, 2021, the contents of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Sep. 18, 2020, is named 126046-03002_SL.txt and is 1,084,118 bytes in size.

### BACKGROUND

A growing body of scientific evidence suggests that probiotic bacteria are beneficial in the treatment or prevention of various diseases or disorders associated with the gut, including, for example, gastrointestinal disorders such as Crohn's disease and inflammatory bowel syndrome. More recently, recombinant bacteria have emerged as a potential new therapeutic treatment modality for gastrointestinal diseases and have also opened the field of bacterial therapies to a large number of other indications, including metabolic diseases, inflammatory diseases, and cancer. One benefit of recombinant bacteria is the ability to specifically target one or more disease mechanisms. For example, for gastrointestinal disorders, bacteria can be engineered to contain genes for the expression of anti-inflammatory agents or agents that aid in the healing of a disrupted gut-barrier, such as the short chain fatty acid butyrate, e.g., as described in International Patent Publication WO2016141108.

Additionally, bacterial therapies have the additional advantage that the size of the bacterial chromosome(s) allows for the insertion of gene(s) for the production and secretion of multiple effectors. Potential secreted polypeptides include signaling molecules, such as cytokines and growth factors, their receptors, and single chain antibodies directed against cell surface molecules, many of which have been proposed as are promising candidates for therapeutic interference in a wide range of indications.

A certain level of technical understanding of approaches to the secretion of heterologous proteins from bacteria can be gained from recombinant production strategies for therapeutic or other proteins. However, effective protocols for generation of recombinant bacteria which produce and secrete biologically active polypeptides in vivo have yet to be established.

Multiple conditions must be met for the successful secretion of effective amounts of biologically active polypeptides. In Gram-negative bacteria, secreted polypeptides have to cross the two membranes and a thin layer of peptidoglycan in the periplasmic space between the inner and outer lipid membranes. Type I, II, III, IV, and V secretion pathways are common among Gram-negative bacteria, and all of these pathways have been exploited for the secretion of recombinant proteins. However, given that secretion of a polypeptide across the inner and outer membranes of a gram-negative bacterium is complex, involving the execution of several steps and the use of different biological factors, a number of complications can arise. For example, problems include incomplete translocation across the inner membrane, insufficient capacity of the export machinery, and proteolytic degradation (Mergulhao et al., Biotechnology Advances 23 (2005) 177-202). In addition, the ability of a polypeptide to be secreted from a Gram-negative bacterium, such as E. coli, depends on the specific polypeptide to be secreted and the biochemical properties thereof, such as formation of correct disulfide bonds, size of protein or levels of expression.

Given the number of factors involved in secreting polypeptides from Gram-negative bacteria, in combination with factors arising from the different biological properties and characteristics of individual polypeptides, e.g., size, dimer formation, secondary and tertiary protein folding, and polypeptide expression levels, secretion of polypeptides from Gram-negative bacteria remains challenging. Further, even if successful secretion is achieved, the polypeptide is not always secreted in a biologically active form.

In view of the difficulties outlined here as well as others, there remains a need for engineering and methods for the successful secretion of biologically active polypeptides.

### SUMMARY

The instant disclosure relates to compositions of recombinant bacteria and methods for secreting therapeutically active epidermal growth factor (EGF) from recombinant bacteria for treatment of diseases or disorders. The recombinant bacteria disclosed herein are capable of high yield production of functionally active EGF molecules, which are secreted as therapeutically active EGF polypeptides.

In some embodiments, the recombinant bacteria are functionally silent until they reach an inducing environment, e.g., a mammalian gut, or the tumor microenvironment wherein expression of EGF is induced. In certain embodiments, the recombinant bacteria are naturally non-pathogenic and may be introduced into the gut in order to reduce gut inflammation and/or enhance gut barrier function and may thereby further ameliorate or prevent an autoimmune disorder. In some embodiments, the recombinant bacteria are tumor targeting and may be introduced into the tumor to stimulate the immune system, combat immune suppression or otherwise fight the cancer. In certain embodiments, the secreted EGF molecule is stably produced by the recombinant bacteria, and/or the recombinant bacteria are stably maintained in vivo and/or in vitro. The disclosure also provides pharmaceutical compositions comprising the recombinant bacteria. Methods of treating diseases are also provided.

In some embodiments, the recombinant bacteria produce EGF under the control of one or more promoters induced by an environmental condition, e.g., an environmental condition found in the mammalian gut, such as an inflammatory condition or a low oxygen condition. In some embodiments, the recombinant bacteria produce one or more therapeutic molecule(s) under the control of one or more promoters induced the tumor microenvironment. In non-limiting exemplary embodiments, the recombinant bacteria produce EGF under the control of an oxygen level-dependent promoter, a reactive oxygen species (ROS)-dependent promoter, a reactive nitrogen species (RNS)-dependent promoter, or a temperature sensitive promoter, and a corresponding transcription factor.

In some embodiments, the recombinant bacterium comprises one or more one gene(s) encoding one or more EGF polypeptides for secretion of an active polypeptide in vivo, wherein the one or more gene sequence(s) for producing the EGF polypeptide is operably linked to a directly or indirectly inducible promoter that is not associated with the gene(s) in nature. In some embodiments, the secretion tags are N terminally or C terminally fused to the EGF polypeptides. For example, the secretion tag may be covalently linked to the N terminus of the polypeptide through a peptide bond or polypeptide linker. Alternatively, the secretion tag may be covalently linked to the C terminus of the polypeptide through a peptide bond or polypeptide linker.

Non-limiting examples of contemplated secretion tags include PhoA, OmpF, ompA, cvaC, TorA, fdnG, dmsA, PelB, tolB, torT, dsbA, GltI, GspD, HdeB, MalE, mglB, OppA, PpiA, lamb, ECOLIN_05715, ECOLIN_16495, ECOLIN_19410, and ECOLIN_19880 secretion signals. In some embodiments, the secretion tag is cleaved after secretion into the extracellular environment. In some embodiments, the secretion tag is PhoA. In some embodiments, the secretion tag is ECOLIN 19410 secretion tag. In some embodiments, the secretion tag is GspD secretion tag. In some embodiments, the secretion tag is HdeB secretion tag. In some embodiments, the secretion tag is torT secretion tag.

In some embodiments, the recombinant bacteria further have one or more mutations or deletions in an outer membrane protein selected from lpp, nlP, tolA, and pal. In some embodiments, the fully or partially deleted or mutated outer membrane protein is pal. In some embodiments, the recombinant bacteria further encode a stabilizing polypeptide. In some embodiments, the EGF polypeptide is covalently fused to the stabilizing polypeptide through a peptide linker or a peptide bond.

In some embodiments, the C terminus of the EGF polypeptide is covalently fused to the N terminus of the stabilizing polypeptide through the peptide linker or peptide bond. In some embodiments, the N terminus of the EGF polypeptide is covalently fused to the C terminus of the stabilizing polypeptide through the peptide linker or peptide bond. In some embodiments, the stabilizing polypeptide comprises an immunoglobulin Fc polypeptide. In some embodiments, the immunoglobulin Fc polypeptide comprises at least a portion of an immunoglobulin heavy chain CH2 constant region. In some embodiments, the immunoglobulin Fc polypeptide comprises at least a portion of an immunoglobulin heavy chain CH3 constant region. In some embodiments, the immunoglobulin Fc polypeptide comprises at least a portion of an immunoglobulin heavy chain CH1 constant region. In some embodiments, the immunoglobulin Fc polypeptide comprises at least a portion of an immunoglobulin variable hinge region. In some embodiments, the immunoglobulin Fc polypeptide comprises at least a portion of an immunoglobulin variable hinge region, immunoglobulin heavy chain CH2 constant region and an immunoglobulin heavy chain CH3 constant region. In some embodiments, the immunoglobulin Fc polypeptide is a human IgA or human IgG Fc polypeptide. In some embodiments, the immunoglobulin Fc polypeptide is a human IgG Fc polypeptide. In some embodiments, the immunoglobulin Fc polypeptide is a human IgA polypeptide. In some embodiments, the linker comprises a glycine rich peptide. In some embodiments, the glycine rich peptide comprises the sequence [GlyGlyGlyGlySer]n where n is 1, 2, 3, 4, 5 or 6 (SEQ ID NO: 1053). In some embodiments, the glycine rich peptide comprises the sequence GGGGSGGGGSGGGGSGGGGS (SEQ ID NO: 509). In some embodiments, the linker comprises GGGGSGGGS (SEQ ID NO: 1054). In some embodiments, the stabilizing polypeptide has the ability to perform an effector function. In some embodiments, the stabilizing polypeptide is able to perform an anti-inflammatory effector function. In some embodiments, the stabilizing polypeptide is able to perform a pro-inflammatory effector function. In some embodiments, the stabilizing polypeptide is a cytokine.

In some embodiments, the stabilizing polypeptide is a multimer. In some embodiments, the stabilizing polypeptide is a dimer. In some embodiments, the gene sequences encoding the stabilizing polypeptide comprise a monomer and a second monomer, wherein the first and second monomer are covalently linked to each other through a peptide bond or a peptide linker.

In some embodiments, the gene sequences are located on a chromosome in the bacterium. In some embodiments, the gene sequences are located on a plasmid in the bacterium. In some embodiments, the bacterium is a probiotic bacterium. In some embodiments, the bacterium is a tumor targeting bacterium. In some embodiments, the bacterium is selected from the group consisting of Bacteroides, Bifidobacterium, Clostridium, Escherichia, Lactobacillus, and Lactococcus. In some embodiments, the bacterium is selected from Clostridium novyi NT, and Clostridium butyricum, and Bifidobacterium longum. In some embodiments, the bacterium is Escherichia coli strain Nissle. In some embodiments, the bacterium is an auxotroph in a gene that is complemented when the bacterium is present in a mammalian gut, e.g., an auxotroph in diaminopimelic acid or an enzyme in the thymine biosynthetic pathway. Pharmaceutically acceptable compositions comprising the bacteria and methods of treating or preventing disorders are also provided.

### BRIEF DESCRIPTION OF THE FIGURES

FIG. 1 depicts a schematic representation of an exemplary bacterium that secretes EGF linked to a signal peptide and under control of an FNR-inducible promoter (left) or a heat-inducible promoter that also expresses a TIS secretion pore (right).

FIG. 2A and FIG. 2B depict schematics of the gene organization of exemplary circuits of the disclosure for the expression of therapeutic polypeptides, which are secreted via a diffusible outer membrane (DOM) system. The therapeutic polypeptide of interest is fused to a prototypical N-terminal Sec-dependent secretion signal or Tat-dependent secretion signal, which is cleaved upon secretion into the periplasmic space. Exemplary secretion tags include sec-dependent PhoA, PelB, OmpF, OmpA, cvaC, and Tat-dependent tags (TorA, FdnG, DmsA). In certain embodiments, the recombinant bacteria comprise deletions in one or more of lpp, pal, tolA, and/or nlpI. Optionally, periplasmic proteases are also deleted, including, but not limited to, degP and ompT, e.g., to increase stability of the polypeptide in the periplasm. A FRT-KanR-FRT cassette is used for downstream integration. Expression is driven by a tet promoter (FIG. 2A) or an inducible promoter, such as oxygen level-dependent promoters (e.g., FNR-inducible promoter, FIG. 2B) or temperature sensitive promoters, promoters induced by inflammation or an inflammatory response (RNS, ROS promoters), and promoters induced by a metabolite that may or may not be naturally present (e.g., can be exogenously added) in the gut or the tumor microenvironment, e.g., arabinose.

FIG. 3 shows an ELISA of EGF secretion across three different EGF fusion proteins in two different bacterial backgrounds grown in either LB (left) or grown in 2YT (right).

FIG. 4 shows an ELISA of bioreactivity for secreted hEGF on pERK and pEGFR in HT-29 cells.

FIG. 5 shows a western blot of phosphorylated downstream targets of EGF after treatment with either EGF, different supernatants, or the presence of AG1478, an EGFR inhibitor.

FIG. 6 shows an ELISA of pEGFR with secreted EGF.

FIG. 7 shows an ELISA of pERK with secreted EGF.

FIG. 8 shows an in vitro activity assay for hEGF production (WT or Δpal/DOM grown in either LB or 2YT medium before induction (30° C.) and 4 hours after induction (37° C.)).

FIG. 9 shows the production of hEGF (pg/ml) in WT or Δpal/DOM grown 2YT media at timepoints of 0 hours, 4 hours after induction and overnight induction.

FIG. 10 shows the production of hEGF (ng/ml) in WT-CI-OmpA-EGF or Δpal/DOM-CI-OmpA-EGF at timepoints of before induction, 4 hours after induction and overnight induction.

FIGS. 11A and 11B depict a schematic (FIG. 11A) and a bar graph showing EGF production (FIG. 11B). FIG. 11A shows a schematic describing four EGF secreting prototype strains. FIG. 11B shows a graph demonstrating EGF production represented as μg EGF produced per 5×10\hat{ }11 cells in 8 hr (mean±SEM). Numbers 1-3 indicate three separate experiments.

FIG. 12 depicts a schematic showing EGF production and secretion by prototype strains over time.

FIGS. 13A and 13B depicts graphs showing prototype strain bioactivity. FIG. 13A depicts a graph showing representative pEGFR signal in the FRET assay in HT-29 cells after 5 mins stimulation across a range of EGF concentrations using rEGF standards or supernatants collected in one of three independent experiments. FIG. 13B depicts a graph showing EC50 (nM EGF) of pEGFR stimulation in HT-29 cells determined using rEGF standards or supernatants collected in three separate experiments. EC50 values are presented as mean±95% CI. Dashed lines indicate ±0.5 log fold-change from the mean of rEGF standards across all three experiments.

FIG. 14 depicts a chart showing EGF detected in colon effluent of naïve mice treated with the prototype strains (1e10 CFU) as indicated at a six-hour time point.

FIG. 15A-15E depict schematics and graphs relating to an in vivo study. FIG. 15A is a schematic outlining the study protocol. Mice received a single oral bolus of EcN WT (SYN094), A-Temp (SYN8062), A-FNR (SYN8063) at 1×10\hat{ }10 CFU. Strain abundance in tissue effluents (small intestine (FIG. 15B), cecum (FIG. 15C), colon (FIG. 15D) and feces (FIG. 15E) were collected and counted at indicated times. For each time point, data represent the average CFU per gram of sample determined from 5 mice samples±standard error of the mean.

FIG. 16A-16E depict graphs relating to an in vivo study in which mice were gavaged at 1×10\hat{ }10 CFU with prototype strains showing EGF secretion in the gastrointestinal contents of naïve mice (stomach effluent (FIG. 16A), small intestine effluent (FIG. 16B), cecum effluent (FIG. 16C), colon effluent (FIG. 16D) and feces (FIG. 16E).

FIG. 17A-17C depict graphs relating to an in vivo study in DSS treated mice. Mice received a single oral bolus of SYN8066 (Chassis B, FNR) and SYN8248 (Chassis B) at 1e10 CFU. Strain abundance in tissue (small intestine effluents (FIG. 17A), cecum effluents (FIG. 17B), and colon contents (FIG. 17C)) were collected and counted at indicated times. For each time point, data represent the average CFU per gram of sample determined from 5 mice samples.

FIG. 18A-18C depict graphs relating to an in vivo study in which DSS treated mice were gavaged at 1e10 CFU with prototype strains showing EGF secretion in the gastrointestinal contents of DSS mice (small intestine effluent (FIG. 18A), cecum effluent (FIG. 18B), and colon contents (FIG. 18C).

FIG. 19 depicts a bar graph showing in vitro EGF production activity of strains SYN8371, SYN8408, and SYN8510 having 1, 2, or 3 integrated copies, respectively of FNR-ompA-EGF, as compared to the plasmid strain SYN8066 having the same construct.

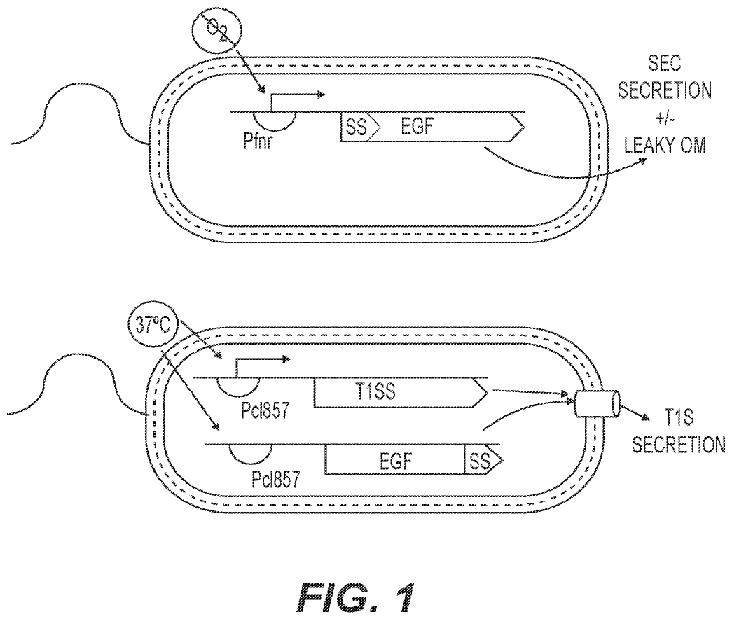

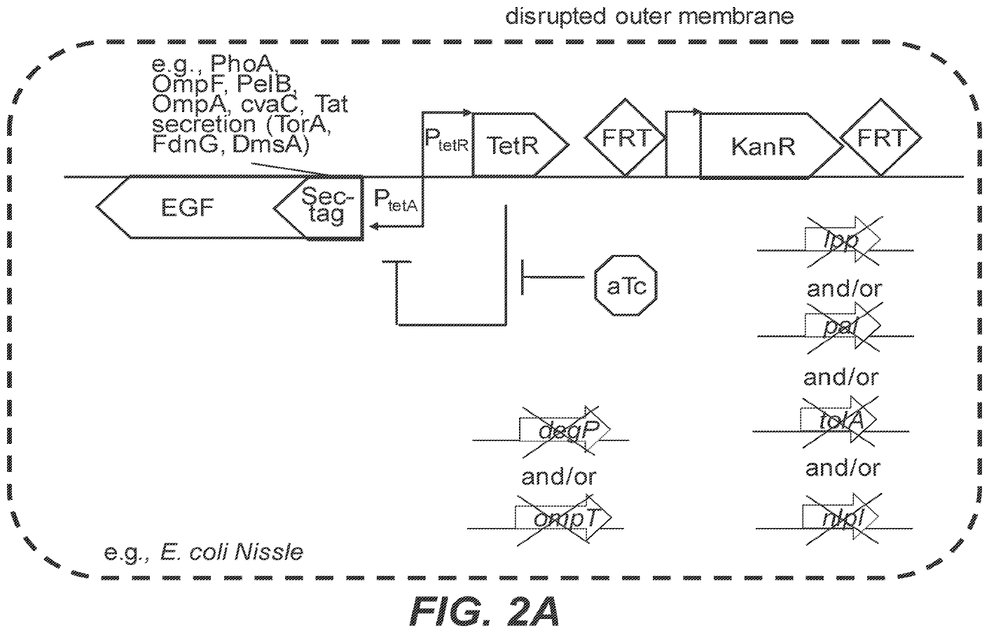

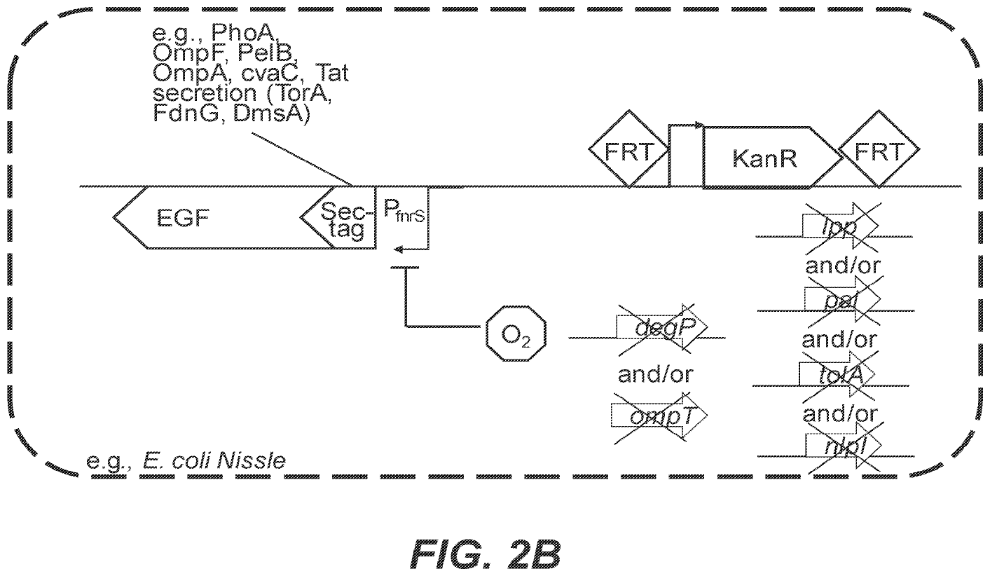

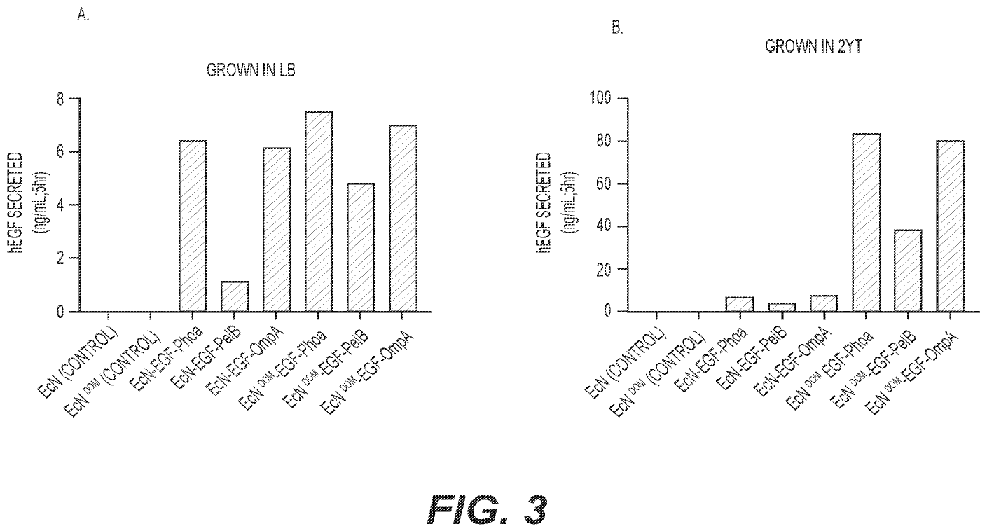

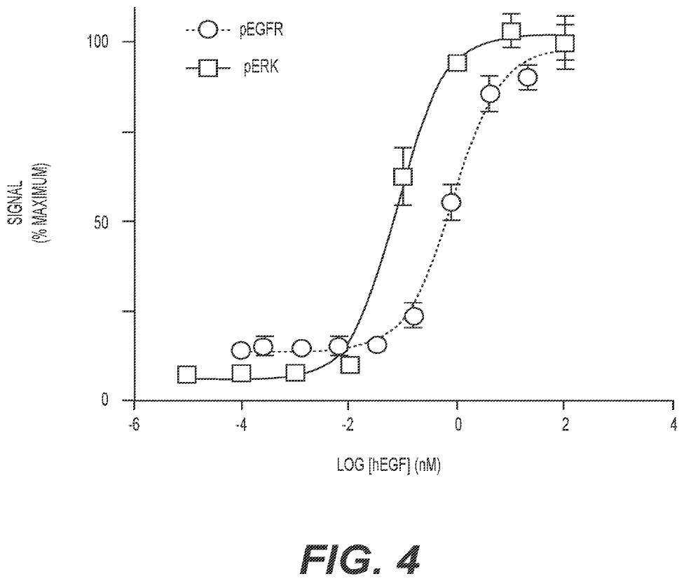

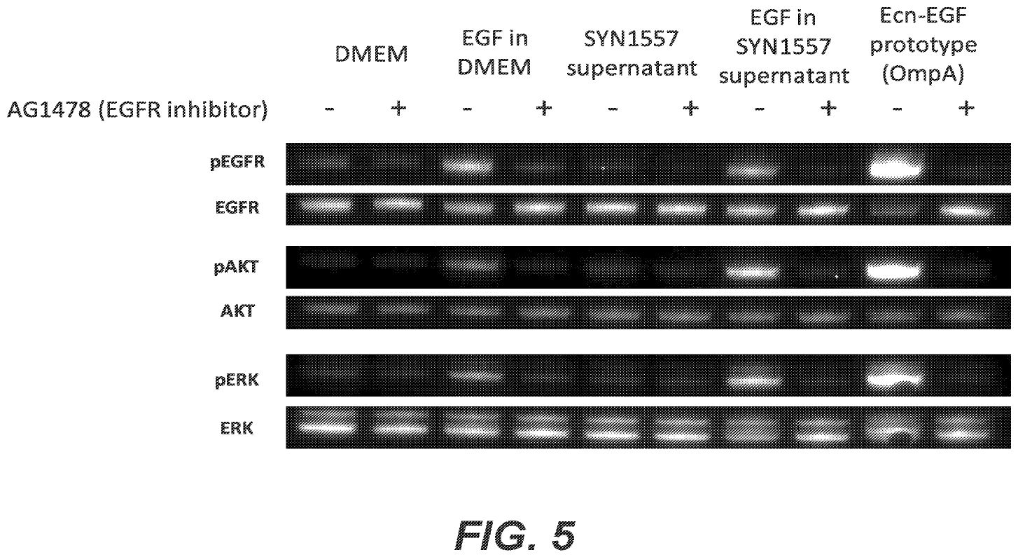

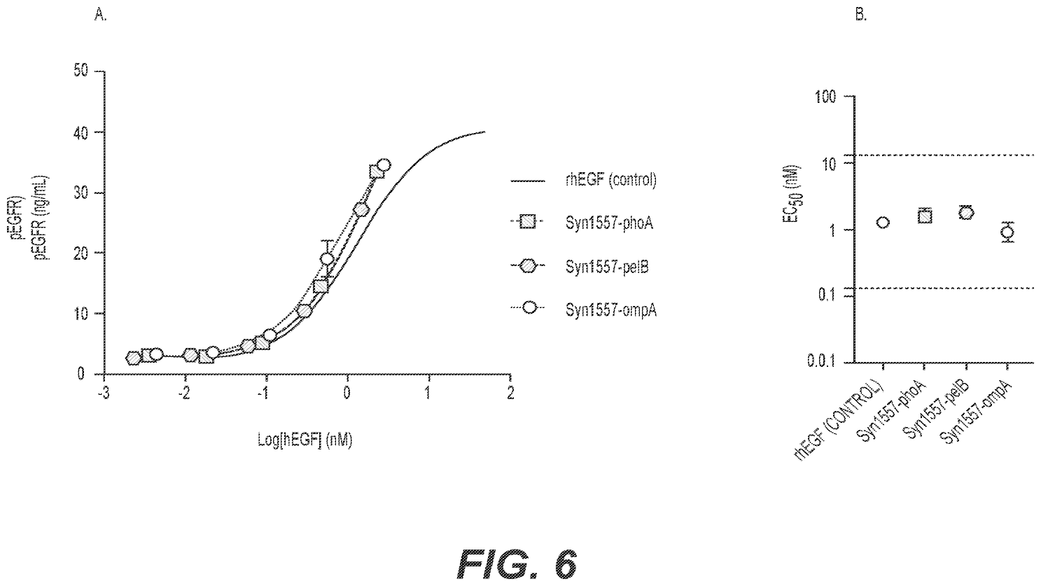

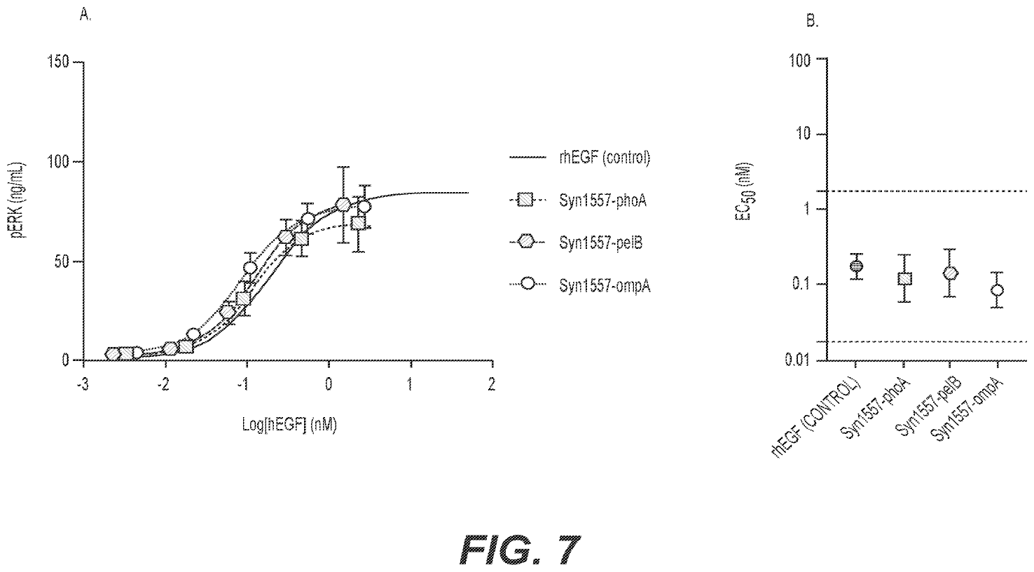

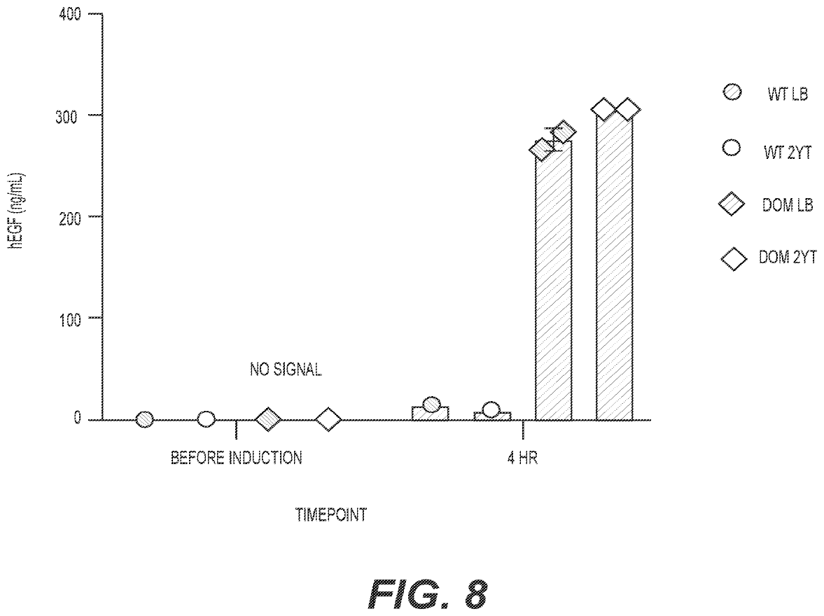

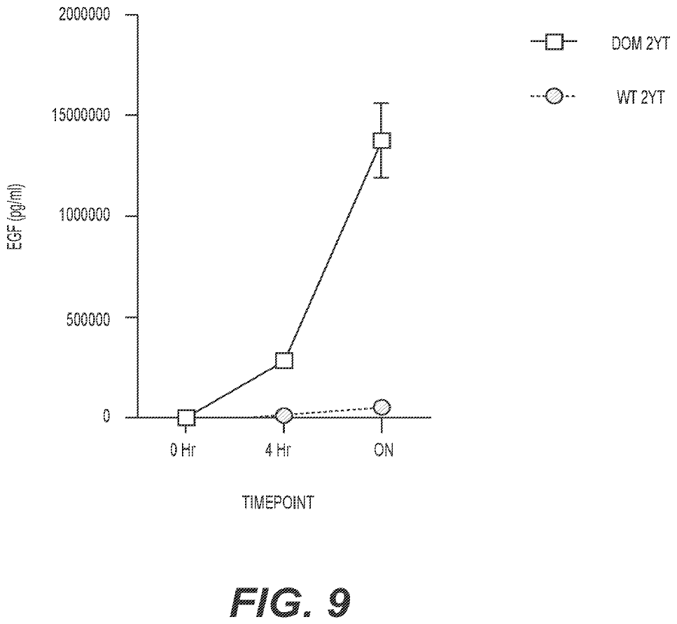

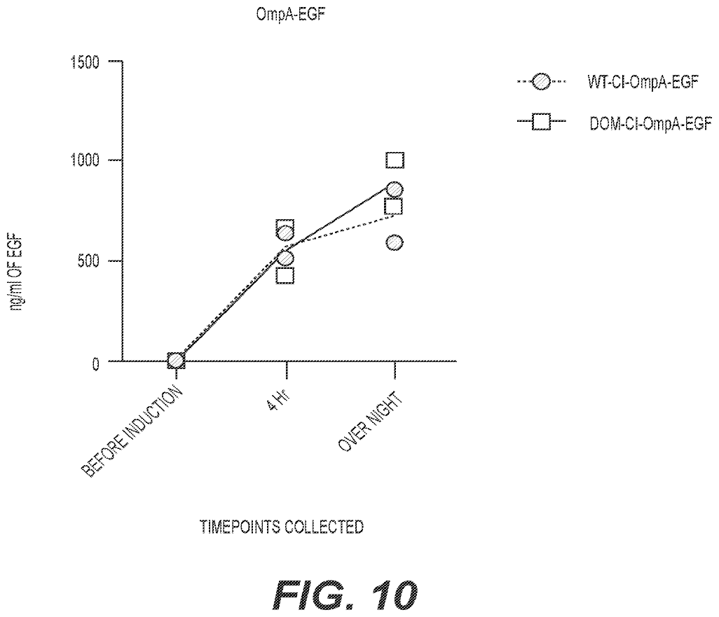

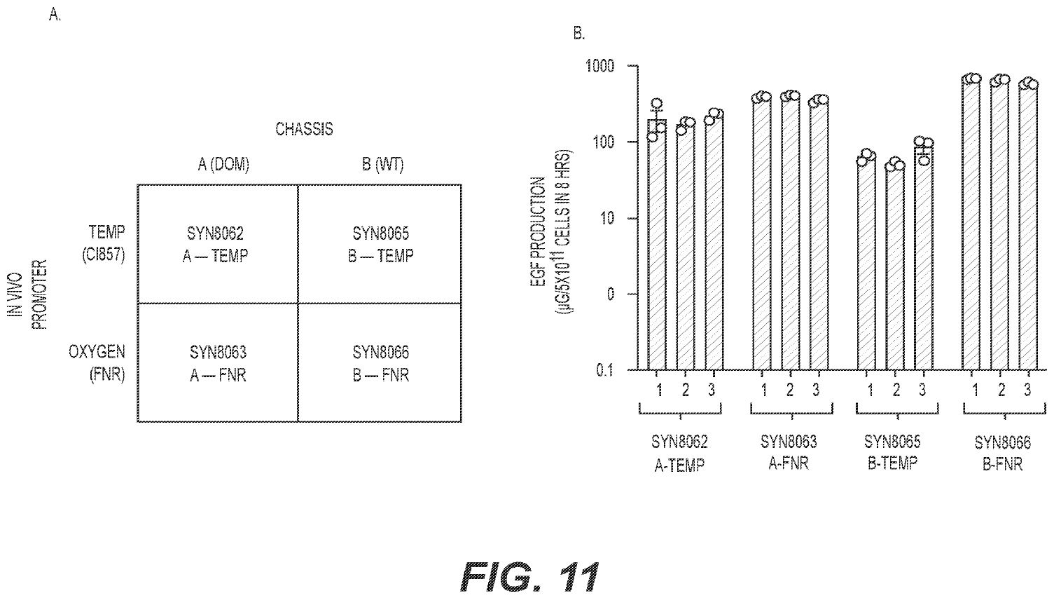

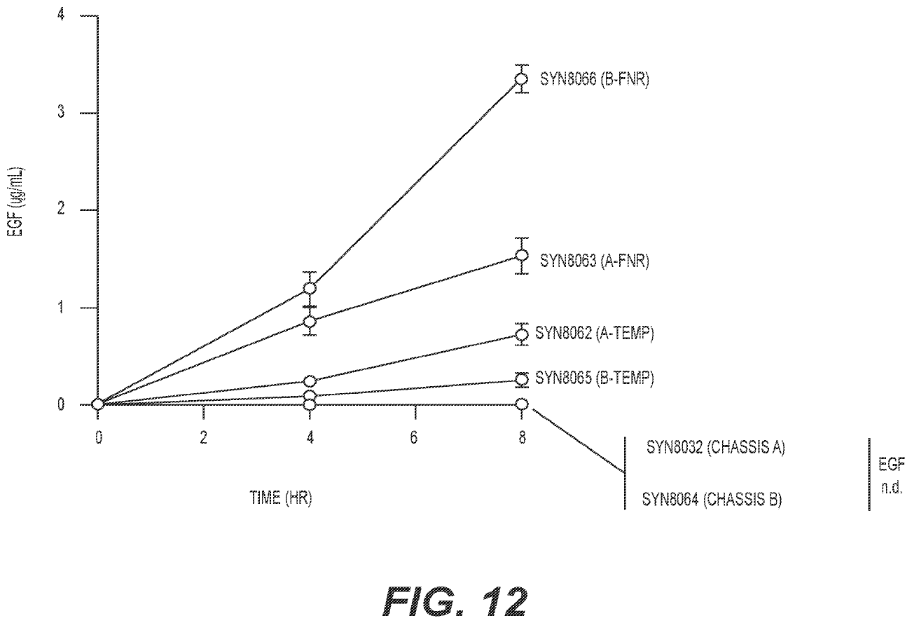

### DETAILED DESCRIPTION

The present disclosure relates to compositions of recombinant bacteria and methods for secreting EGF from recombinant bacteria for treatment of diseases or disorders. The recombinant bacteria disclosed herein are capable of high yield production of functionally active EGF, which is secreted as therapeutically active polypeptide.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “EGF” is used to encompass human, murine, and other species and sources of EGF polypeptides and polynucleotides, including naturally occurring EGF (e.g., naturally occurring EGF isoforms) and non-naturally occurring EGF (e.g., synthetic or engineered variants of EGF). Exemplary EGF sequences are known in the art. See, e.g., Dube et al., Epidermal growth factor receptor inhibits colitis-associated cancer in mice, J Clin Invest 2012; Sinha et al., Epidermal Growth Factor Enemas with Oral Mesalamine for Mild-to-Moderate Left-Sided Ulcerative Colitis or Proctitis, N Engl J Med 2003; Yu et al., Nononcogenic restoration of the intestinal barrier by E. coli-delivered human EGF, JCI Insight 2019; US20200299702A1; WO2013009103A2; the contents of which are hereby incorporated by reference in their entireties.

In some embodiments, a human EGF polypeptide comprises Sequence A (below), a functional fragment and/or variant thereof, or a polypeptide having at least 80%, 85%, 90%, 95%, 96%, 97%, 98%, or 99% identity to Sequence A or a functional fragment and/or variant thereof, e.g., as assessed by an alignment algorithm such as NCBI BLAST. A human EGF polynucleotide encodes a human EGF polypeptide. A non-limiting example of a human EGF polynucleotide comprises Sequence B.

Sequence A (human EGF):
NSDSECPLSHDGYCLHDGVCMYIEALDKYACNCVVGYIGERCQYRDLKWW
ELR
Sequence B (human EGF):
AATAGTGACAGCGAATGTCCGCTGTCGCACGATGGTTATTGCCTTCATGA
TGGGGTGTGCATGTACATTGAGGCATTGGACAAATATGCCTGCAACTGTG
TTGTCGGCTATATCGGCGAACGGTGTCAGTACCGTGATCTGAAATGGTGG
GAATTACGCTAA

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial or viral cell, or bacteria or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function. In certain embodiments, the programmed or engineered bacterial cell has been modified to express one or more proteins, for example, one or more EGF proteins that have a therapeutic activity or serve a therapeutic purpose. The programmed or engineered bacterial cell may additionally have the ability to stop growing or to destroy itself once EGF has been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

In some embodiments, the term EGF “gene” or “gene sequence” is meant to refer to a nucleic acid sequence encoding an EGF effector polypeptide described herein. The nucleic acid sequence may comprise the entire gene sequence or a partial gene sequence encoding a functional molecule. The nucleic acid sequence may be a natural sequence or a synthetic sequence. The nucleic acid sequence may comprise a native or wild-type sequence or may comprise a modified sequence having one or more insertions, deletions, substitutions, or other modifications, for example, the nucleic acid sequence may be codon-optimized.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene that is operably linked to a promoter that is not associated with said gene in nature. For example, in some embodiments, the recombinant bacteria disclosed herein comprise a gene that is operably linked to a directly or indirectly inducible promoter that is not associated with said gene in nature, e.g., an FNR responsive promoter (or other promoter disclosed herein) operably linked to EGF.

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or other promoter disclosed herein.

As used herein, a “gene cassette” or “operon” encoding a biosynthetic pathway refers to the two or more genes for the production of an effector molecule such as EGF. In addition to encoding a set of genes capable of producing said molecule, the gene cassette or operon may also comprise additional transcription and translation elements, e.g., a ribosome binding site.

A regulatory region “operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene encoding EGF, that is joined to a regulatory sequence in a manner which allows expression of the nucleic acid sequence, e.g., the gene encoding the EGF molecule described herein. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes.

A regulatory region or sequence is a nucleic acid that can direct transcription of an EGF gene and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σS promoter (e.g., an osmY promoter (International Genetically Engineered Machine (iGEM) Registry of Standard Biological Parts Name BBa_J45992; BBa_J45993)), a constitutive Escherichia coli σ32 promoter (e.g., htpG heat shock promoter (BBa_J45504)), a constitutive Escherichia coli σ70 promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σA promoter (e.g., promoter veg (BBa_K143013), promoter 43 (BBa_K143013), PliaG (BBa_K823000), PlepA (BBa_K823002), Pveg (BBa_K823003)), a constitutive Bacillus subtilis σB promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa 1712074; BBa_1719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.”

As used herein, “stably maintained” or “stable” bacterium is used to refer to a bacterial host cell carrying non-native genetic material, e.g., a gene encoding one or more EGF molecule(s), which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a recombinant bacterium comprising a gene encoding a encoding a payload, e.g., one or more EGF molecule(s), in which the plasmid or chromosome carrying the gene is stably maintained in the bacterium, such that the payload can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding EGF molecule.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising EGF operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are specific to the tumor microenvironment. In some embodiments, the exogenous environmental conditions are low-oxygen, microaerobic, or anaerobic conditions, such as the environment of the mammalian gut or the tumor microenvironment. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to an inflammatory disease. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR (anaerobic nitrate respiration), and DNR (dissimilatory nitrate respiration regulator). Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

As used herein, a “tunable regulatory region” refers to a nucleic acid sequence under direct or indirect control of a transcription factor and which is capable of activating, repressing, derepressing, or otherwise controlling gene expression relative to levels of an inducer. In some embodiments, the tunable regulatory region comprises a promoter sequence. The inducer may be RNS, or other inducer described herein, and the tunable regulatory region may be a RNS-responsive regulatory region or other responsive regulatory region described herein. The tunable regulatory region may be operatively linked to a gene sequence(s) or gene cassette for the production of one or more payloads, e.g., EGF gene cassette or gene sequence(s). For example, in one specific embodiment, the tunable regulatory region is a RNS-derepressible regulatory region, and when RNS is present, a RNS-sensing transcription factor no longer binds to and/or represses the regulatory region, thereby permitting expression of the operatively linked gene or gene cassette. In this instance, the tunable regulatory region derepresses gene or gene cassette expression relative to RNS levels. Each gene or gene cassette may be operatively linked to a tunable regulatory region that is directly or indirectly controlled by a transcription factor that is capable of sensing at least one RNS.

In some embodiments, the exogenous environmental conditions are the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut or any other organ of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the molecule that activates the inducible promoter is present in the tumor microenvironment. In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorportated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. For example, Table 2 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 2
Oxygen levels

| Compartment | Oxygen Tension |
|---|---|
| stomach | ~60 torr (e.g., 58 +/− 15 torr) |
| duodenum and first part of | ~30 torr (e.g., 32 +/− 8 torr); ~ 20% oxygen |
| jejunum | in ambient air |
| Ileum (mid- small intestine) | ~10 torr; ~6% oxygen in ambient air (e.g., |
| | 11 +/− 3 torr) |
| Distal sigmoid colon | ~ 3 torr (e.g., 3 +/− 1 torr) |
| colon | <2torr |
| Lumen of cecum | <1 torr |
| tumor | <32 torr (most tumors are <15 torr) |

“Microorganism” refers to an organism or microbe of microscopic, submicroscopic, or ultramicroscopic size that typically consists of a single cell. Examples of microorganisms include bacteria, viruses, parasites, fungi, certain algae, yeast, e.g., Saccharomyces, and protozoa. In some aspects, the microorganism is engineered (“engineered microorganism”) to produce one or more therapeutic molecules, e.g., an anti-inflammatory or barrier enhancer molecule. In certain embodiments, the engineered microorganism is an engineered bacterium.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. Nos. 6,835,376; 6,203,797; 5,589,168; 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia Coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the term “modulate” and its cognates means to alter, regulate, or adjust positively or negatively a molecular or physiological readout, outcome, or process, to effect a change in said readout, outcome, or process as compared to a normal, average, wild-type, or baseline measurement. Thus, for example, “modulate” or “modulation” includes up-regulation and down-regulation. A non-limiting example of modulating a readout, outcome, or process is effecting a change or alteration in the normal or baseline functioning, activity, expression, or secretion of a biomolecule (e.g., a protein, enzyme, cytokine, growth factor, hormone, metabolite, short chain fatty acid, or other compound). Another non-limiting example of modulating a readout, outcome, or process is effecting a change in the amount or level of a biomolecule of interest, e.g., in the serum and/or the gut lumen. In another non-limiting example, modulating a readout, outcome, or process relates to a phenotypic change or alteration in one or more disease symptoms. Thus, “modulate” is used to refer to an increase, decrease, masking, altering, overriding or restoring the normal functioning, activity, or levels of a readout, outcome or process (e.g, biomolecule of interest, and/or molecular or physiological process, and/or a phenotypic change in one or more disease symptoms).

As used herein, the terms “modulate” and “treat” a disease and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disorder, as well as those at risk of having, or who may ultimately acquire the disorder. The need for treatment is assessed, for example, by the presence of one or more risk factors associated with the development of a disorder, the presence or progression of a disorder, or likely receptiveness to treatment of a subject having the disorder. Treating autoimmune disorders and/or diseases and conditions associated with gut inflammation and/or compromised gut barrier function may encompass reducing or eliminating excess inflammation and/or associated symptoms, and does not necessarily encompass the elimination of the underlying disease. Treating the diseases described herein may encompass increasing levels EGF and does not necessarily encompass the elimination of the underlying disease.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor and does not necessarily encompass the elimination of the underlying cause of the cancer, e.g., an underlying genetic predisposition.

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute). Those in need of treatment may include individuals already having a particular cancer, as well as those at risk of having, or who may ultimately acquire an autoimmune disorder.

As used herein, “diseases and conditions associated with gut inflammation and/or compromised gut barrier function” include, but are not limited to, inflammatory bowel diseases, diarrheal diseases, and related diseases. “Inflammatory bowel diseases” and “IBD” are used interchangeably herein to refer to a group of diseases associated with gut inflammation, which include, but are not limited to, Crohn's disease, ulcerative colitis, collagenous colitis, lymphocytic colitis, diversion colitis, Bechet's disease, and indeterminate colitis. As used herein, “diarrheal diseases” include, but are not limited to, acute watery diarrhea, e.g., cholera; acute bloody diarrhea, e.g., dysentery; and persistent diarrhea. As used herein, related diseases include, but are not limited to, short bowel syndrome, ulcerative proctitis, proctosigmoiditis, left-sided colitis, pancolitis, and fulminant colitis.

Symptoms associated with the aforementioned diseases and conditions include, but are not limited to, one or more of diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, inflammation of the skin, inflammation of the eyes, inflammation of the joints, inflammation of the liver, and inflammation of the bile ducts.

As used herein, “metabolic diseases” include, but are not limited to, type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.

Symptoms associated with the aforementioned diseases and conditions include, but are not limited to, one or more of weight gain, obesity, fatigue, hyperlipidemia, hyperphagia, hyperdipsia, polyphagia, polydipsia, polyuria, pain of the extremities, numbness of the extremities, blurry vision, nystagmus, hearing loss, cardiomyopathy, insulin resistance, light sensitivity, pulmonary disease, liver disease, liver cirrhosis, liver failure, kidney disease, kidney failure, seizures, hypogonadism, and infertility.

As used herein a “pharmaceutical composition” refers to a preparation of genetically engineered microorganism of the disclosure, e.g., recombinant bacteria, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., inflammation, diarrhea, an autoimmune disorder. A therapeutically effective amount may, for example, be sufficient to treat, prevent, reduce the severity, delay the onset, and/or reduce the risk of occurrence of one or more symptoms of an autoimmune a disorder and/or a disease or condition as described herein. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, “payload” refers to one or more molecules of interest to be produced by a genetically engineered microorganism, such as a bacteria or a virus. In some embodiments, the payload is a therapeutic EGF payload. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS or a cI857 repressor-pR promoter.

In some embodiments, the payload comprises a repressor element, such as a kill switch. In some embodiments, the payload comprises an antibiotic resistance gene or genes. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional treatment” or “conventional therapy” refers to treatment or therapy that is currently accepted, considered current standard of care, and/or used by most healthcare professionals for treating a disease or disorder, e.g., cancer, autoimmune disorders, metabolic diseases, diseases relating to inborn errors of metabolism, neurological or neurodegenerative diseases, or diseases associated with inflammation and/or reduced gut barrier function. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the recombinant bacteria of the current disclosure. A polypeptide may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e., produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

In some embodiments of the disclosure, the recombinant bacteria comprise one or more gene sequence(s) encoding one or more fusion proteins. In some embodiments, the recombinant bacteria express a fusion protein, in which a secretion tag polypeptide is fused to an EGF polypeptide, i.e., the secretion tag is linked to the polypeptide through a peptide bond or a linker. In some embodiments, the recombinant bacteria express an EGF polypeptide which is fused to a stabilizing polypeptide. As used herein “stabilizing polypeptide” extends the half-life of the EGF polypeptide to which it is fused. Non-limiting examples of fusion proteins containing such stabilizing polypeptides include Fc fusion proteins, transferrin fusion proteins, and albumin fusion proteins (Strohl, BioDrugs. 2015; 29(4): 215-239). In some embodiments, the EGF polypeptide is fused to an inert polypeptide to extend the half-life. A non-limiting example of such a polypeptide is XTEN (Schellenberger V, et al. Nat Biotechnol. 2009; 27:1186-1190). Another non-limiting example of a half-life extending polypeptide is CTP. CTP naturally extends protein's half-life in human serum, likely because the negatively charged, heavily sialylated CTP impairs renal clearance. Another non-limiting example of a polypeptide which can be fused to EGF to extend half-life is ELPs, which are repeating peptide units containing sequences commonly found in elastin (repeats of V-P-G-x-G, where x is any amino acid except proline (SEQ ID NO: 1057) (Strohl et al.). Other non-limiting examples of a polypeptide containing a polypeptide repeat sequence which can be fused to EGF are PAS (polymer using three repeating amino acids, proline, alanine and serine) and HAP (glycine-rich HAP).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, 50%, 55%, 60%, 65%, 70%, 75%, 80%, 85%, 90%, 91%, 92%, 93%, 94%, 95%, 96%, 97%, 98%, 99%, or 100%, identical are defined herein as sufficiently similar. Preferably, variants will be sufficiently similar to the amino acid sequence of the peptides of the disclosure. Such variants generally retain the functional activity of the peptides of the present disclosure. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701 and in Chen et al., Adv Drug Deliv Rev. 2013; 65(10): 1357-1369, the contents of which are herein incorporated by reference in its entirety. Table 3 depicts non-limiting examples of linkers known in the art.

TABLE 3
Linkers

| | | | SEQ |
|---|---|---|---|
| | | | ID |
| | | | NO: |
| Increase Stability/Folding | | | |
| scFv+DB2:D24 | 1063 | (GGGGS)3 | 1063 |
| G-CSF-Tf | 1063 | (GGGGS)3 | 1063 |
| HBsAg preS1 | 1063 | (GGGGS)3 | 1063 |
| Myc- Est2p | 1061 | (Gly)8 | 1061 |
| albumin-ANF | 1064 | (Gly)6 | 1064 |
| virus coat | 1065 | (EAAAK)3 | 1065 |
| protein | | | |
| beta-glucanase- | 1066 | (EAAAK)n (n = 1-3) | 1066 |
| xylanase | | | |
| Increase expression | | | |
| hGH-Tf and | 1067 | A(EAAAK)4ALEA(EAAAK)4A | 1067 |
| Tf-hGH | | | |
| G-CSF-Tf and | 1067 | A(EAAAK)4ALEA(EAAAK)4A | 1067 |
| Tf-G-CSF | rigid | | |
| Improve biological activty | | | |
| G-CSF-Tf | 1063 | (GGGGS)3 | 1063 |
| G-CSF-Tf | 1067 | A(EAAAK)4ALEA(EAAAK)4A | 1067 |
| hGH-Tf | 1067 | A(EAAAK)4ALEA(EAAAK)4A | 1067 |
| HSA-IFN-a2b | 1058 | GGGGS | 1058 |
| HSA-IFN-a2b | 1068 | PAPAP | 1068 |
| HSA-IFN-a2b | 1069 | AEAAAKEAAAKA | 1069 |
| PGA-rTHS | 1070 | (GGGGS)n (n = 1, 2, 4) | 1070 |
| interferon-y- | 1071 | (Ala-Pro)n (10 − 34 aa) | 1071 |
| gp120 | | | |
| GSF-S-S-Tf | | disulfide | |
| IFN-a2b-HSA | | disulfide | |
| Enable targeting | | | |
| FIX-albumin | 1072 | VSQTSKLTR↓AETVFPDV | 1072 |
| LAP-IFN-ß | 1073 | PLG ↓ LWA | 1073 |
| MazE-MazF | 1074; 1075 | RVL↓AEA; EDVVCC↓SMSY; | 1074; |
| | | | 1075 |
| | | GGIEGR↓GSC | 1076 |
| Immunotoxins | cleavable | TRHRQPR↓GWE; | 1077 |
| | cleavable | AGNRVRR↓SVG; | 1078 |
| | cleavable | RRRRRRR↓R↓R | 1079 |
| Immunotoxin | cleavable | GFLG↓ | 1080 |
| Alter PK | | | |
| G-CSF-Tf and | dipeptide | LE | |
| hGH-Tf | rigid | A(EAAAK)4ALEA(EAAAK)4A | 1067 |
| | cleavable | Disulfide | |

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g., HlyBD. Non-limiting examples of secretion systems for gram negative bacteria include the type I (e.g., hemolysin secretion system), type II, type III, type III flagellar, type IV, type V, type VI, type VII, type VIII secretion systems and modifications thereof, e.g., modified type III, modified type III flagellar, resistance-nodulation-division (RND) multi-drug efflux pumps, and various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the anti-inflammatory or barrier enhancer molecule(s) into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp functions as the primary ‘staple’ of the bacterial cell wall to the peptidoglycan. TolA-pal and OmpA complexes function similarly to Lpp and are other deletion targets to generate a leaky phenotype. Additionally, leaky phenotypes have been observed when periplasmic proteases, such as degS, degP or nlpI, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

The terms “phage” and “bacteriophage” are used interchangeably herein. Both terms refer to a virus that infects and replicates within a bacterium. As used herein “phage” or bacteriophage” collectively refers to prophage, lysogenic, dormant, temperate, intact, defective, cryptic, and satellite phage, phage tail bacteriocins, tailiocins, and gene transfer agents. As used therein the term “prophage” refers to the genomic material of a bacteriophage, which is integrated into a replicon of the host cell and replicates along with the host. The prophage may be able to produce phages if specifically activated. In some cases, the prophage is not able to produce phages or has never done so (i.e., defective or cryptic prophages). In some cases, prophage also refers to satellite phages. The terms “prophage” and “endogenous phage” are used interchangeably herein. “Endogenous phage” or “endogenous prophage” also refers to a phage that is present in the natural state of a bacterium (and its parental strain). As used herein the term “phage knockout” or “inactivated phage” refers to a phage which has been modified so that it can either no longer produce and/or package phage particles or it produces fewer phage particles than the wild type phage sequence. In some embodiments, the inactivated phage or phage knockout refers to the inactivation of a temperate phage in its lysogenic state, i.e., to a prophage. Such a modification refers to a mutation in the phage; such mutations include insertions, deletions (partial or complete deletion of phage genome), substitutions, inversions, at one or more positions within the phage genome, e.g., within one or more genes within the phage genome. As used herein the adjectives “phage-free”, “phage free” and “phageless” are used interchangeably to characterize a bacterium or strain which contains one or more prophages, one or more of which have been modified. The modification can result in a loss of the ability of the prophage to be induced or release phage particles. Alternatively, the modification can result in less efficient or less frequent induction or less efficient or less frequent phage release as compared to the isogenic strain without the modification. Ability to induce and release phage can be measured using a plaque assay as described herein. As used herein phage induction refers to the part of the life cycle of a lysogenic prophage, in which the lytic phage genes are activated, phage particles are produced and lysis occurs.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Bacteria

The genetically engineered microorganisms, or programmed microorganisms, such as recombinant bacteria of the disclosure are capable of producing EGF. In certain embodiments, the recombinant bacteria are obligate anaerobic bacteria. In certain embodiments, the recombinant bacteria are facultative anaerobic bacteria. In certain embodiments, the recombinant bacteria are aerobic bacteria. In some embodiments, the recombinant bacteria are Gram-positive bacteria. In some embodiments, the recombinant bacteria are Gram-positive bacteria and lack LPS. In some embodiments, the recombinant bacteria are Gram-negative bacteria. In some embodiments, the recombinant bacteria are Gram-positive and obligate anaerobic bacteria. In some embodiments, the recombinant bacteria are Gram-positive and facultative anaerobic bacteria. In some embodiments, the recombinant bacteria are non-pathogenic bacteria. In some embodiments, the recombinant bacteria are commensal bacteria. In some embodiments, the recombinant bacteria are probiotic bacteria. In some embodiments, the recombinant bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the recombinant bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii, Clostridium clusters IV and XIVa of Firmicutes (including species of Eubacterium), Roseburia, Faecalibacterium, Enterobacter, Faecalibacterium prausnitzii, Clostridium difficile, Subdoligranulum, Clostridium sporogenes, Campylobacter jejuni, Clostridium saccharolyticum, Klebsiella, Citrobacter, Pseudobutyrivibrio, and Ruminococcus. In certain embodiments, the recombinant bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis

In some embodiments, the recombinant bacterium is a Gram-positive bacterium, e.g., Clostridium, that is naturally capable of producing high levels of butyrate. In some embodiments, the recombinant bacterium is selected from the group consisting of C. butyricum ZJUCB, C. butyricum 521, C. thermobutyricum ATCC 49875, C. beijerinckii, C. populeti ATCC 35295, C. tyrobutyricum JM1, C. tyrobutyricum CIP 1-776, C. tyrobutyricum ATCC 25755, C. tyrobutyricum CNRZ 596, and C. tyrobutyricum ZJU 8235. In some embodiments, the recombinant bacterium is C. butyricum CBM588, a probiotic bacterium that is highly amenable to protein secretion and has demonstrated efficacy in treating IBD (Kanai et al., 2015). In some embodiments, the recombinant bacterium is Bacillus, a probiotic bacterium that is highly genetically tractable and has been a popular chassis for industrial protein production; in some embodiments, the bacterium has highly active secretion and/or no toxic byproducts (Cutting, 2011).

In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell.

In some embodiments, the recombinant bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007). In some embodiments, the recombinant bacteria are E. coli Nissle and are naturally capable of promoting tight junctions and gut barrier function. In some embodiments, the recombinant bacteria are E. coli and are highly amenable to recombinant protein technologies.

One of ordinary skill in the art would appreciate that the genetic modifications disclosed herein may be adapted for other species, strains, and subtypes of bacteria. It is known, for example, that the clostridial butyrogenic pathway genes are widespread in the genome-sequenced clostridia and related species (Aboulnaga et al., 2013). Furthermore, genes from one or more different species of bacteria can be introduced into one another, e.g., the butyrogenic genes from Peptoclostridium difficile have been expressed in Escherichia coli (Aboulnaga et al., 2013).

In one embodiment, the recombinant bacterial cell does not colonize the subject having the disorder. Unmodified E. coli Nissle and the recombinant bacteria may be destroyed, e.g., by defense factors in the gut or blood serum (Sonnenborn et al., 2009) or by activation of a kill switch, several hours or days after administration. Thus, the recombinant bacteria may require continued administration. Residence time in vivo may be calculated for the recombinant bacteria. In some embodiments, the residence time is calculated for a human subject. In some embodiments, residence time in vivo is calculated for the recombinant bacteria of the disclosure, e.g., as described herein.

In some embodiments, the bacterial cell is a recombinant bacterial cell. In some embodiments, the disclosure comprises a colony of bacterial cells disclosed herein. In another aspect, the disclosure provides a recombinant bacterial culture which comprises bacterial cells disclosed herein.

In some embodiments, the recombinant bacteria comprising a gene sequence encoding EGF further comprise a kill-switch circuit, such as any of the kill-switch circuits provided herein. For example, in some embodiments, the recombinant bacteria further comprise one or more genes encoding one or more recombinase(s) under the control of an inducible promoter, and an inverted toxin sequence. In some embodiments, the recombinant bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the recombinant bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as ParaBAD. In some embodiments, the recombinant bacteria further comprise one or more genes encoding an antitoxin.

In some embodiments, the recombinant bacteria is an auxotroph comprising a gene sequence encoding EGF and further comprises a kill-switch circuit, such as any of the kill-switch circuits described herein.

In some embodiments of the above-described recombinant bacteria, the gene sequence encoding EGF is present on a plasmid in the bacterium. In some embodiments, the gene sequence encoding EGF is present in the bacterial chromosome. In some embodiments, a gene sequence encoding a secretion protein or protein complex, such as any of the secretion systems disclosed herein, for secreting EGF, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

In some embodiments, the genetically engineered bacteria comprise one or more E. coli Nissle bacteriophage sequence(s), and at least one of the bacteriophage sequence(s) is mutated or modified, e.g., to delete the bacteriophage sequence, e.g., an endogenous prophage sequence, in part or whole. In some embodiments, the deletion prevents the bacteria from being able to express infectious bacteriophage particles. Non-limiting examples of such mutations or modifications are described in PCT/US2018/038840, the contents of which are incorporated by reference in their entirety. In some embodiments, the genetically engineered bacteria comprise one or modifications or mutations in one or more of Phage 1, 2 or 3 as described in PCT/US2018/038840. In some embodiments, the genetically engineered bacteria comprise a modification or mutation in Phage 3. In some embodiments, the mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 1064 of PCT/US2018/038840 is deleted from the Phage 3 genome. In one embodiment, a sequence comprising SEQ ID NO: 1064 PCT/US2018/038840 is deleted from the Phage 3 genome.

### PKS Island

In some embodiments, the engineered bacterium further comprises a modified pks island (colibactin island). Non-limiting examples are described in PCT/US2021/061579, the contents of which are herein incorporated by reference in their entirety. Colibactin is a cyclomodulin that is synthetized by enzymes encoded by the pks genomic island. See Fais 2018. The pks genomic island is “highly conserved” in Enterobacteriaceae. Id. In Escherichia coli, a 54-kilobase pks genomic island contains 19 genes, clbA to clbS, and encodes various enzymes that have been described as an “assembly line responsible for colibactin synthesis.” Id. The pks genomic island assembly line for colibactin synthesis includes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. The polyketide synthases, non-ribosomal peptide synthases, and hybrid enzymes “are usually organized in mega-complexes as an assembly line, in which the synthesized compound is transferred from one enzymatic module to the following one.” Id. Colibactin undergoes a prodrug activation mechanism that incorporates an N-terminal structural motif, which is removed during the final stage of biosynthesis.

In some embodiments, the bacterium comprises a partial or full deletion in one or more of clbA, clbB, clbC, clbD, clbE, clbF, clbG, clbH, clbI, clbJ, clbK, clbL, clbM, clbN, clbO, clbP, clbQ, clbR, and clbS or operably linked promoter(s) thereof, e.g., as compared to the microorganism's native clb gene(s) and operably linked promoter(s). In some embodiments, the bacteria produce less colibactin as compared a control microorganism comprising the native or unmodified pks island and/or is less genotoxic compared a control microorganism comprising the native or unmodified pks island.

In some embodiments, the bacterium comprises a modified clb sequence selected from one or more of the clbA, clbB, clbC, clbD, clbE, clbF, clbG, clbH, clbI, clbJ, clbK, clbL, clbM clbN, clbO, clbP, clbQ, clbR, and clbS gene sequences, as compared to a suitable control, e.g., the native pks island in an unmodified bacterium of the same strain and/or subtype. In some embodiments, the modified clb sequence is an insertion, a substitution, and/or a deletion as compared to the control. In some embodiments, the modified clb sequence is a deletion of the clb island, e.g., clbA, clbB, clbC, clbD, clbE, clbF, clbG, clbH, clbI, clbJ, clbK, clbL, clbM clbN, clbO, clbP, clbQ, clbR, and clbS. In one embodiment, the colibactin deletion is the whole island except for the clbS gene, e.g., a deletion of clbA, clbB, clbC, clbD, clbE, clbF, clbG, clbH, clbI, clbJ, clbK, clbL, clbM clbN, clbO, clbP, clbQ, and clbR.

In some embodiments, the modified endogenous colibactin island comprises one or more modified clb sequences selected from clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbl (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), or clbS (SEQ ID NO: 1803) gene. In some embodiments, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbl (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

TABLE 12
Colibactin Nucleotide Sequences

| Description | |
|---|---|
| SEQ ID NO: | Nucleotide Sequence |
| clbA | ATGAGGATTGATATATTAATTGGACATACTAGTTTTTTTCATCAAACC |
| SEQ ID NO: | AGTAGAGATAACTTCCTTCACTATCTCAATGAGGAAGAAATAAAACG |
| 1065 | CTATGATCAGTTTCATTTTGTGAGTGATAAAGAACTCTATATTTTAAG |
| | CCGTATCCTGCTCAAAACAGCACTAAAAAGATATCAACCTGATGTCT |
| | CATTACAATCATGGCAATTTAGTACGTGCAAATATGGCAAACCATTT |
| | ATAGTTTTTCCTCAGTTGGCAAAAAAGATTTTTTTTAACCTTTCCCATA |
| | CTATAGATACAGTAGCCGTTGCTATTAGTTCTCACTGCGAGCTTGGTG |
| | TCGATATTGAACAAATAAGAGATTTAGACAACTCTTATCTGAATATC |
| | AGTCAGCATTTTTTTACTCCACAGGAAGCTACTAACATAGTTTCACTT |
| | CCTCGTTATGAAGGTCAATTACTTTTTTGGAAAATGTGGACGCTCAAA |
| | GAAGCTTACATCAAATATCGAGGTAAAGGCCTATCTTTAGGACTGGA |
| | TTGTATTGAATTTCATTTAACAAATAAAAAACTAACTTCAAAATATAG |
| | AGGTTCACCTGTTTATTTCTCTCAATGGAAAATATGTAACTCATTTCT |
| | CGCATTAGCCTCTCCACTCATCACCCCTAAAATAACTATTGAGCTATT |
| | TCCTATGCAGTCCCAACTTTATCACCACGACTATCAGCTAATTCATTC |
| | GTCAAATGGGCAGAATTGA* |
| clbB | ATGGATAATACCTCTGGAGATTTTCCATGTAATAAGATGGACACGCG |
| SEQ ID NO: | TAAGCAGTTACCGCTAACACCAAGTCAACAGGGGTTTTTATTCCATTC |
| 1066 | CTTAAAGGATAAGAAAAGGAGTAACTACCATGAGCATTTTACATGCA |
| | TTTTTTCTCAGCATGTAGATAGCGCCCACTTCAAGTGGGCGCTGGAAA |
| | CGTTATTTCGAAAGCATGAGTGTTTTCGCACTGATTATAACTGGGAGA |
| | TTGATGAGCGCCCTTGTCAGGTGGTGAAGACCGATGTGTTGCCGGAT |
| | ATATATGTGTTAGACTGTGAGCAAGAGGAAATACGTTTTCTACTAGCT |
| | AATGATGACATTATCATTCCTGTCCCGCAGGATGACGGTATTGATGCT |
| | ATAATTCCTCAACTGCTACAGGCTGATTTAAAATACCCATTTTCCTTG |
| | AAAACGATCCCAGTCCGGGCCTACCTTATTCAGTCAACGAAAGAAAG |
| | TGCTTTTATACTATCATACCATCATATTGTGATGGATGGCTGGAGCTT |
| | ATCCCTTTTCATTAAACAGTTGCTCCAACTCTATGGAGCGGCTGTGGT |
| | CAGTGGTGTGAGGGATGATAGCGCCATTATCCCCTCATCTCTGAAAC |
| | CCCTTGTAGACACACTGTCGGCCCGACGTCACACCTTTCAGCACGACT |
| | ATTGGGCTGCATATCTTCGGGAGGGAACACCAACTTGTATCGTGCCG |
| | CTGTCACAATATCACACAGATACTGAAGCCGAGAACAATTCTTACGT |
| | TAATCAAACAAATCATGTGGAGATCAATTTGTCTCCGGATGTGTGTCA |
| | GAAAATACAGACGCTATGCAGCGATTATCGTATCACCCCCGCAGTAA |
| | TCTTCTATGTGGCCTGGGGCATCCTGCTACAACGTTGGTGCTATGCTG |
| | ACGATGTGTTATTCGGCGCGACAATATCAGGGCGAAATATACCAATT |
| | GATGGTATAGAAGAAACACTAGGGCTATTTATTAACACGTTGCCGCT |
| | GCGTCTGCGTGATGATGGGGCGACACTGTTGCAACACTTACAACGTA |
| | TGCACCAAACACTAATAGCTCACTACAGCAATGAACATGATGCCTTA |
| | GCCAGCATACAACGGTTGGTACATAAAGAAGGTCATGCTGGGGATCT |
| | TTTTAATACCTTAGTGGTGTTGGAGAATTATCCCGTTGATATGACATT |
| | ATTGTCATGCGCGTCGCCCGTGGCAATTCGCCATCTCAGTGTACATGA |
| | ACAAACGCACTATCCGCTGACCTTGACCATCACTCAACAGAAGGGAT |
| | TCCGTTTCAGTATTGCATATGCCCTTAACTACCTGACCAACAACATGG |
| | CGCAAGCGTTGCTGATGCACCTGAGTTATCTGCTTGAACAACTGGTG |
| | GACAATCCGCAGCGCCCCATTGCTGCGTTGGTAAACTTGTCCCCATGC |
| | CAGCAGGCGCAGGTACTTCAACCCTATCTGGAACGCATGGCATGCCG |
| | GGATTGGGATAGTCAATCCAACGTCATCGAACAATTTCATCAAGTTG |
| | CTGCGACTTCGCCAGCACAGGTCGCAGTGGTTGATGAATTGTGCGCG |
| | TTGACCTATTCGGAGTTGGCAGCACAAGCTGAGCAGCTCGCGGCTTA |
| | TTTGGTACAGCAGGGTGTTATGGTTGGCGATACTGTAGGCATAATTA |
| | GCGAACGCCGGGTAAACACCGTGGTTGCCATCATCGCCATCATGTTG |
| | ATCGGGGCTGCTTATGTGCCCATCAGCCCCGACTACCCAGTGGGTAG |
| | GATGCAGGAAATTATTGATGACAGCGGCTTGGCGCTGTTGCTGGTAC |
| | ATGGCAAACCGCTAGATGCATTAAACGTTGCGCAGAGTGACCTCTGT |
| | GCATTTCCCGTCGCGCCCTCGGTGGTATTTCCGGTTATCACACCAGAT |
| | TCTCGCGCTTATGTGATTTATTCATCGGGGTCGACGGGTAAGCCAAAA |
| | GGTATCGCGGTGGCGCACCGCGGTTTATTGCGCCTGATACAAGGCGA |
| | CAGCCCGCTGAAGGTGGAGAGCGGTGAGACAACGCTGCTGACCTGCC |
| | CATTTGAGTTTGATGTTTCGGTGTTTGAGATGTGGTCTACCTTGCTCA |
| | ACCACGGCAAACTAGTATTACTCAGCAAACAGGCATTACTTGATATC |
| | AATCACATTCGCCGCACGATCGCTGATGAACAGGTGGCGCGCGCCTG |
| | GTTTACCTCATCCTTGTTTAACTCCTATGTGGCAGAAGGTGCCGATTT |
| | CTTCGGTATGTTACAACACATTACGGTGGGCGGCGAAGCAGTATCTG |
| | CGTGGCATGTCAACGACGTGATGCAAAAATACCCGCATCTGGTGGTG |
| | ACAAACGGTTATGGGCCGACGGAAAACACTATTTTTACCACCGCATA |
| | TCGTTTCAACGGGTTGCAACCCGCCCGAGTCCCGATAGGATACGCGG |
| | TACCGGGCACCTCGCTCTACATTACCGATCTCCATGGGCATTTGTTGC |
| | CTATCGGTGCCACCGGTGAACTAGTGGCGGGTGGAGTGGGGGTCGCC |
| | ATCGGCTATCAGAACAACCCGGCGCTAAGTGCGACGGTTTTTGTCCCT |
| | GATCCTTTTATTCCCGGCGGTATGATGTACAAAACTGGCGATTATGCC |
| | CGGTTGCTGGATGATGGCTGTGTTGACTGTTTTGGGCGTAAAGACGGT |
| | CAAATTAAGATCAATGGACAACGAATTGAAACCGGAGAGATCGAGC |
| | AGCGCCTGCTGGAGTGCTCCGGCATTATCGAGGCGGTAGTGGTTCCTT |
| | ACCGCGTACGTGAAACGCTGCATATTGCGGCAGTGGTCTGCGTCAAT |
| | GATAGCTATGATGAGGTGGAAGTTCGAGGGCAATTGGCTGACAGATT |
| | GCCGCCATTCGCTATTCCGGAATCCTTGGTGGTGGTGACGGAGATTGC |
| | AAAAAGCCATAGTGGCAAGGCCGACTTGGCGCAGTTGCGGTATCTCC |
| | TGCCCGCAACTCAGTGCAACGCCGTGTCCACCACGATATCAGAGGTG |
| | CATAGTGACATGGAACATGCGCTGCATGCTATCTGGCAACGCGTACT |
| | TGATAGACAAGACATTGATAGCAATGCCTCCTTTTTCGCCCTCGGTGG |
| | CACCTCATTGGATACCATCAGGGTTAAAGGGGATATTAAGCGGCAAC |
| | TTGGCTTGGAGATTGATATTACCGATCTCTTTAAGTACCCAACGCTCA |
| | CGGCGTTAGCACATTTTCTCGATACTGCCGTATCGCCGGAGGATGCA |
| | ATTCCAACGCGTGCTGTTGTCTACAGCGACATGCCGGTGGCGATTGTC |
| | GGTATGGCGGGACGTTTCCCCGGTGCGGCGAATATTGCAGCGTTGTG |
| | GACGCTGGTTGTAGGAGGGGAATCGGGATTAACACTGTTCAGTGATG |
| | AAGAGTTGCGCGCGCATGGTGTGACACCTGACACGCTTAAACAAGCG |
| | AATTATATAAAAACCAAAGGGATTGTTGATGATCACGAATGGTTTGA |
| | TGCGGATTTCTTTGGTTATACTCCCAACGAGGCGGAATGTATGGATCC |
| | GCAAATTCGCTTATTGCATCAGTGCTGCTGGCAAACTCTGGAACACG |
| | CTGGGTGCGATCCTGCCACCTTTACTGGTGCGATTGGCATTTATGCCG |
| | GACTGCTGACATCCCCCCACTGGCTTAATGCGGTAATGCAAGACACT |
| | ACCGACTCTACCGCCCTGTACAAGGCCAGTATCCTGAATATCCATTCC |
| | GTCACAGCATTGATTGCCCATGCGCTTAACCTCACCGGCCCTGCCGTG |
| | ACGCTTGACACCGCCTGCTCCACTTCGGCAGTGGCTATCCACCAGGCC |
| | TGCATCGCACTACGTAACCGGGATTGCGATGCGGCATTGGCGGGCGG |
| | CGTTTCTATCGAGATGCCTGCGTACCGGGGCTATGAATATCATGAAG |
| | GCATGATCAATGCGCGAGATGGTGTCTGTCGTCCTTTCGATAGCCAA |
| | GCCTCTGGCACAGTCACCGGTGATGGCTTAGGAATGCTGCTGCTAAA |
| | ACGTCTGGACGACGCGCTGGCGGATCGGGACTGCATCTATGGTGTGA |
| | TCAAAGGTTCGGCGGTCAATAATGATGGCAATAACAAAATCGGCTAT |
| | ACCGCGCCGAGTGTGATCGGGCAATCAACGGTAATCCGCACCAGTTT |
| | GCGCCGTGCCGGTTTTGATAGCGACAGTATCGGATTGGTGGAAGCGC |
| | ACGGCACCGGTACGGTGCTGGGTGATCCTATTGAGTTACGTGCGCTT |
| | AATGAGGTGTTTGGCCCAACACCTGTTCCGTTTTGTGTGGTTTCAGCG |
| | CTGAAAAGTAATATTGGCCACCTCAACTCCGCTGCGGGAGTGGCGGG |
| | CGTCATCAAAACAACGCTCGCCCTGCATCATCAAGTGTTGCCACCAA |
| | CAGCGCACTTTCGCCAACTCAATCCTGCTATAGATCTATCGCGTTCTG |
| | CATTGTACGTTAACCAGCAAGTCCAACCGTGGCCGTCGACGCGTCCT |
| | CGTCGTGCACTGGTGAGCTCCTTCGGCATCGGCGGCACCAATGCCAG |
| | CATCGCACTAGAAGCACATCAACATGAGGACGACCCTTCAGCGACGG |
| | GGGTACGCGACAGCTATCTGTTGTTGTTCTCCGCTAAAACACCCGCTG |
| | CGTTAGAGTTGCGCGTGGCCTCCACACTGGAATATGTCAAGCATGGA |
| | GTAGGGGTGCGCCTGCCGGATGTGGCTTATACATTGCAAACTGGACG |
| | CACAGCCTTTGACCATCGGCGGGCTTATTTGGTGAGTCGTGGGTCGA |
| | AAATCGATCTCTCCTGTGCCACGATATTGCAAGCGGAAATCTTCAATG |
| | GTCAGCGCACGACAGCGGAGATCTGCTTCATGTTTCCTGGTCAAGGT |
| | AGCCAATATCACGGCATGGCCAGCGCGCTCTATGCTCATCAACCCAT |
| | GTTTCGCCAGCACATGGATCGCTGCTTTGCTGCATTCCAACGCTATTC |
| | GACGGTCGATCTCAAGGCGTTGTTGTTTGACGATGAGGATACGCGGG |
| | ATATTGATCAAACGCAGTTCACACAACCGGCGTTGTTCTGTGTCGAAT |
| | ACAGCCTAGCGCGCACCTTGATTGATCTGGGGATTACGCCGGACAGT |
| | ATGATCGGGCACAGTCTGGGCGAGTATGTTGCGGCCTGTATTGCTGG |
| | CGTATTTACTCTTGAGGATGCGCTGCACGTCATTGAGGCGCGCGGAC |
| | GTTTGATGCAGTCCATGCGTCCCGGTAGCATGATGGCGGTCTACCTTA |
| | GTCGCGAACAGTTGACCCCATGGCTAGCTGCAGAACGGGGTATTGAA |
| | CTGGCAGCTAATAATAGCGCGCATTTTTGCGTAGTCGCGGGCGAGCA |
| | GGCGGCCATTTCCCGTTTGAGCACACGCTTAGTCGAGGGCGGGATAC |
| | AGCACAGGCGCCTGAAAACCTCTCACGCATTCCACTCGGCCATGATG |
| | ACGCCGATGCTGCACGATTTTGCACAGTTGCTGGGGCAAATCCCGAT |
| | GCACGCGCCGCACAAGCGCTTTATATCTAATGTAAGCGGTACATGGA |
| | TTACTGAGGAGCAAGCTACCTCGCCGGATTACTGGGTGCAGCAAGTG |
| | CGCAACGCGGTGCTGTTTAGCGAAGGTGCGGCGCAACTGTTGGTACA |
| | ACCCACGCTGTTTATCGAATGTGGGCCGGGTAATACGCTCTCTACCTT |
| | TATTCAAGGACATAACCAATACAGCGATCAGCCGACCCTGTTGACGC |
| | TACGCAAAGCCAACGCGGCGATCGATGATGAGCACATGTTGCATCGT |
| | ACGCTGGCGGCGCTGTGGGTCAGGGGGGAGAATATTGATTGGAGACG |
| | CTTTAACCAGACGGCACTCGGCAAGCACATTCCATTGCCGGATTACC |
| | CCTTCGAACAGACTTATTACTATCGCTATGGTGCTGCACTTTCCGGTT |
| | ATCGCCAGTATCCAAATCCTCTGCGCCGTCCGCAAGATGAGTGGCTC |
| | CAGCGTGTGCTGTGGCGGATGCACGACACATCCTTGCGGGAGGCGTT |
| | CTATGCGCCGGGCGAATTGATCATCATTATTAGTGCTGACGGCGACA |
| | AGTTACAGCAGACGCTGATGAGTAGTGGTGTCGACAGCATCACAATG |
| | CCGCTGCCGATATCGTCAGAGGACGACGTGTGGGATAATGACCGTAT |
| | TCTGACGCATTTCCACGACATCTGCGCACTTTTAGCACACAAAACCTA |
| | CCGACAGTTACATTGCTTGTATGCTCCCGGTGCGGAGGCAGGATCAT |
| | CGTTGACACAGTCGCTCTCGGGACTTTATCGTGTTGCTCGCTGGTGTA |
| | TGCACAGCACCACGCCGCTGGCGTCCTTAACAGTATTGACCCATGGT |
| | GCGTTTCGCGTACAGGAAGAGGATAATCCCGAACCGACGCTGGCAGC |
| | GTTGTCGGGCGCAGTAAACGTGTTTGCCCAAGAGCTGCACCCGACCG |
| | AAGTACGATTGATCGATATCGATGCGCAGAGCAGTGATGAGAATCTG |
| | AACTTGCTAACCCAGCGTCTGGCCCCGAAACAAGAAACGGTAATGGC |
| | GCTCAGGCAGGGGATGCTTTACCTGCGACGCTTTATCCCGACACGGC |
| | TGTTGGCGCACTTACCCCCTCAAACAGGGTGTATACCGGGCAACGTA |
| | CTGTGGATTATCGGCGGTGAAAAGGGGATTGGCCGCATGATCGGCGA |
| | AGCGCTTGCTCAGCGTGAGGGAGTCCGTGTGGTACTGAGCAGCCGCA |
| | CAGGTTATCACCATGAAGCGGTGCAGCAGGACGCATTAGACGTTATC |
| | CACTGCGACGTGACGCAGGCGGAGGCGGTTAGAGCTTGTCTGGCAAC |
| | TCTGCTCGAACGCTATGGACGGTTGGATGGCGTGATTTTTGCTGCCGA |
| | CGCTACCACCACATTGACACTGCATCAATTGAGCGAATCTGCGCTAC |
| | GCGACACGCTAACGGTGAAAGAACGGGGTACTGCTAATGTGCTGCAT |
| | GCGTTAGCGCAACGGAATCTGCTGGATGAGCGTCTGCTACTGCTGTTC |
| | TGTAACTCGTTGGCTGCCGTGAATGCGGAGATTGGCCAGACAGGCTA |
| | TGCTACCGCCAGCGCCTATTTGGATGCACTGGCACAGCAACTGCGTA |
| | CCCGCTACAAGGTGAATGCGCTCAGTATCGGGTTGGATGCATTGCGT |
| | GAGCAGGGCATGTTGTTGGATGCTATAAACGGCAGTGAATACGATGT |
| | CTTGCGTGGACTGCGCCCATTGATGACGGGAACGTTGCTACAGGCTT |
| | ATAAACAACAAGGGGCTGACACCAGCTACTACGCGCGATTATCCCCC |
| | GAGTCCGATTGGCTGCTGGATGAGCACCGGATATCCGGTATCGCCAC |
| | GCTGCCGGGAACCGGCTATCTGGCGCTGGCGTATGAAGCTCTGCGCC |
| | ATTACTTTGTGCAAGACCAAATCTGCATTGATGAATTGGTCTTTTTGG |
| | CACCGTTGACTGTGATGGACAACTGCAGTGTTGACGTTTTTGTTGACA |
| | TTTCACCTAACGGGCAAGGAGTTAGTGTCGAGGTGAAATCAATGACG |
| | GAGCGCTTTAGTGGCACGTTAACAACCCATGCCAGAGGCAGGGCGAC |
| | GCGTCTGATGGTAGACGATAATGTTGTGTGCGATCTCACGGGGCTGA |
| | TGCGCGAGATGCACACTATCACTCCTCCAACAAAGGAATTATCGAGC |
| | ACGCACTTCCACTATGGGCCGCGCTGGCACAGCGTACAACAACTGTA |
| | TGGCAATACCGCCCAGACTCAGGTTTTCGCAACGCTGGCCCTGCCCA |
| | CCGTTGCCGCTAATGATACGATCGCACTGCACCCTGCGCTGTTGGACA |
| | TCGCCAGCAGTGTTGTCGAACAACTGCCTGGTTTTCATACTGATTCGG |
| | TACCTTTCCTTTATCAGGATTTACGCCTGTACCGCCCGTTGCCGAACA |
| | CCTTACATGTGGCGCTGACTGTCAATCGGCACGATGAGGAGGGTGAC |
| | AGCTACGCTTTCACGCTCTACGACATGGCAGGCGAGATGGTTGCCCG |
| | CTGTGCGGCAATGGTGAAGCGCAAGGTACAGCTCCACATACAAGATG |
| | TCGATGACGACACGCGACTGCGCGTGCCCAGTGCCGATAACTACCAA |
| | CTGCGGCTGGCCGCTGAGGGGGAGGGGGCAGGAAAGCTAGCGTTGT |
| | GCCCTACGCCGCGCTTAGCGCTGGGGGATTCACAAGTAGAGATTGAG |
| | GTACTGGCCACCGGACTGAACTTTAAGGATGTGCTGTTCACCACGGG |
| | ATTGCTCCGGCAGCAGCCGGGTGAGGCTCCGCTGCAATTGGGACTGG |
| | AGTGCGCCGGACGCATTACTCGCGTGGGTAAAAATGTCACTGAATTT |
| | GCCCCGGGAGAGGATGTTATGGCGGTGCTAAACGGTGGTTTTGTCCA |
| | GTACGCACGGGTAGAAAGCGATTGCGTAGTGAGAAAACCAGCCCATT |
| | GTCGCATCGAACAGGCGGCTGCGCTGCCTATCGCATACCTCACCGCC |
| | TATTACGCACTGGTGGTGCGCGCTAATTTGCAACCCGGAGAACGAGT |
| | ATTGATCCACAGTGCGGGGGGGGCGTTGGCTTGGCTGCGCTACATA |
| | TTGCCAAACGCTGCGGAGCACAGATTTTCGCCACAGCAGGTAGCGAG |
| | CAGAAACGCGATTACTTGCTTTCGCTAGGCGTACATGCCGTAGCTGAT |
| | TCACACGACGAACAGTTCGCTGCCACTCTGCTGACCGCATCGGACGG |
| | ACAGGGGATGGATGTGATCCTTAACTCCCTCACAGGCCGTCTGCTTG |
| | ACGCTAGCCTCGCGCTGCTGGCACCGCTGGGCCGTTTTCTTGAGCTGG |
| | GCAGCAAGGACATTGTGGAAGACAAAGCGCTACCGATGCGTTTCTTC |
| | GCCCAAGGCGGCACCTTTATTCCGATTAATTTTCACGCGGCGCATGGT |
| | GCGTTTAGTCGCTACCTGCAACAGATTGTCGCTTGGATAGATGATAAC |
| | ACGCTACCGCTCCTTCCATGCAAATCCGTACCATTGCCCGAGGTTGCA |
| | CGCGCCTTCGCCACCCTGACCACGCCGCAGCATATTGGCAAGGTGGT |
| | GGTAACGCATCGCACTGCGGCAGGCATGGACCGGCTGAACGCCATGA |
| | TAGCAGAAAGGCGCCTCGGCGGCTATGCGCTCAGCATGAGCAATGCC |
| | GAGGTGATGCGCCAATTGTGGCCGATACTGAACACTCGCAGTCCGTG |
| | GGCGCAACTGCTGCTCTCACCTCGGGCGATCGATAGATTAGCGCGGG |
| | GCAACCGGGTGGATCGCGGTGTACCGTCTGCCGCTAACGATACGATT |
| | ACTCAGCAGACAGTGAAAAAGCGGCCCCGTCCGGAAATTGGCGTGCC |
| | TTACAGCCCCGCGACACGTGAAGTGGAACGCGTGCTCTGCCAAATCC |
| | TGGAAGAGTATCTGGGTCTGGATAGAGTAGGCATTGACGACAACTAT |
| | GCCGAATTAGGGGCAACCTCACTCGATATGGTGCAACTGAGTGGGCA |
| | AATGGCGCGTCACTATCCGCAAGTGAGCGTGGTGTCGCTGTACAACC |
| | ACGCCACCGTGCGCCAGCTGGCGACGTTTTGCCAGCCCCCGGAGGGC |
| | GAGTCAAATGCGCCATCACCACAGCCTGCAGTACAGACGAATACGCG |
| | CGCGAATCAGATAGCAAAACGTGCTTTGCAAATTGCGAAAAATACTG |
| | CGCGCAGTCACACGTCTTTGCATTAA* |
| clbC | ATGGAATACGCAAGCGAAATGAACGGCATGGAAATCGCCATTATTGG |
| SEQ ID NO: | TATGGCGGTCCGTTTCCCGCAGTCCCGGACGTTACACGAATTCTGGCA |
| 1067 | TAACATCGTTCAGGGTAAAGAGTGCGTCACCTTCTTTAGCGAGGAGG |
| | AGTTGCTGGCCGAAGGCGTCGAACAGAGTACTCTGGACAACCCGGCC |
| | TATGTACGGGCCAAGCCCTATATCGAGGGCATATGCGACTTTGATGC |
| | TGCATTTTTCGGTTACAGTCACAAAGAGGCGCAGACTCTGGATCCGA |
| | AATCCCGCGTATTACATGAAGTTGCCTACCATGCGCTGGAAGATGCA |
| | GGCTATGCCCAACGTACCAGCGATCTGATCACCGGGGTATTCGTGGG |
| | GGCGTCAGAAGATGTGGATTGGCTACGGCGTTCGCTGTCACAGATTG |
| | GCGGCGATGCGCTGAATCGTTTTGAGTCTGGCATCTATGGTCATAAG |
| | GATCTGCTCGCACATCTCATTGCCTACAGTCTCAATCTCAACGGTCCG |
| | GTGTATAGTCTCTACACCAGTTGTTCGACTTCTCTGAGTGCAACGCAT |
| | ATCGCTTGCCGCAGCTTGTTGTTTGGCGAATGTGATCTGGCGTTGGCG |
| | GGTGGAATTACTATCGATTTACCGCAGAAGTCAGGCTACTTCTGTCAA |
| | CAGGGCATGATCCACTCCACCGATGGCCACTGCCGTCCTTTTGACAGT |
| | CAGGCTTCTGGCACCCTGTTTGGCGATGGCGCGGGTGTGGTTGTGCTT |
| | CGGCGTTTGGAAGATGCTCTGGCAGCGGGCGATCGCATCTATGCGGT |
| | GATCCGGGGTAGCGCGGTCAACAATGACGGTAAACAAAAAATCGGTT |
| | TTGTCGCGCCTGGTCATGAGGGACAGAAGGCGGTCATTTGTGCGGCC |
| | TGTCATCTGGCAGAAGTAAGCCCAGAAAGCATCGGCTACGTTGAAAC |
| | CCACGGTACCGGCACCCGTATTGGCGATCCTATCGAGTTCGCTGCGTT |
| | GACGGAGGCGTTCGATACTTCACACCGCCAATACTGTGCACTAGGGG |
| | CTGTAAAGGCGAATATTGGCCACACCCACGCGGCGGCGGGCGTGGCT |
| | GGGTTAATCAAGACGGCGCTGGTACTTCATCACCGGACCATTCCGCC |
| | GCTCGCCAACTATCAAATGCCTAACTCGAAGCTGGATCTGGCGCATT |
| | CACCGTTTTATATCCCGATACAGCCGCAGGAGTGGCCCGCATCGCGG |
| | ATGCCGCCCCGCGCTGGCGTCAGTTCTTTTGGCATTGGCGGTACTAAT |
| | GTGCATATGATTTTGGAAGGGCTGAATCCTGCGGTGCGCGATGACCA |
| | TGACCAAGTGCGAGCACCGGTGTTTATCCCTCTCTCCGCGCCGTCTTT |
| | CGAGCAGTTGGATGAGCTGACGCAACAGCTTACCCCGTTGCTGGCTA |
| | CCCTTGATGCGTCAACGCTAGCCTACACACAACAAGTGGCGCGCCCC |
| | GTGTTTGATTGCCGCCGAGTGATACAAGTGGAAAACGATGGTACGCA |
| | AGCGATGCTGGCATCGCTGGATAACCTAATGCCCGACGCTCCTTGGG |
| | GCCTACACTGTCCAGATCTACGTACTACGAACGATTGTACTTACGCAC |
| | AGTGGCTGGCGCATTCGGCACATTATCAACGCGAAGCGACTGCTCTG |
| | ACGGCATTACTCGACGGCATGAATATTCCACCCGCTTATTGCCACGCT |
| | GAAACGTGGGCGGCACAAGCGAACAGCAGCCTGCTAATCAGAGGCT |
| | GCCAGACTATCGCCGCGCTAAAAACCTGGATGAACCTGCTACCGACA |
| | TTAACACTGCTGTCGGGTGCTGGAACAGGCCTTTTGCCTGCCGCTGCA |
| | GCCAGTGGCATGATTGCGACGCAAGACGTGTTGCATTTGCTGTGGGA |
| | AATGGAGCAAAAGGCGCTTCATCTCTGGCTTCCTGAGCGCCATGAAC |
| | CTATCCCCGGCTACGTGCTGGCTTGGCAAGGAAATCCCATCACCGAT |
| | GCACAGCGTAACGACAGAGGGTTTTGGAGTGAAGCGCTGTTGGCAGA |
| | TACCCGAGAACTGGGGGAGGGCGTTCACAGTATCAACTGGGTTAGGC |
| | TGCCGCCGGAAATAAGAGAAGACGTTGATGTATTGCGCTACGTGGCG |
| | CAACTGTGGTGTGCAGGTATCAATGTGGATTGGGCCGTGTGGTACGG |
| | CACTCCGCTGCCGCAACGCGGGAGCGCATCAGCATATCCCTTCGCAC |
| | ACAACCACTACCCATTGCCTGGACGGGTAATGGGTAGTGTGGAGACC |
| | CAACCCGAAGCAGGACCCGAAACGCACCACCCTTATCAGGCACGTCC |
| | CGTGTTAAGCGTACCTTTCGTAGCCGCACACTCGCGGGGTATGCAGT |
| | ATATCACAGGTCTGATGGAACTGTTGCTGGAAATATCGCCGGTCGGG |
| | GTGGACGACGACTTTTTTGAATTAGGCGGGCATTCGCTGCTGGTGAC |
| | GCAATTGACCTCCCGTTTAGAACGCGATTTCAACGTACATATCGATCT |
| | TTTGACCCTGATGGAAAACCCCAATCCGCGCAATATCTACGCGCATA |
| | TCGCGGCGCAACTGGGGGGCGAAGACAACCTCGAAATAGCCTGTCAG |
| | TAA* |
| clbD | ATGATGAACGTGGCGGTAATAGGTGCAGGAGTAATGGGAACTGGCGT |
| SEQ ID NO: | CGCTCATAACATGGCGCAATACGGCATATCAACTAACGTTGTGGATA |
| 1068 | TTTCTCAATCTCAGTTGGATAAATGCCGACAGATGATCGAAGCGAAC |
| | TTACGCTTATATAATTTTCACCCGCAGCATAAAAAAAAGACGCATAG |
| | CACAGCGGAGATAATGGAAAATATCCGTTTCACAACTGAGTTGGACG |
| | ACATTGTAGAATGTGATTTGGTGATTGAGAATATTACTGAGGATATA |
| | GAGAAAAAAAATGCACTATACACACGGATGAATACGATCTGCGGCG |
| | CGTCGACAGTGTTTGGTGTTAATACGTCCGCCATATCTATCACTGCGT |
| | TGTCAAAATTAATGCGACATCCGGAGAATGTAGTCGGCGTCCATTTT |
| | ATGAACCCGGTACCGCTGATGCACACCGTCGAATTAATCCGTGGTGT |
| | ACATACTGCAGAGCGTACACTGAACATATTTCACCACTTATTTGCTCA |
| | GTTGAATAAAACCGGCATCGTGGTCAATGATTCTCCGGGCTTTGTCAC |
| | TAATCGTGCCATGATGATTTTTGTTAACGAAGCCATATTTATGGTGCA |
| | GGAGCAGATCGCCCGTGCTGAGGATATCGATACGTTGTTTAAAACCT |
| | GCTTCGGGCACAAGATGGGGCCACTACAAACTGCCGATTTGATTGGC |
| | TTGGATACGATTTTGCAATCGTTGCAGGTGCTATATGAAAGTTTTAAT |
| | GATGATAAGTATCGCCCCAGCTTTTTATTAAAGAAAATGGTCGATGC |
| | AGGGTATTTGGGCGTGAAATCAGGGCAGGGGTTTTATCGCTATCAAC |
| | AGACGTACGCCGAGCAGTGA* |
| clbE | ATGAAAAAGCAAGATATGAAAGCCGCCATTCGGGAATTTCTTTCACG |
| SEQ ID NO: | CTCATTACGTGGGCATACGTTGAACGATGATGACGATATTTTTTCTCT |
| 1069 | CGGGCTTGTCCATTCGTTATTTACTGTGCAAATCATACTGTTTATAGA |
| | AAAAAATTTTCAGGTTGAGCTGGAAGTGAGTGAGTTGAAAACAGAAC |
| | AGATTGCTACCGTCAATAAAATAGTGGAGCTCATTCAGCGACAAACA |
| | GGCCTGGAGTAA* |
| clbF | ATGTGCACTGAAAATTATGAGCTGGCTCAGCAAGAGGCAGTCCTGTT |
| SEQ ID NO: | TGCCAAACAACATCTCGCCCTGGCTGCACAGAATATTGAACGTCAGC |
| 1070 | AGTTTATTGTGCCAGACATTATTTCTTGCGTGGCGCAGGCCGGGTACT |
| | TAGGTGCGTCAATCCCCCAAAAATATGGCGGACGAGGTTACGATTCT |
| | TATCAACTGTGCGCTTTGCATGAAGTGATGGCTGGGGTTCACGGTTCG |
| | TTAGAAAATCTCATAACAGTGACTGGCATGGTCAGCACGCTGCTGCA |
| | ACGCGTGGGCAGCGCAGCGCAAAAAGCCCATTATTTGCCAAAGCTGG |
| | CAACGGGGGAATTGATCGGCGCGATTGCCCTCACAGAGCCGAATATC |
| | GGCAGTGACTTAGTGAACGTGGAAACAGAATTACAGCAGGATGGCG |
| | ACGGTTGGCGGCTGAACGGGAAAAAGAAATGGATCACGCTAGGGCA |
| | GATTGCTGATTTTTTTATTGTTTTAATCCACTGTGGTAATCAATTAGCG |
| | ACAGTGCTTATCGATCGCAATACCGACGGCTTTACTATCACTCCACTC |
| | AACGACATGTTGGGGTTACGCGGCAATATGTTGGCAGAGCTGCATTT |
| | TAATGACTGCCGCCTGAAGGAGGATGCATTGCTCGGTCCGCTCACCC |
| | CTGGGGTACCGCTGGCGGTGAATTTTGCGCTCAATGAAGGGCGGTTC |
| | ACCACCGCTTGTGGCAGCCTAGGATTATGTCGGGCCGCTGTGGATGT |
| | GGCAGCGCGCTACATCCGCCAGCGTAAACAGTTCAAGCGGCGGTTAT |
| | TTTCTCATGGCATTGTTCAGCACTTGTTTGCCACAATGCTGACCCAAA |
| | CGCGCAGTGCGCAACTAATGTGCTTCAGCGCGGCGGAATACCGCGAA |
| | ACGCTGCACCCGGCCATGATAAACCAAATCCTGATGGCGAAGTATGT |
| | CGCTTCCAAAGCTGCGGTGGACGTGGCCGGGAAGGCGGTGCAACTGC |
| | TCGGTGCGAATGGTTGCCATGCCGATTATGCCGTCGAACGTTACTACC |
| | GCGACGCCAAAATCATGGAAATCATCGAAGGGACATCGCAAATTCAT |
| | GAAATCCAAATTGCGATGAATTACATGATGGGGAGTGAAGCATGA* |
| clbG | ATGACGAAGGATGTCGCACTGATGTTCCCTGGCTCCGGTTCGCAATAT |
| SEQ ID NO: | GTAGGCATGGCACGGTGGCTGTATGAGCGTTATCCGCAGGTGCGTAC |
| 1071 | TCTGTTTGATGAAGCTAGCCAGATCACGGAACGGGACATGGCAGCGC |
| | TGTGCCTGTCGGGTACTTTGGTACAACTTGCTGAGCCGACGGCAATG |
| | GCGCTGGCTATCTATACCACCAGCGTGGCCCACTTTGTCGCGTGGCAG |
| | CAGTTTTTGGCGCAAACCGGTGTCCATGTCAACCTACGTTATATGTTG |
| | GGTCATAGTTTGGGCGAATATGCTGCGCTGACCTGTAGTGGTGCGCT |
| | GAGCTTTTCCCAAGCACTGGCGCTGGTGGCGATGCGTAGCCGTTTAG |
| | CCAGTGAGATCGCCCGCGAAATGGACGCGAGTACCACTATCATCAAG |
| | CAAGGGAATCAGGCACTGGTGGCAGCGGCGTGCGAGGTGGCGGAGC |
| | GTGAAACCCGACAGCAGGTCGGTATTGCCTGCTTCAATTCGCCGCAA |
| | CAGTTTATGTTATCCGGACAAAACTCGGCCATTATCGCCGCCGAGCA |
| | ATACCTGCTGGATCATGATCGCCAAGTCGAAGTAGTGCCGCTGATTG |
| | GTGGTGTTCCTTACCACAGCCCACTATTGAAACCTTGCGGACAGCAAT |
| | TGCGCAAGGCGCTGGATCGCTGCGAATGGCGACGTCCGTGCTGCCCA |
| | GTGATCTCCAATGTCAACGCTCAACCCTATCCCGATACGGTCACGCCA |
| | GTCCAGTGGCTAGAACAACAGCTCTCTCAGCCAGTGCAGTGGCAGCG |
| | CTCTCTCACCTACCTGACAGGACACTTGTCACCGATCGCGATAGAGAT |
| | CGGTCCGCAAAGTGTGCTGAAAAACCTGCTGCTGGAGAATCGCTATC |
| | CAGCACCAGTTTACGCATTTGACAATCGTCATGATCGTGCGCAACTA |
| | GCATTGGTGCTGGGGGATAACATGGCGGTGAAGACCGATCCGGAAGC |
| | CGTGCGCCGCCAACGCATCACGCTGCTCACTAACGCCCTGACAGCCA |
| | CACGCCATCACCGCGCCGCCGATGTGGCTGCCAGTGCGCAATTGAAA |
| | GAATTGCTCAGTCGTTTTTTTGAGCGCATCCAGCAGATTGAGCAGCGC |
| | GGCACATCCAGCGAAGAGGACATTGCTTTTTTGCATGAGCTTCTTGAA |
| | CAGGGATTTCAACTCAAAGGCAGTAGTCAGGCAGAAATCGACGCCTG |
| | CCACGCGCGGTTGGCTTCCGACAACGGCGGACAGGCGTAA* |
| clbH | ATGGAACAGCAAGGGATTATGAGACAGTTGCCTACCGACGACCAAAC |
| SEQ ID NO: | GATAGTCGACTATTTGTATCGTATCGCCGGAGAATATGGGGAAAAAG |
| 1072 | CCGCTGTATTGATGGGGGACGCGGCGCTGAGCTATCACGATCTTAAT |
| | GCACGCTCTAACCAACTGGCGCATTATCTGCGTGGGCTGGGGATCGG |
| | CGAGGATCGTGTGGTAGCTATCCGCCTGCCGCGCGGCATGGCAATGC |
| | TGATTGCCATTTTCGCTATTGTAAAAGCTGGTGGTGCCTATTTACCGC |
| | TGGCGTACAATGCACCGCGCAGCCGCATTGAAAATATACTGAGCAAC |
| | AGCGGCGCTGTTTGTCTGATCGGTACTGACGATGGTGATCGCTGGCC |
| | GATTCCTCGCGTCGAAATCGACAGCGCGGCGGTCAGTGCCATGCCAA |
| | CGACGGATCTGCGTTACCGACCACATGCGCGGCAACTGGCGTATATT |
| | ATTTACACCTCCGGTTCCACCGGTGTCCCTAAAGGGGTGGCGACGGA |
| | ACATGCAGCGCTGCTAAACCGAATTGTCTGGATGCAGAACGCTTATC |
| | CTATCAGTTCTCAGGACGTGCTGTTTCAGAAGACGGTGTACACCTTTG |
| | ACGTCTCTGTCTGGGAGATGTTCTGGTGGGCGATGTACGGCGCATCTG |
| | TAGTGCTGTTACCGTCCGGACTGGAGAGCGATCCGCGAACCTTGGCT |
| | CGACTGATTCAGCGTCACCGCGTGTCGGTTGTGCACTTTGTCCCTTCG |
| | ATGCTGAACCTGTTTGTCGAGTATCTGGAGATGAAACAGGATCCTCG |
| | TTTGACCGCCTCATTGCGATTAGTGTTTTCCAGCGGTGAGAAACTCAC |
| | GGTCCACAGTGTGGCTCGCTTTTATCAATCGGTGGCGCAGGGTGATCT |
| | TATAAATCTCTATGGCCCGACCGAAGCAGCAATCGATGTCAGTCATC |
| | ACCGCTGCCTGCGCGGGTACGACTACGACGATATCCCTATCGGTCAA |
| | GCGATTGACGGTTGCCGACTCTATGTGCTGGATGACCATGGTAATCC |
| | GGTAGCAGACGGCGAAGAGGGCGAACTGTATCTCGCTGGTATCGGGC |
| | TGGCACGTGGCTATCTCAACAACGTGGCGTTAACTGATCGCTGTTTTA |
| | CTATACATCCAACTTTGCGCCATTTAGGGAAACCGGAGCGGCTGTAT |
| | AAAACTGGAGATCTGGTGTGGCGCGACGGGGAAAGCCAACAAATTC |
| | ATTACATTGGCCGTAATGATTTTCAGATTAAAATTAGAGGGTTGCGCG |
| | TTGAATTGGGAGAAATCGAAGCCCATGCGATGCGTTTCCCGGGGGTA |
| | CAGCAGGCAGTCGTGGTGGCGGATCAGGATGATCCCGACAATCAGTT |
| | GATTTACGCTTTTGTCGTCAGTAGCGTGCCGCTCAATTTGGCGGCCTT |
| | AATGGACGCACTGTCCAAAAACTTGCCTGCCTACATGCTGCCGAACC |
| | GTTTGTTGGCAATGTCAGAGTTACCACTCTCCGACAATGGCAAGTGCT |
| | GTCGTAAAACGTTGCTCGACTTGGCGCGGGCGTACTCAGCCAGTCGC |
| | GTCGATTTACGTGAAACTCCCGCCGTGCGCTACCTACCGTTGTCATCG |
| | GCTCAATCGTCGATGTGGTTTATGCAACAATTGGCGCCGCATACTGCA |
| | CTATACAATAACCCCACCGCCTTGCTGCTTGAAGGAGAACTGGATCG |
| | CACGCGGATGGACGGCGCGATTCGTCAATTGATGAGTCGGCATACTC |
| | TGTTACGTGCTATGGCGGAAACCCACAATGGACAACCAGTATTGGCG |
| | GTGCCTCAGTGCGTATCGTCGCAGGCGTTGCTGACCATAGTGCCACTC |
| | CCCTCGGTGAGTGATGATAACGCGCTACAGGCGATGATCAACCAGCG |
| | TGCGGCACACCCCATGCCCTTAACGTCAGGCACACCGCTGTGCCGGT |
| | TTGAACTGTTGACGCTCGATGACGATCGTAGCGTATTGTTGATTCATC |
| | TGCACCACATCATCAGTGATGGCTGGTCGAAAGGCGTTTTGTTACGC |
| | GAATTGCAGGCCGCATATAACGGTGAATCGCTGACGCCAGAACCGTT |
| | GTTGGAGTATGCCGACTACATGGAGTACCAAGAAGAGTGGCGTCAGA |
| | GTGACGCCTATCAGGACGCGATGCGTTACTGGCAAAATACCCTGGCG |
| | GGGACGTTGCCGATTCTTGATATCCCCACCGATCAACCGCGTCAGAA |
| | GGTGGCACGTTACCAAGGCGCGTTTGTTGCCTTCGCATTGTCTGCCAA |
| | CACATGCGAGCGAGTGTTGGCGGCGGCGCGTGCGCAGCGCGTGTCGT |
| | TGTATAACTATCTGCTGACCGCCTTCGTCCTCCTGCTGCATCGTAATG |
| | CGCGTCAACAGGAATACATCGTCGGTATGCCGATTGCTGCGCGGCTG |
| | ACGAAAGAACAGGAGCATATGATCGCGCCGCTGGTCAACGTACTACC |
| | GCTGCGCTTACCTCTTGACGAAGCGGCATCGTTTTCCGAGTTAGTACA |
| | GACGATCAGAGGTATTCTGTTTGCCGCTTTCAGGCACCAGCGTCTCGA |
| | ATTTACCGACATAGTGCGCGCAGTGAATGTGGATCGCAGCGCCGGAC |
| | ATTTCCCAATATATCAGTGCATGTTCCAACTCGACAATATGCCTCTGG |
| | CGAGCCCGACACTAAACGGTGTCAACGTTACACCATTATTGTTGGAT |
| | ACCAGTGCATCTCAGGTGGATATTTCGCTAAGTATGCAGCATATCGAT |
| | GGGCGCATCACCGGCACTTTCGAATACGACGCTGGGTTATACAGTGC |
| | GGATCGTATTCAGCACCTGGTGGCGCAGTGGAGAGAACTGCTAGATG |
| | AGGCCAGCAGCCAACCCACGCAGTTAGTCCGCGATTTGATTCGTTTC |
| | ACCCCTCGAGAGCACGCTTGGCTGGCGCGGCACAACGCCACTGAAGT |
| | TGCTCTGCCGCCCGTAGATAACCTGCTGGCGCTAGTGTTACCGCACTG |
| | CCAGCAGCGCCCTACACAGGTGGCTTTGCGCCACGCTGACGACGCCA |
| | TGACCTATGGCGAATTGCAACAGGCCACGATGCAGATGTGTACTTGG |
| | CTGCGTGCGCAAGGCGTAAAACGCGGAGAATCTGTCGCACTGCAACT |
| | GCCTTTTTGTTTCGAATTGATTATCGCACAATTGGCGATCCTCTCTTTG |
| | GGGGCGAGCTATGTGCCGCTGGATGGTAACGCGCCAGCGGCCCGCAA |
| | TGCCCTGATCTTGGCGCAGGCAACGCCGTGCATGCTGCTGGTGGCGC |
| | AACCGCTGGAATCCCCTCATGGGCTGACGATACCTTGGGTGCTAGTG |
| | CCTGACTGGCGTAGCCTGCTGACAGAAATACCGAACCTGCCAGTTAG |
| | TGTTGCGCCAGATGCTTTAGATTGCGATGCGGTGGTGATCTTTACCTC |
| | CGGTACTACCGGGCAGCCCAAAGGCGTCCGACTCAGCCAACGTAATC |
| | TGGTCAACTTAACCGCATCCTTTATATCCAGCTATCAAGTGACGCATC |
| | AGGATGTTTTGCTACCCATTACCTCAGTCGCGTCTGCTAGCTTTGTCG |
| | GGGAGGTACTGCCGCTGCTCGCCGCTGGCGGTACCTTAGTATTGGCG |
| | CAAAAAGCGCAGAGTTTGGATAGTGATGCGCTCATTGCGCTGCTGGC |
| | ATCTCAGCGGGTGACTATCCTGAGCACCACCCCATCGCTTTCTGCCAG |
| | CCTCTCTGTGCTGGCTCAGTCGATGGGATCGCTGCGCTTGTTTCTGTG |
| | CGGTGGTGAAGCGTTAGAGTATGAGCAGATAGCGCCGCTTCTGCCGC |
| | ACATGGCAGTGGTTAACGGTTATGGCCTGACCGAAAGCGGTATCTGC |
| | TCGACCTACTTTCCTGTCGCAAAGCGTAGGGAGCAAGAAACGGGAGC |
| | ATTGCCCATTGGCCGCCCGATTCAGAACACCCAAGCTTATGTGGTGG |
| | ATGCATATAATCGTTTGGTACCGCCAGGTGCCTGTGGAGAACTCTGTT |
| | TCTCTGGTCTGGGCATTTCGCCCGGCTACCTTGATGCACGACAGGATC |
| | CCGAGCGCTTTGTCGAGTTGCCGGAATACCCTGGCGTTCGGGTGCTG |
| | AAAACTGGCGATCGTGCTCGTTGGGCAACCGACGGGATGTTGTTCTA |
| | TCTCGGCAGACAAGATCGCCAAGTGCAGATCCGTGGATACCGAGTTG |
| | AACTGGGCGACATCGAAAGCCTACTGAAACAGCATCCGGATATTGCT |
| | GATGCGTGGGTCGATGTGCGACGCAATGCGGCGGCAACGCCGCTACT |
| | AGTGGCCTTCTATTGCAGCGTCAACGGCGTTGCGCTGGATGCTCAGC |
| | AATTGCGCGTATGGCTCAGCCTGCGGCTTCCGTTGCATATGCTGCCGT |
| | TGCTCTACGTGCCGCTGAGCGCCATGCCGCTAGGTGTAAACGGCAAA |
| | ATCGATCCCCAGTGCCTGCCGCTGGTCGATCTGCGGCAGTTGGAAGG |
| | GCCGGGCGAGTATGTCCCTCCGGCAACCGAACTGGAACAGCGTCTGG |
| | CGGAGATCTGGCAGCAGTTGCTTGGCCTGGAGCGTGTCGGCACCACC |
| | ACAAATTTCTTCGATCTCGGTGGACACTCACTTCTGCTAGTACAGATG |
| | CAGCAATACATCGGGCAGCAGTGCGGTCAGCACGTGGCGTTGGTTGA |
| | CCTGCTGCGCTTCACTACCATCAAACGCTTGGCGGAATTTCTGCTGGC |
| | CCCGGACGCAGCGCAAGGGACCACAGGAGATCAGACACAACTGCGT |
| | GCGGCGAAGCAGCGTTTGGCCTTTGGTCACACGCGCTGGGCAGCCAC |
| | AACCGACAGTCATCACTGA |
| clbI | ATGGCAGAGAATGATTTTGGTATAGCTATCATTGGGATGGCGGGGCG |
| SEQ ID NO: | TTTCCCTCAAGCCGATACGGTACAGGCGTTTTGGGAAAATCTTCTGGC |
| 1073 | AAGCCGAGAGTGCATTTCCTTTTACAGCGATGAGGAACTGCTGGCGA |
| | TGGGAATCAGTCCTGAATTTGTTCAGCACCCAGATTACGTCAAGGCC |
| | AAAGGGGAGGTAGCTGACATCGATAAATTTGATGCTGCTTTCTTCGG |
| | TATCGCACCCCGAGAGGCGGAGCTGATGGATCCTCAGCACCGTGTCC |
| | TGTTGGAAACCGCGTGGGCAGCCTTCGAAGATGCGGGCTATGTGGCT |
| | GCCGATTATCCGGGGGATGTGGGTATTTTTGCTGGCAAAAGCATGGA |
| | TTCCTATTTGATGCTGAACTTGATGCCGCACTTTAAACGCGTTTTCTCT |
| | TCTGGCAGTTTGCAGGCGGCCATTGGTAACGACAAAGACAGTATCAC |
| | TACTACCATCGCCTATCACCTCAACCTGCGCGGCCCGGCGATCACGGT |
| | GCAGACTTCCTCATCGACATCCTTGGTGGCGGTGTGCGTGGCATGCCA |
| | AAGTCTGTTGACTTGGCAGTGTGACATGGCGATTGCTGGCGGAGTAA |
| | CGCTGGGGCCACCAGCAAAAACCGGCTACTTGTCGCAGGAAGGTGGG |
| | ATCACCGCCGCCGATGGCCACTGTCGGGCGTTTTCCGATAACAGCAG |
| | CGGATTTGTGCCCGGCACCGGCGCGGGGCTGGTGGTGTTAAAGCGTG |
| | TTGATGAAGCGCTGCGTGATGGCGATAACATCTATGCGGTCATCAAA |
| | GGATTTGCCGTCAACAACGACGGTTCGGAGAAGATCAGTTATACCGC |
| | GCCGAGCGTGGATGCCCAAGCGCGCGCCATTGCTCAGGCACAGCGGT |
| | TGGCAGGTTTGACACCGCAGGATATCACTTATGTGGAAGCCCACGGT |
| | ACCGGTACGCGTTTGGGCGATCCGGTTGAGTTTTCTGCGCTAAGCCAA |
| | GCCTTTGCGGGCGCGTCACAAAAGCAGTACTGTGCGCTGGGGTCGGT |
| | AAAAACCAATATCGGCCATTTGGATACGGCAGCGGGCGTCGCAGGGT |
| | TGATAAAAACCGCGCTGGCAGTGCAGCAGGGAATAATTCCCGCAACA |
| | TTGCACTTTGAACGGCCCAATGCACAGATCGATCTGACCAATAGTCC |
| | GTTTTACATCAACACTACCTGTCAACCGTGGCAACCGGAAAGCGGCA |
| | TCCGTCGCGCGGGCGTCACCTCGCTTGGCATGGGCGGCACCAATGCC |
| | CATGTTGTGCTGGAACAAGCGCCAGCCGTTGACTTGCAAGCGCGAGC |
| | GCCTGTACCTGCCTACAGTATTTTGCCGTTTTCTGCCAAGACCGACAG |
| | TGCCTTATCCTCGGGGCTGGCGCGTTTTGCCGATTTTCTGCAACATGA |
| | ATCGCTGCCGGATAGGCGGGATCTGGCGTGGACACTCTCACAGGGAC |
| | GTAAAGCCTTCGCACATCGCGCCGCGCTCGTAACCAGAGATCTACAT |
| | GCTGCCGGGACGCTGTTGCAGCAGGCCGCGACAGCGCCGTTTGCTCG |
| | TGGTGTAGCGCAGACACAGCTCGGGCTGGGGCTGTTGTTTTCCGGGC |
| | AGGGTAGCCAATATCAGCGCATGGGCCATCAGCTCTATCAGGTTTGG |
| | CCGGCCTACGCCGATGCCTTCGACCGTTGCGCGACGTTACTCGAGCGT |
| | GAATACCAACTGGATATCCGCCATGAGTTGTTCAGAGCAGAAGTCTC |
| | GTTAGCCCAAGGCGAACGCCTTGCGCAAACCTGCCTCACACAACCGC |
| | TGCTGTTCAGCGTCGAGTATGCCTTAGCCCAATTGTGGCTCAGTTGGG |
| | GAATCACGCCAACGGTAATGATTGGTCATTCGCTGGGCGAATGGGTG |
| | GCAGCGACGTTGGCAGGCGTGTTCTCTTTAGAGGATGCGTTGCGCTTG |
| | GTGGCGCGTCGAGCAGAGCTGATGCATCAGGCACCAAGCGGTGCCAT |
| | GTTGATGGTGGCGCTGCCCGAAGCACAGATTCGCGCACTGATTACCG |
| | CCCCGCTGGCGATTGCCGCTGTTAACGCCCCTGACTATTCGGTTATCG |
| | CCGGGCCTACGTCGGAGATCCTCGCCGTCAGCCAGCGTCTGACGGAG |
| | CAGAACATCATCAACAAACGATTGCATACCTCTCATGCCTTCCACTCT |
| | AGCATGATGCAAGATGCGGCGCAGGCGCTACGTCAGGCATTTGAGAA |
| | TGTACGACTGAACCCGCCGACGCTCACCATCATCTCTACCGTAACCG |
| | GCGCGCACGTCAGTGCCGACACTCTCACCACACCGGACTACTGGATT |
| | GAACAGATGCTGATGCCTGTGCAGTTCTCCGCTGCACTGCAAGAGGC |
| | GCAAGCTACATTTGATGTCGATTTTCTGGAAATCGGGCCAGGTGCCA |
| | CCCTGACCCAGCTGACCAACGGTCATGCATTAGGTGATCGTCTCGCGT |
| | TCAGCTCGCTGCCAGCAGGTGCCCGCAGCAGCGATGAGCACAAACAT |
| | ATCTTAGATACCGTGGCAGCGCTTTGGGTGCGAGGGCATAACATCGA |
| | TTTGTCTGCGTTTGCAGGTGAGCAACCGCGCCGTGTCTCTCTACCGAC |
| | CTACGCCTTTGACAAAATCCGTTACTGGGTCGACAGTCCAGAAGAAC |
| | AGAGGAGCGCCGTAACGCCGGTGGCGGACGCGGGAAGTGTCATCCCT |
| | AGCGAACCGTCGGTGCGCCGTCAGCCGCGTCCGGCGTTTTCGGTGCC |
| | TTACGCGGCTCCAGAGAGCAAAACGCAGCGCGGCTTAGTGGCGATTT |
| | GTGAAGCATTGCTGGGAATTGACGGCTTGGGCATTGACGACAACTTC |
| | TTCGAAGCTGGCGGACATTCATTGATGCTGGGCATGTTGCTGGCGCA |
| | GGTACAGGAACGGTTCGCCGTCACTCTTTCCTTTTTCGACGTGATGGA |
| | GGATGCCAGCGTGCGCGCGTTAGCGCAGTTGGTCGAGCAGGAGCAGC |
| | AGGATGATGGAGGGTCCGCACTTGCCGTGCTAGTTAACGACATGATT |
| | AATGAGTGA* |
| clbJ | ATGACGATACATCATGCCGCATTGGCGCGAATGTTACCGGCGGAAAA |
| SEQ ID NO: | AAAGGAAAAGCTATTACGACAGTTGGCGCAATCAGGCGTGTCGCCCA |
| 1074 | GCCGCATTCCCATCATCAAAGCGGATCCGGCGCAGGCAATCCCGCTG |
| | TCGTTTAATCAGGAGCGGTTGTGGTTTTTGCAAAAATACGACAGTACC |
| | GCCACTAACTACAACCTTTATGTGGTGTATCGTTTGCATGGCGTGGTG |
| | GATATGCCGATGCTCACGGAGGCATTGCGCCATGTGCAAGCACGCCA |
| | CGCCATCTTGCGTACCCGTATCATAGTACGTAACGATCGGCCTTGCCA |
| | GGTGATAGATGATGCGTCTTCGTTAGTTCTCGATACGGTCACGCTGGC |
| | AGCGCAGGCCCCAACTTCAGCGCTAGATGCGGTAATTCAGCAGGTGA |
| | TAAACACCCGGTTCGATCTGGCACGCGGTCCTCTGTGGGGGGTAACT |
| | CAGATTATTCAACCCGATCAAGGTTGTCATTTGGTGTTTTGCGCGCAC |
| | CATATCATCATCGACGGTATCTCGTTACGGCTATTGTTCGATGAGTTA |
| | CAGCAGCAGTATGCTCGATTGCATGCTGGTAATGAAACGTCGCTGCC |
| | TCCGCCGCCGCTGCAATATGCCGATTATGCCTTCTGGCAGCGCGAATG |
| | GTTCCAGGATACCTTGCTGGCGAACGAACTTGCCTACTGGCGTGCGC |
| | GTTTGCAAGACGCGCCTCTGCTGTCGACATTTCCATCGCTGCACCCGC |
| | GCCCGGCACAACCCTCTACACATGGTTCCCGGTTCAGCATCACCCTGG |
| | ATGAAACATTAAGCCTCGCGTTGAAACACGTAGCCCGCACGCAGGAA |
| | ACAACGCCGTTCGTGCTGATGCTGACTGCCTTCCAACTGGTGCTGATG |
| | CGTTACGCTCAGCAACAGCGATTGGTGATAGGCATGCCAGTATCGGG |
| | GCGCATTCGCCCGGAATTGCAGAGTAGCATTGGTTATTATGCCAGCA |
| | CTGCGGTTATCTACACCGATTTTAATGGGGTTGAGGTAGGGCGCGAG |
| | GCGCTCCAGCGTGTGAAGGCCAGCGTGAAAGAGACGCAGGGGCGCC |
| | AGCAACTGCCGTTTGAAAACTTGGTGAATATGCTGGATCTCCCTCGCA |
| | GCCTTTCCCATTCACCGTTGTTCCAGATCCTCTATATCTACCATAACC |
| | ACGTGACCCCACGCGCTTTTACCTTGGCTGGTGCGTATTGGGAGCAG |
| | GTGACGTATCACAATCAGACCGTCAAATACGACATGACAGTCGAGGT |
| | GTTCCAAAACGACGCCACGTTCGACGTCTCCTTTGAGTACGATTTGGG |
| | GTTATACGATGCTGATGTGGTGAAGCAGATTGCCGAGGCGCTGCGTC |
| | AGCACTGCTTATCGTTGACATCATCACTGGAGACCCCGATAGGGGCG |
| | ATCCCTCTGCATGCACCGGAGACCGCAACGCCGCGGCGTGATCCGCT |
| | CAACGCCACTAACGTCCCGTGGCTCGGGCCGCAGGATGTGCTGCGTA |
| | TCATTGAACAGCGCTGTGTGCAGCACCCAAAGCAACTGGCAATACAG |
| | CAGCATGACGGCACACTGACCTACGCTGAGCTCTGGGCGCGTGTGCA |
| | GTTCATCGCGATGCGTTTTCGAGCGCATGGCATACAACCGGGCGATC |
| | GTATTGGCGTGCTGTTGCCACGTCACAGGGATGTGATTGCAACCATGT |
| | TGGCGACGTGGTTTGTGGGGGCGTGCTACGTGCCGTTCGATATTCATC |
| | AGCCTGCCGCGCGTTTGCAACGCCTTATGCAACGCGCGCGTTTGGTCT |
| | GTCTGGTGGTCCGTCAGCCGGGAGAGTGGGGTGAAATTGTACAACTG |
| | TCGTTGCCGGAATTGATGCAGGACATGTCGAATGCGATCCGGTATTCT |
| | ACACCTTGCGCGCTGTTGCCGGATATGCAGGCCTACCTGTTGTTTACC |
| | TCCGGCAGTACCGGTGAACCTAAAGGCGTGTGCGTTGTTCACCGCGG |
| | GTTGCTGAACTTGTTGTTGGATATGCAGCGTACCTTTGCGGTTGGCTC |
| | GCAAGACCGGCTGCTCTCGGTGACGACGCCAACATTTGATATCTCATT |
| | CTTGGAGTATCTGTTGCCGCTGATCTCCGGTGCGAGTCTCTATCTGAC |
| | AGAGGCGGAACGCGCCGCAGACAGCTTCCGTATGATTCCGCTGATTG |
| | CCGACTATCGACCAACGCTGATGCAGGCGACGCCCTCGTTCTGGCAC |
| | GGGCTGTTGATGGCGGGTTGGCGTGGCGACCCGGAACTATGTGTGCT |
| | GGCAGGCGGTGAAGCGCTGCCAACGAAAGTGGCGGAAGAACTGTTG |
| | CGCTGTTGTGGTTCATTGTGGAACCTCTACGGTCCCACCGAAACAACC |
| | ATTTGGTCGCTGAAATCGCAGATAACCCAAGCGGAAAACATCACCCT |
| | CGGCGCTCCCATTGCCAATACCCGTATATACATTCTGGACAATGAGG |
| | GCCATCCAGTGCCGCAAGGCGTTGACGGCGAGCTTTACATCGCCGGG |
| | GATGGTGTGGCGCAAGGGTATGATGGGCAGCCTGAGTTGAACGCACA |
| | GTTCTTCTTGTCAGAACCAGGGGTCCCCGGTGGCAGGATGTTCCGCAC |
| | TGGCGATCTGGTCAGGAGTGATGCGCAGGGGCAGTTGTTTTTTGTTGG |
| | GCGCAAAGATAGCCAGATCAAACTGCGCGGTTATCGTATTGAGTTAG |
| | GCGAAATTGAACGGACGTTGGCACGGCATCCGCATGTGGACGCCGCG |
| | GTGGTGGCCTGTATTGAAAGAGCACCGTTACACAAAGCACTGGCAGC |
| | ATTCATCATCACCAGTGAGCCTCCCTCGCTATTCGAGCAACTGAAAA |
| | ACGAGCTTCGGCAGCAACTGCCAGACTATATGGTGCCAACGCTGTGG |
| | CAGAGGGTGGCCGACTTCCCGAACACTGACAACGGTAAAATCGATCG |
| | TAAGCGGTTAGCGGAAAATTTCGTTGCCGATAGCTCCCTTGTGTCGCC |
| | GCAGACGCAAGCGCTGAGCGACACGGAACAGATGTTGCTGGCGCTGT |
| | GGATGCGCTATTTGCCGATAAAAAACGTCGATCCTGAGTGCGATTTCT |
| | TTCGCTTGGGGGGACATTCGCTGTTGGCGGTAACGTTGGTAGCAGAG |
| | ATCAACCGCACTTTTCACTGCGCCTTGACCCTGAAGGACATTTTCCAC |
| | TACTCCACACTACGGGCACTGAGTGCGCGTATTGCACAGCAATCTAT |
| | CACGGACGCTGCCGCGTCTCAGGATGACTGGGTGATAGTGCACGACC |
| | CTGAGCATCGTCATCAACCGTTCCCATTGACGGATGTTCAGCGCGCCT |
| | ACTGGCTTGGACGACAGACGGGTGCTACCTCGATCGCGACCCACATC |
| | TACCATGAATTTGACGTAGAACACTTTAATGTTACGCGTTTTACCCAT |
| | GCGGTGAATGCGCTGATCGCTCGCCATGAAATGCTACGTGCGCGGGT |
| | ACTCCCCGACGGTACTCAGCAGATTCTGGCGCAAGTGCCGGCGTATC |
| | AGTTAGAGCAGCGCGATCTGAGTGCTTTGTCCCCTAACGCACGAAAC |
| | GATGCCTTGATGGCGATCCGCGATCGGCTGTCGCATCATGTGCATCCC |
| | GCAGATCGTTGGCCGCTGTTTGATTTCAGTTATTCGGCTTGCACGGCG |
| | CAACATGGCCGCTTGCATTTCAGTCTCGATCTGCTGATTGCCGATGCT |
| | CTGAGTATGCGCACGCTACAGCAGGAGTTGATGATGCTGTACCGTGA |
| | GCCCCATGTGTCACTGCCGTTGCTACCGTTCTCTTTTCGTGACTACGT |
| | GCAGGCGCTGTTGGTAGAGCAGGCGAGTGAAGCCTATGCACGCGATC |
| | AGGCCTATTGGCAACGGGCGCTGCCGCAGCTGTATGGCCCACCAACG |
| | CTGCCCGTACAGGGCGATTTGGCGCAACTGTCTGCGATCAGGTTCGT |
| | ACGTCGCCGTCATCGGCTGTCAGCCCACAACTGGGGAGTGCTGAGCG |
| | CGCTGGCCCAACGCACACGTATCACCAAGACGGCATTGTTGTTGACA |
| | GTCTTTAGCCAAGTGCTGGCACGTTGGAGCCTTAGCCCGACGTTTACG |
| | CTCAATCTGACGTTGTTCAACCGCCCGCAGGGTTACCCCAACGCAGA |
| | GGCAGTTATTGGTGATTTTACCGCTGTCAGCTTGCTGAATGTTTGTTA |
| | CGACAGCCAGCACTCTTATGCCCACAACGCTCAGCGTATTCAGGTGC |
| | AACTGTGGGAAGATCTCGAACATCGTCGTTTCAGTGGGATCCGCGCC |
| | AGCGAGGCGCTGATCCATAGCGGTCGTTTCCATGCGCCGATGCCGGT |
| | GGTATTCACTAGTATGTTGGATATCGACGGGGAGACGACTGCGCAAG |
| | ACCCTCGGGACACAACCCGTTTTACTCTGTGTCCGGACGCCAATATTA |
| | CCCAAACACCGCAGGTGTGGCTCGATCACCAGGTGATCGAGTTGGCT |
| | GGGGAGTTGCATTTCAACTGGGACGCGGTCGAGCAACTGTTTGATAC |
| | CACGCTGCTGGATCAGATGTTTGGTGCTTATTGTCATGCGCTGCAGGC |
| | GCTGGTTGCCATGCCGCAAAGTTGGTGGGGGGTAAATAGTTCTCTGG |
| | CGCTGCCCACCGTTAGTGCACCGGTCACGCAGGCTCCTGCACCTACG |
| | GCCTTGTTGCACCATGGATTACTGCGTCAGGCAGCACTGACGCCACA |
| | GGAAACTGCGCTGATCAGTCCTATCCGTGAATTGACCTATCGCCAACT |
| | GTCGACGGCGGCGGATCATGTGGCCCGCGCCCTGTTAGCGCTGGGCG |
| | TGCAGCATGGCGACCGCGTGGCGGTGGTGATGGAAAAAGGCTGGCA |
| | GCAGATTGCCGCCGTACACGGCATTTTACGACTGGGTGCGGTCTATCT |
| | GCCAGTGGATCCGGTGCTACCGCCACAGCGTCGCCAGCTTTTGCTGA |
| | CGGTGGGCGAGGTGCGGGTACAAGTAACGCAGCCGGGTCTCACGCA |
| | ATTGGAGCCGTCGCTGCCCGTGCTGATCATCGACGACGGAATGCTGG |
| | ACACGCCTGCTGCGCCGTTGCCTGAAGTGGCTGGGGATGTCACGGAT |
| | CTGGCCTATATCATTTTCACTTCCGGCTCCACCGGTACCCCGAAAGGA |
| | GTGATGATCGACCACCGTGCGGCCATGAACACGCTGGAAGACATCAA |
| | CGAACGCTTTGGCCTCAATGCGCAGGATAGGGTGTTCGGGCTGTCAT |
| | CATTGAGCTTTGACCTGTCGGTTTACGATGCCTTTGCGCCTTTTATGGT |
| | GGGTGCAGCGCTGGTACTGCCGGAAGCAGGACGGGAAAAAGATCCG |
| | CGTCATTGGCAGACAGTTATGGCACACGGTCATGTAAGCGTCTGGAA |
| | TGCAGTGCCCGCACTGATGCAGATGCTGTGCGAATACCACAGCGGCG |
| | ATCGGATGAGTTATCCGACGTTGCGTCTGGCACTGTTGAGCGGCGAC |
| | TGGATCCCGCTAACGTTACCGGAGCAGATGCGCGAGCGGCTCAATGA |
| | AACGATGGACATCATCAGTCTGGGTGGAGCGACCGAGTGCGCCATCT |
| | GGTCGGTCTACTACCCGATAGGTGAGGTGGAATCGACGTGGACCAGT |
| | ATTCCCTACGGTCGGGGCCTGCGCAACCAGCCAGTATACGTGCTAAA |
| | TGCGCAACTGGAGGAATGTCCGGTCGGGGTGGAAGGAGAGATTTGCA |
| | TTGGCGGGATGGGGCTGGCACAAGGCTACCTGAACGACGCAGAGAA |
| | AACGGCGGCGAGCTTTGTCTGGCGCGAAGCGAGTGGTGAGCGAATTT |
| | ACCGCACTGGGGATCGCGGGCGCTACTTTGCTGACGGGCAAGTCGCC |
| | TTTTTGGGGCGCAACGATACCCAAGTGAAGGTGAATGGTTACCGTAT |
| | CGAACTGGGGGAAGTCAAAAGCCACCTTGAACAGCTCGACAGCGTA |
| | GGGAGTGCCGCCGTGGTGTGCCACCAGGGACAGCTGTATGCCTTTAT |
| | CACTGCCGCAGAAAACCTGCATCCTGACGATACTGACGCCCTGTTAG |
| | CGCGTGTTCGTGCTCAGTTAGCCGTGCAGTTGCCGTATTACCTGCTGC |
| | CCCAACATTTCTTTCTTCTCAAGGTGCTGCCGATGACAGGCAACGGCA |
| | AAATTGATCAGGCGGCAATGGTTCAAGAGGTTATCCAACGTATGTCG |
| | CAATCTACATCACAGAAGTCGAGGGCCCTCGCGCACGCCTCGCCCTA |
| | TGAACAGCAGGTGGCCGCTCTCTGGTGCGAGGTACTACAACGAGAAC |
| | AGATCGGACTGAATGACAACTTTTTTGAAGCGGGGGGCGGCTCAATC |
| | CAGATCGTGCTGTTGCATCGCCGTATTGAGGAGATTTTTAAGGTTACG |
| | GTACCTATTGCTGAGCTATTTCGCTTAACCACCGTGAAAAGAATTGCC |
| | GGTTATTTGCAGGCTATGCAGGACAATGCACGGGCGGTGAATCAAAC |
| | ACAGCAGCGTGATGCTTCCCGATCCCGCGCCCAGCAACGACTTGTAC |
| | GTCGTCACCAGCGTCAGCGTTAA |
| clbK | ATGACTTACAGTGAAAGCGATATTGCCATTGTTGGCATGAACTGCCG |
| SEQ ID NO: | GTACCCCGGCGTGCACAGCGTTGCGGCATTCGAAACGGTGCTTCGCA |
| 1075 | CCGGATGCAACATTTTGGACCCCAAAGTGACCCCGAGCAACGGCCAC |
| | AATCACATTACGCTGAACAACGTCTATGAGCACATGGCGGAGTTCGA |
| | TGCCAACTTTTTTGGCTACAGCCGTGCAGAAGCAGAAATCATGGATC |
| | CACAGCAGCGCGTATTTCTGACCTGTGCTTGGGAGATGTTTGAACAA |
| | AGCGGTTACAACCCGAAGCAGCACGATGCGCGCGTCGGCCTATATGC |
| | GGGCGTGAGTACCAGCTTCTATTTGCTTACTCACCTGATGAACAACCC |
| | AGACAAGCTTGCACAACTCGGTGGTCTCCAAATCATGGTCGGCAATG |
| | ACAAAGATCATTTGACGTCCCAACTGGCTTACCGCCTGAATATTACCG |
| | GCCCTTGCGTGACGGTTCAGGCCAGTTGTGCCACCTCGCTGGTGGCA |
| | GTGCATCTGGCCTGCGAAGGGCTGCTGAGCGGTCAGTGCGATATGGC |
| | GCTAGCGGGTGGCGTCACGTTTCGCATGGAGGAGCAGCGCAGCTATG |
| | AATCGCACGGTGATGGGCTGCAAGCAGAGGATGGCCTGATTCATACT |
| | TTTGATGCGCAGGCGAGCGGTACGGTCTACAGCAGTGGTTTGGGCAT |
| | GGTGCTGCTCAAACGGGCTACAGACGCGCAGGTGCAAGGGGATAAC |
| | ATCCTTGCCGTCATCAAGGGAAGCGCCATCAACAATGATGGCGGTGC |
| | GCGCAGTGGTTATACCGTGCCGGGTGTTGATGGTCAGGAAGCTGTGA |
| | TGATTGAGGCGCATAGCTTGGCGGAGGTGACGCCGCAGCAAATTCAG |
| | TATCTAGAACTGCATGGCAGCGGCACGCCGTTGGGTGACGCTATCGA |
| | ATTCGCGGCCATCAAACGTGTGTTTGGGACGCCAGCGCCGAATGCGA |
| | CACCGTGGCGTCTGGGGGCTGTGAAACCTAACGTGGGTCACGTAGAA |
| | ATGGCATCTGGCATCACCAGTCTCATAAAAACGGTGCTGAGTCTTACT |
| | AACCGGGTGTTTTACCCTACGCTGAATTTTCAACGGGCTAATCCGCAA |
| | CTGGGGTTGGAGGACAGCCCGTTTGAAGTGGTGTCCCGTTTAACGCC |
| | TTGGCCGGAGGGCACCACGCCACGCACCGCTGGTGTGAGTGCGTTCG |
| | GATTGGGCGGCACCAACGCACATCTGGTGGTACAAGCGCCGTTATCG |
| | ACGCCGCAGGCGAGGGCGCAGCAGATGGGGCCTTGTGTCGTTGTACT |
| | CTCGGCAAAAAACCACAATGCCCTGGAACAGATGCAAAACGCCCTGC |
| | TGGCGAAACTGGCCGCACATCCAGAGATACGCTTACAGGACGTGGCT |
| | TACACCCTGCGTCACGGGCGTTTCTCCGCCCCGGTACGTAAGTGTGTC |
| | ATCGCAGAGAATTGCACCCAGCTAGCCCGACAACTCCGCGACGCACC |
| | GATGGTGGAGGCAACCACGGGTTGCACGATCTACTGGAGATTGGGGC |
| | ACCGTTTCGTTGTTGCACTCGAGACGCTTAGTGACTGGCTGGCGTGCT |
| | CTGAGGTGTTGTCACAGGCGGTGGGACAACTGCTCGAGCATTTTCCG |
| | CTCGAACCAGCCTGTCTGCAAGATCTCTCCCCTGCGCAACGGACGTTT |
| | ATCAGCCAGTATGCGCTGATCGCGTTGATTGACGAACGGGAAACGTT |
| | GAATGTGGTGCTATGCGGCGACGGTGATGGTGGCTATGCTGCCGCCG |
| | TACTACGAGGAGACTGCACACTAGAGCAAGCATGGCACAGGTTGAAT |
| | GCGGGCCAACCGTTTGATGATGTGCCGACGAATCCCCTCTTGCAGCC |
| | CGATGTCTGCTCTTTGATGCTGGACGACGCGGCGAGTGATGCCAATC |
| | GCACCGCGCTAGAGGCGTTAGGGCAGTTATGGTTGGCTGGCGTCAGC |
| | CTAGATTGGCGCTGGGTGGATGCGGCGGAGCGTATGTTGGGCAGTCA |
| | ACGCATCGCCCTGCCGGGGACCGTTTTTACACCGCAGCGCTATTGGGT |
| | CGAGGCCGTACGGCCTGCCACGTTCTCCCACGAATCGTCGAATAATC |
| | TCTTATCCCGCGCAACAAAATCCGATATTATTGCAGTGGTGACGGAG |
| | ATATGGGAACGCACGCTTGGTGTCAGCATTGATGATCACCACGCCAG |
| | CTTCTTCGAACTGGGTGGGCATTCGCTGTTAGCGTCAACCATTTTATA |
| | TGATATTCAGCAACGTTACGGCATCACCTGTACGCTAAGCGCATTCTT |
| | TGCCGATCCCACCATCGAGGGATTAAGTTGCTATCTGCTCGAACAAG |
| | GCGGCAGTGAGACAGCGGTTTCAGCATTGCCTGATACGGTCTTTGCC |
| | CCTGACCAGCAGCACCTACCTTTTCCGCTGACTGACGTGCAACAGGC |
| | CTATTGGGTTGGGCGGCGAAAATCCTTGGGATTGGGCAATATTTCCA |
| | CCCATATCTATGTGGAATATGAACTACAGGGGCTAGATGAAACAGCG |
| | TTCAACCGCGCGCTCAATGCGGTGATTGCGCGGCACAGCATGTTGCG |
| | TGCAATTGTCAACGATGACGGTATGCAGCAGATATTGCCAAACGTGC |
| | CGGAATACCACGTTGCCTTCTATACCACCCAGTGCGAGGATGCGTTTC |
| | AACAGCGTTGCCGTGAGCTGCGTGACACCCTCTCACATCAGATGATT |
| | GATTGTAGTCGCTGGCCTCTGTTTCAGATGGAGGTCGTGGTTGATCCG |
| | CAGCAAAAAGCCCGGCTACATGTATCTATCGACCTGCTGATAGCCGA |
| | TGCATGGAGTCTGGAGTTGTTCATTCGGGAACTAGCTTATCACTACCG |
| | CCATCCGCAGGCGGCATTGCCTACATTGACGTACAGTTTCCGCGACTA |
| | TGTGCTGACCTTAAAATCCTACGAGAAAACGCCGCAGTTTGAACGGG |
| | CACGTGACTACTGGCGCGCCCGCATCGAAACTCTGCCGCCGGGGCCA |
| | CGATTGCCGCTGCGTACCGACCCCACGAAGCTGGAAAACCCCACATT |
| | CGTGCGCCGTAGCTATTGTCTATCTCGTGCTATCTGGCAGCGCTTGAA |
| | AACGCAGGCGGGTCAGATGAGTATCACGCCTACTACACTGCTGCTCA |
| | CGGGGTTTGCTCAGGTGTTGGCGCGCTTTAGTTCCTCGCCGCACTTTA |
| | GCCTTAACCTGACGCTGTTTAATCGTCTGCCGTTGCACGCAGATATCA |
| | ATCACTTAATTGGCGATTTTACTGCACTGACGTTGCTGGAGATTGATA |
| | TGTCGCAGGGAGAAACCTTGCAGGCGCGGGCAAACGTGATCCACTCT |
| | CAACTGTGGCGCGATTTGGATAACCGCTTGTTTGGCGGTATCCAGGTC |
| | TCTCGACTGCTGGTACAAACTCACCGCGATCCGGCGAAATCGGTGAT |
| | TCCTATCGTGTTCACCAGCTTGCTCAATCAATATGAGGCGAGCTGGGA |
| | AACGGATGATACGCTGTTCAACCAACCGCAGGATGATCTGTACAGTA |
| | TTTCACAGACACCGCAGGTGTGGCTCGATCATCAGGTAATGGAGCGT |
| | AACGGGGAATTGCACTTTAATTGGGACGTGGTGGAACAATTGTTTGA |
| | ACCGGCGCTAATGGATCAGATGTTCCAGTGTTATTGCCAACTCCTGCA |
| | CGCCCTAGCCCAGCGACCACAGCTCTGGCATGAGACTCAAGATGTGC |
| | TGGCGCTGCCCACCGTTAGTGCACCGGTCACGCAGGCTCCTGCACCT |
| | ACGGCCTTGTTGCACCATGGGTTACTGCGTCAGGCAGCACTGACGCC |
| | ACAGGAAACTGCGCTGATCAGTCCTATCCGTGAATTGACCTATCGCC |
| | AACTGTCGACGGCGGCGGATCATGTGGCCCGCGCCCTGTTAGCGCTG |
| | GGCGTGCAGCATGGCGACCGCGTGGCGGTGGTGATGGAAAAAGGCT |
| | GGCAGCAGATTGCCGCCGTACACGGCATTTTACGACTGGGTGCGGTC |
| | TATCTGCCAGTGGATCCGGTGCTACCGCCACAGCGTCGCCAGCTTTTG |
| | CTGACGGTGGGCGAGGTGCGGGTACAAGTAACGCAGCCGGGTCTCAC |
| | GCAATTGGAGCCGTCGCTGCCCGTGCTGATCATCGACGACGGAATGC |
| | TGGACACGCCTGCTGCGCCGTTGCCTGAAGTGGCTGGGGATGTCACG |
| | GATCTGGCCTATATCATTTTCACTTCCGGCTCCACCGGTACCCCGAAA |
| | GGAGTGATGATCGACCACCGTGCGGCCATGAACACACTGGAAGACAT |
| | CAACGAACGCTTTGGCCTCAATGCGCAGGATAGGGTGTTCGGGCTGT |
| | CATCATTGAGCTTTGACCTGTCGGTTTACGATGCCTTTGCGCCTTTTAT |
| | GGTGGGTGCAGCGCTGGTACTGCCGGAAGCAGGACGGGAAAAAGAT |
| | CCGCGTCATTGGCAGACAGTTATGGCACACGGTCATGTAAGCGTCTG |
| | GAATGCAGTGCCCGCACTGATGCAGATGCTGTGCGAATACCACAGCG |
| | GCGATCGGATGAGTTATCCGACGTTGCGTCTGGCACTGTTGAGCGGC |
| | GACTGGATCCCGCTAACGTTACCGGAGCAGATGCGCGAGCGGCTCAA |
| | TGAAACGATGGACATCATCAGTCTGGGTGGAGCGACCGAGTGCGCCA |
| | TCTGGTCGGTCTACTACCCGATAGGTGAGGTGGAATCGACGTGGACC |
| | AGTATTCCCTACGGTCGGGGCCTGCGCAACCAGCCAGTATACGTGCT |
| | AAATGCGCAACTGGAGGAATGTCCGGTCGGGGTGGAAGGAGAGATT |
| | TGCATTGGCGGGATGGGGCTGGCACAAGGCTACCTGAACGACGCAGA |
| | GAAAACGGCGGCGAGCTTTGTCTGGCGCGAAGCGAGTGGTGAGCGA |
| | ATTTACCGCACTGGGGATCGCGGGCGCTACTTTGCTGACGGGCAAGT |
| | CGCCTTTTTGGGGCGCAACGATACCCAAGTGAAGGTGAATGGTTACC |
| | GTATCGAACTGGGGGAAATCGAGCGCTGCATTGCGCGACATCCCGAT |
| | GTGGAGCAGTCAGTGGTGGTGGCAGTGGGTAATTCTCAACATCGTCG |
| | GCTGGTCGCTTTTGCCAAACTGCACGATCGCCACCAGGCGCAGGCAT |
| | TGCAAGCTAAGGAAGCGGAGGCGGCGGCACTGGCGCAGGGTATTATT |
| | GTGAATCCGGCACAGCGTCTAGCGTTCAAACTCAAGGAGCCACATAT |
| | TCGCGCGCTGGATGGTCTGGGCATTGCACTGACGGCACCGGCGGATA |
| | GCACACGTTACATCAAACGCCGCAGCTATCGTCATTTCAGCGCGCAA |
| | AAAACCACGCTGGCACAGTTGGGGCAATTGCTGTCGGGCTTGGGGCA |
| | GATGCGTCTACCCGGTCTACCTTTTGCCAAGTATGCCTATGCGTCCGC |
| | CGGGGGGCTATACCCGGTGCAAACCTACGTGTACCTGCATCCAGACA |
| | AGATCGAAGAGGGAGTATCCGGTATTTACTACTTCGACCCGCGACAG |
| | AGCTGTCTTATGCCGGTAGCACCAGAAGTCGAGCTGAACAGTGGTTT |
| | TCATGCCGGACCTAATCAGTCTATTGCCGATCGGGCGGCATTCACGCT |
| | GTTTATGGTGGCTGATATGGCGGTGATCTCGCCATTCTATGGGCAAGA |
| | GGCAGCTTGGCACTTCTCGGTGATGGAAGCAGGTACTCTCTGCCATTT |
| | ACTGGAAGAAGATGCGCCGCGCTACGGATTGGGGCTGTGCCAACTTG |
| | GGATGGCAGACTTTTCCGCTGTGGCATCGCATTTTCAATTGTCGCCAC |
| | ATCATCGCTATGTCCATTGCACCGTGGGGGGCGCGATAGGGCAAGAG |
| | GCGGCAAGTGCTGCAGCATTGCTGCGCGATTTCTCCACCTATGAGAA |
| | ACCGAAGGAAACCGCTGCGCCGCTGGACATGCAGAGCTACAAAGAT |
| | GCCATGCTGCGCGGCCTGCGTCAGCAACTGCCTGACTATATGGTGCC |
| | GAGTGATCTGATGTTAGCGACCGATTTTCCGTTAACCGCTAACGGCA |
| | AGCTAGATCGGCAAAAATTACAGCTGCAGGGCGAACAAATTGCCCAC |
| | CAGCGTGACGGCGTGGGTCCAATCCAGGTGGACAGTGCGTTACAACA |
| | GCGGCTGGTGGCGCTCTGGCAGGAGGTACTCGGCGTGAGCCACGTGT |
| | CGGCCGAAGACGATTTCTTCTCGCTGGGGGGCAGTTCTATAGAATTG |
| | GTGCGTATTCAGCAGGCACTTGAGGCGATTATCGGGCAGGAGATTCC |
| | CATTGTCGATCTGTTCCGTCTGCCAACCATCGCGGATGTGGCGCGTTA |
| | CCTTGATGAGCAACTGCACAATCTACCCGCCGCCCACGATATCGTATT |
| | AGCGCAGGCTGAGGTTTCGCAGGTCAGCGCTGCGCGCGAGAATCTGG |
| | CGTTGCGGCGTAAACGTGCCCAACAGGGAGAAAAGGGCGATGAGTG |
| | A* |
| clbL | ATGAGTGAGCAGAGCTATCGTAGCGCAGGGACACTGTTGGCACAGTT |
| SEQ ID NO: | GGCGTCCGGAGAAACCACCTCAGTGGCGTTGGTGAATCACTATTTTTC |
| 1076 | ACGTATGGCGCAGTTTAACAAGCCGTTGAATGCGGTGGTGCAGCAGC |
| | ACTATGCGTTGGCGTTGGAGGCCGCCGCGCGCGCCGATCGTGAGCGA |
| | CTGGAGGGGCGCGCAAGGGGCGTGTTGCACGGATTACCTTGTACTGT |
| | TAAGGAGTCGTTTGACGTGCAAGGGTGGTTAACCACGTCCGGTGCCC |
| | ATTATCTGAAAGATAACCGGGCGACGCAGGATGCTCCTTCCATCGCA |
| | CGCTTACGCGCTGCCGGGGCGATCCTGATGGGAAAAACCAATGTGCC |
| | GATGATGACTGCCGATTGGCAAACCTATAACGATCTGTATGGCACCA |
| | CCCATAACCTGTGGGATAGGCAACGTTCACCCGGCGGATCGTCCGGA |
| | GGAGCGGCGGTGGCGGTGGCGGCGGATTTCACCCCCGTGGAATTTGG |
| | CAGCGATTTGTTTGGCTCACTGCGCATTCCCGCACACTACACAGGTGT |
| | CTATGCCCATCGTTGTAGCTTGGGGTTGATGTCGGTCAGAGGACATGT |
| | GCCCGGTGGTGGTCCGCAAGCGACTGATGAGCCGGATCTCTCGACGG |
| | CGGGCCCCATGGCGCGCAGCGCGGCGGATCTGCGTCTGATGATGCGC |
| | GCATTGAGCACATTCTGGGTGGAACCGCCGCGTATTCCCGATTTTAGC |
| | CGTTATCAGGCCAAAGCAAACTACCGCGTGTGCACGTGGTTTAGTGC |
| | GCCCCACCATGAGATAGATCAACAGATCGCACAGCGTTTTCAATCGT |
| | TTATCGACAAGTTACGCGCACAGCCTGGCGTGGAGGTTGATGATGCT |
| | ATGCCCGCCGATATCGATCCCGACGCACTGTTTGATATAGCAGTCAA |
| | ACTCAGTGGTCGGCTGGTAAGTACGGCGCTCAATGGACGCCAGCGTC |
| | TTACAGCCGGGCTGGCTGCGCTGGGCTTCAGGCTTGTGGGCAAGCTG |
| | GCGGATGTGCCTGAGGGGATAACGAGCTATTACCAGGGCATGCTGAA |
| | AGACAGTGGCGAACAGCGGAATACAGACAAACTCCGCCACGAATAC |
| | AGCCGGGTGATAGAAACCCTTTTCGCGCGTTATGACGTGTTGTTGACA |
| | CCTGTCAGCCCGGTGCTGGCGTTTGCGCACATGCAGCAGCCGGTGCG |
| | TAAGCGCAAGCTTATCGTCAATGGCGAACCGCAAGATTACAACGAAC |
| | ATTTGTTCTGGAACATGTTAGCAACGGTTTTTGGTTTGCCTGCCACCG |
| | TTTACCCATTGGCGAAAACGATGGATGAGCTTCCGTGCGGCATACAG |
| | ATCATTTCCGGGCATTTTCACGATGATGTGACGATTAACTTTGCTGAG |
| | TTCTGCGAAAGCATCAGTGGCGGATTCACGGTACCGGAAGGGTACTA |
| | G |
| clbM | ATGACGGTGAATAAGCTTGAGCCAGACAACGGCACGCCGAACGATG |
| SEQ ID NO: | AGCTGTTCACGGTGGCGGGTATGTTTGACGGCTCGCTGTATAAATTAC |
| 1077 | TCCTGCGCATGGCGTTGCCGATGTTTGTCGGTATGTTAACGCAGGTGA |
| | CCTATGCCATCGCCGATATTTTTTGGCTGAGCCACATTGATGTCACCA |
| | ACAGCGGCATCATTGCCGGTGTGGGGCTGGTTTTCCCCGTTGGAATG |
| | GGGTTATTTGCCATCGCCAACGGCATTCAAATCGGTATGGGATCTTTG |
| | CTGTCCCGCGCCATCGGCATGCAGCGGTTGGATCGTGCACAGCGGAT |
| | TTTGTCGGTCGGTATCATCATCGCGCTATTCTTTGCGATCGTGATCAC |
| | CGTACTTGGCTATGTTTATGCCCAGCCGCTATTGCGCTCACTGGGGGC |
| | CACGAAGAGCATCATCGGCTACGCAACGGAGTTCTATTATTATTCGCT |
| | GCTGACCGTGTTCAGCATCATGTTGATTGGCGTCATGATGGGGCTGTT |
| | TCAAGGCGCGGGTAAGATCATGGTCATCATGAAGGCCTCATTGTTGG |
| | GGGCGTTGGTAAATATTATGCTCGATCCAATCATGATCTTTGTATTCG |
| | ATTTTGGTGTGAAAGGGGTGGCATTGGCGTCGTTTCTGGCGCAACTG |
| | AGTATGGTCGCCTATTTCATCTACACATTGATGGGATTACATATCGGG |
| | TTGAGTATACGCATCGCGTTACGCCCATTCTCCTGGAAAATCTACCGA |
| | GAGTTTCTCTCCGTCGGCATGGCGCAGATGCTGATGCAACTGATCATT |
| | GCGGTCGGCATAGTGATTTATAATTTTTTTATCGTCAGGCTGGACGTA |
| | AACGCGATGGCAGCGTTTACGCTCACTGGTCGCATCGACTATTTCATC |
| | ATCACGCCGATGCTGGCTATCGCGACCGCGTTGCTGACTGTGGTGGG |
| | GCAAAACTGGGGACACGGAAATGTCACCCGCACGCTGAATGCCTATT |
| | GGGCCGCCGTCGCGCTGGCCTTCTCTATTGTGCTGGTGTTAGCCGTTA |
| | TGCATATTGTACTGGCACCCTGGATGTATCCCCTGTTTACTCGTGTTG |
| | TTGCCGTGAGTGACTATGCGGTACTGCAAACCCGTATCATGGCACTG |
| | GCACTACCCTTTGTCGCTATCAGCCTGTTAGCAAGTGAATATTATCAG |
| | GCCATTGGTAAGCCTTGGTATTCGGTGTTGTTGACGCTGATGCGTCAC |
| | GTGTTTATATCTGTCCCTGTCGTATACCTGCTGGCGATTGTGTTAGAG |
| | ATGCGTATTACCGGCGTCTATTTTGGGGCGATGAGCGGCACTTTTGTG |
| | GCTGCGTTGTTGGCGTGGCGTCTGCTGCGTCTTTCACCTCGTCTGCTG |
| | CGATGGAATCAGGAGGCGGTGCGCTCCCAACACTTGGATATGGAGGT |
| | GGCACCATGA |
| clbN | ATGATGTCGGGCAATCCGTTGTCGTGGCCACAGGAACAGTGCCACAT |
| SEQ ID NO: | CATTGATCAACTGTATCCTTACAGTGCCGTAAACATCATTGGTGGTGT |
| 1078 | GGTCACCATTGAGGGAATTGTCGATCTGCCCCGACTACACGCGGCCA |
| | TCCAGTCCGCCATTCGGCAATTTGATGCGTTGCGTATGTGGTTTGTGA |
| | TGGGAGAGGAGAGTGAAGTGGTCAGCCAAGTGCAGCCTTATCACTGG |
| | CGCGATATCCGCCATTTGACTTTTTCCCCAGATTATGACAAGGAGAAC |
| | CTACGCCCAGCGGCGATCGAAACATTTGTTGATGAGTGGTTCCGGCA |
| | GCCCTTTACCCTGCTGGCGCACGATTTGTTCGAGTTTGTCACCTTTAC |
| | GTGCGGTGAACAATACAGCGGTTATTTATTTAAAGCCCATCACGGCA |
| | TTGCTGATGGTTGGTCAATGGCGTTGTTGTCAAACCATGTCAAGCGAG |
| | CATACGAACAGCAGGACGTACCAGATGATGCGTCACCAGCCTATAGC |
| | GCATTTCTGGCGCAGCAGCAATCTTATCAGGCGTCAACGCGCTTTGCC |
| | GTTGATCGTGGCTGGTGGCGTGACTATATCGACGAATACCGCGACTG |
| | CTTTCCAGACAGTTCCCCCATCGTTACTACTGAGGGCATCAGTTGTTC |
| | CACTTGGCTTGAGCCAGCCATGATTAACCGTCTCTACAGGTTGTGTAA |
| | TCGTTATGGCTGTACGCTTAATACGCTGTTTATTGCACTCTTCGCTCTC |
| | TATCGCGCCAGAGTATGGGGGGAGGAAAAAGGCGTAATAGGCGTGC |
| | CGTTAGCCAATCGGCATACTCGCGAGGCGCGGCGTTGCTTTGGCATG |
| | TTTACTAATCAATTGCCGCTGGCCTATCGACTGGTGCGCACGGAACG |
| | ATTCTGTGAACGGGTGGCCTTTTTCCAGCGCGAACTGAAACGCGGTTT |
| | TAAACACAGCAAATATCCCATTACCTTATTCAACCAGGATTTGGCGG |
| | AACAGGGGGGAGGGAAGTTGCGGGCGTTCGATTATTGCGTTAATTAT |
| | TATAACTTTACCTATGAACGCCACATTGCAGGTGCCGCGCAACGCGT |
| | GGAGAGTTACTACAGCGGCGAGCAGTCCTATAAGCTACAGATTGTTT |
| | TGCAAACTGTTAATAATCATAAAGAAAGCCTTAGGTTGAGTCTGGAG |
| | GCGCTGCGTAGCGCTTTCACTCCGCACCAACTGACAGCGATGAAAAA |
| | TGGGCTGTTGGATTTAGTCACTGCATTGGATCGGCAACCTGACGCTCG |
| | TCTAGGCGATCTCGAAGTCTATCCTGCGCCGCACGTCGCATTGGCATG |
| | TGGCTCACTCAAACCCTCATTTACTTCCCGATTTGCAGCGCAAGTTGT |
| | GGAGCATGGTGATCGCACAGCACTAATTGATAATGAACAGTCTTTGA |
| | CTTATCGGCAGTTGGATGACGCTGTTGAGCGGGTTGCGCGTTATCTGC |
| | GCCAACAGGGGATCGGACGCGGTCAAGTGGTTGGGATTATCGCTGAG |
| | CATAGCGCTCAAACGGTGATGGTTATTTATGGTATTTTGCGCTGCGGC |
| | GCGGCATTTCTGCCGCTAAATCCCGCGTTGCCAACGACCCGCCTTTAC |
| | GCCATGTGTCGTAAAGCGCAGGTAGCGCATATCCTTTACGATCCGGC |
| | GATGCATGAATTGACGCAGGCGCTGGCGTTTCCCGCGTCCAGCTTGCT |
| | CCAGGCCTTAGCCACTTCGGCGCTTGCTAGAGAACCTTGGCCAGCTAT |
| | TGAACCGCAGGATCTGGCATATGTGCTGTTTACCTCCGGATCGACCG |
| | GCGAGCCTAAGGGCGTGCAAGTCTCTCATGGCAATCTGGCTAACTAT |
| | CTCCATTTCGCCGCCGAACGTTACTTTACAGCGCAGGACCGGGCTGC |
| | GCTCTACTCTTCGCTGTCCTTCGATTTGACCATCACTACGCTGTTCGCC |
| | CCCTTATGTGTGGGCGCTAGCATCAGCGTTTGTCGGCACGCCGAGAG |
| | CGAAACATTGCTGCGCATGGCCGTGGTTGATCAGCCCAACACAGTGA |
| | TCAAACTGACGCCTGCACACCTGCGGTTGTTGTGTGCGGCTGGCATA |
| | AGCAGTGAGCAGATTCGTACCTTGGTGGTTGGCGGGGAGGATTTCAA |
| | GCGGGATCTGGCGCGTAAGGCGGCTGCTCTCTTTCCTCAGGCCGTGAT |
| | CTATAACGAATATGGGCCGACAGAGGCCACCGTTGGTTGCATGATCT |
| | ATCGCTACACCGGGCAAGAAACGCTGCCTTCATTGCCCATCGGTATG |
| | GCGATCGACGGTTGCCAGGTAGCAATTTGCTCCCCTTGGGGCTGTCCG |
| | GTACCGGAAGGCGAAACTGGTGAATTGGTGATTTATGGCGCGTCAGT |
| | GACGCAGGGCTATATCGATGCGCCGCAACAAACGGCCGCCGCCTACC |
| | TTAAAGATACCAATGGGGTGATGATTGGCTATCGCAGCGGTGATATT |
| | GGCTACGCCATCGCTCCTAATACGCTGGTTTATCAGGGACGTAAAGA |
| | CGATCAGGTCAAAATTAATGGCTATCGTATTGAGCTGTGCGAAATCG |
| | AACAAGCGTTGTTGAGCGCACCTCAGGTAGAAAGTGCGGCGGTGGCG |
| | GTGATTGATGATGTGCAGGGGCAGCACAGCGGGTTGCTAGCCTGCGT |
| | GACACCGTCATCTGTTGATGTAGCTACCGTAATGCAACATCTGCGTCA |
| | GCAATTACCCACCTACATGCAACCCAAGCAGTGCTGTGCTATCGCCC |
| | AACTGCCGCTATCGCACAATGGCAAGGTGGACGTGCGTCAGATGGTC |
| | GCAACGGTCCGAAACACCGCGCCTGCATCGGGTAGCGAGCGGCTGGG |
| | AGATGCGGCGATAAGGCATTCAGTTCGGGTATGTGTGGAAGGGGCGC |
| | TGGAGCAAACTGAGTTTGATGATAACGAAAATCTTTATGTCTTGGGG |
| | TTGGACTCCATCAAGAGTATCCAGATTGCAGCGCAGCTCAGGCACCA |
| | CGGCTGGACGATGTCAGCGGTGCAGGTGATGGAGTGTGGAACAGTGA |
| | ACGCCATCTGTGAGTTTCTCGCTAGTCATACAACGGTGTCACAGCTCG |
| | CGCAGTATGCTCATAACACCCGTATCGATCTACCCGCATTGCGTTGGT |
| | TTACGCAGTTGGCGTTGCCAGTGCCCAATGTTTATAACCATGTCATTG |
| | TGCTGAAAGTGTTGCCGGGCTGCCCGCTCGAGCAGTTACACAACAGG |
| | CTGCATACATTAATCCAGCAGCAGCCAGCTTTGCACAGTGCGCTGGA |
| | CGCTGAGGGGCGGTTGCTAGTATGCGACCCTAATGTGTGTTATCCCA |
| | ACGAGGTGCTCACGGAATATTCGACGGCGCAGTGGACGTTGGCAGAG |
| | GTGATCGCTCAGTGCAATAGCATGCTTGACGTGACAAATGGTCGAGT |
| | CTTTACGGCAGCTCTGTTACACGCCCCGCAGCCTGCGTCTAGCACGCT |
| | GGTACTCTGTGCCCATCACTTGTGCGTCGATATGCACTCTTGGTATTT |
| | AATCCTCAGCACGCTGGACGCCGTCAGCACTGTCAATGGCACCAGCA |
| | ACAGCGGACTGCATCGCTGGAATGACTATCTGGCGAGCAAAACGGTA |
| | GATTCCGCCACACACGAAAGCTGGCGCACAGTATGTCAAACGCTACC |
| | GCTGCATTTCCCACCCGTGTCATTGCCAGATGACTCATTACCTCGTAC |
| | GCGCGCTTGGCGTGAAGACTTTCGCCATCCTTGCGTCAGGAGGTTGTT |
| | TGAATCCAGTGGTAATACCGCGTACAGCGCCGAAACCTATGTGCTAA |
| | CGGCATTGGCGCTGGTGCTGCGTTACTACAGCGAAGAGCCTTGGTGT |
| | CGCATCGAGATGGAAGGCATGGGACGCGGCTGCTGGCCCGATGAGCC |
| | TGACGTTGCGGATACGGTCGGGTGGTTTACTCTATTTTATCCTTGGGC |
| | GATCCCTCTGCATGGCGATATGGCGACACTGTTATCTGCGATCGCTTC |
| | CGATTTGGCAAAACGCACGCATGGTGGGGGAGATTACGGTCTGCTGC |
| | AAATGCGGCATGCACCGGAAGACTCTCTTGCGCAAGGGATCCGAATG |
| | AATTACATCGGTGTGCAGGCGCAACCTTCACTCCGCTATTTTCATATT |
| | GATCACTTTAATAGTGATATCTATACCGCGCCTGAAAACGCGCTCGG |
| | CTGCGTGCTGGAGTTTAATATTGCGCGCTCGGCAGCTGATGGTCTCTC |
| | CTTCCACTGCCGTTTCGATCCCACGCGCATTGCGCTGAATGATGTGCA |
| | ACTCCTGCTGGCGCGCTACAAGAACAGCCTCACTGATCTGGATGCCT |
| | GGCTGTGCCAACACAGCGCCACGCTGACGGGCGCGCCGACTTTGTGG |
| | ACACTATAA |
| clbO | ATGGCAAAGGATGATTTTACCTGTGGCTCACTGGATATTGCCATTATT |
| SEQ ID NO: | GGCATGAGCGGGCGTTTTTCCGGTGCGGAATCGGTGCCCGAGTGGTG |
| 1079 | GGATAAGTTGCTGGCGGGTGAGGAGTTCACTCAACCGACATGCACAG |
| | AAGACGATAATGGGAACCCTTGGATTAGGCTGCGCAATATAATCACC |
| | GCTCCCTATGACTTCGATGCTGCATTTTTCAATATTCCGCCTGGCGAA |
| | GCCCTGTTGATGGATCCGCAACAACGAATATTTCTTGAATGCTGCTAC |
| | AACGCTCTTGAGCATGCGGGCTATATTCCCACGCAACTTAAGCGAGT |
| | CGGCGTCTATGGGGCGACCTACGCCAATAACTATTTTATCGATCGCGT |
| | CTATCCATACCTGAAGATGAGCGGCGATCACCATTATCTTCAGGCGC |
| | AGATTGGTAACGAAAAGGATTACTTGTGTGCTCAGGTTGCCTACAAG |
| | TTGGGGTTTACCGGACCCGCTGTGAGTGTACAGACCGCCTGTTCCAGT |
| | TCGTTGGTGGCAGCATATTTAGCATGCGAGGGGTTATTGACTTTTCAA |
| | GCCGATGTTGCGTTGGCAGGTGGGGTCACCTTGGGTTTTTTACAGGCG |
| | CACGGCTACAGTCCGCAAGGTGATAAGCTGGTATCGCAAGACGGACA |
| | CTGCGCCCCGTTTAGCGCTGAGGCGACCGGCACGGTATACAGCAGCG |
| | GTGCGGGAGTTGTGGTGCTAAAACGTCTTGAGGATGCACTACGCGAC |
| | CAAGACAGAGTGTATGCGGTGATTAAAGGAGGTGCGGTAAATAACG |
| | ACGGCGGGCGTCGGCTGGGGTTTGTCGCCCCCAGCGTAGAAGGACAG |
| | GTAGAAGCGATCAATACGGCGCTTGCCGCCGCAGAAGTCGTACCGAC |
| | TGATATCGCACTGATCGAAACGCACGGTACCGGTACCCCGTTGGGTG |
| | ATGAGATTGAGCTTGAAGCTCTGCACCGGGTTTTCGCACCGGCATGT |
| | GCGCCGCACAGCATTCAATTGGGTGCGGTAAAAGCCAACTTGGGTCA |
| | CCTGGGAGTCGCTTCCGGTATCGTCAGCTTAATGAAAACCGCACTGA |
| | CGCTATATACTGGTTTAGTTCCCCCGCAGATCAACCTAGTAAATAAAC |
| | ATAAAAAACTGTTGCAGCCAGCCTCGCCATTTTATCTCAGTGATGTAG |
| | TGACTTCTGTCCCACAAACGAAAAGGATCCATGCCACCGTTAGTTCGT |
| | TTGGGCTGGGAGGGACCAATGCTCATTTGGTTCTGCAAAACTGGTGC |
| | GAGACGCCTGCGCAAGCGGTGCAAGAAAATGAGCGCCGCCTGTTTTT |
| | CTTCAGCGCCAAAACGCCATTAGCCCTTAGACAACAGTTGGATGCTC |
| | ATTATCACGCTCTGGCGACCTATGCGGAAGCAGATAAAGATCGTATC |
| | GCCTATACTCTCGCGCAACGCCGGGCACATTTCCCGTATCGCTGTGCG |
| | CTGGCGGCAGACAGTGTGGTAGCGTTGCGTGCAAGTTTGGCGAAACT |
| | GCGGGATGCTGACATGTCTTTTACCCCTATCAATATGGAAACTACCTT |
| | GGTGTTCCTATATCCGGACCGGGACGATAAGCTGGAGAGTGCCCTGA |
| | CGCATTTGCTGGCTTGTCAACCTAACTTGCGCCAGCGGCACCAGCGCC |
| | TGTCTCAGGATGTTGCACAAATCTGCGAACCAGCCGATTGGACGCCA |
| | GCATTGCGTCAGTTTATCCAGCAGGTGAGTCTTAGTGAATGGCTGATC |
| | GAACAAAGTATCTCGCCTGTGCAGCACATCGGTTACCTGACGGGGGC |
| | TGCAGCAGCGCAGTATGTTGCGCGCATCATCTCACTGGAAAACGCTG |
| | TTCAGCAGGTGATTGTGGCTGAAACGACACCGGAGCAGACGCTGGCC |
| | GGCAACAGTGAACTGAGCGAAATATTGGCTAATCTGGCGGTGACGGA |
| | GGGAACGCTGATGCTGGAAATCGGTCGCGCGGGGACGTTTTCCATAT |
| | TGTATCACCAGCATGCGCAGTGGGTTGGGCAAACAGTATTTTCCCCC |
| | ATGCTGAATACGGATACGCCTGAGGACATCCTGCCGCTGTTGGGCAC |
| | TCTTTGGCAACGAGGAGTGACTATTTGTTTACCGGAAATGCCAGCCGT |
| | GCAGACTATAGGCTTACCCGGTTATTCGTTTGATCGCGTCCGGTATGA |
| | AATTCAGTCCAGTGATGCGAGAGAGAACGCTATGTTACCAGTGAGTT |
| | ATTTGTCGGTCAGCGACTTTGTGGAGAAAACCTGGCGTTCATTGTTAT |
| | GCATCGATCATTATGACGAACACGCGGTTATTTTCGAATACGGAGCG |
| | ACCTCGATGCACGTTATCTCTTTCGTCGACAGCTGTAACCACATTTAT |
| | AAAATCGGACTGACTGCCGCCGATATTTATGCCAGACCCGCTATCCG |
| | TGAGCACAGTGAATTTATCTCCGAATGTGTTGATGGTATTTTATGA |
| clbP | ATGACAATAATGGAACACGTTAGCATTAAAACATTATATCATCTCCT |
| SEQ ID NO: | GTGCTGTATGCTGCTCTTTATTTCCGCTATGTGCGCTTTGGCGCAAGA |
| 1080 | ACATGAGCCTATCGGGGCGCAAGATGAGCGCCTGTCGACATTAATTC |
| | ACCAACGGATGCAGGAGGCCAAGGTCCCAGCCCTTTCCGTAAGTGTG |
| | ACCATTAAGGGGGTACGTCAGCGATTTGTCTACGGTGTTGCCGATGT |
| | GGCTAGTCAGAAAGCGAATACTCTAGACACAGTTTACGAGCTGGGAT |
| | CGATGAGTAAGGCGTTTACCGGACTTGTGGTGCAAATACTGATTCAG |
| | GAAGGCAGACTCCGGCAAGGGGATGATATCATTACCTATCTGCCGGA |
| | AATGCGCTTGAATTATCAGGGAAAACCTGCTTCCCTGACCGTGGCTG |
| | ATTTCCTTTATCATACATCAGGATTGCCTTTTTCAACACTGGCTCGGCT |
| | GGAAAACCCTATGCCTGGGAGCGCTGTGGCACAGCAACTGCGCAACG |
| | AGAATCTGCTGTTTGCGCCGGGTGCGAAGTTTAGCTATGCCTCCGCCA |
| | ATTATGATGTGTTGGGCGCGGTGATTGAAAATGTGACGGGAAAAACC |
| | TTTACAGAGGTCATTGCGGAACGACTCACGCAGCCGCTGGGCATGTC |
| | GGCGACTGTGGCAGTTAAGGGGGATGAGATTATTGTCAACAAGGCAA |
| | GCGGCTATAAACTGGGATTCGGCAAACCCGTTCTGTTTCATGCGCCTC |
| | TGGCCCGGAACCATGTTCCTGCCGCCTATATCCATAGCACTCTGCCTG |
| | ATATGGAAATATGGATAGACGCCTGGTTGCACAGAAAGGCTTTGCCG |
| | GCAACGCTGCGTGAGGCGATGAGTAACAGTTGGCGTGGTAATAGTGA |
| | TGTTCCGCTTGCCGCAGACAATCGTATCCTCTATGCCAGCGGTTGGTT |
| | TATCGACCAGAATCAAGGCCCTTACATCAGTCACGGTGGGCAGAATC |
| | CAAACTTTTCTTCTTGCATTGCGTTGCGACCGGATCAGCAGATTGGCA |
| | TTGTTGCGCTGGCAAATATGAATTCGAATCTGATACTACAGCTTTGCG |
| | CGGATATCGATAATTATCTGCGCATTGGCAAATATGCTGACGGCGCT |
| | GGTGATGCAATTACAGCCACCGATACCCTTTTCGTCTACCTCACGTTG |
| | TTGCTGTGTTTTTGGGGGGCGGTGGTTGTAGTGCGCGGTGCTTTCCGT |
| | GTTTATCGCGCAACGGCGCATGGCCCTGGAAAACAGCAGAGGTTACG |
| | TTTACGCGTACGTGACTATATCATCGCCTTGGCGGTTCCTGGGCTCGT |
| | GGCCGCCATGCTCTATGTCGCACCGGGTATACTATCTCCAGGACTTGA |
| | CTGGCGTTTTATCTTGGTATGGGGTCCATCGAGCGTGTTGGCGATACC |
| | GTTCGGAATTATCCTGTTAGCTTTCGTTCTGACATTAAATCATCAAAT |
| | TAAACGAATTCTATTACACAACAAGGAGTGGGACGATGAGTAA |
| clbQ | ATGAGTAATATCAGTTTGTATTGTTTGCCATATTCAGGTGGTTCTGCC |
| SEQ ID NO: | GCCATGTATTATAAATGGCGTAGCGTGCTGTCGGACAATATTACTTTG |
| 1081 | CGGCCTTTAGAACCTGCGGGGAGGGGAACTAGAATACGCCAGCCGCT |
| | GTGTCTTACGATGGTGGATGCCGTCGCTGACCTTTATCAACAATTTGT |
| | GAAACACTACACAGGTGGAGACTACGCCATTTTTGGGCATAGTCTCG |
| | GAGGGATCATGGCCTTCGAACTGGTGCATTATATTCTCGATCATGGAC |
| | ATGACATGCCATGCGCGCTGTTTTTTTCCGGCTGTCGCCCACCCGATC |
| | GGGCCTCTCATGAAGTAATACTGCATACCTTGCCCGATCAGGCGTTTA |
| | TGGAAGAGATCGTCAAGCTGGGCGGAACTCCGGTTGATGTCTTTCGT |
| | AATAAAGAGTTAATGACAATTTTCACCCCCATCATTAAAAACGATTA |
| | TCGGCTCTATGAGCAGTATGTATTTCAGGCCAAGGCGCGCACATTAA |
| | CCTGTCCGATCGTGCTATTTCATGGCGATGCTGACAATCTGGTAATGC |
| | AGGATGAATTACTTGCATGGGAAAAATTCACCACACGAAAGACGCGG |
| | ACTATTATATTTCCTGCTGCCGATCATTTTTTTGTCGATAAGCATTTTG |
| | AACAGGTGGTAGGTTATGTGAACCAGACGATTGAATCACTCGAAATA |
| | GTAGGGTAG |
| clbR | ATGGATAAGTTCAAAGAAAAAAACCCGTTATCTCTGCGTGAAAGACA |
| SEQ ID NO: | AGTATTGCGCATGCTGGCACAAGGTGATGAGTACTCTCAAATATCAC |
| 1082 | ATAATCTTAACATATCAATAAACACAGTAAAGTTTCATGTGAAAAAC |
| | ATCAAACATAAAATACAAGCTCGGAATACGAATCACGCTATACACAT |
| | TGCTAACAGGAATGAGATTATCTAA |
| clbS | ATGGCTGTTCCATCATCAAAAGAAGAGTTAATTAAAGCTATTAATAG |
| SEQ ID NO: | TAATTTTTCTTTATTAAATAAGAAGCTAGAATCTATTACGCCCCAACT |
| 1083 | CGCCTTTGAACCTCTATTGGAAGGGCACGCGAAGGGGACTACGATTA |
| | GCGTAGCGAATCTGGTTTCCTATCTGATTGGCTGGGGAGAGCTGGTGT |
| | TACACTGGCATGACCAAGAGGCAAAAGGAAAAACTATTATTTTTCCT |
| | GAGGAAGGATTTAAATGGAATGAATTGGGGCGTTTAGCACAGAAATT |
| | CTACCGTGACTATGAGGATATTACAGAGTACGAAGTTTTATTGGCAC |
| | GGTTAAAGGAAAATAAGCAGCAACTCGTGGCTTTGATTGAACGATTC |
| | AGTAACGACGAGCTTTACGGTAAACCTTGGTATAATAAATGGACCCG |
| | AGGTCGTATGATTCAATTTAATACCGCCTCGCCTTATAAAAATGCTTC |
| | GGGGAGGTTAAATAAACTGCAGAAATGTCTTGCAGAATAG |

TABLE 13
Colibactin Amino Acid Sequences

| Description | |
|---|---|
| SEQ ID | |
| NO: | Amino Acid Sequence |
| clbA | MRIDILIGHTSFFHQTSRDNFLHYLNEEEIKRYDQFHFVSDKELYILSRILL |
| SEQ ID | KTALKRYQPDVSLQSWQFSTCKYGKPFIVFPQLAKKIFFNLSHTIDTVAVAI |
| NO: 1084 | SSHCELGVDIEQIRDLDNSYLNISQHFFTPQEATNIVSLPRYEGQLLFWKMW |
| | TLKEAYIKYRGKGLSLGLDCIEFHLTNKKLTSKYRGSPVYFSQWKICNSFLA |
| | LASPLITPKITIELFPMQSQLYHHDYQLIHSSNGON* |
| clbB | MDNTSGDFPCNKMDTRKQLPLTPSQQGFLFHSLKDKKRSNYHEHFTCIFSQH |
| SEQ ID | VDSAHFKWALETLFRKHECFRTDYNWEIDERPCQVVKTDVLPDIYVLDCEQE |
| NO: 1085 | EIRFLLANDDIIIPVPQDDGIDAIIPQLLQADLKYPFSLKTIPVRAYLIQST |
| | KESAFILSYHHIVMDGWSLSLFIKQLLQLYGAAVVSGVRDDSAIIPSSLKPL |
| | VDTLSARRHTFQHDYWAAYLREGTPTCIVPLSQYHTDTEAENNSYVNQTNHV |
| | EINLSPDVCQKIQTLCSDYRITPAVIFYVAWGILLQRWCYADDVLFGATISG |
| | RNIPIDGIEETLGLFINTLPLRLRDDGATLLQHLQRMHQTLIAHYSNEHDAL |
| | ASIQRLVHKEGHAGDLFNTLVVLENYPVDMTLLSCASPVAIRHLSVHEQTHY |
| | PLTLTITQQKGFRFSIAYALNYLTNNMAQALLMHLSYLLEQLVDNPQRPIAA |
| | LVNLSPCQQAQVLQPYLERMACRDWDSQSNVIEQFHQVAATSPAQVAVVDEL |
| | CALTYSELAAQAEQLAAYLVQQGVMVGDTVGIISERRVNTVVAIIAIMLIGA |
| | AYVPISPDYPVGRMQEIIDDSGLALLLVHGKPLDALNVAQSDLCAFPVAPSV |
| | VFPVITPDSRAYVIYSSGSTGKPKGIAVAHRGLLRLIQGDSPLKVESGETTL |
| | LTCPFEFDVSVFEMWSTLLNHGKLVLLSKQALLDINHIRRTIADEQVARAWF |
| | TSSLFNSYVAEGADFFGMLQHITVGGEAVSAWHVNDVMQKYPHLVVTNGYGP |
| | TENTIFTTAYRENGLQPARVPIGYAVPGTSLYITDLHGHLLPIGATGELVAG |
| | GVGVAIGYQNNPALSATVFVPDPFIPGGMMYKTGDYARLLDDGCVDCFGRKD |
| | GQIKINGQRIETGEIEQRLLECSGIIEAVVVPYRVRETLHIAAVVCVNDSYD |
| | EVEVRGQLADRLPPFAIPESLVVVTEIAKSHSGKADLAQLRYLLPATQCNAV |
| | STTISEVHSDMEHALHAIWQRVLDRQDIDSNASFFALGGTSLDTIRVKGDIK |
| | RQLGLEIDITDLFKYPTLTALAHFLDTAVSPEDAIPTRAVVYSDMPVAIVGM |
| | AGRFPGAANIAALWTLVVGGESGLTLFSDEELRAHGVTPDTLKQANYIKTKG |
| | IVDDHEWFDADFFGYTPNEAECMDPQIRLLHQCCWQTLEHAGCDPATFTGAI |
| | GIYAGLLTSPHWLNAVMQDTTDSTALYKASILNIHSVTALIAHALNLTGPAV |
| | TLDTACSTSAVAIHQACIALRNRDCDAALAGGVSIEMPAYRGYEYHEGMINA |
| | RDGVCRPFDSQASGTVTGDGLGMLLLKRLDDALADRDCIYGVIKGSAVNNDG |
| | NNKIGYTAPSVIGQSTVIRTSLRRAGFDSDSIGLVEAHGTGTVLGDPIELRA |
| | LNEVFGPTPVPFCVVSALKSNIGHLNSAAGVAGVIKTTLALHHQVLPPTAHF |
| | RQLNPAIDLSRSALYVNQQVQPWPSTRPRRALVSSFGIGGTNASIALEAHQH |
| | EDDPSATGVRDSYLLLFSAKTPAALELRVASTLEYVKHGVGVRLPDVAYTLQ |
| | TGRTAFDHRRAYLVSRGSKIDLSCATILQAEIFNGQRTTAEICFMFPGQGSQ |
| | YHGMASALYAHQPMFRQHMDRCFAAFQRYSTVDLKALLFDDEDTRDIDQTQF |
| | TQPALFCVEYSLARTLIDLGITPDSMIGHSLGEYVAACIAGVFTLEDALHVI |
| | EARGRLMQSMRPGSMMAVYLSREQLTPWLAAERGIELAANNSAHFCVVAGEQ |
| | AAISRLSTRLVEGGIQHRRLKTSHAFHSAMMTPMLHDFAQLLGQIPMHAPHK |
| | RFISNVSGTWITEEQATSPDYWVQQVRNAVLFSEGAAQLLVQPTLFIECGPG |
| | NTLSTFIQGHNQYSDQPTLLTLRKANAAIDDEHMLHRTLAALWVRGENIDWR |
| | RFNQTALGKHIPLPDYPFEQTYYYRYGAALSGYRQYPNPLRRPQDEWLQRVL |
| | WRMHDTSLREAFYAPGELIIIISADGDKLQQTLMSSGVDSITMPLPISSEDD |
| | VWDNDRILTHFHDICALLAHKTYRQLHCLYAPGAEAGSSLTQSLSGLYRVAR |
| | WCMHSTTPLASLTVLTHGAFRVQEEDNPEPTLAALSGAVNVFAQELHPTEVR |
| | LIDIDAQSSDENLNLLTQRLAPKQETVMALRQGMLYLRRFIPTRLLAHLPPQ |
| | TGCIPGNVLWIIGGEKGIGRMIGEALAQREGVRVVLSSRTGYHHEAVQQDAL |
| | DVIHCDVTQAEAVRACLATLLERYGRLDGVIFAADATTTLTLHQLSESALRD |
| | TLTVKERGTANVLHALAQRNLLDERLLLLFCNSLAAVNAEIGQTGYATASAY |
| | LDALAQQLRTRYKVNALSIGLDALREQGMLLDAINGSEYDVLRGLRPLMTGT |
| | LLQAYKQQGADTSYYARLSPESDWLLDEHRISGIATLPGTGYLALAYEALRH |
| | YFVQDQICIDELVFLAPLTVMDNCSVDVFVDISPNGQGVSVEVKSMTERFSG |
| | TLTTHARGRATRLMVDDNVVCDLTGLMREMHTITPPTKELSSTHFHYGPRWH |
| | SVQQLYGNTAQTQVFATLALPTVAANDTIALHPALLDIASSVVEQLPGFHTD |
| | SVPFLYQDLRLYRPLPNTLHVALTVNRHDEEGDSYAFTLYDMAGEMVARCAA |
| | MVKRKVQLHIQDVDDDTRLRVPSADNYQLRLAAEGEGAGKLALCPTPRLALG |
| | DSQVEIEVLATGLNFKDVLFTTGLLRQQPGEAPLQLGLECAGRITRVGKNVT |
| | EFAPGEDVMAVLNGGFVQYARVESDCVVRKPAHCRIEQAAALPIAYLTAYYA |
| | LVVRANLQPGERVLIHSAAGGVGLAALHIAKRCGAQIFATAGSEQKRDYLLS |
| | LGVHAVADSHDEQFAATLLTASDGQGMDVILNSLTGRLLDASLALLAPLGRF |
| | LELGSKDIVEDKALPMRFFAQGGTFIPINFHAAHGAFSRYLQQIVAWIDDNT |
| | LPLLPCKSVPLPEVARAFATLTTPQHIGKVVVTHRTAAGMDRLNAMIAERRL |
| | GGYALSMSNAEVMRQLWPILNTRSPWAQLLLSPRAIDRLARGNRVDRGVPSA |
| | ANDTITQQTVKKRPRPEIGVPYSPATREVERVLCQILEEYLGLDRVGIDDNY |
| | AELGATSLDMVQLSGQMARHYPQVSVVSLYNHATVRQLATFCQPPEGESNAP |
| | SPQPAVQTNTRANQIAKRALQIAKNTARSHTSLH* |
| clbC | MEYASEMNGMEIAIIGMAVRFPQSRTLHEFWHNIVQGKECVTFFSEEELLAE |
| SEQ ID | GVEQSTLDNPAYVRAKPYIEGICDFDAAFFGYSHKEAQTLDPKSRVLHEVAY |
| NO: 1086 | HALEDAGYAQRTSDLITGVFVGASEDVDWLRRSLSQIGGDALNRFESGIYGH |
| | KDLLAHLIAYSLNLNGPVYSLYTSCSTSLSATHIACRSLLFGECDLALAGGI |
| | TIDLPQKSGYFCQQGMIHSTDGHCRPFDSQASGTLFGDGAGVVVLRRLEDAL |
| | AAGDRIYAVIRGSAVNNDGKQKIGFVAPGHEGQKAVICAACHLAEVSPESIG |
| | YVETHGTGTRIGDPIEFAALTEAFDTSHRQYCALGAVKANIGHTHAAAGVAG |
| | LIKTALVLHHRTIPPLANYQMPNSKLDLAHSPFYIPIQPQEWPASRMPPRAG |
| | VSSFGIGGTNVHMILEGLNPAVRDDHDQVRAPVFIPLSAPSFEQLDELTQQL |
| | TPLLATLDASTLAYTQQVARPVFDCRRVIQVENDGTQAMLASLDNLMPDAPW |
| | GLHCPDLRTTNDCTYAQWLAHSAHYQREATALTALLDGMNIPPAYCHAETWA |
| | AQANSSLLIRGCQTIAALKTWMNLLPTLTLLSGAGTGLLPAAAASGMIATQD |
| | VLHLLWEMEQKALHLWLPERHEPIPGYVLAWQGNPITDAQRNDRGFWSEALL |
| | ADTRELGEGVHSINWVRLPPEIREDVDVLRYVAQLWCAGINVDWAVWYGTPL |
| | PQRGSASAYPFAHNHYPLPGRVMGSVETQPEAGPETHHPYQARPVLSVPFVA |
| | AHSRGMQYITGLMELLLEISPVGVDDDFFELGGHSLLVTQLTSRLERDFNVH |
| | IDLLTLMENPNPRNIYAHIAAQLGGEDNLEIACQ* |
| clbD | MMNVAVIGAGVMGTGVAHNMAQYGISTNVVDISQSQLDKCRQMIEANLRLYN |
| SEQ ID | FHPQHKKKTHSTAEIMENIRFTTELDDIVECDLVIENITEDIEKKNALYTRM |
| NO: 1087 | NTICGASTVFGVNTSAISITALSKLMRHPENVVGVHFMNPVPLMHTVELIRG |
| | VHTAERTLNIFHHLFAQLNKTGIVVNDSPGFVTNRAMMIFVNEAIFMVQEQI |
| | ARAEDIDTLFKTCFGHKMGPLQTADLIGLDTILQSLQVLYESFNDDKYRPSF |
| | LLKKMVDAGYLGVKSGQGFYRYQQTYAEQ* |
| clbE | MKKQDMKAAIREFLSRSLRGHTLNDDDDIFSLGLVHSLFTVQIILFIEKNFQ |
| SEQ ID | VELEVSELKTEQIATVNKIVELIQRQTGLE* |
| NO: 1088 | |
| clbF | MCTENYELAQQEAVLFAKQHLALAAQNIERQQFIVPDIISCVAQAGYLGASI |
| SEQ ID | PQKYGGRGYDSYQLCALHEVMAGVHGSLENLITVTGMVSTLLQRVGSAAQKA |
| NO: 1089 | HYLPKLATGELIGAIALTEPNIGSDLVNVETELQQDGDGWRLNGKKKWITLG |
| | QIADFFIVLIHCGNQLATVLIDRNTDGFTITPLNDMLGLRGNMLAELHFNDC |
| | RLKEDALLGPLTPGVPLAVNFALNEGRFTTACGSLGLCRAAVDVAARYIRQR |
| | KQFKRRLFSHGIVQHLFATMLTQTRSAQLMCFSAAEYRETLHPAMINQILMA |
| | KYVASKAAVDVAGKAVQLLGANGCHADYAVERYYRDAKIMEIIEGTSQIHEI |
| | QIAMNYMMGSEA* |
| clbG | MTKDVALMFPGSGSQYVGMARWLYERYPQVRTLFDEASQITERDMAALCLSG |
| SEQ ID | TLVQLAEPTAMALAIYTTSVAHFVAWQQFLAQTGVHVNLRYMLGHSLGEYAA |
| NO: 1090 | LTCSGALSFSQALALVAMRSRLASEIAREMDASTTIIKQGNQALVAAACEVA |
| | ERETRQQVGIACFNSPQQFMLSGQNSAIIAAEQYLLDHDRQVEVVPLIGGVP |
| | YHSPLLKPCGQQLRKALDRCEWRRPCCPVISNVNAQPYPDTVTPVQWLEQQL |
| | SQPVQWQRSLTYLTGHLSPIAIEIGPQSVLKNLLLENRYPAPVYAFDNRHDR |
| | AQLALVLGDNMAVKTDPEAVRRQRITLLTNALTATRHHRAADVAASAQLKEL |
| | LSRFFERIQQIEQRGTSSEEDIAFLHELLEQGFQLKGSSQAEIDACHARLAS |
| | DNGGQA* |
| clbH | MEQQGIMRQLPTDDQTIVDYLYRIAGEYGEKAAVLMGDAALSYHDLNARSNQ |
| SEQ ID | LAHYLRGLGIGEDRVVAIRLPRGMAMLIAIFAIVKAGGAYLPLAYNAPRSRI |
| NO: 1091 | ENILSNSGAVCLIGTDDGDRWPIPRVEIDSAAVSAMPTTDLRYRPHARQLAY |
| | IIYTSGSTGVPKGVATEHAALLNRIVWMQNAYPISSQDVLFQKTVYTFDVSV |
| | WEMFWWAMYGASVVLLPSGLESDPRTLARLIQRHRVSVVHFVPSMLNLFVEY |
| | LEMKQDPRLTASLRLVFSSGEKLTVHSVARFYQSVAQGDLINLYGPTEAAID |
| | VSHHRCLRGYDYDDIPIGQAIDGCRLYVLDDHGNPVADGEEGELYLAGIGLA |
| | RGYLNNVALTDRCFTIHPTLRHLGKPERLYKTGDLVWRDGESQQIHYIGRND |
| | FQIKIRGLRVELGEIEAHAMRFPGVQQAVVVADQDDPDNQLIYAFVVSSVPL |
| | NLAALMDALSKNLPAYMLPNRLLAMSELPLSDNGKCCRKTLLDLARAYSASR |
| | VDLRETPAVRYLPLSSAQSSMWFMQQLAPHTALYNNPTALLLEGELDRTRMD |
| | GAIRQLMSRHTLLRAMAETHNGQPVLAVPQCVSSQALLTIVPLPSVSDDNAL |
| | QAMINQRAAHPMPLTSGTPLCRFELLTLDDDRSVLLIHLHHIISDGWSKGVL |
| | LRELQAAYNGESLTPEPLLEYADYMEYQEEWRQSDAYQDAMRYWQNTLAGTL |
| | PILDIPTDQPRQKVARYQGAFVAFALSANTCERVLAAARAQRVSLYNYLLTA |
| | FVLLLHRNARQQEYIVGMPIAARLTKEQEHMIAPLVNVLPLRLPLDEAASFS |
| | ELVQTIRGILFAAFRHQRLEFTDIVRAVNVDRSAGTPCALLPDMQAYLLFTS |
| | GSTGEPKGVCVVHRGLLNLLLDMQRTFAVGSQDRLLSVTTPTFDISFLEYLL |
| | PLISGASLYLTEAERAADSFRMIPLIADYRPTLMQATPSFWHGLLMAGWRGD |
| | PELCVLAGGEALPTKVAEELLRCCGSLWNLYGPTETTIWSLKSQITQAENIT |
| | LGAPIANTRIYILDNEGHPVPQGVDGELYIAGDGVAQGYDGQPELNAQFFLS |
| | EPGVPGGRMFRTGDLVRSDAQGQLFFVGRKDSQIKLRGYRIELGEIERTLAR |
| | HPHVDAAVVACIERAPLHKALAAFIITSEPPSLFEQLKNELRQQLPDYMVPT |
| | LWQRVADFPNTDNGKIDRKRLAENFVADSSLVSPQTQALSDTEQMLLALWMR |
| | YLPIKNVDPECDFFRLGGHSLLAVTLVAEINRTFHCALTLKDIFHYSTLRAL |
| | SARIAQQSITDAAASQDDWVIVHDPEHRHQPFPLTDVQRAYWLGRQTGATSI |
| | ATHIYHEFDVEHFNVTRFTHAVNALIARHEMLRARVLPDGTQQILAQVPAYQ |
| | LEQRDLSALSPNARNDALMAIRDRLSHHVHPADRWPLFDFSYSACTAQHGRL |
| | HFSLDLLIADALSMRTLQQELMMLYREPHVSLPLLPFSFRDYVQALLVEQAS |
| | EAYARDQAYWQRALPQLYGPPTLPVQGDLAQLSAIRFVRRRHRLSAHNWGVL |
| | SALAQRTRITKTALLLTVFHFPIYQCMFQLDNMPLASPTLNGVNVTPLLLDT |
| | SASQVDISLSMQHIDGRITGTFEYDAGLYSADRIQHLVAQWRELLDEASSQP |
| | TQLVRDLIRFTPREHAWLARHNATEVALPPVDNLLALVLPHCQQRPTQVALR |
| | HADDAMTYGELQQATMQMCTWLRAQGVKRGESVALQLPFCFELIIAQLAILS |
| | LGASYVPLDGNAPAARNALILAQATPCMLLVAQPLESPHGLTIPWVLVPDWR |
| | SLLTEIPNLPVSVAPDALDCDAVVIFTSGTTGQPKGVRLSQRNLVNLTASFI |
| | SSYQVTHQDVLLPITSVASASFVGEVLPLLAAGGTLVLAQKAQSLDSDALIA |
| | LLASQRVTILSTTPSLSASLSVLAQSMGSLRLFLCGGEALEYEQIAPLLPHM |
| | AVVNGYGLTESGICSTYFPVAKRREQETGALPIGRPIQNTQAYVVDAYNRLV |
| | PPGACGELCFSGLGISPGYLDARQDPERFVELPEYPGVRVLKTGDRARWATD |
| | GMLFYLGRQDRQVQIRGYRVELGDIESLLKQHPDIADAWVDVRRNAAATPLL |
| | VAFYCSVNGVALDAQQLRVWLSLRLPLHMLPLLYVPLSAMPLGVNGKIDPQC |
| | LPLVDLRQLEGPGEYVPPATELEQRLAEIWQQLLGLERVGTTTNFFDLGGHS |
| | LLLVQMQQYIGQQCGQHVALVDLLRFTTIKRLAEFLLAPDAAQGTTGDQTQL |
| | RAAKQRLAFGHTRWAATTDSHH* |
| clbI | MAENDFGIAIIGMAGRFPQADTVQAFWENLLASRECISFYSDEELLAMGISP |
| SEQ ID | EFVQHPDYVKAKGEVADIDKFDAAFFGIAPREAELMDPQHRVLLETAWAAFE |
| NO: 1092 | DAGYVAADYPGDVGIFAGKSMDSYLMLNLMPHFKRVFSSGSLQAAIGNDKDS |
| | ITTTIAYHLNLRGPAITVQTSSSTSLVAVCVACQSLLTWQCDMAIAGGVTLG |
| | PPAKTGYLSQEGGITAADGHCRAFSDNSSGFVPGTGAGLVVLKRVDEALRDG |
| | DNIYAVIKGFAVNNDGSEKISYTAPSVDAQARAIAQAQRLAGLTPQDITYVE |
| | AHGTGTRLGDPVEFSALSQAFAGASQKQYCALGSVKTNIGHLDTAAGVAGLI |
| | KTALAVQQGIIPATLHFERPNAQIDLTNSPFYINTTCQPWQPESGIRRAGVT |
| | SLGMGGTNAHVVLEQAPAVDLQARAPVPAYSILPFSAKTDSALSSGLARFAD |
| | FLQHESLPDRRDLAWTLSQGRKAFAHRAALVTRDLHAAGTLLQQAATAPFAR |
| | GVAQTQLGLGLLFSGQGSQYQRMGHQLYQVWPAYADAFDRCATLLEREYQLD |
| | IRHELFRAEVSLAQGERLAQTCLTQPLLFSVEYALAQLWLSWGITPTVMIGH |
| | SLGEWVAATLAGVFSLEDALRLVARRAELMHQAPSGAMLMVALPEAQIRALI |
| | TAPLAIAAVNAPDYSVIAGPTSEILAVSQRLTEQNIINKRLHTSHAFHSSMM |
| | QDAAQALRQAFENVRLNPPTLTIISTVTGAHVSADTLTTPDYWIEQMLMPVQ |
| | FSAALQEAQATFDVDFLEIGPGATLTQLTNGHALGDRLAFSSLPAGARSSDE |
| | HKHILDTVAALWVRGHNIDLSAFAGEQPRRVSLPTYAFDKIRYWVDSPEEQR |
| | SAVTPVADAGSVIPSEPSVRRQPRPAFSVPYAAPESKTQRGLVAICEALLGI |
| | DGLGIDDNFFEAGGHSLMLGMLLAQVQERFAVTLSFFDVMEDASVRALAQLV |
| | EQEQQDDGGSALAVLVNDMINE* |
| clbJ | MTIHHAALARMLPAEKKEKLLRQLAQSGVSPSRIPIIKADPAQAIPLSFNQE |
| SEQ ID | RLWFLQKYDSTATNYNLYVVYRLHGVVDMPMLTEALRHVQARHAILRTRIIV |
| NO: 1093 | RNDRPCQVIDDASSLVLDTVTLAAQAPTSALDAVIQQVINTRFDLARGPLWG |
| | VTQIIQPDQGCHLVFCAHHIIIDGISLRLLFDELQQQYARLHAGNETSLPPP |
| | PLQYADYAFWQREWFQDTLLANELAYWRARLODAPLLSTFPSLHPRPAQPST |
| | HGSRFSITLDETLSLALKHVARTQETTPFVLMLTAFQLVLMRYAQQQRLVIG |
| | MPVSGRIRPELQSSIGYYASTAVIYTDFNGVEVGREALQRVKASVKETQGRQ |
| | QLPFENLVNMLDLPRSLSHSPLFQILYIYHNHVTPRAFTLAGAYWEQVTYHN |
| | QTVKYDMTVEVFQNDATFDVSFEYDLGLYDADVVKQIAEALRQHCLSLTSSL |
| | ETPIGAIPLHAPETATPRRDPLNATNVPWLGPQDVLRIIEQRCVQHPKQLAI |
| | QQHDGTLTYAELWARVQFIAMRFRAHGIQPGDRIGVLLPRHRDVIATMLATW |
| | FVGACYVPFDIHQPAARLQRLMQRARLVCLVVRQPGEWGEIVQLSLPELMQD |
| | MSNAIRYSSQVLARWSLSPTFTLNLTLFNRPQGYPNAEAVIGDFTAVSLLNV |
| | CYDSQHSYAHNAQRIQVQLWEDLEHRRFSGIRASEALIHSGRFHAPMPVVFT |
| | SMLDIDGETTAQDPRDTTRFTLCPDANITQTPQVWLDHQVIELAGELHFNWD |
| | AVEQLFDTTLLDQMFGAYCHALQALVAMPQSWWGVNSSLALPTVSAPVTQAP |
| | APTALLHHGLLRQAALTPQETALISPIRELTYRQLSTAADHVARALLALGVQ |
| | HGDRVAVVMEKGWQQIAAVHGILRLGAVYLPVDPVLPPQRRQLLLTVGEVRV |
| | QVTQPGLTQLEPSLPVLIIDDGMLDTPAAPLPEVAGDVTDLAYIIFTSGSTG |
| | TPKGVMIDHRAAMNTLEDINERFGLNAQDRVFGLSSLSFDLSVYDAFAPFMV |
| | GAALVLPEAGREKDPRHWQTVMAHGHVSVWNAVPALMQMLCEYHSGDRMSYP |
| | TLRLALLSGDWIPLTLPEQMRERLNETMDIISLGGATECAIWSVYYPIGEVE |
| | STWTSIPYGRGLRNQPVYVLNAQLEECPVGVEGEICIGGMGLAQGYLNDAEK |
| | TAASFVWREASGERIYRTGDRGRYFADGQVAFLGRNDTQVKVNGYRIELGEV |
| | KSHLEQLDSVGSAAVVCHQGQLYAFITAAENLHPDDTDALLARVRAQLAVQL |
| | PYYLLPQHFFLLKVLPMTGNGKIDQAAMVQEVIQRMSQSTSQKSRALAHASP |
| | YEQQVAALWCEVLQREQIGLNDNFFEAGGGSIQIVLLHRRIEEIFKVTVPIA |
| | ELFRLTTVKRIAGYLQAMQDNARAVNQTQQRDASRSRAQQRLVRRHQRQR* |
| clbK | MTYSESDIAIVGMNCRYPGVHSVAAFETVLRTGCNILDPKVTPSNGHNHITL |
| SEQ ID | NNVYEHMAEFDANFFGYSRAEAEIMDPQQRVFLTCAWEMFEQSGYNPKQHDA |
| NO: 1094 | RVGLYAGVSTSFYLLTHLMNNPDKLAQLGGLQIMVGNDKDHLTSQLAYRLNI |
| | TGPCVTVQASCATSLVAVHLACEGLLSGQCDMALAGGVTFRMEEQRSYESHG |
| | DGLQAEDGLIHTFDAQASGTVYSSGLGMVLLKRATDAQVQGDNILAVIKGSA |
| | INNDGGARSGYTVPGVDGQEAVMIEAHSLAEVTPQQIQYLELHGSGTPLGDA |
| | IEFAAIKRVFGTPAPNATPWRLGAVKPNVGHVEMASGITSLIKTVLSLTNRV |
| | FYPTLNFQRANPQLGLEDSPFEVVSRLTPWPEGTTPRTAGVSAFGLGGTNAH |
| | LVVQAPLSTPQARAQQMGPCVVVLSAKNHNALEQMQNALLAKLAAHPEIRLQ |
| | DVAYTLRHGRFSAPVRKCVIAENCTQLARQLRDAPMVEATTGCTIYWRLGHR |
| | FVVALETLSDWLACSEVLSQAVGQLLEHFPLEPACLQDLSPAQRTFISQYAL |
| | IALIDERETLNVVLCGDGDGGYAAAVLRGDCTLEQAWHRLNAGQPFDDVPTN |
| | PLLQPDVCSLMLDDAASDANRTALEALGQLWLAGVSLDWRWVDAAERMLGSQ |
| | RIALPGTVFTPQRYWVEAVRPATFSHESSNNLLSRATKSDIIAVVTEIWERT |
| | LGVSIDDHHASFFELGGHSLLASTILYDIQQRYGITCTLSAFFADPTIEGLS |
| | CYLLEQGGSETAVSALPDTVFAPDQQHLPFPLTDVQQAYWVGRRKSLGLGNI |
| | STHIYVEYELQGLDETAFNRALNAVIARHSMLRAIVNDDGMQQILPNVPEYH |
| | VAFYTTQCEDAFQQRCRELRDTLSHQMIDCSRWPLFQMEVVVDPQQKARLHV |
| | SIDLLIADAWSLELFIRELAYHYRHPQAALPTLTYSFRDYVLTLKSYEKTPQ |
| | FERARDYWRARIETLPPGPRLPLRTDPTKLENPTFVRRSYCLSRAIWQRLKT |
| | QAGQMSITPTTLLLTGFAQVLARFSSSPHFSLNLTLFNRLPLHADINHLIGD |
| | FTALTLLEIDMSQGETLQARANVIHSQLWRDLDNRLEGGIQVSRLLVQTHRD |
| | PAKSVIPIVFTSLLNQYEASWETDDTLFNQPQDDLYSISQTPQVWLDHQVME |
| | RNGELHFNWDVVEQLFEPALMDQMFQCYCQLLHALAQRPQLWHETQDVLALP |
| | TVSAPVTQAPAPTALLHHGLLRQAALTPQETALISPIRELTYRQLSTAADHV |
| | ARALLALGVQHGDRVAVVMEKGWQQIAAVHGILRLGAVYLPVDPVLPPQRRQ |
| | LLLTVGEVRVQVTQPGLTQLEPSLPVLIIDDGMLDTPAAPLPEVAGDVTDLA |
| | YIIFTSGSTGTPKGVMIDHRAAMNTLEDINERFGLNAQDRVFGLSSLSFDLS |
| | VYDAFAPFMVGAALVLPEAGREKDPRHWQTVMAHGHVSVWNAVPALMQMLCE |
| | YHSGDRMSYPTLRLALLSGDWIPLTLPEQMRERLNETMDIISLGGATECAIW |
| | SVYYPIGEVESTWTSIPYGRGLRNQPVYVLNAQLEECPVGVEGEICIGGMGL |
| | AQGYLNDAEKTAASFVWREASGERIYRTGDRGRYFADGQVAFLGRNDTQVKV |
| | NGYRIELGEIERCIARHPDVEQSVVVAVGNSQHRRLVAFAKLHDRHQAQALQ |
| | AKEAEAAALAQGIIVNPAQRLAFKLKEPHIRALDGLGIALTAPADSTRYIKR |
| | RSYRHFSAQKTTLAQLGQLLSGLGQMRLPGLPFAKYAYASAGGLYPVQTYVY |
| | LHPDKIEEGVSGIYYFDPRQSCLMPVAPEVELNSGFHAGPNQSIADRAAFTL |
| | FMVADMAVISPFYGQEAAWHFSVMEAGTLCHLLEEDAPRYGLGLCQLGMADF |
| | SAVASHFQLSPHHRYVHCTVGGAIGQEAASAAALLRDFSTYEKPKETAAPLD |
| | MQSYKDAMLRGLRQQLPDYMVPSDLMLATDFPLTANGKLDRQKLQLQGEQIA |
| | HQRDGVGPIQVDSALQQRLVALWQEVLGVSHVSAEDDFFSLGGSSIELVRIQ |
| | QALEAIIGQEIPIVDLFRLPTIADVARYLDEQLHNLPAAHDIVLAQAEVSQV |
| | SAARENLALRRKRAQQGEKGDE* |
| clbL | MSEQSYRSAGTLLAQLASGETTSVALVNHYFSRMAQFNKPLNAVVQQHYALA |
| SEQ ID | LEAAARADRERLEGRARGVLHGLPCTVKESFDVQGWLTTSGAHYLKDNRATQ |
| NO: 1095 | DAPSIARLRAAGAILMGKTNVPMMTADWQTYNDLYGTTHNLWDRQRSPGGSS |
| | GGAAVAVAADFTPVEFGSDLFGSLRIPAHYTGVYAHRCSLGLMSVRGHVPGG |
| | GPQATDEPDLSTAGPMARSAADLRLMMRALSTFWVEPPRIPDFSRYQAKANY |
| | RVCTWFSAPHHEIDQQIAQRFQSFIDKLRAQPGVEVDDAMPADIDPDALFDI |
| | AVKLSGRLVSTALNGRQRLTAGLAALGFRLVGKLADVPEGITSYYQGMLKDS |
| | GEQRNTDKLRHEYSRVIETLFARYDVLLTPVSPVLAFAHMQQPVRKRKLIVN |
| | GEPQDYNEHLFWNMLATVFGLPATVYPLAKTMDELPCGIQIISGHFHDDVTI |
| | NFAEFCESISGGFTVPEGY* |
| clbM | MTVNKLEPDNGTPNDELFTVAGMFDGSLYKLLLRMALPMFVGMLTQVTYAIA |
| SEQ ID | DIFWLSHIDVTNSGIIAGVGLVFPVGMGLFAIANGIQIGMGSLLSRAIGMQR |
| NO: 1096 | LDRAQRILSVGIIIALFFAIVITVLGYVYAQPLLRSLGATKSIIGYATEFYY |
| | YSLLTVFSIMLIGVMMGLFQGAGKIMVIMKASLLGALVNIMLDPIMIFVFDF |
| | GVKGVALASFLAQLSMVAYFIYTLMGLHIGLSIRIALRPFSWKIYREFLSVG |
| | MAQMLMQLIIAVGIVIYNFFIVRLDVNAMAAFTLTGRIDYFIITPMLAIATA |
| | LLTVVGQNWGHGNVTRTLNAYWAAVALAFSIVLVLAVMHIVLAPWMYPLFTR |
| | VVAVSDYAVLQTRIMALALPFVAISLLASEYYQAIGKPWYSVLLTLMRHVFI |
| | SVPVVYLLAIVLEMRITGVYFGAMSGTFVAALLAWRLLRLSPRLLRWNQEAV |
| | RSQHLDMEVAP* |
| clbN | MMSGNPLSWPQEQCHIIDQLYPYSAVNIIGGVVTIEGIVDLPRLHAAIQSAI |
| SEQ ID | RQFDALRMWFVMGEESEVVSQVQPYHWRDIRHLTFSPDYDKENLRPAAIETF |
| NO: 1097 | VDEWFRQPFTLLAHDLFEFVTFTCGEQYSGYLFKAHHGIADGWSMALLSNHV |
| | KRAYEQQDVPDDASPAYSAFLAQQQSYQASTRFAVDRGWWRDYIDEYRDCFP |
| | DSSPIVTTEGISCSTWLEPAMINRLYRLCNRYGCTLNTLFIALFALYRARVW |
| | GEEKGVIGVPLANRHTREARRCFGMFTNQLPLAYRLVRTERFCERVAFFQRE |
| | LKRGFKHSKYPITLFNQDLAEQGGGKLRAFDYCVNYYNFTYERHIAGAAQRV |
| | ESYYSGEQSYKLQIVLQTVNNHKESLRLSLEALRSAFTPHQLTAMKNGLLDL |
| | VTALDRQPDARLGDLEVYPAPHVALACGSLKPSFTSRFAAQVVEHGDRTALI |
| | DNEQSLTYRQLDDAVERVARYLRQQGIGRGQVVGIIAEHSAQTVMVIYGILR |
| | CGAAFLPLNPALPTTRLYAMCRKAQVAHILYDPAMHELTQALAFPASSLLQA |
| | LATSALAREPWPAIEPQDLAYVLFTSGSTGEPKGVQVSHGNLANYLHFAAER |
| | YFTAQDRAALYSSLSFDLTITTLFAPLCVGASISVCRHAESETLLRMAVVDQ |
| | PNTVIKLTPAHLRLLCAAGISSEQIRTLVVGGEDFKRDLARKAAALFPQAVI |
| | YNEYGPTEATVGCMIYRYTGQETLPSLPIGMAIDGCQVAICSPWGCPVPEGE |
| | TGELVIYGASVTQGYIDAPQQTAAAYLKDTNGVMIGYRSGDIGYAIAPNTLV |
| | YQGRKDDQVKINGYRIELCEIEQALLSAPQVESAAVAVIDDVQGQHSGLLAC |
| | VTPSSVDVATVMQHLRQQLPTYMQPKQCCAIAQLPLSHNGKVDVRQMVATVR |
| | NTAPASGSERLGDAAIRHSVRVCVEGALEQTEFDDNENLYVLGLDSIKSIQI |
| | AAQLRHHGWTMSAVQVMECGTVNAICEFLASHTTVSQLAQYAHNTRIDLPAL |
| | RWFTQLALPVPNVYNHVIVLKVLPGCPLEQLHNRLHTLIQQQPALHSALDAE |
| | GRLLVCDPNVCYPNEVLTEYSTAQWTLAEVIAQCNSMLDVTNGRVFTAALLH |
| | APQPASSTLVLCAHHLCVDMHSWYLILSTLDAVSTVNGTSNSGLHRWNDYLA |
| | SKTVDSATHESWRTVCQTLPLHFPPVSLPDDSLPRTRAWREDFRHPCVRRLF |
| | ESSGNTAYSAETYVLTALALVLRYYSEEPWCRIEMEGMGRGCWPDEPDVADT |
| | VGWFTLFYPWAIPLHGDMATLLSAIASDLAKRTHGGGDYGLLQMRHAPEDSL |
| | AQGIRMNYIGVQAQPSLRYFHIDHFNSDIYTAPENALGCVLEFNIARSAADG |
| | LSFHCRFDPTRIALNDVQLLLARYKNSLTDLDAWLCQHSATLTGAPTLWTL* |
| clbO | MAKDDFTCGSLDIAIIGMSGRFSGAESVPEWWDKLLAGEEFTQPTCTEDDNG |
| SEQ ID | NPWIRLRNIITAPYDFDAAFFNIPPGEALLMDPQQRIFLECCYNALEHAGYI |
| NO: 1098 | PTQLKRVGVYGATYANNYFIDRVYPYLKMSGDHHYLQAQIGNEKDYLCAQVA |
| | YKLGFTGPAVSVQTACSSSLVAAYLACEGLLTFQADVALAGGVTLGFLQAHG |
| | YSPQGDKLVSQDGHCAPFSAEATGTVYSSGAGVVVLKRLEDALRDQDRVYAV |
| | IKGGAVNNDGGRRLGFVAPSVEGQVEAINTALAAAEVVPTDIALIETHGTGT |
| | PLGDEIELEALHRVFAPACAPHSIQLGAVKANLGHLGVASGIVSLMKTALTL |
| | YTGLVPPQINLVNKHKKLLQPASPFYLSDVVTSVPQTKRIHATVSSFGLGGT |
| | NAHLVLQNWCETPAQAVQENERRLFFFSAKTPLALRQQLDAHYHALATYAEA |
| | DKDRIAYTLAQRRAHFPYRCALAADSVVALRASLAKLRDADMSFTPINMETT |
| | LVFLYPDRDDKLESALTHLLACQPNLRQRHQRLSQDVAQICEPADWTPALRQ |
| | FIQQVSLSEWLIEQSISPVQHIGYLTGAAAAQYVARIISLENAVQQVIVAET |
| | TPEQTLAGNSELSEILANLAVTEGTLMLEIGRAGTFSILYHQHAQWVGQTVF |
| | SPMLNTDTPEDILPLLGTLWQRGVTICLPEMPAVQTIGLPGYSFDRVRYEIQ |
| | SSDARENAMLPVSYLSVSDFVEKTWRSLLCIDHYDEHAVIFEYGATSMHVIS |
| | FVDSCNHIYKIGLTAADIYARPAIREHSEFISECVDGIL* |
| clbP | MTIMEHVSIKTLYHLLCCMLLFISAMCALAQEHEPIGAQDERLSTLIHQRMQ |
| SEQ ID | EAKVPALSVSVTIKGVRQRFVYGVADVASQKANTLDTVYELGSMSKAFTGLV |
| NO: 1099 | VQILIQEGRLRQGDDIITYLPEMRLNYQGKPASLTVADFLYHTSGLPFSTLA |
| | RLENPMPGSAVAQQLRNENLLFAPGAKFSYASANYDVLGAVIENVTGKTFTE |
| | VIAERLTQPLGMSATVAVKGDEIIVNKASGYKLGFGKPVLFHAPLARNHVPA |
| | AYIHSTLPDMEIWIDAWLHRKALPATLREAMSNSWRGNSDVPLAADNRILYA |
| | SGWFIDQNQGPYISHGGQNPNFSSCIALRPDQQIGIVALANMNSNLILQLCA |
| | DIDNYLRIGKYADGAGDAITATDTLFVYLTLLLCFWGAVVVVRGAFRVYRAT |
| | AHGPGKQQRLRLRVRDYIIALAVPGLVAAMLYVAPGILSPGLDWRFILVWGP |
| | SSVLAIPFGIILLAFVLTLNHQIKRILLHNKEWDDE* |
| clbQ | MSNISLYCLPYSGGSAAMYYKWRSVLSDNITLRPLEPAGRGTRIRQPLCLTM |
| SEQ ID | VDAVADLYQQFVKHYTGGDYAIFGHSLGGIMAFELVHYILDHGHDMPCALFF |
| NO: 1100 | SGCRPPDRASHEVILHTLPDQAFMEEIVKLGGTPVDVFRNKELMTIFTPIIK |
| | NDYRLYEQYVFQAKARTLTCPIVLFHGDADNLVMQDELLAWEKFTTRKTRTI |
| | IFPAADHFFVDKHFEQVVGYVNQTIESLEIVG* |
| clbR | MDKFKEKNPLSLRERQVLRMLAQGDEYSQISHNLNISINTVKFHVKNIKHKI |
| SEQ ID | QARNTNHAIHIANRNEII* |
| NO: 1101 | |
| clbS | MAVPSSKEELIKAINSNFSLLNKKLESITPQLAFEPLLEGHAKGTTISVANL |
| SEQ ID | VSYLIGWGELVLHWHDQEAKGKTIIFPEEGFKWNELGRLAQKFYRDYEDITE |
| NO: 1102 | YEVLLARLKENKQQLVALIERFSNDELYGKPWYNKWTRGRMIQFNTASPYKN |
| | ASGRLNKLQKCLAE* |

### Secreted EGF Polypeptides

In some embodiments, the recombinant bacteria are capable of producing EGF, particularly human EGF. EGF mediates signaling pathways by binding Epidermal Growth Factor Receptor (EGFR). The binding of EGF to EGFR promotes phosphorylation of EGFR and subsequent phosphorylation of AKT and ERK. Once cleaved and secreted, EGF functions as a cytokine to ameliorate inflammatory signals. For example, EGF can enhance goblet cell-associated mucosal integrity, while diminished EGF can be associated with inflammatory disorders. EGF plays a role in inflammatory bowel disease, with murine and human studies suggesting a protective and restorative role in disease pathogenesis and activity.

In some embodiments, the recombinant bacteria comprise a nucleic acid sequence encoding a polypeptide of Sequence A or a functional fragment thereof. In some embodiments, recombinant bacteria comprise a nucleic acid sequence that is at least about 80%, 85%, 90%, 95%, or 99% homologous to a nucleic acid sequence encoding a polypeptide of Sequence A or a functional fragment thereof.

In some embodiments, the recombinant bacteria comprise a nucleic acid sequence encoding a fusion protein which comprises a polypeptide tag of Sequence C (below) or a functional fragment thereof. In some embodiments, recombinant bacteria comprise a gene sequence encoding a fusion protein which comprises a polypeptide tag of that is at least about 80%, 85%, 90%, 95%, or 99% identity to a polypeptide tag comprising Sequence C or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with Sequence C. In some specific embodiments, the polypeptide tag comprises Sequence C.

Sequence C (PelB):
MKYLLPTAAAGLLLLAAQPAMA

In some embodiments, the recombinant bacteria comprise a nucleic acid sequence encoding a fusion protein which comprises a polypeptide tag of Sequence D (below) or a functional fragment thereof. In some embodiments, recombinant bacteria comprise a nucleic acid sequence encoding a fusion protein which comprises a polypeptide tag of that is at least about 80%, 85%, 90%, 95%, or 99% identity to a polypeptide tag comprising Sequence D or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with Sequence D. In some specific embodiments, the polypeptide tag comprises Sequence D.

Sequence D (PhoA):
MKQSTIALALLPLLFTPVTKA

In some embodiments, the recombinant bacteria comprise a nucleic acid sequence encoding a fusion protein which comprises a polypeptide tag of Sequence E (below) or a functional fragment thereof. In some embodiments, recombinant bacteria comprise a gene sequence encoding a fusion protein which comprises a polypeptide tag of that is at least about 80%, 85%, 90%, 95%, or 99% identity to a polypeptide tag comprising Sequence E or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with Sequence E. In some specific embodiments, the polypeptide tag comprises Sequence E.

Sequence E (OmpA):
MKKTAIAIAVALAGFATVAQA

In some embodiments, the recombinant bacteria comprise a nucleic acid sequence encoding a fusion protein which comprises a polypeptide tag of Sequence F (below) or a functional fragment thereof. In some embodiments, recombinant bacteria comprise a gene sequence encoding a fusion protein which comprises a polypeptide tag of that is at least about 80%, 85%, 90%, 95%, or 99% identity to a polypeptide tag comprising Sequence F or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with Sequence F. In some specific embodiments, the polypeptide tag comprises Sequence F.

Sequence F (LARD3):
IEGRGSDGNDLIQGGKGADFIEGGKGNDTIRDNSGHNTFLFSGHFGQDRI
IGYQPTDRLVFQGADGSTDLRDHAKAVGADTVLSFGADSVTLVGVGLGGL
WSEGVLIS

In some embodiments, the recombinant bacteria comprise a nucleic acid sequence encoding a fusion protein which comprises a polypeptide tag of Sequence G (below) or a functional fragment thereof. In some embodiments, recombinant bacteria comprise a gene sequence encoding a fusion protein which comprises a polypeptide tag of that is at least about 80%, 85%, 90%, 95%, or 99% identity to a polypeptide tag comprising Sequence G or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with Sequence G. In some specific embodiments, the polypeptide tag comprises Sequence G.

Sequence G (HylA):
STYGSQDNLNPLINEISKIISAAGNFDVKEERSAASLLQLSGNASDFSYG
RNSITLTASA

In some embodiments, the recombinant bacteria comprise a nucleic acid sequence encoding an PelB-EGF fusion protein of (Sequence H below) or a functional fragment thereof. In some embodiments, recombinant bacteria comprise a gene sequence encoding a PelB-EGF fusion protien that is at least about 80%, 85%, 90%, 95%, or 99% identity to PelB-EGF fusion protein comprising Sequence H or a functional fragment thereof. In some embodiments, the PelB-EGF fusion protein has at least about 85%, 86%, 87%, 88%, 89%, 90%, 91%, 92%, 93%, 94%, 95%, 96%, 97%, 98%, or 99% identity with Sequence H. In some specific embodiments, the PelB-EGF fusion protein comprises Sequence F.

Sequence H (PelB-EGF):
MKYLLPTAAAGLLLLAAQPAMANSDSECPLSHDGYCLHDGVCMYIEALDK
YACNCVVGYIGERCQYRDLKWWELR

In some embodiments, the recombinant bacteria comprise nucleic acid sequence encoding an PhoA-EGF fusion protein of (Sequence I below) or a functional fragment thereof. In some embodiments, recombinant bacteria comprise a gene sequence encoding a PhoA-EGF fusion protein that is at least about 80%, 85%, 90%, 95%, or 99% identity to PhoA-EGF fusion protein comprising Sequence I or a functional fragment thereof. In some embodiments, the PhoA-EGF fusion protein has at least about 85%, 86%, 87%, 88%, 89%, 90%, 91%, 92%, 93%, 94%, 95%, 96%, 97%, 98%, or 99% identity with Sequence I. In some specific embodiments, the PhoA-EGF fusion protein comprises Sequence I.

Sequence I (PhoA-EGF):
MKQSTIALALLPLLFTPVTKANSDSECPLSHDGYCLHDGVCMYIEALDKY
ACNCVVGYIGERCQYRDLKWWELR

In some embodiments, the recombinant bacteria comprise nucleic acid sequence encoding an OmpA-EGF fusion protein of (Sequence J below) or a functional fragment thereof. In some embodiments, recombinant bacteria comprise a gene sequence encoding a OmpA-EGF fusion protein that is at least about 80%, 85%, 90%, 95%, or 99% identity to OmpA-EGF fusion protein comprising Sequence J or a functional fragment thereof. In some embodiments, the OmpA-EGF fusion protein has at least about 85%, 86%, 87%, 88%, 89%, 90%, 91%, 92%, 93%, 94%, 95%, 96%, 97%, 98%, or 99% identity with Sequence J. In some specific embodiments, the OmpA-EGF fusion protein comprises Sequence J.

Sequence J (OmpA-EGF):
MKKTAIAIAVALAGFATVAQANSDSECPLSHDGYCLHDGVCMYIEALDKY
ACNCVVGYIGERCQYRDLKWWELR

In some embodiments, the recombinant bacteria comprise nucleic acid sequence encoding an EGF-LARD3 fusion protein of (Sequence K below) or a functional fragment thereof. In some embodiments, recombinant bacteria comprise a gene sequence encoding an EGF-LARD3 fusion protein that is at least about 80%, 85%, 90%, 95%, or 99% identity to EGF-LARD3 fusion protein comprising Sequence K or a functional fragment thereof. In some embodiments, the EGF-LARD3 fusion protein has at least about 85%, 86%, 87%, 88%, 89%, 90%, 91%, 92%, 93%, 94%, 95%, 96%, 97%, 98%, or 99% identity with Sequence K. In some specific embodiments, the EGF-LARD3 fusion protein comprises Sequence K.

Sequence K (EGF-LAKDS):
MNSDSECPLSHDGYCLHDGVCMYIEALDKYACNCVVGYIGERCQYRDLKW
WELRIEGRGSDGNDLIQGGKGADFIEGGKGNDTIRDNSGHNTFLFSGHFG
QDRIIGYQPTDRLVFQGADGSTDLRDHAKAVGADTVLSFGADSVTLVGVG
LGGLWSEGVLIS

In some embodiments, the recombinant bacteria comprise nucleic acid sequence encoding an OmpA-EGF fusion protein of (Sequence L below) or a functional fragment thereof. In some embodiments, recombinant bacteria comprise a gene sequence encoding a OmpA-EGF fusion protein that is at least about 80%, 85%, 90%, 95%, or 99% identity to OmpA-EGF fusion protein comprising Sequence L or a functional fragment thereof. In some embodiments, the OmpA-EGF fusion protein has at least about 85%, 86%, 87%, 88%, 89%, 90%, 91%, 92%, 93%, 94%, 95%, 96%, 97%, 98%, or 99% identity with Sequence L. In some specific embodiments, the OmpA-EGF fusion protein comprises Sequence L.

Sequence L (EGF-HylA):
MNSDSECPLSHDGYCLHDGVCMYIEALDKYACNCVVGYIGERCQYRDLKW
WELRSTYGSQDNLNPLINEISKIISAAGNFDVKEERSAASLLQLSGNASD
FSYGRNSITLTASAIn

some embodiments, the recombinant bacteria comprise a nucleic acid sequence encoding a polypeptide set forth in Table 4 or a functional fragment thereof. In some embodiments, recombinant bacteria comprise a nucleic acid sequence that is at least about 80%, 85%, 90%, 95%, or 99% homologous to a nucleic acid sequence encoding a polypeptide set forth in Table 4 or a functional fragment thereof.

In some embodiments, the recombinant bacteria comprise a nucleic acid sequence set forth in Table 5 or a functional fragment thereof. In some embodiments, recombinant bacteria comprise a nucleic acid sequence that is at least about 80%, 85%, 90%, 95%, or 99% homologous to a nucleic acid sequence set forth in Table 5 or a functional fragment thereof.

TABLE 4
Non-limiting EGF constructs

| Description | SEQUENCE |
|---|---|
| Construct comprising | MKYLLPTAAAGLLLLAAQPAMANSDSECPLSHDGYCLHDGVCMYI |
| secretion tag pelB, | EALDKYACNCVVGYIGERCQYRDLKWWELR |
| and E. coli codon- | |
| optimized human | |
| EGF | |
| Construct comprising | MKKTAIAIAVALAGFATVAQANSDSECPLSHDGYCLHDGVCMYIE |
| secretion tag, OmpA, | ALDKYACNCVVGYIGERCQYRDLKWWELR |
| and E. coli codon- | |
| optimized human | |
| EGF | |
| Construct comprising | MKQSTIALALLPLLFTPVTKANSDSECPLSHDGYCLHDGVCMYIE |
| secretion tag, PhoA, | ALDKYACNCVVGYIGERCQYRDLKWWELR |
| and E. coli codon- | |
| optimized human | |
| EGF | |
| Construct comprising | MNSDSECPLSHDGYCLHDGVCMYIEALDKYACNCVVGYIGERCQY |
| E. coli codon- | RDLKWWELRIEGRGSDGNDLIQGGKGADFIEGGKGNDTIRDNSGH |
| optimized human | NTFLFSGHFGQDRIIGYQPTDRLVFQGADGSTDLRDHAKAVGADT |
| EGF and secretion | VLSFGADSVTLVGVGLGGLWSEGVLIS |
| tag, LARD3 | |
| Construct comprising | MNSDSECPLSHDGYCLHDGVCMYIEALDKYACNCVVGYIGERCQY |
| E. coli codon- | RDLKWWELRSTYGSQDNLNPLINEISKIISAAGNFDVKEERSAAS |
| optimized human | LLQLSGNASDFSYGRNSITLTASA |
| EGF and secretion | |
| tag, HylA | |
| pelB Secretion Tag | MKYLLPTAAAGLLLLAAQPAMA |
| OmpA Secretion Tag | MKKTAIAIAVALAGFATVAQA |
| PhoA Secretion Tag | MKQSTIALALLPLLFTPVTKA |
| LARD3 Secretion | IEGRGSDGNDLIQGGKGADFIEGGKGNDTIRDNSGHNTFLFSGHF |
| Tag | GQDRIIGYQPTDRLVFQGADGSTDLRDHAKAVGADTVLSFGADSV |
| | TLVGVGLGGLWSEGVLIS |
| HylA Secretion Tag | STYGSQDNLNPLINEISKIISAAGNFDVKEERSAASLLQLSGNAS |
| | DFSYGRNSITLTASA |
| E. coli codon- | NSDSECPLSHDGYCLHDGVCMYIEALDKYACNCVVGYIGERCQYR |
| optimized human | DLKWWELR |
| EGF | |

TABLE 5
Non-limiting EGF constructs

| Description | SEQUENCE |
|---|---|
| Construct | atgaaatatctgttgcccacggctgccgcgggtctgctgctgctgg |
| comprising | cagcgcaaccggctatggcaAATAGTGACAGCGAATGTCCGCTGTC |
| secretion tag pelB, | GCACGATGGTTATTGCCTTCATGATGGGGTGTGCATGTACATTGAG |
| and E. coli codon- | GCATTGGACAAATATGCCTGCAACTGTGTTGTCGGCTATATCGGCG |
| optimized human | AACGGTGTCAGTACCGTGATCTGAAATGGTGGGAATTACGCtaa |
| EGF | |
| Construct | ATGAAGAAAACCGCAATTGCAATCGCCGTCGCTCTGGCGGGGTTCG |
| comprising | CTACGGTCGCCCAAGCCAATAGTGACAGCGAATGTCCGCTGTCGCA |
| secretion tag, | CGATGGTTATTGCCTTCATGATGGGGTGTGCATGTACATTGAGGCA |
| OmpA, and E. coli | TTGGACAAATATGCCTGCAACTGTGTTGTCGGCTATATCGGCGAAC |
| codon-optimized | GGTGTCAGTACCGTGATCTGAAATGGTGGGAATTACGCtaa |
| human EGF | |
| Construct | atgaaacaaagcactattgcactggcactcttaccgttactgttta |
| comprising | cccctgtgacaaaagcgAATAGTGACAGCGAATGTCCGCTGTCGCA |
| secretion tag, | CGATGGTTATTGCCTTCATGATGGGGTGTGCATGTACATTGAGGCA |
| PhoA, and E. coli | TTGGACAAATATGCCTGCAACTGTGTTGTCGGCTATATCGGCGAAC |
| codon-optimized | GGTGTCAGTACCGTGATCTGAAATGGTGGGAATTACGCtaa |
| human EGF | |
| Construct | ATGAATAGTGACAGCGAATGTCCGCTGTCGCACGATGGTTATTGCC |
| comprising E. coli | TTCATGATGGGGTGTGCATGTACATTGAGGCATTGGACAAATATGC |
| codon-optimized | CTGCAACTGTGTTGTCGGCTATATCGGCGAACGGTGTCAGTACCGT |
| human EGF and | GATCTGAAATGGTGGGAATTACGCATTGAGGGCCGGggttctgacg |
| secretion tag, | gtaatgatcttatccaaggggtaagggcgcggacttcattgaaggc |
| LARD3 | ggcaaaggtaatgatacaatccgcgataactccggtcacaacacct |
| | ttttgttctcagggcattttggtcaggatcgtattataggatatca |
| | gccgaccgatcggctggtattccagggcgctgacggcagcacggat |
| | ctgcgcgaccatgcgaaagccgttggagcagatacggtgctgagtt |
| | ttggcgccgattcggttactctcgtcggggggggttaggaggcctg |
| | tggagcgagggtgtgctgattagttaa |
| Construct | ATGAATAGTGACAGCGAATGTCCGCTGTCGCACGATGGTTATTGCC |
| comprising E. coli | TTCATGATGGGGTGTGCATGTACATTGAGGCATTGGACAAATATGC |
| codon-optimized | CTGCAACTGTGTTGTCGGCTATATCGGCGAACGGTGTCAGTACCGT |
| human EGF and | GATCTGAAATGGTGGGAATTACGCtcaacttatgggagccaggaca |
| secretion tag, HylA | atcttaatccattaattaatgaaatcagcaaaatcatttcagctgc |
| | aggtaacttcgatgttaaggaggaaagatctgccgcttctttattg |
| | cagttgtccggtaatgccagtgatttttcatatggacggaactcaa |
| | taactttgacagcatcagcataa |
| pelB Secretion Tag | atgaaatatctgttgcccacggctgccgcgggtctgctgctgctgg |
| | cagcgcaaccggctatggca |
| OmpA Secretion | ATGAAGAAAACCGCAATTGCAATCGCCGTCGCTCTGGCGGGGTTCG |
| Tag | CTACGGTCGCCCAAGCC |
| PhoA Secretion | atgaaacaaagcactattgcactggcactcttaccgttactgttta |
| Tag | cccctgtgacaaaagcg |
| LARD3 Tag | attgagggccggggttctgacggtaatgatcttatccaaggcggta |
| | agggcgcggacttcattgaaggcggcaaaggtaatgatacaatccg |
| | cgataactccggtcacaacacctttttgttctcagggcattttggt |
| | caggatcgtattataggatatcagccgaccgatcggctggtattcc |
| | agggcgctgacggcagcacggatctgcgcgaccatgcgaaagccgt |
| | tggagcagatacggtgctgagttttggcgccgattcggttactctc |
| | gtcggggggggttaggaggcctgtggagcgagggtgtgctgattag |
| | ttaa |
| HlyA Tag | tcaacttatgggagccaggacaatcttaatccattaattaatgaaa |
| | tcagcaaaatcatttcagctgcaggtaacttcgatgttaaggagga |
| | aagatctgccgcttctttattgcagttgtccggtaatgccagtgat |
| | ttttcatatggacggaactcaataactttgacagcatcagcataa |
| E. coli codon- | AATAGTGACAGCGAATGTCCGCTGTCGCACGATGGTTATTGCCTTC |
| optimized human | ATGATGGGGTGTGCATGTACATTGAGGCATTGGACAAATATGCCTG |
| EGF | CAACTGTGTTGTCGGCTATATCGGCGAACGGTGTCAGTACCGTGAT |
| | CTGAAATGGTGGGAATTACGCTAA |

In some embodiments, the recombinant bacteria are capable of producing EGF under inducing conditions, e.g., under a condition(s) associated with inflammation. In some embodiments, the recombinant bacteria are capable of producing EGF in low-oxygen conditions.

In some embodiments, the recombinant bacteria comprise one or more gene sequence(s) selected from PhoA-EGF, OmpF-EGF, and TorA-EGF.

In some embodiments, the recombinant bacteria comprise gene(s) encoding an ATP binding cassette transporter or a portion thereof, e.g., one, two, three, or more subunits of an ATP binding cassette transporter as described herein. ATP binding cassette transporters are known in the art and described herein.

In some embodiments, the recombinant bacteria comprise nucleic acid sequence encoding an ATP binding cassette transporter of Sequence M.1, M.2, and/or M.3 below or a functional fragment thereof. In some embodiments, recombinant bacteria comprise a gene sequence encoding a ATP binding cassette transporter that is at least about 80%, 85%, 90%, 95%, or 99% identity to ATP binding cassette transporter comprising M.1, M.2, and/or M.3 or a functional fragment thereof. In some embodiments, the ATP binding cassette transporter has at least about 85%, 86%, 87%, 88%, 89%, 90%, 91%, 92%, 93%, 94%, 95%, 96%, 97%, 98%, or 99% identity with Sequence M.1, M.2, and/or M.3. In some specific embodiments, the ATP binding cassette transporter protein comprises Sequence M.1, M.2, and/or M.3.

Sequence M.1 (prtD)
MNASSERDRSLFGVLRQFRRSFWSVGIFSAVINVLMLAPSVYMLQVYDRV
LASGNGITLLMLTLLMAGLCAFMGALEWVRSLLVVRLGTRIDLALNQDVF
NAAFARNLEAGDGRAGLALTDLTLLRQFITGNALFAFFDVPWFPLFLLVL
FLLHPWLGMLALGGTVVLVALAWLNQHLTNQPLAEANQQSQQATHLADAQ
LRNADVIEAMGMLGNLRRRWLARHYRFISLQNLASERAAAVGGASKYSRI
ALQSLMLGLGALLAIDGKITPGMMIAGSILVGRVLSPIDQLIGVWKQWSS
ARIAWQRLTRLIAAYPPRPAAMALPAPEGHLSVEQVSLRTVQGNTRLQNI
HFSLQAGETLVILGASGSGKSSLARLLVGAQSPTQGKVRLDGADLNQVDK
NTFGPTIGYLPQDVQLFKGSLAENIARFGDADPEKVVAAAKLAGVHELIL
SLPNGYDTELGDGGGGLSGGQRQRIGLARAMYGDPCLLILDEPNASLDSE
GDQALMQAIVALQKRGATVVLITHRPALTTLAQKILILHEGQQQRMGLAR
DVLTELQQRSAANQARMNPTAAMPQ
Sequence M.2 (prtE)
MTGMDITTQDELNEAAMRDRASRDEERALRLGWWLVLAGFGGFLLWALLA
PLDKGVAVQGNVVVSGNRKVIQHMQGGIVDRIQVKDGDRVAAGQVLLTLN
AVDARTTSEGLGSQYDQLIAREARLLAEQRNQSSLAATPRLAQARQRPEM
AAIIALQEDLLRSRQQSLKLEIDGVRASIDGLETSLGALQKVMSSKQSEQ
ATLSQQLQGLRPLAADNYVPRNKMLETERLFAQVSGELAQTSGEVGRTRR
DIQQQKLRIAQRQQEYDKEVNSELSDVQAKLNEVISQREKADFNLANVQV
RAPVAGTVVDMKIFTEGGVIAPGQVMMDIVPEDQPLLVDGRIPVEMVDKV
WSGLPVELQFTAFSQSTTPRVPGTVTLLSADRLVDEKDGTPYYGLRIQVS
EEGKRSLHGLEIKPGMPVQGFVRTGERSFINYLFKPLMDRMHLALTEE
Sequence M.3 (prtF)
MRRKAVLLTVVLSLSCGSAQAMGLLDAWELALRNDAQLRAAGFERDAGQE
EVAIGRAGLLPSLQYTYGANYSHSKVTQRDRTLNNTTKRDYDNYVSTLTL
RQPLLDYAAWARYQQGVTRKLMADQRFRDRSQDLMVRLYQSWSEALLAQE
KLMLLDAQRRAYQEQLALNRRLLAAGEGTQTDLRETEARYTVTEAQRIEQ
EDTLDAAMTDLENMMGSPLQIQDLSPLALDTLPDNVTENRSLSQWRELTV
RHNAKLAVQRENVDYSRYEIERNRAGHLPTLDLVASTRNSLSESEYNYNQ
KYDTQTVGLQVRVPLYSGGAVSASMRQAAAEYQQSQAELDNQTRQTFAEL
RRQFNLCANGAAKIRAWQMSVAAAEEAIRATRQSVAGGERINLDVLMAEQ
EWYNARRELTEVKYRWLQAWLNLRYTAGTLNEQDMMQLAAWFQSAPVINK
TGINKTGINAATGNKTN

In some embodiments, the recombinant bacteria comprise a gene sequence encoding a linker fusion protein which comprises SEQ ID NO: 498 or a functional fragment thereof. In some embodiments, recombinant bacteria comprise a gene sequence encoding a linker polypeptide that has at least about 80%, 85%, 90%, 95%, or 99% identity to linker polypeptide comprising SEQ ID NO: 498 or a functional fragment thereof. In some embodiments, the linker polypeptide has at least about 85%, 86%, 87%, 88%, 89%, 90%, 91%, 92%, 93%, 94%, 95%, 96%, 97%, 98%, or 99% identity with SEQ ID NO: 498. In some specific embodiments, the linker polypeptide comprises SEQ ID NO: 498. In some embodiments, the gene sequence encoding ECOLIN 19410 secretion tag further comprises SEQ ID NO: 498. In some embodiments, the recombinant bacteria comprise gene sequence encoding a linker fusion protein. In certain embodiments, the linker fusion protein gene sequence has at least about 80%, 85%, 90%, 95%, or 99% identity with SEQ ID NO: 494. In some embodiments, the linker gene sequence has at least about 85%, 86%, 87%, 88%, 89%, 90%, 91%, 92%, 93%, 94%, 95%, 96%, 97%, 98%, or 99% identity with SEQ ID NO: 494. In some specific embodiments, the linker gene sequence comprises SEQ ID NO: 494.

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the PhoA secretion tag comprises SEQ ID NO: 385. In some embodiments, the PhoA secretion tag comprises SEQ ID NO: 386.

In some embodiments, the recombinant bacteria produce 0% to 2%, 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 80%, 80% to 90%, or 90% to 100% more EGF than unmodified bacteria of the same bacterial subtype under the same conditions. In yet another embodiment, the recombinant bacteria produce 1.0-1.2-fold, 1.2-1.4-fold, 1.4-1.6-fold, 1.6-1.8-fold, 1.8-2-fold, or two-fold more EGF than unmodified bacteria of the same bacterial subtype under the same conditions. In yet another embodiment, the recombinant bacteria produce three-fold, four-fold, five-fold, six-fold, seven-fold, eight-fold, nine-fold, ten-fold, fifteen-fold, twenty-fold, thirty-fold, forty-fold, or fifty-fold, more EGF, than unmodified bacteria of the same bacterial subtype under the same conditions.

In any of these embodiments, the EGF constructs described herein may further comprise a secretion tag. Non-limiting examples of secretion tags are described herein. The secretion tag is at the N-terminus or at the C-terminus.

In any of these embodiments, the recombinant bacteria may further comprise gene sequences encoding a secretion tag. Non-limiting examples of such secretion tags are described herein and include PhoA, OmpF, cvaC, TorA, fdnG, dmsA, PelB, the ECOLIN_05715 secretion signal, ECOLIN_16495 secretion signal, ECOLIN_19410 secretion signal, and the ECOLIN_19880 secretion signal. In some embodiments, the secretion tag is PhoA. In some embodiments, the secretion tag is ECOLIN 19410. The recombinant bacteria may further comprise one or more mutations to outer membrane proteins, i.e., to generate a diffusible outer membrane phenotype (DOM). Non-limiting examples of such outer membrane proteins are described herein and include lpp, nlP, tolA, and pal. In one embodiment, the recombinant bacteria comprise a deletion or mutation in pal. In some embodiments, the recombinant bacterium, e.g., Gram-negative bacterium, e.g., E. coli Nissle, may be engineered by deleting the gene encoding the periplasmic protein pal to create a diffusible outer membrane (DOM) phenotype, e.g., to result in a “leaky membrane” bacterium and increase the rate of diffusion of periplasmic proteins to the external environment without compromising cell growth properties.

In some embodiments, the genetically engineered are capable of producing and secreting EGF. In some embodiments, the EGF gene is functionally replaced, modified, and/or mutated in order to enhance stability and/or increase EGF production or secretion. In some embodiments, the recombinant bacteria are capable of expressing and secreting EGF in low-oxygen conditions, in the presence of certain molecules or metabolites, in the presence of molecules or metabolites associated with inflammation or an inflammatory response, or in the presence of some other metabolite that may or may not be present in the gut, such as arabinose. Exemplary chemical inducers are described herein.

In one embodiment, the EGF gene is directly operably linked to a first promoter. In another embodiment, the EGF gene is indirectly operably linked to a first promoter. In one embodiment, the promoter is not operably linked with the EGF gene in nature.

In some embodiments, the EGF gene is expressed under the control of a constitutive promoter. In another embodiment, the EGF gene is expressed under the control of an inducible promoter. In some embodiments, the EGF gene is expressed under the control of a promoter that is directly or indirectly induced by exogenous environmental conditions. In one embodiment, the EGF gene is expressed under the control of a promoter that is directly or indirectly induced by low-oxygen or anaerobic conditions, wherein expression of the EGF gene is activated under low-oxygen or anaerobic environments, such as the environment of the mammalian gut. In one embodiment, the EGF gene is expressed under the control of a temperature-sensitive promoter, e.g., a promoter that is directly or indirectly induced by a temperature between 37° C. and 42° C. Inducible promoters are described in more detail herein.

The EGF gene may be present on a plasmid or chromosome in the bacterial cell. In one embodiment, the EGF gene is located on a plasmid in the bacterial cell. In another embodiment, the EGF gene is located in the chromosome of the bacterial cell. In yet another embodiment, a native copy of the EGF gene is located in the chromosome of the bacterial cell. The EGF gene may be expressed on a low-copy plasmid or a high-copy plasmid. The high-copy plasmid may be useful for increasing expression of EGF.

In particular embodiments, the bacterium comprises a gene sequence encoding EGF operably linked to a thermoregulated promoter, e.g., cI857; a gene sequence encoding an N-terminal OmpA secretion tag operably linked to the gene sequence encoding EGF; a modification, e.g., knockout, in the Phage 3 genome; a modification, e.g., knockout, in the colibactin pks island; a thymidine auxotrophy, as disclosed herein.

In particular embodiments, the bacterium comprises a gene sequence encoding EGF operably linked to a thermoregulated promoter, e.g., cI857; a gene sequence encoding an N-terminal OmpA secretion tag operably linked to the EGF gene sequence; a modification, e.g., knockout, in the Phage 3 genome; a modification, e.g., knockout, in the colibactin pks island; a thymidine auxotrophy; and ¦pal (diffusible outer membrane), as disclosed herein.

In particular embodiments, the bacterium comprises a gene sequence encoding EGF operably linked to an oxygen level-dependent promoter, e.g., FNR-responsive promoter; a gene sequence encoding an N-terminal OmpA secretion tag operably linked to the EGF gene sequence; a modification, e.g., knockout, in the Phage 3 genome; a modification, e.g., knockout, in the colibactin pks island; a thymidine auxotrophy, as disclosed herein.

In particular embodiments, the bacterium comprises a gene sequence encoding EGF operably linked to an oxygen level-dependent promoter, e.g., FNR-responsive promoter; a gene sequence encoding an N-terminal OmpA secretion tag operably linked to the EGF gene sequence; a modification, e.g., knockout, in the Phage 3 genome; a modification, e.g., knockout, in the colibactin pks island; a thymidine auxotrophy; and Δpal (diffusible outer membrane), as disclosed herein.

In particular embodiments, the gene sequences encoding the EGF constructs comprising gene sequences encoding EGF operably linked to an inducible promoter, e.g., an oxygen level-dependent or temperature sensitive promoter, and the gene sequence encoding an N-terminal OmpA secretion tag operably linked to the EGF gene sequence may be integrated into the bacterial chromosome. In some embodiments, the bacterium comprises a single integrated copy of such an EGF gene sequence (i.e., OmpA-EGF). In some embodiments, the genetically engineered bacterium comprises multiple integrated copies of the EGF gene sequence. The multiple copies may be present at the same genomic integration site, e.g., arranged in tandem. In some embodiments, each copy of the EGF gene sequence may be operably linked to the same copy of the same promoter. In some embodiments, each copy of the EGF gene sequence may be operably linked to a different copy of the same promoter or different promoters. Alternatively, multiple copies of EGF gene sequences may be integrated at multiple distinct genomic integration sites, wherein each copy of EGF gene sequence is operably linked to a distinct instance of the promoter. In some embodiments, the promoters are multiple instances of the same promoter. In some embodiments, the promoters are multiple instances of different promoters. In some embodiments, the promoters are inducible promoters, such as a low oxygen inducible FNR promoter, a temperature sensitive promoter, or an IPTG inducible promoter.

In a particular embodiment, the bacterium comprises two copies of EGF gene sequences, wherein the two copies of the gene sequences are integrated at two distinct integration sites, and wherein each copy of the EGF gene sequence is operably linked to a separate instance of the same promoter. In a particular embodiment, the bacterium comprises three copies of the EGF gene sequences, wherein the three copies of the gene sequences each are integrated at three distinct integration sites, and wherein each copy of the EGF gene is operatively linked to a separate instance of the same promoter. In some embodiments, the promoter is an inducible promoter, e.g., a low oxygen inducible promoter (e.g., FNR promoter), a temperature sensitive promoter, or an IPTG inducible promoter.

In any of these embodiments, the EGF gene sequences may be operably linked to a gene sequence encoding an N-terminal OmpA secretion tag.

In any of these embodiments, the bacterium may further comprise one or more of (1) a knockout in the Phage 3 genome; (2) a modification, e.g., knockout, in the colibactin pks island; (3) a thymidine auxotrophy; and (4) Δpal (diffusible outer membrane), as disclosed herein.

In some embodiments, the recombinant bacteria are capable of secreting about 0.1, 0.2, 0.3, 0.4, 0.5, 0.6, 0.7, 0.8, 0.9, or 1.0 ug EGF/5e11 cells over 4 hours under inducing conditions, e.g., under low oxygen or anaerobic inducing conditions.

In some embodiments, the recombinant bacteria are capable of secreting about 1, 1.5, 2, 2.5, 3, 3.5, 4, 4.5, 5, 5.5, 6, 6.5, 7, 7.5, 8, 8.5, 9, 9.5, or 10 ug EGF/5e11 cells over 4 hours under inducing conditions, e.g., under low oxygen or anaerobic inducing conditions.

In some embodiments, the recombinant bacteria are capable of secreting about 10, 20, 30, 40, 50, 60, 70, 80, 90, 100, 110, or 120 ug EGF/5e11 cells over 4 hours under inducing conditions, e.g., under low oxygen or anaerobic inducing conditions.

In some embodiments, the recombinant bacteria are capable of secreting at least about 110, 120, 130, 140, 150, 160, 170, 180, 190, or 200 g EGF/5e11 cells over 4 hours under inducing conditions, e.g., under low oxygen or anaerobic inducing conditions.

In some embodiments, the recombinant bacteria are capable of secreting at least about 210, 220, 230, 240, 250, 260, 270, 280, 290, or 300 μg EGF/5e11 cells over 4 hours under inducing conditions, e.g., under low oxygen or anaerobic inducing conditions.

In some embodiments, the recombinant bacteria are capable of secreting at least about 350, 400, 450, 500, 550, 600, 650, 700, 750, or 800 μg EGF/5e11 cells over 4 hours under inducing conditions, e.g., under low oxygen or anaerobic inducing conditions.

In some embodiments, the recombinant bacteria are capable of secreting about 0.1, 0.2, 0.3, 0.4, 0.5, 0.6, 0.7, 0.8, 0.9, or 1.0 ug EGF/5e11 cells over 8 hours under inducing conditions, e.g., under low oxygen or anaerobic inducing conditions.

In some embodiments, the recombinant bacteria are capable of secreting about 1, 1.5, 2, 2.5, 3, 3.5, 4, 4.5, 5, 5.5, 6, 6.5, 7, 7.5, 8, 8.5, 9, 9.5, or 10 ug EGF/5e11 cells over 8 hours under inducing conditions, e.g., under low oxygen or anaerobic inducing conditions.

In some embodiments, the recombinant bacteria are capable of secreting about 10, 20, 30, 40, 50, 60, 70, 80, 90, 100, 110, or 120 ug/mL EGF/5e11 cells over 8 hours under inducing conditions, e.g., under low oxygen or anaerobic inducing conditions.

In some embodiments, the recombinant bacteria are capable of secreting at least about 40, 50, 60, 70, 80, 90, or 100 μg EGF/5e11 cells over 8 hours under inducing conditions, e.g., under low oxygen or anaerobic inducing conditions.

In some embodiments, the recombinant bacteria are capable of secreting at least about 110, 120, 130, 140, 150, 160, 170, 180, 190, or 200 g EGF/5e11 cells over 8 hours under inducing conditions, e.g., under low oxygen or anaerobic inducing conditions.

In some embodiments, the recombinant bacteria are capable of secreting at least about 210, 220, 230, 240, 250, 260, 270, 280, 290, or 300 μg EGF/5e11 cells over 8 hours under inducing conditions, e.g., under low oxygen or anaerobic inducing conditions.

In some embodiments, the recombinant bacteria are capable of secreting at least about 310, 320, 330, 340, 350, 360, 370, 380, 390, or 400 μg EGF/5e11 cells over 8 hours under inducing conditions, e.g., under low oxygen or anaerobic inducing conditions.

In some embodiments, the recombinant bacteria are capable of secreting at least about 410, 420, 430, 440, 450, 460, 470, 480, 490, or 500 μg EGF/5e11 cells over 8 hours under inducing conditions, e.g., under low oxygen or anaerobic inducing conditions.

In some embodiments, the recombinant bacteria are capable of secreting at least about 510, 520, 530, 540, 550, 560, 570, 580, 590, or 600 μg EGF/5e11 cells over 8 hours under inducing conditions, e.g., under low oxygen or anaerobic inducing conditions.

In some embodiments, the recombinant bacteria are capable of secreting at least about 610, 620, 630, 640, 650, 660, 670, 680, 690, or 700 μg EGF/5e11 cells over 8 hours under inducing conditions, e.g., under low oxygen or anaerobic inducing conditions.

In some embodiments, the recombinant bacteria are capable of secreting at least about 710, 720, 730, 740, 750, 760, 770, 780, 790, or 800 μg EGF/5e11 cells over 8 hours under inducing conditions, e.g., under low oxygen or anaerobic inducing conditions.

In some embodiments, the recombinant bacteria are capable of secreting at least about 350, 400, 450, 500, 550, 600, 650, 700, 750, or 800 μg EGF/5e11 cells over 8 hours under inducing conditions, e.g., under low oxygen or anaerobic inducing conditions.

In some embodiments, the recombinant bacteria are capable of secreting about 0.1, 0.2, 0.3, 0.4, 0.5, 0.6, 0.7, 0.8, 0.9, or 1.0 ug EGF/5e11 cells over 5 hours under inducing conditions, e.g., under low oxygen or anaerobic inducing conditions.

In some embodiments, the recombinant bacteria are capable of secreting about 1, 1.5, 2, 2.5, 3, 3.5, 4, 4.5, 5, 5.5, 6, 6.5, 7, 7.5, 8, 8.5, 9, 9.5, or 10 ug EGF/5e11 cells over 5 hours under inducing conditions, e.g., under low oxygen or anaerobic inducing conditions.

In some embodiments, the recombinant bacteria are capable of secreting about 10, 20, 30, 40, 50, 60, 70, 80, 90, 100, 110, or 120 ug EGF/5e11 cells over 5 hours under inducing conditions, e.g., under low oxygen or anaerobic inducing conditions.

In some embodiments, the recombinant bacteria are capable of secreting at least 110, 120, 130, 140, 150, 160, 170, 180, 190, or 200 μg EGF/5e11 cells over 5 hours under inducing conditions, e.g., under low oxygen or anaerobic inducing conditions.

In any of the embodiments above, inducing conditions may be low oxygen or anaerobic inducing conditions or, alternatively, inducing conditions may be a temperature between about 37° C. and 42° C. In some embodiments, inducing conditions may be the presence of an inducer, such as IPTG. In some specific embodiments, the inducing conditions are low oxygen or anaerobic inducing conditions. In some specific embodiments, the inducing conditions are a temperature between about 37° C. and about 42° C.

### Inducible Regulatory Regions

Herein the terms “payload” “polypeptide of interest” or “polypeptides of interest”, “protein of interest”, “proteins of interest”, “payloads” “effector molecule”, “effector” refers to one or more effector molecules described herein and/or one or more enzyme(s) or polypeptide(s) that function as enzymes for the production and secretion of such effector molecules, e.g., EGF.

In some embodiments, the bacterial cell comprises a stably maintained plasmid or chromosome carrying the gene(s) encoding payload (s), such that the payload(s) can be expressed in the host cell, and the host cell is capable of survival and/or growth in vitro, e.g., in medium, and/or in vivo, e.g., in the gut. In some embodiments, bacterial cell comprises two or more distinct payloads or operons, e.g., two or more payload genes. In some embodiments, bacterial cell comprises three or more distinct transporters or operons, e.g., three or more payload genes. In some embodiments, bacterial cell comprises 4, 5, 6, 7, 8, 9, 10, or more distinct payloads or operons, e.g., 4, 5, 6, 7, 8, 9, 10, or more payload genes.

Additional effector molecules, e.g., therapeutic polypeptides, which can be secreted are described in PCT/US2017/013072, filed Jan. 11, 2017; PCT/US2017/016609, filed Feb. 3, 2017; PCT/US2016/039444, filed Jun. 24, 2016; PCT/US2016/069052, filed Dec. 28, 2016; PCT/US2017/012946, filed Jan. 11, 2017; PCT/US2017/017552, filed Feb. 10, 2017; PCT/US2017/017563, filed Feb. 10, 2017, the contents of which is herein incorporated by reference in its entirety.

In some embodiments, the recombinant bacteria comprise multiple copies of the same payload gene(s). In some embodiments, the gene encoding the payload is present on a plasmid and operably linked to a directly or indirectly inducible promoter. In some embodiments, the gene encoding the payload is present on a plasmid and operably linked to a constitutive promoter. In some embodiments, the gene encoding the payload is present on a plasmid and operably linked to a promoter that is induced under low-oxygen or anaerobic conditions. In some embodiments, the gene encoding the payload is present on a plasmid and operably linked to a temperature sensitive promoter. In some embodiments, the gene encoding the payload is present on plasmid and operably linked to a promoter that is induced by exposure to tetracycline or arabinose, or another chemical or nutritional inducer described herein.

In some embodiments, the gene encoding the payload is present on a chromosome and operably linked to a directly or indirectly inducible promoter. In some embodiments, the gene encoding the payload is present on a chromosome and operably linked to a constitutive promoter. In some embodiments, the gene encoding the payload is present in the chromosome and operably linked to a promoter that is induced under low-oxygen or anaerobic conditions. In some embodiments, the gene encoding the payload is present in the chromosome and operably linked to a temperature sensitive promoter. In some embodiments, the gene encoding the payload is present on chromosome and operably linked to a promoter that is induced by exposure to tetracycline or arabinose, or another chemical or nutritional inducer described herein.

In some embodiments, the recombinant bacteria comprise two or more payloads, all of which are present on the chromosome. In some embodiments, the recombinant bacteria comprise two or more payloads, all of which are present on one or more same or different plasmids. In some embodiments, the recombinant bacteria comprise two or more payloads, some of which are present on the chromosome and some of which are present on one or more same or different plasmids.

In any of the nucleic acid embodiments described above, an EGF payload is operably linked to one or more directly or indirectly inducible promoter(s). In some embodiments, the one or more payload(s) are operably linked to a directly or indirectly inducible promoter that is induced under exogeneous environmental conditions, e.g., conditions found in the gut, e.g., induced by metabolites found in the gut, or other specific conditions. In some embodiments, the one or more payload(s) are operably linked to a directly or indirectly inducible promoter that is temperature sensitive or induced under low-oxygen or anaerobic conditions, or induced under inflammatory conditions (e.g., RNS, ROS), as described herein. In some embodiments, the one or more payload(s) are operably linked to a directly or indirectly inducible promoter that is induced under immunosuppressive conditions, e.g., as found in the tumor, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, or others described herein. In some embodiments, the two or more payloads are all linked to a constitutive promoter, as described herein.

In some embodiments, the promoter is induced under in vivo conditions, e.g., the gut, as described herein. In some embodiments, the promoter is induced under in vitro conditions, e.g., various cell culture and/or cell manufacturing conditions, as described herein.

In some embodiments, the promoter that is operably linked to the gene encoding the payload is directly induced by exogenous environmental conditions (e.g., in vivo and/or in vitro and/or production/manufacturing conditions).

In some embodiments, the promoter is directly or indirectly induced by exogenous environmental conditions specific to the gut of a mammal, e.g., to the small intestine of a mammal. In some embodiments, the promoter is directly or indirectly induced by low-oxygen or anaerobic conditions such as the environment of the mammalian gut. In some embodiments, the promoter is a temperature sensitive promoter. In some embodiments, the promoter is directly or indirectly induced by molecules or metabolites that are specific to the tumor, a particular tissue or the gut of a mammal. In some embodiments, the promoter is directly or indirectly induced by a molecule that is co-administered with the bacterial cell.

### FNR Dependent Regulation

The recombinant bacteria comprise a gene or gene cassette for producing EGF, wherein the gene or gene cassette is operably linked to a directly or indirectly inducible promoter that is controlled by exogenous environmental condition(s). In some embodiments, the inducible promoter is an oxygen level-dependent promoter and EGF is expressed in low-oxygen, microaerobic, or anaerobic conditions. For example, in low oxygen conditions, the oxygen level-dependent promoter is activated by a corresponding oxygen level-sensing transcription factor, thereby driving production of EGF.

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the recombinant bacteria comprise a gene or gene cassette for producing a payload under the control of an oxygen level-dependent promoter. In a more specific aspect, the recombinant bacteria comprise a gene or gene cassette for producing a payload under the control of an oxygen level-dependent promoter that is activated under low-oxygen or anaerobic environments, such as the environment of the mammalian gut.

In certain embodiments, the bacterial cell comprises a gene encoding a payload expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. Exemplary FNR responsive promoters include, but are not limited to, SEQ ID NO: 151-167. FNR promoter sequences are known in the art, and any suitable FNR promoter sequence(s) may be used in the recombinant bacteria.

In some embodiments, the recombinant bacteria comprise one or more of: SEQ ID NOS: 151-157, nirB1 promoter (SEQ ID NO: 158), nirB2 promoter (SEQ ID NO: 159), nirB3 promoter (SEQ ID NO: 160), ydfZ promoter (SEQ ID NO: 161), nirB promoter fused to a strong ribosome binding site (SEQ ID NO: 162), ydfZ promoter fused to a strong ribosome binding site (SEQ ID NO: 163), fnrS, an anaerobically induced small RNA gene (fnrS1 promoter SEQ ID NO: 164 or fnrS2 promoter SEQ ID NO: 165), nirB promoter fused to a crp binding site (SEQ ID NO: 166), and fnrS fused to a crp binding site (SEQ ID NO: 167). In some embodiments, the FNR-responsive promoter is at least about 80%, 85%, 90%, 95%, or 99% homologous to the sequence of any one of SEQ ID NO: 151-167.

In some embodiments, multiple distinct FNR nucleic acid sequences are inserted in the recombinant bacteria. In alternate embodiments, the recombinant bacteria comprise a gene encoding a payload expressed under the control of an alternate oxygen level-dependent promoter, e.g., DNR (Trunk et al., 2010) or ANR (Ray et al., 1997). In these embodiments, expression of the payload gene is particularly activated in a low-oxygen or anaerobic environment, such as in the gut, e.g., a mammalian gut, e.g., a human gut. In some embodiments, gene expression is further optimized by methods known in the art, e.g., by optimizing ribosomal binding sites and/or increasing mRNA stability.

In another embodiment, the recombinant bacteria comprise a gene sequence encoding EGF, expressed under the control of anaerobic regulation of arginine deiminiase and nitrate reduction transcriptional regulator (ANR). In P. aeruginosa, ANR is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT----ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

In other embodiments, the one or more gene sequence(s) for producing a payload are expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Gorke and Stnlke, 2008). In some embodiments, the gene or gene cassette for producing EGF is controlled by an oxygen level-dependent promoter fused to a CRP binding site. In some embodiments, the one or more gene sequence(s) for a payload are controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and transcription of the gene or gene cassette for producing an payload is repressed. In some embodiments, an oxygen level-dependent promoter (e.g., an FNR promoter) fused to a binding site for a transcriptional activator is used to ensure that the gene or gene cassette for producing a payload is not expressed under anaerobic conditions when sufficient amounts of glucose are present, e.g., by adding glucose to growth media in vitro.

In some embodiments, the recombinant bacteria comprise an oxygen level-dependent promoter from a different species, strain, or substrain of bacteria. In some embodiments, the recombinant bacteria comprise an oxygen level-sensing transcription factor, e.g., FNR, ANR or DNR, from a different species, strain, or substrain of bacteria. In some embodiments, the recombinant bacteria comprise an oxygen level-sensing transcription factor and corresponding promoter from a different species, strain, or substrain of bacteria. The heterologous oxygen-level dependent transcriptional regulator and/or promoter increases the transcription of genes operably linked to said promoter, e.g., one or more gene sequence(s) for producing the payload(s) in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the recombinant bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the payload, in a low-oxygen or anaerobic environment, as compared to the wild-type promoter under the same conditions. In some embodiments, the recombinant bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the payload, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006). In some embodiments, both the oxygen level-sensing transcriptional regulator and corresponding promoter are mutated relative to the wild-type sequences from bacteria of the same subtype in order to increase expression of the payload in low-oxygen conditions.

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the payload are present on different plasmids. In some embodiments, the gene encoding the oxygen level-sensing transcriptional regulator and the gene encoding the payload are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the payload are present on different chromosomes. In some embodiments, the gene encoding the oxygen level-sensing transcriptional regulator and the gene encoding the payload are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the payload. In some embodiments, expression of the transcriptional regulator is controlled by the same promoter that controls expression of the payload. In some embodiments, the transcriptional regulator and the payload are divergently transcribed from a promoter region

### RNS-Dependent Regulation

In some embodiments, the recombinant bacteria comprise a gene encoding a payload that is expressed under the control of an inducible promoter. In some embodiments, the recombinant bacterium that expresses a payload under the control of a promoter that is activated by inflammatory conditions. In one embodiment, the gene for producing the payload is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO·), peroxynitrite or peroxynitrite anion (ONOO—), nitrogen dioxide (·NO₂), dinitrogen trioxide (N2O3), peroxynitrous acid (ONOOH), and nitroperoxycarbonate (ONOOCO2-) (unpaired electrons denoted by ·). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., a payload gene sequence(s), e.g., any of the payloads described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 6.

TABLE 6
Examples of RNS-sensing transcription
factors and RNS-responsive genes

| RNS-sensing | Primarily | Examples of responsive |
|---|---|---|
| transcription | capable of | genes, promoters, and/ |
| factor: | sensing: | or regulatory regions: |
| NsrR | NO | norB, aniA, nsrR, hmpA, ytfE, |
| | | ygbA, hcp, hcr, nrfA, aox |
| NorR | NO | norVW, norR |
| DNR | NO | norCB, nir, nor, nos |

In some embodiments, the recombinant bacteria comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of a payload, thus controlling expression of the payload relative to RNS levels. For example, the tunable regulatory region is a RNS-inducible regulatory region, and the payload is a payload, such as any of the payloads provided herein; when RNS is present, e.g., in an inflamed tissue, a RNS-sensing transcription factor binds to and/or activates the regulatory region and drives expression of the payload gene or genes. Subsequently, when inflammation is ameliorated, RNS levels are reduced, and production of the payload is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The recombinant bacteria may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012). In certain embodiments, the recombinant bacteria comprise a RNS-inducible regulatory region from norVW that is operatively linked to a gene or genes, e.g., one or more payload gene sequence(s). In the presence of RNS, a NorR transcription factor senses RNS and activates to the norVW regulatory region, thereby driving expression of the operatively linked gene(s) and producing the payload(s).

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The recombinant bacteria may comprise any suitable RNS-responsive regulatory region from a gene that is activated by DNR. Genes that are capable of being activated by DNR are known in the art (see, e.g., Castiglione et al., 2009; Giardina et al., 2008). In certain embodiments, the recombinant bacteria comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more payloads. In some embodiments, the DNR is Pseudomonas aeruginosa DNR. Any suitable transcriptional regulator that is controlled by exogenous environmental conditions and corresponding regulatory region may be used. Non-limiting examples include ArcA/B, ResD/E, NreA/B/C, and AirSR, and others are known in the art.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The recombinant bacteria may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010). In certain embodiments, the recombinant bacteria comprise a RNS-derepressible regulatory region from norB that is operatively linked to a gene or genes, e.g., a payload gene or genes. In the presence of RNS, an NsrR transcription factor senses RNS and no longer binds to the norB regulatory region, thereby derepressing the operatively linked a payload gene or genes and producing the encoding a payload(s).

In some embodiments, it is advantageous for the recombinant bacteria to express a RNS-sensing transcription factor that does not regulate the expression of a significant number of native genes in the bacteria. In some embodiments, the recombinant bacterium expresses a RNS-sensing transcription factor from a different species, strain, or substrain of bacteria, wherein the transcription factor does not bind to regulatory sequences in the recombinant bacterium. In some embodiments, the recombinant bacterium is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the recombinant bacteria.

In these embodiments, the recombinant bacteria may comprise a two-repressor activation regulatory circuit, which is used to express a payload. The two-repressor activation regulatory circuit comprises a first RNS-sensing repressor and a second repressor, which is operatively linked to a gene or gene cassette, e.g., encoding a payload. In one aspect of these embodiments, the RNS-sensing repressor inhibits transcription of the second repressor, which inhibits the transcription of the gene or gene cassette. Examples of second repressors useful in these embodiments, include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., a payload gene or genes is expressed.

A RNS-responsive transcription factor may induce, derepress, or repress gene expression depending upon the regulatory region sequence used in the recombinant bacteria. One or more types of RNS-sensing transcription factors and corresponding regulatory region sequences may be present in recombinant bacteria. In some embodiments, the recombinant bacteria comprise one type of RNS-sensing transcription factor, e.g., NsrR, and one corresponding regulatory region sequence, e.g., from norB. In some embodiments, the recombinant bacteria comprise one type of RNS-sensing transcription factor, e.g., NsrR, and two or more different corresponding regulatory region sequences, e.g., from norB and aniA. In some embodiments, the recombinant bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the recombinant bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the recombinant bacteria comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of EGF. In some embodiments, expression of the RNS-sensing transcription factor is controlled by the same promoter that controls expression of EGF. In some embodiments, the RNS-sensing transcription factor and EGF are divergently transcribed from a promoter region.

In some embodiments, the recombinant bacteria comprise a gene for a RNS-sensing transcription factor from a different species, strain, or substrain of bacteria. In some embodiments, the recombinant bacteria comprise a RNS-responsive regulatory region from a different species, strain, or substrain of bacteria. In some embodiments, the recombinant bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the recombinant bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the recombinant bacteria comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing EGF are present on different plasmids. In some embodiments, the gene encoding the RNS-sensing transcription factor and the gene or gene cassette for producing EGF are present on the same plasmid. In some embodiments, the gene encoding the RNS-sensing transcription factor is present on a chromosome. In some embodiments, the gene encoding the RNS-sensing transcription factor and the gene or gene cassette for producing EGF are present on different chromosomes. In some embodiments, the gene encoding the RNS-sensing transcription factor and the gene or gene cassette for producing EGF are present on the same chromosome.

In some embodiments, the recombinant bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the payload in the presence of RNS, as compared to the wild-type regulatory region under the same conditions. In some embodiments, the recombinant bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the payload in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the payload in the presence of RNS.

In some embodiments, the gene or gene cassette for producing the e molecule(s) is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of one or more encoding a payload gene(s) may be integrated into the bacterial chromosome. Having multiple copies of the gene or gen(s) integrated into the chromosome allows for greater production of the payload(s) and also permits fine-tuning of the level of expression. Alternatively, different circuits described herein, such as any of the secretion or exporter circuits, in addition to the EGF gene(s) or gene cassette(s) could be integrated into the bacterial chromosome at one or more different integration sites to perform multiple different functions.

In some embodiments, the recombinant bacteria produce at least one payload in the presence of RNS to reduce local gut inflammation by at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold as compared to unmodified bacteria of the same subtype under the same conditions. Inflammation may be measured by methods known in the art, e.g., counting disease lesions using endoscopy; detecting T regulatory cell differentiation in peripheral blood, e.g., by fluorescence activated sorting; measuring T regulatory cell levels; measuring cytokine levels; measuring areas of mucosal damage; assaying inflammatory biomarkers, e.g., by qPCR; PCR arrays; transcription factor phosphorylation assays; immunoassays; and/or cytokine assay kits (Mesoscale, Cayman Chemical, Qiagen).

In some embodiments, the recombinant bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of payload in the presence of RNS than unmodified bacteria of the same subtype under the same conditions. Certain unmodified bacteria will not have detectable levels of the payload. In embodiments using genetically modified forms of these bacteria, payload will be detectable in the presence of RNS.

### ROS-Dependent Regulation

In some embodiments, the recombinant bacteria comprise a gene for producing a payload that is expressed under the control of an inducible promoter. In some embodiments, the recombinant bacterium that expresses a payload under the control of a promoter that is activated by conditions of cellular damage. In one embodiment, the gene for producing the payload is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H₂O₂), organic peroxide (ROOH), hydroxyl ion (OH—), hydroxyl radical (·OH), superoxide or superoxide anion (·O2-), singlet oxygen (IO2), ozone (O3), carbonate radical, peroxide or peroxyl radical (·O2-2), hypochlorous acid (HOCl), hypochlorite ion (OCl—), sodium hypochlorite (NaOCl), nitric oxide (NO·), and peroxynitrite or peroxynitrite anion (ONOO—) (unpaired electrons denoted by ·). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more payload(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes. Alternatively, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 7.

TABLE 7
Examples of ROS-sensing transcription
factors and ROS-responsive genes

| ROS-sensing | Primarily | Examples of responsive |
|---|---|---|
| transcription | capable of | genes, promoters, and/ |
| factor: | sensing: | or regulatory regions: |
| OxyR | H₂O₂ | ahpC; ahpF; dps; dsbG; fhuF; |
| | | flu; fur; gor; grxA; hemH; katG; |
| | | oxyS; sufA; sufB; sufC; sufD; |
| | | sufE; sufS; trxC; uxuA; yaaA; |
| | | yaeH; yaiA; ybjM; ydcH; ydeN; |
| | | ygaQ; yljA; ytfK |
| PerR | H₂O₂ | katA; ahpCF; mrgA; zoaA; fur; |
| | | hemAXCDBL; srfA |
| OhrR | Organic peroxides | ohrA |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; |
| | | xylC; gabD1; rluC2; cgtS9; azlC; |
| | | narKGHJI; rosR |

In some embodiments, the recombinant bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of a payload, thus controlling expression of the payload relative to ROS levels. For example, the tunable regulatory region is a ROS-inducible regulatory region, and the molecule is a payload; when ROS is present, e.g., in an inflamed tissue, a ROS-sensing transcription factor binds to and/or activates the regulatory region and drives expression of the gene sequence for the payload, thereby producing the payload. Subsequently, when inflammation is ameliorated, ROS levels are reduced, and production of the payload is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H₂O₂ detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The recombinant bacteria may comprise any suitable ROS-responsive regulatory region from a gene that is activated by OxyR. Genes that are capable of being activated by OxyR are known in the art (see, e.g., Zheng et al., 2001; Dubbs et al., 2012). In certain embodiments, the recombinant bacteria comprise a ROS-inducible regulatory region from oxyS that is operatively linked to a gene, e.g., a payload gene. In the presence of ROS, e.g., H2O2, an OxyR transcription factor senses ROS and activates to the oxyS regulatory region, thereby driving expression of the operatively linked payload gene and producing the payload. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The recombinant bacteria may comprise any suitable ROS-responsive regulatory region from a gene that is activated by SoxR. Genes that are capable of being activated by SoxR are known in the art (see, e.g., Koo et al., 2003). In certain embodiments, the recombinant bacteria comprise a ROS-inducible regulatory region from soxS that is operatively linked to a gene, e.g., a payload. In the presence of ROS, the SoxR transcription factor senses ROS and activates the soxS regulatory region, thereby driving expression of the operatively linked a payload gene and producing the payload.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H2O2 but it shows much higher reactivity for organic hydroperoxides” (Duarte et al., 2010). The recombinant bacteria may comprise any suitable ROS-responsive regulatory region from a gene that is repressed by OhrR. Genes that are capable of being repressed by OhrR are known in the art (see, e.g., Dubbs et al., 2012). In certain embodiments, the recombinant bacteria comprise a ROS-derepressible regulatory region from ohrA that is operatively linked to a gene or gene cassette, e.g., a payload gene. In the presence of ROS, e.g., NaOCl, an OhrR transcription factor senses ROS and no longer binds to the ohrA regulatory region, thereby derepressing the operatively linked payload gene and producing the payload.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the recombinant bacteria comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 1085)” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The recombinant bacteria may comprise any suitable ROS-responsive regulatory region from a gene that is repressed by RosR. Genes that are capable of being repressed by RosR are known in the art (see, e.g., Bussmann et al., 2010). In certain embodiments, the recombinant bacteria comprise a ROS-derepressible regulatory region from cgtS9 that is operatively linked to a gene or gene cassette, e.g., a payload. In the presence of ROS, e.g., H2O2, a RosR transcription factor senses ROS and no longer binds to the cgtS9 regulatory region, thereby derepressing the operatively linked payload gene and producing the payload.

In some embodiments, it is advantageous for the recombinant bacteria to express a ROS-sensing transcription factor that does not regulate the expression of a significant number of native genes in the bacteria. In some embodiments, the recombinant bacterium expresses a ROS-sensing transcription factor from a different species, strain, or substrain of bacteria, wherein the transcription factor does not bind to regulatory sequences in the recombinant bacterium. In some embodiments, the recombinant bacterium is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the recombinant bacteria.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 1086)) residing within and near the promoter sequences of PerR-controlled genes” (Marinho et al., 2014). PerR is capable of binding a regulatory region that “overlaps part of the promoter or is immediately downstream from it” (Dubbs et al., 2012). The recombinant bacteria may comprise any suitable ROS-responsive regulatory region from a gene that is repressed by PerR. Genes that are capable of being repressed by PerR are known in the art (see, e.g., Dubbs et al., 2012).

In these embodiments, the recombinant bacteria may comprise a two-repressor activation regulatory circuit, which is used to express a payload. The two-repressor activation regulatory circuit comprises a first ROS-sensing repressor, e.g., PerR, and a second repressor, e.g., TetR, which is operatively linked to a gene or gene cassette, e.g., a payload. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., a payload. In the absence of PerR binding (which occurs in the absence of ROS), tetR is transcribed, and TetR represses expression of the gene or gene cassette, e.g., a payload. In the presence of PerR binding (which occurs in the presence of ROS), tetR expression is repressed, and the gene or gene cassette, e.g., a payload, is expressed.

A ROS-responsive transcription factor may induce, derepress, or repress gene expression depending upon the regulatory region sequence used in the recombinant bacteria. For example, although “OxyR is primarily thought of as a transcriptional activator under oxidizing conditions. OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The recombinant bacteria may comprise any suitable ROS-responsive regulatory region from a gene that is repressed by OxyR. In some embodiments, OxyR is used in a two-repressor activation regulatory circuit, as described above. Genes that are capable of being repressed by OxyR are known in the art (see, e.g., Zheng et al., 2001). Or, for example, although RosR is capable of repressing a number of genes, it is also capable of activating certain genes, e.g., the narKGHJI operon. In some embodiments, the recombinant bacteria comprise any suitable ROS-responsive regulatory region from a gene that is activated by RosR. In some embodiments, the recombinant bacteria comprise any suitable ROS-responsive regulatory region from a gene that is activated by PerR.

One or more types of ROS-sensing transcription factors and corresponding regulatory region sequences may be present in recombinant bacteria. For example, “OhrR is found in both Gram-positive and Gram-negative bacteria and can coreside with either OxyR or PerR or both” (Dubbs et al., 2012). In some embodiments, the recombinant bacteria comprise one type of ROS-sensing transcription factor, e.g., OxyR, and one corresponding regulatory region sequence, e.g., from oxyS. In some embodiments, the recombinant bacteria comprise one type of ROS-sensing transcription factor, e.g., OxyR, and two or more different corresponding regulatory region sequences, e.g., from oxyS and katG. In some embodiments, the recombinant bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the recombinant bacteria comprise two or more types of ROS-sensing transcription factors and one corresponding regulatory region sequence.

In some embodiments, recombinant bacteria comprise nucleic acid sequences comprising OxyR binding sites. In some embodiments, recombinant bacteria comprise a nucleic acid sequence that is at least about 80%, 85%, 90%, 95%, or 99% homologous to the DNA sequence of SEQ ID NO: 168, SEQ ID NO: 169, or SEQ ID NO: 170, or SEQ ID NO: 171, or a functional fragment thereof. In some embodiments, the regulatory region sequence is at least about 80%, 85%, 90%, 95%, or 99% homologous to the sequence of SEQ ID NO: 168, SEQ ID NO: 169, SEQ ID NO: 170, and/or SEQ ID NO: 171.

In some embodiments, the recombinant bacteria comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of EGF. In some embodiments, expression of the ROS-sensing transcription factor is controlled by the same promoter that controls expression of EGF. In some embodiments, the ROS-sensing transcription factor and EGF are divergently transcribed from a promoter region.

In some embodiments, the recombinant bacteria comprise a gene for a ROS-sensing transcription factor from a different species, strain, or substrain of bacteria. In some embodiments, the recombinant bacteria comprise a ROS-responsive regulatory region from a different species, strain, or substrain of bacteria. In some embodiments, the recombinant bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the recombinant bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the recombinant bacteria comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing EGF are present on different plasmids. In some embodiments, the gene encoding the ROS-sensing transcription factor and the gene or gene cassette for producing EGF are present on the same plasmid. In some embodiments, the gene encoding the ROS-sensing transcription factor is present on a chromosome. In some embodiments, the gene encoding the ROS-sensing transcription factor and the gene or gene cassette for producing EGF are present on different chromosomes. In some embodiments, the gene encoding the ROS-sensing transcription factor and the gene or gene cassette for producing EGF are present on the same chromosome.

In some embodiments, the recombinant bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the payload in the presence of ROS, as compared to the wild-type regulatory region under the same conditions. In some embodiments, the recombinant bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the payload in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the payload in the presence of ROS.

In some embodiments, the gene or gene cassette for producing the payload is present on a plasmid and operably linked to a promoter that is induced by ROS. In some embodiments, the gene or gene cassette for producing the payload is present in the chromosome and operably linked to a promoter that is induced by ROS. In some embodiments, the gene or gene cassette for producing the payload is present on a chromosome and operably linked to a promoter that is induced by exposure to tetracycline. In some embodiments, the gene or gene cassette for producing the payload is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the recombinant bacteria may comprise multiple copies of the gene(s) capable of producing a payload(s). In some embodiments, the gene(s) capable of producing a payload(s) is present on a plasmid and operatively linked to a ROS-responsive regulatory region. In some embodiments, the gene(s) capable of producing a payload is present in a chromosome and operatively linked to a ROS-responsive regulatory region.

Thus, in some embodiments, the recombinant bacteria produce one or more payloads under the control of an oxygen level-dependent promoter, a reactive oxygen species (ROS)-dependent promoter, or a reactive nitrogen species (RNS)-dependent promoter, and a corresponding transcription factor.

In some embodiments, the recombinant bacteria comprise a stably maintained plasmid or chromosome carrying a gene for producing a payload, such that the payload can be expressed in the host cell, and the host cell is capable of survival and/or growth in vitro, e.g., in medium, and/or in vivo. In some embodiments, a bacterium may comprise multiple copies of the gene encoding the payload. In some embodiments, the gene encoding the payload is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the payload is expressed on a high-copy plasmid. In some embodiments, the high-copy plasmid may be useful for increasing expression of the payload. In some embodiments, the gene encoding the payload is expressed on a chromosome.

### Propionate and Other Promoters

In some embodiments, the recombinant bacteria comprise the gene or gene cassette for producing EGF polypeptides, expressed under the control of an inducible promoter that is responsive to specific molecules or metabolites in the environment, e.g., the tumor microenvironment, a specific tissue, or the mammalian gut. For example, the short-chain fatty acid propionate is a major microbial fermentation metabolite localized to the gut (Hosseini et al., 2011). In one embodiment, the gene or gene cassette for producing EGF is under the control of a propionate-inducible promoter. In a more specific embodiment, the gene or gene cassette for producing EGF is under the control of a propionate-inducible promoter that is activated by the presence of propionate in the mammalian gut. Any molecule or metabolite found in the mammalian gut, in a healthy and/or disease state, may be used to induce payload expression. Non-limiting examples of inducers include propionate, bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese. In alternate embodiments, the gene or gene cassette for producing EGF is under the control of a pBAD promoter, which is activated in the presence of the sugar arabinose.

In some embodiments, the gene or gene cassette for producing the EGF is present on a plasmid and operably linked to a promoter that is induced under low-oxygen or anaerobic conditions. In some embodiments, the gene or gene cassette for producing the EGF is present on a plasmid and operably linked to a temperature sensitive promoter. In some embodiments, the gene or gene cassette for producing EGF is present in the chromosome and operably linked to a promoter that is induced under low-oxygen or anaerobic conditions. In some embodiments, the gene or gene cassette for producing the EGF is present in the chromosome and operably linked to a temperature sensitive promoter. In some embodiments, the gene or gene cassette for producing EGF is present on a plasmid and operably linked to a promoter that is induced by molecules or metabolites that are specific to the mammalian gut. In some embodiments, the gene or gene cassette for producing EGF is present on a chromosome and operably linked to a promoter that is induced by molecules or metabolites that are specific to the tumor and/or the mammalian gut. In some embodiments, the gene or gene cassette for producing EGF is present on a chromosome and operably linked to a promoter that is induced by exposure to tetracycline. In some embodiments, the gene or gene cassette for producing EGF is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the recombinant bacteria comprise a stably maintained plasmid or chromosome carrying the gene or gene cassette for producing EGF, such that the gene or gene cassette can be expressed in the host cell, and the host cell is capable of survival and/or growth in vitro, e.g., in medium, and/or in vivo, e.g., in the gut. In some embodiments, a bacterium may comprise multiple copies of the gene or gene cassette for producing EGF. In some embodiments, gene or gene cassette for producing the payload is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, gene or gene cassette for producing EGF is expressed on a high-copy plasmid. In some embodiments, the high-copy plasmid may be useful for increasing gene or gene cassette expression. In some embodiments, gene or gene cassette for producing EGF is expressed on a chromosome.

In some embodiments, the recombinant bacteria comprise a regulatory region comprising a propionate promoter, which is induced in the mammalian gut. In some embodiments, the propionate promoter sequence is at least about 80%, 85%, 90%, 95%, or 99% homologous to the sequence of SEQ ID NO: 172.

### Other Inducible Promoters

In some embodiments, the gene encoding EGF is present on a plasmid and operably linked to a promoter that is induced by one or more nutritional and/or chemical inducer(s) and/or metabolite(s). In some embodiments, the gene encoding EGF is present in the chromosome and operably linked to a promoter that is induced by one or more nutritional and/or chemical inducer(s) and/or metabolite(s).

In some embodiments, the bacterial cell comprises a stably maintained plasmid or chromosome carrying the one or more gene sequences(s), inducible by one or more nutritional and/or chemical inducer(s) and/or metabolite(s), encoding EGF, such that EGF can be expressed in the host cell, and the host cell is capable of survival and/or growth in vitro, e.g., in medium, and/or in vivo, e.g., in the gut. In some embodiments, bacterial cell comprises two or more distinct copies of the one or more gene sequences(s) encoding EGF, which is controlled by a promoter inducible one or more nutritional and/or chemical inducer(s) and/or metabolite(s). In some embodiments, the recombinant bacteria comprise multiple copies of the same one or more gene sequences(s) encoding EGF, which is controlled by a promoter inducible one or more nutritional and/or chemical inducer(s) and/or metabolite(s). In some embodiments, the one or more gene sequences(s) encoding EGF, is present on a plasmid and operably linked to a directly or indirectly inducible promoter inducible by one or more nutritional and/or chemical inducer(s) and/or metabolite(s). In some embodiments, the one or more gene sequences(s) encoding EGF, is present on a chromosome and operably linked to a directly or indirectly inducible by one or more nutritional and/or chemical inducer(s) and/or metabolite(s).

In some embodiments, expression of EGF is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with EGF prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In some embodiments, expression of one or more EGF molecules is driven directly or indirectly by one or more arabinose inducible promoter(s) in vivo. In some embodiments, the promoter is directly or indirectly induced by a chemical and/or nutritional inducer and/or metabolite which is co-administered with the recombinant bacteria, e.g., arabinose.

The genes of arabinose metabolism are organized in one operon, AraBAD, which is controlled by the PAraBAD promoter. The PAraBAD (or Para) promoter suitably fulfills the criteria of inducible expression systems. PAraBAD displays tighter control of payload gene expression than many other systems, likely due to the dual regulatory role of AraC, which functions both as an inducer and as a repressor. Additionally, the level of ParaBAD-based expression can be modulated over a wide range of L-arabinose concentrations to fine-tune levels of expression of the payload. However, the cell population exposed to sub-saturating L-arabinose concentrations is divided into two subpopulations of induced and uninduced cells, which is determined by the differences between individual cells in the availability of L-arabinose transporter (Zhang et al., Development and Application of an Arabinose-Inducible Expression System by Facilitating Inducer Uptake in Corynebacterium glutamicum; Appl. Environ. Microbiol. August 2012 vol. 78 no. 16 5831-5838). Alternatively, inducible expression from the ParaBad can be controlled or fine-tuned through the optimization of the ribosome binding site (RBS), as described herein.

In one embodiment, the arabinose inducible promoter drives the expression of a construct comprising EGF, jointly with a second promoter, e.g., a second constitutive or inducible promoter. In some embodiments, two promoters are positioned proximally to the construct and drive its expression, wherein the arabinose inducible promoter drives expression under a first set of exogenous conditions, and the second promoter drives the expression under a second set of exogenous conditions. In a non-limiting example, the first and second conditions may be two sequential culture conditions (i.e., during preparation of the culture in a flask, fermenter or other appropriate culture vessel, e.g., arabinose and IPTG). In another non-limiting example, the first inducing conditions may be culture conditions, e.g., including arabinose presence, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, the one or more arabinose promoters drive expression of EGF, in combination with the FNR promoter driving the expression of the same gene sequence(s).

In some embodiments, EGF is knocked into the arabinose operon and are driven by the native arabinose inducible promoter.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 174. In some embodiments, the arabinose inducible construct further comprises a gene encoding AraC, which is divergently transcribed from the same promoter as EGF. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 174. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 175.

In some embodiments, the recombinant bacteria comprise one or more gene sequence(s) which are inducible through a rhamnose inducible system. The genes rhaBAD are organized in one operon which is controlled by the rhaP BAD promoter. The rhaP BAD promoter is regulated by two activators, RhaS and RhaR, and the corresponding genes belong to one transcription unit which divergently transcribed in the opposite direction of rhaBAD. In the presence of L-rhamnose, RhaR binds to the rhaP RS promoter and activates the production of RhaR and RhaS. RhaS together with L-rhamnose then bind to the rhaP BAD and the rhaP T promoter and activate the transcription of the structural genes. In contrast to the arabinose system, in which AraC is provided and divergently transcribed in the gene sequence(s), it is not necessary to express the regulatory proteins in larger quantities in the rhamnose expression system because the amounts expressed from the chromosome are sufficient to activate transcription even on multi-copy plasmids. Therefore, only the rhaP BAD promoter is cloned upstream of the gene that is to be expressed. Full induction of rhaBAD transcription also requires binding of the CRP-cAMP complex, which is a key regulator of catabolite repression. Alternatively, inducible expression from the rhaBAD can be controlled or fine-tuned through the optimization of the ribosome binding site (RBS), as described herein.

In one embodiment, expression of EGF is driven directly or indirectly by one or more rhamnose inducible promoter(s). In one embodiment, expression of the payload is driven directly or indirectly by a rhamnose inducible promoter.

In some embodiments, the rhamnose inducible promoter is useful for or induced during in vivo expression of EGF. In some embodiments, expression of EGF is driven directly or indirectly by one or more rhamnose inducible promoter(s) in vivo. In some embodiments, the promoter is directly or indirectly induced by a molecule that is co-administered with the recombinant bacteria, e.g., rhamnose.

In one embodiment, the rhamnose inducible promoter drives the expression of a construct comprising EGF jointly with a second promoter, e.g., a second constitutive or inducible promoter. In some embodiments, two promoters are positioned proximally to the construct and drive its expression, wherein the rhamnose inducible promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 176.

In some embodiments, the recombinant bacteria comprise one or more gene sequence(s) which are inducible through an Isopropyl β-D-1-thiogalactopyranoside (IPTG) inducible system or other compound which induced transcription from the Lac Promoter. IPTG is a molecular mimic of allolactose, a lactose metabolite that activates transcription of the lac operon. In contrast to allolactose, the sulfur atom in IPTG creates a non-hydrolyzable chemical blond, which prevents the degradation of IPTG, allowing the concentration to remain constant. IPTG binds to the lac repressor and releases the tetrameric repressor (lacd) from the lac operator in an allosteric manner, thereby allowing the transcription of genes in the lac operon. Since IPTG is not metabolized by E. coli, its concentration stays constant and the rate of expression of Lac promoter-controlled is tightly controlled, both in vivo and in vitro. IPTG intake is independent on the action of lactose permease, since other transport pathways are also involved. Inducible expression from the PLac can be controlled or fine-tuned through the optimization of the ribosome binding site (RBS), as described herein. Other compounds which inactivate LacI, can be used instead of IPTG in a similar manner.

In one embodiment, expression of EGF is driven directly or indirectly by one or more IPTG inducible promoter(s). In some embodiments, the IPTG inducible promoter is useful for or induced during in vivo expression of EGF. In some embodiments, expression of EGF is driven directly or indirectly by one or more IPTG inducible promoter(s) in vivo. In some embodiments, the promoter is directly or indirectly induced by a molecule that is co-administered with the recombinant bacteria, e.g., IPTG.

In one embodiment, the IPTG inducible promoter drives the expression of a construct comprising EGF jointly with a second promoter, e.g., a second constitutive or inducible promoter. In some embodiments, two promoters are positioned proximally to the construct and drive its expression, wherein the IPTG inducible promoter drives expression under a first set of exogenous conditions, and the second promoter drives the expression under a second set of exogenous conditions.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 177. In some embodiments, the IPTG inducible construct further comprises a gene encoding lacI, which is divergently transcribed from the same promoter EGF. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 177. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 180.

In some embodiments, the recombinant bacteria comprise one or more gene sequence(s) which are inducible through a tetracycline inducible system. The initial system Gossen and Bujard (Tight control of gene expression in mammalian cells by tetracycline-responsive promoters. Gossen M & Bujard H. PNAS, 1992 Jun. 15; 89(12):5547-51) developed is known as tetracycline off: in the presence of tetracycline, expression from a tet-inducible promoter is reduced. Tetracycline-controlled transactivator (tTA) was created by fusing tetR with the C-terminal domain of VP16 (virion protein 16) from herpes simplex virus. In the absence of tetracycline, the tetR portion of tTA will bind tetO sequences in the tet promoter, and the activation domain promotes expression. In the presence of tetracycline, tetracycline binds to tetR, precluding tTA from binding to the tetO sequences. Next, a reverse Tet repressor (rTetR), was developed which created a reliance on the presence of tetracycline for induction, rather than repression. The new transactivator rtTA (reverse tetracycline-controlled transactivator) was created by fusing rTetR with VP16. The tetracycline on system is also known as the rtTA-dependent system.

In one embodiment, expression of EGF is driven directly or indirectly by one or more tetracycline inducible promoter(s). In some embodiments, the tetracycline inducible promoter is useful for or induced during in vivo expression of EGF. In some embodiments, expression of EGF and/or transcriptional regulator(s), e.g., FNRS24Y, is driven directly or indirectly by one or more tetracycline inducible promoter(s) in vivo. In some embodiments, the promoter is directly or indirectly induced by a molecule that is co-administered with the recombinant bacteria, e.g., tetracycline

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the bolded sequences of SEQ ID NO: 181 (tet promoter is in bold). In some embodiments, the tetracycline inducible construct further comprises a gene encoding AraC, which is divergently transcribed from the same promoter as EGF. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 182 in italics (Tet repressor is in italics). In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 182 in italics (Tet repressor is in italics).

In some embodiments, the recombinant bacteria comprise one or more gene sequence(s) whose expression is controlled by a temperature sensitive mechanism. Thermoregulators are advantageous because of strong transcriptional control without the use of external chemicals or specialized media (see, e.g., Nemani et al., Magnetic nanoparticle hyperthermia induced cytosine deaminase expression in microencapsulated E. coli for enzyme-prodrug therapy; J Biotechnol. 2015 Jun. 10; 203: 32-40, and references therein). Thermoregulated protein expression using the mutant cI857 repressor and the pL and/or pR phage λ promoters have been used to engineer recombinant bacterial strains. The EGF gene cloned downstream of the λ promoters can then be efficiently regulated by the mutant thermolabile cI857 repressor of bacteriophage λ. At temperatures below 37° C., cI857 binds to the oL or oR regions of the pR promoter and blocks transcription by RNA polymerase. At higher temperatures, the functional cI857 dimer is destabilized, binding to the oL or oR DNA sequences is abrogated, and mRNA transcription is initiated. An exemplary construct is depicted in in the figures and examples. Inducible expression from the temperature sensitive promoter can be controlled or further fine-tuned through the optimization of the ribosome binding site (RBS), as described herein.

In one embodiment, expression of EGF is driven directly or indirectly by one or more thermoregulated promoter(s). In some embodiments, thermoregulated promoter is useful for or induced during in vivo expression of EGF. In some embodiments, expression of EGF is driven directly or indirectly by one or more thermoregulated promoter(s) in vitro.

In some embodiments, expression of EGF is driven directly or indirectly by one or more thermoregulated promoter(s) during in vitro growth, preparation, or manufacturing of the strain prior to in vivo administration. In some embodiments, it may be advantageous to shup off production of EGF. This can be done in a thermoregulated system by growing the strain at lower temperatures, e.g., 30° C. Expression can then be induced by elevating the temperature to 37° C. and/or 42° C. In some embodiments, thermoregulated promoter(s) are induced in culture, e.g., grown in a flask, fermenter or other appropriate culture vessel, e.g., used during cell growth, cell expansion, fermentation, recovery, purification, formulation, and/or manufacture. In some embodiments, the cultures, which are induced by temperatures between 37° C. and 42° C., are grown aerobically. In some embodiments, the cultures, which are induced by induced by temperatures between 37° C. and 42° C., are grown anaerobically.

In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 183. In some embodiments, thermoregulated construct further comprises a gene encoding mutant cI857 repressor, which is divergently transcribed from the same promoter as EGF. In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 184. In some embodiments, the recombinant bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 185.

In some instances, thermoregulators may be advantageous because of strong transcriptional control without the use of external chemicals or specialized media. Thermoregulated protein expression using the mutant cI857 repressor and the pL and/or pR phage a promoters have been used to engineer recombinant bacterial strains. For example, a gene of interest cloned downstream of the a promoters can be efficiently regulated by the mutant thermolabile cI857 repressor of bacteriophage a. At temperatures below 37° C., cI857 binds to the oL or oR regions of the pR promoter and inhibits transcription by RNA polymerase. At higher temperatures, the functional cI857 dimer is destabilized, binding to the oL or oR DNA sequences is abrogated, and mRNA transcription is initiated.

In certain instances, it may be advantageous to reduce, diminish, or shut off production of one or more protein(s) of interest. This can be done in a thermoregulated system by growing a bacterial strain at temperatures at which the temperature regulated system is not optimally active. Temperature regulated expression can then be induced as desired by changing the temperature to a temperature where the system is more active or optimally active.

For example, a thermoregulated promoter may be induced in culture, e.g., grown in a flask, fermenter or other appropriate culture vessel, e.g., used during cell growth, cell expansion, fermentation, recovery, purification, formulation, and/or manufacture. Bacteria comprising gene sequences or gene cassettes either indirectly or directly operably linked to a temperature sensitive system or promoter may, for example, could be induced by temperatures between 37° C. and 42° C. In some instances, the cultures may be grown aerobically. Alternatively, the cultures are grown anaerobically.

In some embodiments, the bacteria described herein comprise one or more gene sequence(s) or gene cassette(s) which are directly or indirectly operably linked to a temperature regulated promoter. In some embodiments, the gene sequence(s) or gene cassette(s) are induced in vitro during growth, preparation, or manufacturing of the strain prior to in vivo administration. In some embodiments, the gene sequence(s) are induced upon or during in vivo administration. In some embodiments, the gene sequence(s) are induced during in vitro growth, preparation, or manufacturing of the strain prior to in vivo administration and upon or during in vivo administration. In some embodiments, the genetically engineered bacteria further comprise gene sequence (s) encoding a transcription factor which is capable of binding to the temperature sensitive promoter. In some embodiments, the transcription factor is a repressor of transcription.

In some embodiments, the thermoregulated promoter drives the expression of one or more protein(s) of interest from a low-copy plasmid or a high copy plasmid. In some embodiments, the thermoregulated promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 209. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 213. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 216. In some embodiments, the thermoregulated construct further comprises a gene encoding mutant cI857 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 210. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 212. In some embodiments, the thermoregulated construct further comprises a gene encoding mutant cI38 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 214. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 215.

SEQ ID NOs: 209, 210, and 212-16 are shown in Table 14.

TABLE 14
Inducible promoter construct sequences

| Description | Sequence |
|---|---|
| Region comprising | ACGTTAAATCTATCACCGCAAGGGATAAATATCTAACACC |
| Temperature sensitive | GTGCGTGTTGACTATTTTACCTCTGGCGGTGATAATGGTT |
| promoter | GCATAGCTGTCACCGGATGTGCTTTCCGGTCTGATGAGTC |
| SEQ ID NO: 209 | CGTGAGGACGAAACAGCCTCTACAAATAATTTTGTTTAAA |
| | ACAACACCCACTAAGATAACTCTAGAAATAATTTTGTTTA |
| | ACTTTAAGAAGGAGATATACAT |
| mutant cI857 repressor | TCAGCCAAACGTCTCTTCAGGCCACTGACTAGCGATAACT |
| nucleotide sequence | TTCCCCACAACGGAACAACTCTCATTGCATGGGATCATTG |
| SEQ ID NO: 210 | GGTACTGTGGGTTTAGTGGTTGTAAAAACACCTGACCGCT |
| | ATCCCTGATCAGTTTCTTGAAGGTAAACTCATCACCCCCA |
| | AGTCTGGCTATGCAGAAATCACCTGGCTCAACAGCCTGCT |
| | CAGGGTCAACGAGAATTAACATTCCGTCAGGAAAGCTTGG |
| | CTTGGAGCCTGTTGGTGCGGTCATGGAATTACCTTCAACC |
| | TCAAGCCAGAATGCAGAATCACTGGCTTTTTTGGTTGTGC |
| | TTACCCATCTCTCCGCATCACCTTTGGTAAAGGTTCTAAG |
| | CTTAGGTGAGAACATCCCTGCCTGAACATGAGAAAAAACA |
| | GGGTACTCATACTCACTTCTAAGTGACGGCTGCATACTAA |
| | CCGCTTCATACATCTCGTAGATTTCTCTGGCGATTGAAGG |
| | GCTAAATTCTTCAACGCTAACTTTGAGAATTTTTGTAAGC |
| | AATGCGGCGTTATAAGCATTTAATGCATTGATGCCATTAA |
| | ATAAAGCACCAACGCCTGACTGCCCCATCCCCATCTTGTC |
| | TGCGACAGATTCCTGGGATAAGCCAAGTTCATTTTTCTTT |
| | TTTTCATAAATTGCTTTAAGGCGACGTGCGTCCTCAAGCT |
| | GCTCTTGTGTTAATGGTTTCTTTTTTGTGCTCAT |
| mutant cI857 repressor | MSTKKKPLTQEQLEDARRLKAIYEKKKNELGLSQESVADK |
| polypeptide sequence | MGMGQSGVGALFNGINALNAYNAALLTKILKVSVEEFSPS |
| SEQ ID NO: 212 | IAREIYEMYEAVSMQPSLRSEYEYPVFSHVQAGMFSPKLR |
| | TFTKGDAERWVSTTKKASDSAFWLEVEGNSMTAPTGSKPS |
| | FPDGMLILVDPEQAVEPGDFCIARLGGDEFTFKKLIRDSG |
| | QVFLQPLNPQYPMIPCNESCSVVGKVIASQWPEETFG |
| Pr/Pl promoter | ACGTTAAATCTATCACCGCAAGGGATAAATATCTAACACC |
| SEQ ID NO: 213 | GTGCGTGTTGACTATTTTACCTCTGGCGGTGATAATGGTT |
| | GCAT |
| mutant cI38 repressor | Atgagcacaaaaaagaaaccattaacacaagagcagcttg |
| nucleotide sequence | aggacgcacgtcgccttaaagcaatttatgaaaaaaagaa |
| SEQ ID NO: 214 | aaatgaacttggcttatcccaggaatctgtcgcagacaag |
| | atggggatggggcagtcaggcgttggtgccttatttaatg |
| | gcatcaatgcattaaatgcttataacgccgcatcgcttac |
| | aagaattctcaaagttagcgttgaagaatttagcccttca |
| | atcgccagagaaatctacgagatgtatgaagcggttagta |
| | tgcagccgtcacttagaagtgagtatgagtaccctgtttt |
| | ttctcatgttcaggcagggatgctctcacctgagcttaga |
| | acctttaccaaaggtggtgcggaaaggtgggtaagcacaa |
| | ccaaaaaagccagtgattctgcattctggcttgaggttga |
| | aggtaattccatgacagcaccaacaggctccaagccaagc |
| | tttcctgacggaatgttaattctcgttgaccctgagcagg |
| | ctgttgagccaggtgatttctgcatagccagactcggggg |
| | tggtgagtttaccttcaagaaactgatcagggatagcggt |
| | caggtgtttttacaaccactaaacccacagtacccaatga |
| | tcccatgcaatgagagttgttccgttgtggggaaagttat |
| | cgctagtcagtggcctgaagagacgtttggctga |
| mutant cI38 repressor | MSTKKKPLTQEQLEDARRLKAIYEKKKNELGLSQESVADK |
| polypeptide sequence | MGMGQSGVGALFNGINALNAYNAASLTRILKVSVEEFSPS |
| SEQ ID NO: 215 | IAREIYEMYEAVSMQPSLRSEYEYPVFSHVQAGMLSPELR |
| | TFTKGGAERWVSTTKKASDSAFWLEVEGNSMTAPTGSKPS |
| | FPDGMLILVDPEQAVEPGDFCIARLGGGEFTFKKLIRDSG |
| | QVFLQPLNPQYPMIPCNESCSVVGKVIASQWPEETFG |
| Temperature sensitive | aaatctatcaccgcaagggataaatatctaacaccgtgcg |
| promoter | tgttgactattttacctctggcggtgataatggttgcATa |
| SEQ ID NO: 216 | gctgtcaccggatgtgctttccggtctgatgagtccgtga |
| | ggacgaaacagcctctacaaataattttgtttaaAACAAC |
| | ACCCACTAAGATAAGGTAGAAAC |

The following is an exemplary construct comprising a temperature sensitive promoter-ompA-EGF and mutant cI857 repressor driven by temperature sensitive promoter in reverse orientation: TCAGCCAAACGTCTCTTCAGGCCACTGACTAGCGATAACTTTCCCCACAACGGAACAA CTCTCATTGCATGGGATCATTGGGTACTGTGGGTTTAGTGGTTGTAAAAACACCTGAC CGCTATCCCTGATCAGTTTCTTGAAGGTAAACTCATCACCCCCAAGTCTGGCTATGCA GAAATCACCTGGCTCAACAGCCTGCTCAGGGTCAACGAGAATTAACATTCCGTCAGG AAAGCTTGGCTTGGAGCCTGTTGGTGCGGTCATGGAATTACCTTCAACCTCAAGCCAG AATGCAGAATCACTGGCTTTTTTGGTTGTGCTTACCCATCTCTCCGCATCACCTTTGGT AAAGGTTCTAAGCTTAGGTGAGAACATCCCTGCCTGAACATGAGAAAAAACAGGGTA CTCATACTCACTTCTAAGTGACGGCTGCATACTAACCGCTTCATACATCTCGTAGATTT CTCTGGCGATTGAAGGGCTAAATTCTTCAACGCTAACTTTGAGAATTTTTGTAAGCAA TGCGGCGTTATAAGCATTTAATGCATTGATGCCATTAAATAAAGCACCAACGCCTGAC TGCCCCATCCCCATCTTGTCTGCGACAGATTCCTGGGATAAGCCAAGTTCATTTTTCTT TTTTTCATAAATTGCTTTAAGGCGACGTGCGTCCTCAAGCTGCTCTTGTGTTAATGGTT TCTTTTTTGTGCTCATACGTTAAATCTATCACCGCAAGGGATAAATATCTAACACCGTG CGTGTTGACTATTTTACCTCTGGCGGTGATAATGGTTGCATaataattttgtttaactttaagaaggaggta tacatATGAAGAAAACCGCAATTGCAATCGCCGTCGCTCTGGCGGGGTTCGCTACGGTCG CCCAAGCCAATAGTGACAGCGAATGTCCGCTGTCGCACGATGGTTATTGCCTTCATGA TGGGGTGTGCATGTACATTGAGGCATTGGACAAATATGCCTGCAACTGTGTTGTCGGC TATATCGGCGAACGGTGTCAGTACCGTGATCTGAAATGGTGGGAATTACGC (SEQ ID NO: BB). In some embodiments, the bacterium disclosed herein comprises a sequence that is 80%, 85%, 90%, 91%, 92%, 93%, 94%, 95%, 96%, 97%, 98%, 99%, or 100% identical to SEQ ID NO: BB or a fragment thereof.
The following is an exemplary temperature sensitive promoter (SEQ ID NO: 213): ACGTTAAATCTATCACCGCAAGGGATAAATATCTAACACCGTGCGTGTTGACTATTTT ACCTCTGGCGGTGATAATGGTTGCAT. In some embodiments, the bacterium disclosed herein comprises a sequence that is 80%, 85%, 90%, 91%, 92%, 93%, 94%, 95%, 96%, 97%, 98%, 99%, or 100% identical to SEQ ID NO: 213 or a fragment thereof.

In some embodiments, the recombinant bacteria comprise one or more gene sequence(s) which are indirectly inducible through a system driven by the PssB promoter. The Pssb promoter is active under aerobic conditions, and shuts off under anaerobic conditions.

This promoter can be used to express an EGF gene under aerobic conditions. This promoter can also be used to tightly control the expression of a gene product such that it is only expressed under anaerobic conditions. In this case, the oxygen induced PssB promoter induces the expression of a repressor, which represses the expression of an EGF gene. As a result, the EGF gene is only expressed in the absence of the repressor, i.e., under anaerobic conditions. This strategy has the advantage of an additional level of control for improved fine-tuning and tighter control.

In one embodiment, expression of EGF is indirectly regulated by a repressor expressed under the control of one or more PssB promoter(s). In some embodiments, induction of the RssB promoter(s) indirectly drives the in vivo expression of EGF. In some embodiments, induction of the RssB promoter(s) indirectly drives the expression of EGF during in vitro growth, preparation, or manufacturing of the strain prior to in vivo administration.

Bacteria comprising one or more sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences disclosed herein are also contemplated. In some embodiments, the bacterium comprises one or more sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with a promoter disclosed herein, e.g., a thermoregulated promoter or a promoter induced under low-oxygen or anerobic conditions. In some embodiments, the bacterium comprises one or more sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with a secretion molecule disclosed herein, e.g., nucleotide or amino acid sequence, e.g., ompA. In some embodiments, the bacterium comprises one or more sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with an EGF disclosed herein, e.g., nucleotide or amino acid sequence.

In a non-limiting example, this strategy can be used to control expression of thyA and/or dapA, e.g., to make a conditional auxotroph. The chromosomal copy of dapA or ThyA is knocked out. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can, for example be employed to allow survival of bacteria under anaerobic conditions, e.g., the gut, but prevent survival under aerobic conditions (biosafety switch). In some embodiments, the recombinant bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 188. In some embodiments, the inducible promoters, as described above, drive the expression of EGF from a low-copy plasmid or a high copy plasmid or a biosafety system plasmid described herein. In some embodiments, the inducible promoters drive the expression of EGF from a construct which is integrated into the bacterial chromosome.

### Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, e.g., the gut, as described herein. In some embodiments, the promoters is active under in vitro conditions, e.g., various cell culture and/or cell manufacturing conditions, as described herein. In some embodiments, the constitutive promoter is active under in vivo conditions, e.g., the gut, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the gut of a mammal. In some embodiments, the constitutive promoter is active in exogenous environmental conditions specific to the small intestine of a mammal. In some embodiments, the constitutive promoter is active in low-oxygen or anaerobic conditions such as the environment of the mammalian gut. In some embodiments, the constitutive promoter is active in the presence of molecules or metabolites that are specific to the gut of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions.

Bacterial constitutive promoters are known in the art. Exemplary constitutive promoters include E. coli σ70, such as BBa_114034 (SEQ ID NO: 189), BBa_1732021 (SEQ ID NO: 190), BBa_1742126 (SEQ ID NO: 191), BBa_J01006 (SEQ ID NO: 192), BBa_J23100 (SEQ ID NO: 193), BBa_J23101 (SEQ ID NO: 194), BBa_J23102 (SEQ ID NO: 195), BBa_J23103 (SEQ ID NO: 196), BBa_J23104 (SEQ ID NO: 197), BBa_J23105 (SEQ ID NO: 198), BBa_J23106 (SEQ ID NO: 199), BBa_J23107 (SEQ ID NO: 200), BBa_J23108 (SEQ ID NO: 201), BBa_J23109 (SEQ ID NO: 202), BBa_J23110 (SEQ ID NO: 203), BBa_J23111 (SEQ ID NO: 204), BBa_J23112 (SEQ ID NO: 205), BBa_J23113 (SEQ ID NO: 206), BBa_J23114 (SEQ ID NO: 207), BBa_J23115 (SEQ ID NO: 208), BBa_J23116 (SEQ ID NO: 209), BBa_J23117 (SEQ ID NO: 210), BBa_J23118 (SEQ ID NO: 211), BBa_J23119 (SEQ ID NO: 212), BBa_J23150 (SEQ ID NO: 213), BBa_J23151 (SEQ ID NO: 214), BBa_J44002 (SEQ ID NO: 215), BBa_J48104 (SEQ ID NO: 216), BBa_J54200 (SEQ ID NO: 217), BBa_J56015 (SEQ ID NO: 218), BBa_J64951 (SEQ ID NO: 219), BBa_K088007 (SEQ ID NO: 220), BBa_K119000 (SEQ ID NO: 221), BBa_K119001 (SEQ ID NO: 222), BBa_K1330002 (SEQ ID NO: 223), BBa_K137029 (SEQ ID NO: 224), BBa_K137030 (SEQ ID NO: 225), BBa_K137031 (SEQ ID NO: 226), BBa_K137032 (SEQ ID NO: 227), BBa_K137085 (SEQ ID NO: 228), BBa_K137086 (SEQ ID NO: 229), BBa_K137087 (SEQ ID NO: 230), BBa_K137088 (SEQ ID NO: 231), BBa_K137089 (SEQ ID NO: 232), BBa_K137090 (SEQ ID NO: 233), BBa_K137091 (SEQ ID NO: 234), BBa_K1585100 (SEQ ID NO: 235), BBa_K1585101 (SEQ ID NO: 236), BBa_K1585102 (SEQ ID NO: 237), BBa_K1585103 (SEQ ID NO: 238), BBa_K1585104 (SEQ ID NO: 239), BBa_K1585105 (SEQ ID NO: 240), BBa_K1585106 (SEQ ID NO: 241), BBa_K1585110 (SEQ ID NO: 242), BBa_K1585113 (SEQ ID NO: 243), BBa_K1585115 (SEQ ID NO: 244), BBa_K1585116 (SEQ ID NO: 245), BBa_K1585117 (SEQ ID NO: 246), BBa_K1585118 (SEQ ID NO: 247), BBa_K1585119 (SEQ ID NO: 248), BBa_K1824896 (SEQ ID NO: 249), BBa_K256002 (SEQ ID NO: 250), BBa_K256018 (SEQ ID NO: 251), BBa_K256020 (SEQ ID NO: 252), BBa_K256033 (SEQ ID NO: 253), BBa_K292000 (SEQ ID NO: 254), BBa_K292001 (SEQ ID NO: 255), BBa_K418000 (SEQ ID NO: 256), BBa_K418002 (SEQ ID NO: 257), BBa_K418003 (SEQ ID NO: 258), BBa_K823004 (SEQ ID NO: 259), BBa_K823005 (SEQ ID NO: 260), BBa_K823006 (SEQ ID NO: 261), BBa_K823007 (SEQ ID NO: 262), BBa_K823008 (SEQ ID NO: 263), BBa_K823010 (SEQ ID NO: 264), BBa_K823011 (SEQ ID NO: 265), BBa_K823013 (SEQ ID NO: 266), BBa_K823014 (SEQ ID NO: 267), BBa_M13101 (SEQ ID NO: 268), BBa_M13102 (SEQ ID NO: 269), BBa_M13103 (SEQ ID NO: 270), BBa_M13104 (SEQ ID NO: 271), BBa_M13105 (SEQ ID NO: 272), BBa_M13106 (SEQ ID NO: 273), BBa_M13108 (SEQ ID NO: 274), BBa_M13110 (SEQ ID NO: 275), BBa_M31519 (SEQ ID NO: 276), BBa_R1074 (SEQ ID NO: 277), BBa_R1075 (SEQ ID NO: 278), BBa_S03331 (SEQ ID NO: 279), BBa_114018 (SEQ ID NO: 280), and BBa_114033 (SEQ ID NO: 281). Exemplary constitutive promoters include E. coli 6S promoters, e.g., BBa_J45992 (SEQ ID NO: 282), and BBa_J45993 (SEQ ID NO: 283). Exemplary constitutive promoters further include constitutive E. coli 632 promoters, e.g, BBa_J45504 (SEQ ID NO: 284), BBa_K1895002 (SEQ ID NO: 285), and BBa_K1895003 (SEQ ID NO: 286). Exemplary constitutive promoters further include constitutive B. subtilis σA promoters, e.g., BBa_K780003 (SEQ ID NO: 287), BBa_K823000 (SEQ ID NO: 288), BBa_K823002 (SEQ ID NO: 289), and BBa_K823003 (SEQ ID NO: 290), BBa_K14301 (SEQ ID NO: 291), BBa_K143013 (SEQ ID NO: 292). Exemplary constitutive promoters further include constitutive B. subtilis σB promoters, e.g., BBa_K143010 (SEQ ID NO: 291), BBa_K143011 (SEQ ID NO: 292), BBa_K143013 (SEQ ID NO: 293). Exemplary constitutive promoters further include BBa_K112706 (SEQ ID NO: 294) and BBa_K112707 (SEQ ID NO: 295) promoters.

Exemplary promoters from Bacteriophage T7 or SP6 or various prokaryotes include BBa_K143010 (SEQ ID NO: 293), BBa_K143011 (SEQ ID NO: 294), BBa_K143013 (SEQ ID NO: 295), BBa_I712074 (SEQ ID NO: 296), BBa_I719005 (SEQ ID NO: 297), BBa_J34814 (SEQ ID NO: 298), BBa_J64997 (SEQ ID NO: 299), BBa_K113010 (SEQ ID NO: 300), BBa_K113011 (SEQ ID NO: 301), BBa_K113012 (SEQ ID NO: 302), BBa_K1614000 (SEQ ID NO: 303), BBa_R0085 (SEQ ID NO: 304), BBa_R0180 (SEQ ID NO: 305), BBa_R0181 (SEQ ID NO: 306), BBa_R0182 (SEQ ID NO: 307), BBa_R0183 (SEQ ID NO: 308), BBa_Z0251 (SEQ ID NO: 309), BBa_Z0252 (SEQ ID NO: 310), BBa_Z0253 (SEQ ID NO: 311), BBa_J64998 (SEQ ID NO: 312), BBa_K112706 (SEQ ID NO: 313), BBa_K112707 (SEQ ID NO: 314). Exemplary promoters from yeast and various eukaryotes include BBa_I766557 (SEQ ID NO: 315), BBa_J63005 (SEQ ID NO: 316), BBa_K105027 (SEQ ID NO: 317), BBa_K105028 (SEQ ID NO: 318), BBa_K105029 (SEQ ID NO: 319), BBa_K105030 (SEQ ID NO: 320), BBa_K105031 (SEQ ID NO: 321), BBa_K122000), SEQ ID NO: 322), BBa_K124000 (SEQ ID NO: 323), BBa_K124002 (SEQ ID NO: 324), BBa_K319005 (SEQ ID NO: 325), BBa_M31201 (SEQ ID NO: 326), BBa_I766555 (SEQ ID NO: 327), BBa_I766556 (SEQ ID NO: 328), BBa_I712004 (SEQ ID NO: 329), and BBa_K076017 (SEQ ID NO: 330).

Additional exemplary promoters are listed in Table 8.

TABLE 8
Exemplary constitutive promoters

| Name | Description |
|---|---|
| Plpp | The Plpp promoter is a natural promoter taken from the Nissle |
| SEQ ID NO: 331 | genome. In situ it is used to drive production of lpp, which is known to |
| | be the most abundant protein in the cell. Also, in some previous |
| | RNAseq experiments I was able to confirm that the lpp mRNA is one of |
| | the most abundant mRNA in Nissle during exponential growth. |
| PapFAB46 | See, e.g., Kosuri, S., Goodman, D. B. & Cambray, G. Composability of |
| SEQ ID NO: 332 | regulatory sequences controlling transcription and translation in |
| | Escherichia coli. in 1-20 (2013). doi:10.1073/pnas. |
| PJ23101 + UP | UP element helps recruit RNA polymerase |
| element | |
| SEQ ID NO: 333 | (ggaaaatttttttaaaaaaaaaac (SEQ ID NO: 1087)) |
| PJ23107 + UP | UP element helps recruit RNA polymerase |
| element | |
| SEQ ID NO: 334 | (ggaaaatttttttaaaaaaaaaac (SEQ ID NO: 1087)) |
| PSYN23119 | UP element at 5′ end; consensus -10 region is TATAAT; the consensus |
| SEQ ID NO: 335 | -35 is TTGACA; the extended -10 region is generally TGNTATAAT |
| | (TGGTATAAT in this sequence) |

Bacterial constitutive promoters are known in the art. In some embodiments, the 1 constitutive promoter is at least about 80%, 85%, 90%, 95%, or 99% homologous to the sequence of any one of SEQ ID NOs: 187-343.

### Ribosome Binding Sites

In some embodiments, ribosome binding sites are added, switched out or replaced. By testing a few ribosome binding sites, expression levels can be fine-tuned to the desired level. Various RBS are suitable for prokaryotic expression and can be used to achieve the desired expression levels (See, e.g., Registry of standard biological parts). Exemplary ribosome binding sites include those derived from Master sequence SEQ ID NO: 336. Non limiting examples of such ribosome binding sites include BBa_J61100, BBa_J61101, BBa_J61102, BBa_J61103, BBa_J61104, BBa_J61105, BBa_J61106, BBa_J61107, BBa_J61108, BBa_J61109, BBa_J61110, BBa_J61111, BBa_J61112, BBa_J61113, BBa_J61114, BBa_J61115, BBa_J61116, BBa_J61117, BBa_J61118, BBa_J61119, BBa_J61120, BBa_J61121, BBa_J61122, BBa_J61123, BBa_J61124, BBa_J61125, BBa_J61126, BBa_J61127, BBa_J61128, BBa_J6112, BBa_J61130, BBa_J61131, BBa_J61132, BBa_J61133, BBa_J61134, BBa_J61135, BBa_J61136, BBa_J61137, BBa_J61138, BBa_J61139, BBa_B0029, BBa_B0030, BBa_B0031, BBa_B0032, BBa_B0033, BBa_B0034, BBa_B0035, and BBa_B0064 (SEQ ID NO: 336-384).

### Nucleic Acids

In some embodiments, the disclosure provides novel nucleic acids for producing and secreting EGF. In some embodiments, the nucleic acid encodes one or more EGF or EGF fusion protein polypeptides. Thus, in some embodiments, the nucleic acid comprises gene sequence(s) encoding one or more EGF or EGF fusion protein polypeptides. In some embodiments, the one or more EGF or EGF fusion protein polypeptide(s) comprises a polypeptide sequence selected from any of Sequences A, C, D, E, F, G, H, I, J, K, or L a functional fragment and/or variant thereof, or a polypeptide having at least 80%, 85%, 90%, 95%, 96%, 97%, 98%, or 99% identity thereof, e.g., as assessed by an alignment algorithm such as NCBI BLAST. In some embodiments, the nucleic acid comprises one or more EGF or EGF fusion protein cassettes. In some embodiments, the EGF fusion nucleic acid comprises a polynucleotide selected from Sequences B, N, O, P, Q, R, S, T U, V, or W (see below), a functional fragment and/or variant thereof, or a polynucleotide having at least 80%, 85%, 90%, 95%, 96%, 97%, 98%, or 99% identity thereof, e.g., as assessed by an alignment algorithm such as NCBI BLAST.

Sequence N (PelB-EGF):
atgaaatatctgttgcccacggctgccgcgggtctgctgctgctggcagc
gcaaccggctatggcaAATAGTGACAGCGAATGTCCGCTGTCGCACGATG
GTTATTGCCTTCATGATGGGGTGTGCATGTACATTGAGGCATTGGACAAA
TATGCCTGCAACTGTGTTGTCGGCTATATCGGCGAACGGTGTCAGTACCG
TGATCTGAAATGGTGGGAATTACGCTAA
Sequence O (PhoA-EGF):
atgaaacaaagcactattgcactggcactcttaccgttactgtttacccc
tgtgacaaaagcgAATAGTGACAGCGAATGTCCGCTGTCGCACGATGGTT
ATTGCCTTCATGATGGGGTGTGCATGTACATTGAGGCATTGGACAAATAT
GCCTGCAACTGTGTTGTCGGCTATATCGGCGAACGGTGTCAGTACCGTGA
TCTGAAATGGTGGGAATTACGCTAA
Sequence P (OmpA-EGF):
ATGAAGAAAACCGCAATTGCAATCGCCGTCGCTCTGGCGGGGTTCGCTAC
GGTCGCCCAAGCCAATAGTGACAGCGAATGTCCGCTGTCGCACGATGGTT
ATTGCCTTCATGATGGGGTGTGCATGTACATTGAGGCATTGGACAAATAT
GCCTGCAACTGTGTTGTCGGCTATATCGGCGAACGGTGTCAGTACCGTGA
TCTGAAATGGTGGGAATTACGCTAA
Sequence Q (EGF-LARD3):
ATGAATAGTGACAGCGAATGTCCGCTGTCGCACGATGGTTATTGCCTTCA
TGATGGGGTGTGCATGTACATTGAGGCATTGGACAAATATGCCTGCAACT
GTGTTGTCGGCTATATCGGCGAACGGTGTCAGTACCGTGATCTGAAATGG
TGGGAATTACGCATTGAGGGCCGGggttctgacggtaatgatcttatcca
aggcggtaagggcgcggacttcattgaaggggcaaaggtaatgatacaat
ccgcgataactccggtcacaacacctttttgttctcagggcattttggtc
aggatcgtattataggatatcagccgaccgatcggctggtattccagggc
gctgacggcagcacggatctgcgcgaccatgcgaaagccgttggagcaga
tacggtgctgagttttggcgccgattoggttactctcgtcggggtggggt
taggaggcctgtggagcgagggtgtgctgattagttaa
Sequence R (EGF-HylA):
ATGAATAGTGACAGCGAATGTCCGCTGTCGCACGATGGTTATTGCCTTCA
TGATGGGGTGTGCATGTACATTGAGGCATTGGACAAATATGCCTGCAACT
GTGTTGTCGGCTATATCGGCGAACGGTGTCAGTACCGTGATCTGAAATGG
TGGGAATTACGCtcaacttatgggagccaggacaatcttaatccattaat
taatgaaatcagcaaaatcatttcagctgcaggtaacttcgatgttaagg
aggaaagatctgccgcttctttattgcagttgtccggtaatgccagtgat
ttttcatatggacggaactcaataactttgacagcatcagcataa

In some embodiments, the nucleic acid comprises gene sequence encoding a human EGF polypeptide linked to an FNR-responsive promoter. In certain embodiments, the human EGF polypeptide has at least about 80%, 85%, 90%, 95%, or 99% identity with Sequence A (EGF). In some embodiments, the FNR-responsive promoter is at least about 80%, 85%, 90%, 95%, or 99% homologous to the sequence of any one of SEQ ID NO: 151-167. In some embodiments, the human EGF polypeptide has at least about 85%, 86%, 87%, 88%, 89%, 90%, 91%, 92%, 93%, 94%, 95%, 96%, 97%, 98%, or 99% identity with Sequence A. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167. In some specific embodiments, the human EGF polypeptide comprises Sequence A. In other specific embodiments, the human EGF polypeptide consists of Sequence A. In some embodiments, the nucleic acid comprises a gene sequence linked to an FNR-responsive element.

In some embodiments, the nucleic acid comprises gene sequence encoding a human EGF polypeptide linked to a temperature sensitive promoter construct that further comprises a gene encoding mutant cI857 repressor. In certain embodiments, the human EGF polypeptide has at least about 80%, 85%, 90%, 95%, or 99% identity with Sequence A (EGF). In some embodiments, the temperature sensitive promoter construct comprises a gene encoding mutant cI857 repressor that is at least about 80%, 85%, 90%, 95%, or 99% homologous to the sequence of any one of SEQ ID NO: 183-185. In some embodiments, the human EGF polypeptide has at least about 85%, 86%, 87%, 88%, 89%, 90%, 91%, 92%, 93%, 94%, 95%, 96%, 97%, 98%, or 99% identity with Sequence A. In some embodiments, the temperature sensitive promoter construct comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185. In some specific embodiments, the human EGF polypeptide comprises Sequence A. In other specific embodiments, the human EGF polypeptide consists of Sequence A. In some embodiments, the nucleic acid comprises a gene sequence linked to a temperature sensitive element.

In certain embodiments, the nucleic acid comprising the human EGF gene sequence has at least about 80%, 85%, 90%, 95%, or 99% identity with Sequence B (EGF) linked to an FNR-responsive element. In some embodiments, the nucleic acid comprising the human EGF gene sequence has at least about 85%, 86%, 87%, 88%, 89%, 90%, 91%, 92%, 93%, 94%, 95%, 96%, 97%, 98%, or 99% identity with Sequence B. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167. In some specific embodiments, the nucleic acid comprising the human EGF gene sequence comprises Sequence B with an FNR-responsive element of any one of SEQ ID NO: 151-167. In other specific embodiments the nucleic acid comprising the human EGF gene sequence consists of Sequence B with an FNR-responsive element of any one of SEQ ID NO: 151-167.

In certain embodiments, the nucleic acid comprising the human EGF gene sequence has at least about 80%, 85%, 90%, 95%, or 99% identity with Sequence B (EGF) linked to a temperature sensitive promoter construct that further comprises a gene encoding mutant cI857 repressor. In some embodiments, the nucleic acid comprising the human EGF gene sequence has at least about 85%, 86%, 87%, 88%, 89%, 90%, 91%, 92%, 93%, 94%, 95%, 96%, 97%, 98%, or 99% identity with Sequence B. In some embodiments, the temperature sensitive promoter construct further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185. In some specific embodiments, the nucleic acid comprising the human EGF gene sequence comprises Sequence B with a temperature sensitive element of any one of SEQ ID NO: 183-185. In other specific embodiments the nucleic acid comprising the human EGF gene sequence consists of Sequence B with a temperature sensitive element of any one of SEQ ID NO: 183-185.

In some embodiments, the nucleic acid comprises a gene sequence encoding a PelB polypeptide. In certain embodiments, the PelB polypeptide has at least about 80%, 85%, 90%, 95%, or 99% identity with Sequence C (PelB). In some embodiments, the PelB polypeptide has at least about 85%, 86%, 87%, 88%, 89%, 90%, 91%, 92%, 93%, 94%, 95%, 96%, 97%, 98%, or 99% identity with Sequence C. In some specific embodiments, the PelB polypeptide comprises Sequence C. In other specific embodiments, the PelB polypeptide consists of Sequence C. In some embodiments, the nucleic acid comprises a gene sequence. In certain embodiments, the nucleic acid comprising the PelB gene sequence has at least about 80%, 85%, 90%, 95%, or 99% identity with Sequence S (see below). In some embodiments, the nucleic acid comprising the PelB gene sequence has at least about 85%, 86%, 87%, 88%, 89%, 90%, 91%, 92%, 93%, 94%, 95%, 96%, 97%, 98%, or 99% identity with Sequence S. In some specific embodiments, the nucleic acid comprising the PelB gene sequence comprises Sequence S. In other specific embodiments the nucleic acid comprising the PelB gene sequence consists of Sequence S.

Sequence S (PelB):
atgaaatatctgttgcccacggctgccgcgggtctgctgctgctggcag
cgcaaccggctatggca

In some embodiments, the nucleic acid comprises a gene sequence encoding a PhoA polypeptide. In certain embodiments, the PhoA polypeptide has at least about 80%, 85%, 90%, 95%, or 99% identity with Sequence D (PhoA). In some embodiments, the PhoA polypeptide has at least about 85%, 86%, 87%, 88%, 89%, 90%, 91%, 92%, 93%, 94%, 95%, 96%, 97%, 98%, or 99% identity with Sequence D. In some specific embodiments, the PhoA polypeptide comprises Sequence D. In other specific embodiments, the PhoA polypeptide consists of Sequence D. In some embodiments, the nucleic acid comprises a gene sequence. In certain embodiments, the nucleic acid comprising the PhoA gene sequence has at least about 80%, 85%, 90%, 95%, or 99% identity with Sequence T (see below). In some embodiments, the nucleic acid comprising the PhoA gene sequence has at least about 85%, 86%, 87%, 88%, 89%, 90%, 91%, 92%, 93%, 94%, 95%, 96%, 97%, 98%, or 99% identity with Sequence T. In some specific embodiments, the nucleic acid comprising the PhoA gene sequence comprises Sequence T. In other specific embodiments the nucleic acid comprising the PhoA gene sequence consists of Sequence T.

Sequence T (PhoA):
atgaaacaaagcactattgcactggcactcttaccgttactgtttaccc
ctgtgacaaaagcg

In some embodiments, the nucleic acid comprises a gene sequence encoding a OmpA polypeptide. In certain embodiments, the OmpA polypeptide has at least about 80%, 85%, 90%, 95%, or 99% identity with Sequence E (OmpA). In some embodiments, the OmpA polypeptide has at least about 85%, 86%, 87%, 88%, 89%, 90%, 91%, 92%, 93%, 94%, 95%, 96%, 97%, 98%, or 99% identity with Sequence E. In some specific embodiments, the OmpA polypeptide comprises Sequence E. In other specific embodiments, the OmpA polypeptide consists of Sequence E. In some embodiments, the nucleic acid comprises a gene sequence. In certain embodiments, the nucleic acid comprising the OmpA gene sequence has at least about 80%, 85%, 90%, 95%, or 99% identity with Sequence U (See below). In some embodiments, the nucleic acid comprising the OmpA gene sequence has at least about 85%, 86%, 87%, 88%, 89%, 90%, 91%, 92%, 93%, 94%, 95%, 96%, 97%, 98%, or 99% identity with Sequence U. In some specific embodiments, the nucleic acid comprising the OmpA gene sequence comprises Sequence U. In other specific embodiments the nucleic acid comprising the OmpA gene sequence consists of Sequence U.

Sequence U (OmpA):
ATGAAGAAAACCGCAATTGCAATCGCCGTCGCTCTGGCGGGGTTCGCTA
CGGTCGCCCAAGCC

In some embodiments, the nucleic acid comprises a gene sequence encoding a LARD3 polypeptide. In certain embodiments, the LARD3 polypeptide has at least about 80%, 85%, 90%, 95%, or 99% identity with Sequence F (LARD3). In some embodiments, the LARD3 polypeptide has at least about 85%, 86%, 87%, 88%, 89%, 90%, 91%, 92%, 93%, 94%, 95%, 96%, 97%, 98%, or 99% identity with Sequence F. In some specific embodiments, the LARD3 polypeptide comprises Sequence F. In other specific embodiments, the LARD3 polypeptide consists of Sequence F. In some embodiments, the nucleic acid comprises a gene sequence. In certain embodiments, the nucleic acid comprising the LARD3 gene sequence has at least about 80%, 85%, 90%, 95%, or 99% identity with Sequence V (See below). In some embodiments, the nucleic acid comprising the LARD3 gene sequence has at least about 85%, 86%, 87%, 88%, 89%, 90%, 91%, 92%, 93%, 94%, 95%, 96%, 97%, 98%, or 99% identity with Sequence V. In some specific embodiments, the nucleic acid comprising the LARD3 gene sequence comprises Sequence V. In other specific embodiments the nucleic acid comprising the LARD3 gene sequence consists of Sequence V.

Sequence V (LARD3):
attgagggccggggttctgacggtaatgatcttatccaaggcggtaagg
gcgcggacttcattgaaggcggcaaaggtaatgatacaatccgcgataa
ctccggtcacaacacctttttgttctcagggcattttggtcaggatcgt
attataggatatcagccgaccgatcggctggtattccagggcgctgacg
gcagcacggatctgcgcgaccatgcgaaagccgttggagcagatacggt
gctgagttttggcgccgattcggttactctcgtcggggtggggttagga
ggcctgtggagcgagggtgtgctgattagttaa

In some embodiments, the nucleic acid comprises a gene sequence encoding a HylA polypeptide. In certain embodiments, the HylA polypeptide has at least about 80%, 85%, 90%, 95%, or 99% identity with Sequence G (HylA). In some embodiments, the HylA polypeptide has at least about 85%, 86%, 87%, 88%, 89%, 90%, 91%, 92%, 93%, 94%, 95%, 96%, 97%, 98%, or 99% identity with Sequence G. In some specific embodiments, the HylA polypeptide comprises Sequence G. In other specific embodiments, the HylA polypeptide consists of Sequence G. In some embodiments, the nucleic acid comprises a gene sequence. In certain embodiments, the nucleic acid comprising the HylA gene sequence has at least about 80%, 85%, 90%, 95%, or 99% identity with Sequence W (See below). In some embodiments, the nucleic acid comprising the HylA gene sequence has at least about 85%, 86%, 87%, 88%, 89%, 90%, 91%, 92%, 93%, 94%, 95%, 96%, 97%, 98%, or 99% identity with Sequence W. In some specific embodiments, the nucleic acid comprising the HylA gene sequence comprises Sequence W. In other specific embodiments the nucleic acid comprising the HylA gene sequence consists of Sequence W.

Sequence W (HylA):
tcaacttatgggagccaggacaatcttaatccattaattaatgaaatca
gcaaaatcatttcagctgcaggtaacttcgatgttaaggaggaaagatc
tgccgcttctttattgcagttgtccggtaatgccagtgatttttcatat
ggacggaactcaataactttgacagcatcagcataa

In some embodiments, the nucleic acid comprises a gene sequence encoding an ATP-binding cassette transporter polypeptide. In certain embodiments, the ATP-binding cassette transporter polypeptide has at least about 80%, 85%, 90%, 95%, or 99% identity with Sequence M.1, M.2, and/or M.3. In some embodiments, the ATP-binding cassette transporter polypeptide has at least about 85%, 86%, 87%, 88%, 89%, 90%, 91%, 92%, 93%, 94%, 95%, 96%, 97%, 98%, or 99% identity with Sequence M.1, M.2, and/or M.3. In some specific embodiments, the HylA polypeptide comprises Sequence G. In other specific embodiments, the HylA polypeptide consists of Sequence G. In some embodiments, the nucleic acid comprises a gene sequence. In certain embodiments, the nucleic acid comprising the ATP-binding cassette transporter gene sequence has at least about 80%, 85%, 90%, 95%, or 99% identity with Sequence X.1, X.2, and/or X.3 (See below). In some embodiments, the nucleic acid comprising the ATP-binding cassette transporter gene sequence has at least about 85%, 86%, 87%, 88%, 89%, 90%, 91%, 92%, 93%, 94%, 95%, 96%, 97%, 98%, or 99% identity with Sequence X.1, X.2, and/or X.3. In some specific embodiments, the nucleic acid comprising the ATP-binding cassette transporter gene sequence comprises Sequence X.1, X.2, and/or X.3. In other specific embodiments the nucleic acid comprising the ATP-binding cassette transporter gene sequence consists of Sequence X.1, X.2, and/or X.3.

Sequence X.1 (prtD):
atgaatgcttcctctgagcgagatcgttcgcttttcggcgtgctgagacagttccgccgcagtttctggagcgtcgggattttcagcg
ccgtcatcaacgtgctgatgttagcgccttcggtatatatgctgcaggtgtatgaccgggtgctggcatcaggtaacggtatcaccctgctcatg
ctgactttactgatggcgggactgtgcgcatttatgggcgcgttagagtgggttcgtagtctgcttgtagtgaggctgggtacccgtatcgatct
cgcgctcaatcaggatgtctttaatgcggcatttgcccgtaatctggaggcaggtgatggccgcgccgggctggcgctgaccgacttaacgctg
cttcgccagttcatcaccggcaacgcgcttttcgccttcttcgatgtgccatggtttccgctgtttctactggttctgtttttgctgcacccatg
gctgggaatgctcgcgctgggaggcactgtggtgctagttgcgttagcctggctgaaccagcacctgaccaaccaaccgcttgctgaagccaatc
agcaatcacaacaggcgacccacttagctgacgctcagttgcgcaatgccgacgttattgaagcaatggggatgctgggtaacctgcgtcgacg
ctggcttgcgcgtcattaccgttttatttccctgcagaatcttgccagcgagcgtgccgccgcggttggcggtgcgtccaaatacagccgtattg
cgttgcaatcattgatgctggggctgggagcactgctggcgatcgacggtaagatcacaccagggatgatgatcgccggtagcattttagttgg
tcgggtactgagtccgatcgatcagttaataggggtatggaaacagtggagctctgcgcgcattgcctggcaacgcttaacccggctgatcgc
cgcatatccgccgcgcccggcggcaatggctttacctgcgccggaagggcatctcagtgtggaacaggtctcacttcgcacggtgcaggga
aatacccgactgcaaaacatacatttttcgttgcaggctggcgaaacgctggtcattctcggcgcatctggttccggcaaatcctcgctggcgcg
tttgctggtcggcgcgcagagtccgacacaaggtaaagttcgtctggatggcgctgatttgaatcaggtcgacaaaaacacgtttggtccgacc
attggttatttgccccaggacgtgcagttatttaagggctcgctcgctgaaaacatcgcacgttttggcgatgccgatccggaaaaagttgtagc
ggcagcaaaacttgcgggggtacatgaactcattctgtctctacccaacggttatgacaccgaattgggtgatggcggcggcggtttgtctggc
ggccagagacagcgtatcggtctggcgcgcgctatgtacggcgatccttgtctgctgattctggatgaaccaaacgccagccttgacagogaa
ggcgatcaagccctaatgcaggcgattgtcgcattgcagaaacgcggtgccactgttgtgttgattactcatcgcccggctttaacaacgctgg
ctcaaaagattctgattctccacgagggccaacaacagcgtatgggactggcgcgcgatgtgctgacagaactgcaacagcgcagcgcagc
caatcaggcacggatgaaccccacggccgcaatgcctcaataa
Sequence X.2 (prtE):
atgacaggcatggatataaccactcaggatgaattaaacgaagcagcaatgcgcgatcgcgcttcacgtgatgaagagcgcgca
ttacggctgggctggtggctggtgctcgcgggtttcggtggttttctgctgtgggcattactggcgccgttagataaaggggtggcggtgcaag
gcaacgtggtggtttcaggcaatcgtaaggtgatccaacacatgcagggcggcattgtcgatcggattcaggtgaaagacggcgaccgcgtt
gccgcaggtcaggttctcctgacgctgaatgcggtagatgcccgtaccaccagcgaagggctgggttcccaatatgatcaattaattgctcgc
gaagccagactgctggcggaacagcgcaatcaatcgagtctggcggctacgccacgactggcgcaggcgcgtcagagaccggaaatggc
tgccattattgcgttgcaggaagatctacttcgtagccgtcagcaatcgctaaaactggaaatagatggagtccgcgccagcatcgatggactg
gagacttcgcttggcgcattgcaaaaggtgatgagttctaaacagtccgagcaggcaacgttgtctcagcaattacagggcctccggccgctg
gcggcggataactatgttccgcgtaacaagatgctggaaactgagcgtttgtttgctcaggtctctggcgaactggcccagacaagcggcgag
gtagggcgaacccgacgtgatattcagcagcaaaaattgaggatcgcccagcgtcagcaggaatacgacaaagaggtcaacagcgaattgt
ccgacgtacaagccaaactgaatgaggttatcagtcagcgcgagaaagcggatttcaatctggccaacgttcaggtccgtgcacctgttgccg
gcaccgtggtggatatgaagatcttcaccgaaggtggtgtgatcgcacccgggcaggtcatgatggacatcgttcccgaagaccaaccgctg
cttgttgatgggcgcattcctgtggaaatggtggacaaagtatggtcaggtcttccagtggaactccaattcaccgcgttttcacaaagcacaac
gccgcgtgtaccgggaacggtcaccctgctttccgctgaccggctggtagacgaaaaagacggcactccgtactacggtctccgcattcagg
tgagcgaagaaggcaaacgcagtttgcatggtctggaaattaaaccaggtatgccggttcaggggtttgtccgcaccggagagcgttcgtttat
caattatctgtttaaacctctgatggatcgcatgcatcttgcgttaacggaagagtaa
Sequence X.3 (prtF):
atgcgaaggaaagcggtactactgacggttgttttatctctgtcatgcggcagcgcccaggcgatgggcctcctggatgcatggg
aacttgcacttogtaatgacgcacaactgcgggcggccggtttcgaacgagacgccggtcaggaagaggtggcgatcggtcgcgctgggtt
gctgccatcgctgcaatatacctatggcgcaaattacagtcattccaaggtgacgcaacgcgatcggacgctgaataacacgactaaacgtga
ctacgataactacgtatctacgcttacgctgcgtcagccgctgctggactatgcagcctgggcgcgctatcagcagggcgtcacccgcaaact
tatggctgatcagaggtttcgtgatcgtagtcaggacctgatggtgcgtttgtaccagtcctggagcgaagcattgctggcgcaggagaagctt
atgctcctcgatgcgcagcgccgtgcctatcaggagcagttagcgctgaaccgtcgcttattggctgcgggggaaggaactcaaacagacct
gcgggaaacggaagcacgttacaccgtcactgaagcccaacgtatcgaacaggaagataccctggatgccgctatgacagatctcgaaaac
atgatgggttcgccgctacaaattcaggatctgagtccacttgcgctggatacgttgccggataacgtaaccgaaaatcgctcgttatcacaatg
gcgcgaactgactgtgcggcacaatgcaaagctcgcggttcaacgtgaaaacgtcgattacagccgctatgaaattgagcgtaacagagccg
gccatctgcccacactggacctcgtggcgtccacccgcaacagcttgtccgaatctgaatataactacaaccagaaatacgacacccagacc
gttggtttacaggtgagagtgccgctgtatagcggcggcgccgtttcggcatcaatgcgtcaggcagcggcggaataccagcaaagtcaggc
ggagctggataaccagacccgtcagacctttgcggaattacgccgtcagttcaacctgtgtgctaatggggccgcaaaaatccgagcctggc
agatgagcgtggcagctgctgaggaagcgatccgcgcgacgcggcaaagcgtcgccggcggcgagcgcattaatctggacgttctgatgg
cggaacaggagtggtataacgcccgccgcgagctgacagaagtcaaatatcgctggttacaggcctggctgaatttgcgttataccgccgga
acactgaatgagcaagatatgatgcaattggctgcctggtttcaatctgcgcctgtaattaataaaactggtataaataaaaccggaattaacgc
agctaccggtaataaaactaattaa

In some embodiments, the nucleic acid comprises gene encoding a human EGF polypeptide fused to a PelB secretion tag, optionally wherein the gene is operably linked to an FNR-inducible promoter. In certain embodiments, the human EGF fusion protein has at least about 80%, 85%, 90%, 95%, or 99% identity with Sequence H (PelB-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence H. In some specific embodiments, the human EGF fusion protein comprises Sequence H. In some embodiments, the gene encoding the fusion is operably linked to an FNR-responsive promoter having at least about 80%, 85%, 90%, 95%, or 99% homologous to the sequence of any one of SEQ ID NO: 151-167. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167.

In some embodiments, the nucleic acid comprises gene encoding a human EGF polypeptide fused to a PelB secretion tag, optionally wherein the gene is operably linked to a temperature sensitive promoter construct that further comprises a gene encoding mutant cI857 repressor. In certain embodiments, the human EGF fusion protein has at least about 80%, 85%, 90%, 95%, or 99% identity with Sequence H (PelB-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence H. In some specific embodiments, the human EGF fusion protein comprises Sequence H. In some embodiments, the gene encoding the fusion is operably linked to a temperature sensitive promoter construct that further comprises a gene encoding mutant cI857 repressor that having at least about 80%, 85%, 90%, 95%, or 99% homologous to the sequence of any one of SEQ ID NO: 183-185. In some embodiments, the temperature sensitive promoter construct that further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185.

In some embodiments, the nucleic acid comprises gene encoding a human EGF polypeptide fused to a PhoA secretion tag, optionally wherein the gene is operably linked to an FNR-inducible promoter. In certain embodiments, the human EGF fusion protein has at least about 80%, 85%, 90%, 95%, or 99% identity with Sequence I (PhoA-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence I. In some specific embodiments, the human EGF fusion protein comprises Sequence I. In some embodiments, the gene encoding the fusion is operably linked to an FNR-responsive promoter having at least about 80%, 85%, 90%, 95%, or 99% homologous to the sequence of any one of SEQ ID NO: 151-167. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167.

In some embodiments, the nucleic acid comprises gene encoding a human EGF polypeptide fused to a PhoA secretion tag, optionally wherein the gene is operably linked to a temperature sensitive promoter construct that further comprises a gene encoding mutant cI857 repressor. In certain embodiments, the human EGF fusion protein has at least about 80%, 85%, 90%, 95%, or 99% identity with Sequence I (PhoA-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence I. In some specific embodiments, the human EGF fusion protein comprises Sequence I. In some embodiments, the gene encoding the fusion is operably linked to a temperature sensitive promoter construct that further comprises a gene encoding mutant cI857 repressor that having at least about 80%, 85%, 90%, 95%, or 99% homologous to the sequence of any one of SEQ ID NO: 183-185. In some embodiments, the Temperature sensitive promoter construct that further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185.

In some embodiments, the nucleic acid comprises gene encoding a human EGF polypeptide fused to a OmpA secretion tag, optionally wherein the gene is operably linked to an FNR-inducible promoter. In certain embodiments, the human EGF fusion protein has at least about 80%, 85%, 90%, 95%, or 99% identity with Sequence J (OmpA-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence J. In some specific embodiments, the human EGF fusion protein comprises Sequence J. In some embodiments, the gene encoding the fusion is operably linked to an FNR-responsive promoter having at least about 80%, 85%, 90%, 95%, or 99% homologous to the sequence of any one of SEQ ID NO: 151-167. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167.

In some embodiments, the nucleic acid comprises a gene encoding a human EGF polypeptide fused to a OmpA secretion tag, optionally wherein the gene is operably linked to an temperature sensitive promoter construct that further comprises a gene encoding mutant cI857 repressor. In certain embodiments, the human EGF fusion protein has at least about 80%, 85%, 90%, 95%, or 99% identity with Sequence J (OmpA-EGF). In some embodiments, the human EGF fusion protein has at least about 85%, 86%, 87%, 88%, 89%, 90%, 91%, 92%, 93%, 94%, 95%, 96%, 97%, 98%, or 99% identity with Sequence J. In some specific embodiments, the human EGF fusion protein comprises Sequence J. In some embodiments, the gene encoding the fusion is operably linked to a temperature sensitive promoter construct that further comprises a gene encoding mutant cI857 repressor that having at least about 80%, 85%, 90%, 95%, or 99% homologous to the sequence of any one of SEQ ID NO: 183-185. In some embodiments, the temperature sensitive promoter construct that further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185.

In some embodiments, the nucleic acid comprises gene encoding a human EGF polypeptide fused to a LARD3 secretion tag, optionally wherein the gene is operably linked to an FNR-inducible promoter. In certain embodiments, the human EGF fusion protein has at least about 80%, 85%, 90%, 95%, or 99% identity with Sequence K (EGF-LARD3). In some embodiments, the human EGF fusion protein has at least about 85%, 86%, 87%, 88%, 89%, 90%, 91%, 92%, 93%, 94%, 95%, 96%, 97%, 98%, or 99% identity with Sequence K. In some specific embodiments, the human EGF fusion protein comprises Sequence K. In some embodiments, the gene encoding the fusion is operably linked to an FNR-responsive promoter having at least about 80%, 85%, 90%, 95%, or 99% homologous to the sequence of any one of SEQ ID NO: 151-167. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167.

In some embodiments, the nucleic acid comprises a gene encoding a human EGF polypeptide fused to a LARD3 secretion tag, optionally wherein the gene is operably linked to an temperature sensitive promoter construct that further comprises a gene encoding mutant cI857 repressor. In certain embodiments, the human EGF fusion protein has at least about 80%, 85%, 90%, 95%, or 99% identity with Sequence K (EGF-LARD3). In some embodiments, the human EGF fusion protein has at least about 85%, 86%, 87%, 88%, 89%, 90%, 91%, 92%, 93%, 94%, 95%, 96%, 97%, 98%, or 99% identity with Sequence K. In some specific embodiments, the human EGF fusion protein comprises Sequence K. In some embodiments, the gene encoding the fusion is operably linked to a temperature sensitive promoter construct that further comprises a gene encoding mutant cI857 repressor that having at least about 80%, 85%, 90%, 95%, or 99% homologous to the sequence of any one of SEQ ID NO: 183-185. In some embodiments, the temperature sensitive promoter construct that further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185.

In some embodiments, the nucleic acid comprises gene encoding a human EGF polypeptide fused to a HylA secretion tag, optionally wherein the gene is operably linked to an FNR-inducible promoter. In certain embodiments, the human EGF fusion protein has at least about 80%, 85%, 90%, 95%, or 99% identity with Sequence L (EGF-HylA). In some embodiments, the human EGF fusion protein has at least about 85%, 86%, 87%, 88%, 89%, 90%, 91%, 92%, 93%, 94%, 95%, 96%, 97%, 98%, or 99% identity with Sequence L. In some specific embodiments, the human EGF fusion protein comprises Sequence L. In some embodiments, the gene encoding the fusion is operably linked to an FNR-responsive promoter having at least about 80%, 85%, 90%, 95%, or 99% homologous to the sequence of any one of SEQ ID NO: 151-167. In some embodiments, the FNR-responsive promoter has at least about 85%, 86%, 87%, 88%, 89%, 90%, 91%, 92%, 93%, 94%, 95%, 96%, 97%, 98%, or 99% identity with the sequence of any one of SEQ ID NO: 151-167.

In some embodiments, the nucleic acid comprises a gene encoding a human EGF polypeptide fused to a HylA secretion tag, optionally wherein the gene is operably linked to a temperature sensitive promoter construct that further comprises a gene encoding mutant cI857 repressor. In certain embodiments, the human EGF fusion protein has at least about 80%, 85%, 90%, 95%, or 99% identity with Sequence L (EGF-HylA). In some embodiments, the human EGF fusion protein has at least about 85%, 86%, 87%, 88%, 89%, 90%, 91%, 92%, 93%, 94%, 95%, 96%, 97%, 98%, or 99% identity with Sequence L. In some specific embodiments, the human EGF fusion protein comprises Sequence L. In some embodiments, the gene encoding the fusion is operably linked to a temperature sensitive promoter construct that further comprises a gene encoding mutant cI857 repressor that having at least about 80%, 85%, 90%, 95%, or 99% homologous to the sequence of any one of SEQ ID NO: 183-185. In some embodiments, the temperature sensitive promoter construct that further comprises a gene encoding mutant cI857 repressor that has at least about 85%, 86%, 87%, 88%, 89%, 90%, 91%, 92%, 93%, 94%, 95%, 96%, 97%, 98%, or 99% identity with the sequence of any one of SEQ ID NO: 183-185.

In some embodiments, the nucleic acid comprises gene sequence encoding a Fc (IgA) polypeptide. In certain embodiments, the Fc (IgA) polypeptide has at least about 80%, 85%, 90%, 95%, or 99% identity with SEQ ID NO: 499. In some embodiments, the Fc (IgA) polypeptide has at least about 85%, 86%, 87%, 88%, 89%, 90%, 91%, 92%, 93%, 94%, 95%, 96%, 97%, 98%, or 99% identity with SEQ ID NO: 499. In some specific embodiments, the Fc (IgA) polypeptide comprises SEQ ID NO: 499. In other specific embodiments, the Fc (IgA) polypeptide consists of SEQ ID NO: 499. In some embodiments, the nucleic acid comprises a gene sequence. In certain embodiments, the nucleic acid comprising the Fc (IgA) gene sequence has at least about 80%, 85%, 90%, 95%, or 99% identity with SEQ ID NO: 527. In some embodiments, the nucleic acid comprising the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 527. In some specific embodiments, the nucleic acid comprising the Fc (IgA) gene sequence comprises SEQ ID NO: 527. In other specific embodiments the nucleic acid comprising the Fc (IgA) gene sequence consists of SEQ ID NO: 527.

In some embodiments, the nucleic acid comprises a gene sequence. In certain embodiments, the nucleic acid comprising the Fc (IgA) gene sequence has at least about 80%, 85%, 90%, 95%, or 99% identity with SEQ ID NO: 528. In some embodiments, the nucleic acid comprising the Fc (IgA) gene sequence has at least about 85%, 86%, 87%, 88%, 89%, 90%, 91%, 92%, 93%, 94%, 95%, 96%, 97%, 98%, or 99% identity with SEQ ID NO: 528. In some specific embodiments, the nucleic acid comprising the Fc (IgA) gene sequence comprises SEQ ID NO: 528. In other specific embodiments the nucleic acid comprising the Fc (IgA) gene sequence consists of SEQ ID NO: 528.

In some embodiments, the nucleic acid comprises gene sequence encoding a Linker polypeptide. In certain embodiments, the Linker polypeptide has at least about 80%, 85%, 90%, 95%, or 99% identity with SEQ ID NO: 509. In some embodiments, the Linker polypeptide has at least about 85%, 86%, 87%, 88%, 89%, 90%, 91%, 92%, 93%, 94%, 95%, 96%, 97%, 98%, or 99% identity with SEQ ID NO: 509. In some specific embodiments, the Linker polypeptide comprises SEQ ID NO: 509. In other specific embodiments, the Linker polypeptide consists of SEQ ID NO: 509. In some embodiments, the nucleic acid comprises a gene sequence. In certain embodiments, the nucleic acid comprising the LINKER gene sequence has at least about 80%, 85%, 90%, 95%, or 99% identity with SEQ ID NO: 524. In some embodiments, the nucleic acid comprising the Linker gene sequence has at least about 85%, 86%, 87%, 88%, 89%, 90%, 91%, 92%, 93%, 94%, 95%, 96%, 97%, 98%, or 99% identity with SEQ ID NO: 524. In some specific embodiments, the nucleic acid comprising the Linker gene sequence comprises SEQ ID NO: 524. In other specific embodiments the nucleic acid comprising the linker gene sequence consists of SEQ ID NO: 524. In some embodiments, the nucleic acid comprises gene sequence encoding a EGF-linker Fc (IgA) polypeptide.

In some embodiments, the nucleic acid comprises gene sequence encoding a PhoA-EGF-Fc (hIgA) polypeptide.

In some embodiments, the nucleic acid comprises gene sequence encoding a PelB-EGF-Fc (hIgA) polypeptide.

In some embodiments, the nucleic acid comprises gene sequence encoding a OmpA-EGF-Fc (hIgA) polypeptide.

In some embodiments, the nucleic acid comprises gene sequence encoding a ECOLIN 19410-EGF polypeptide.

In some embodiments, the nucleic acid comprises gene sequence encoding a ECOLIN 19410-EGF-Fc (hIgA) polypeptide.

In some embodiments, the nucleic acid comprises gene sequence encoding an EGF-LARD3-Fc (hIgA) polypeptide.

In some embodiments, the nucleic acid comprises gene sequence encoding an EGF-HylA-Fc (hIgA) polypeptide. In some embodiments, the nucleic acid comprises gene sequence encoding a mutated EGF polypeptide. In certain embodiments, the mutated EGF polypeptide has at least about 80%, 85%, 90%, 95%, or 99% identity with Sequence A. In some embodiments, the mutated EGF polypeptide has at least about 85%, Sequence A. In some specific embodiments, the mutated EGF polypeptide comprises Sequence A. In other specific embodiments, the mutated EGF polypeptide consists of Sequence A. In some embodiments, the nucleic acid comprises a gene sequence. In certain embodiments, the nucleic acid comprising the mutated EGF gene sequence has at least about 80%, 85%, 90%, 95%, or 99% identity with Sequence B. In some embodiments, the nucleic acid comprising the mutated EGF gene sequence has at least about 85%, 86%, 87%, 88%, 89%, 90%, 91%, 92%, 93%, 94%, 95%, 96%, 97%, 98%, or 99% identity with Sequence B. In some specific embodiments, the nucleic acid comprising the mutated EGF gene sequence comprises Sequence B. In other specific embodiments the nucleic acid comprising the mutated EGF gene sequence consists of Sequence B.

In some embodiments, the nucleic acid comprises gene sequence encoding a mutated EGF-linker Fc (IgA) polypeptide.

In some embodiments, the nucleic acid comprises gene sequence encoding a PhoA-mutated EGF polypeptide.

In some embodiments, the nucleic acid comprises gene sequence encoding a PhoA-mutated EGF-Fc (hIgA) polypeptide.

In some embodiments, the nucleic acid comprises gene sequence encoding a PelB-mutated EGF polypeptide.

In some embodiments, the nucleic acid comprises gene sequence encoding a PelB-mutated EGF-Fc (hIgA) polypeptide.

In some embodiments, the nucleic acid comprises gene sequence encoding a OmpA-mutated EGF polypeptide.

In some embodiments, the nucleic acid comprises gene sequence encoding a OmpA-mutated EGF-Fc (hIgA) polypeptide.

In some embodiments, the nucleic acid comprises gene sequence encoding a the ECOLIN 19410-mutated EGF polypeptide.

In some embodiments, the nucleic acid comprises gene sequence encoding an EGF-LARD3-mutated polypeptide.

In some embodiments, the nucleic acid comprises gene sequence encoding an EGF-LARD3-mutated-Fc (hIgA) polypeptide.

In some embodiments, the nucleic acid comprises gene sequence encoding an EGF-HylA-mutated polypeptide.

In some embodiments, the nucleic acid comprises gene sequence encoding an EGF-HylA-mutated-Fc (hIgA) polypeptide.

In any of the above embodiments, the nucleic acid may further comprise one or more of the following sequences: (1) promoter, (2) enhancer, (3) regulatory sequence, (4) ribosome binding site—nonlimiting examples of RBS are provided herein and include SEQ ID NO: 336-384, (5) secretion tag, non-limiting examples of secretion tags are provided herein and include SEQ ID NO: 385-394 (6) leader sequence, (7) auxotrophy, (8) antibiotic resistance.

In any of these embodiments, the nucleic acid may be functionally replaced, modified, and/or mutated in order to enhance stability and/or increase polypeptide production or secretion. In some embodiments, the nucleic acid is expressed and secreted in low-oxygen conditions, in the presence of certain molecules or metabolites, in the presence of molecules or metabolites associated with inflammation or an inflammatory response, or in the presence of some other metabolite that may or may not be present in the gut, such as arabinose. Exemplary chemical inducers are described herein. In some embodiments, the nucleic acid is directly operably linked to a first promoter. In some embodiments, the nucleic acid is indirectly operably linked to a first promoter. In one embodiment, the promoter is not operably linked with the nucleic acid in nature.

In some embodiments, nucleic acid is expressed under the control of a constitutive promoter. Non-limiting examples constitutive promoters are provided herein and include SEQ ID NO: 189-335.

In another embodiment, the nucleic acid is expressed under the control of an inducible promoter. In some embodiments, the nucleic acid is expressed under the control of a promoter that is directly or indirectly induced by exogenous environmental conditions. In one embodiment, the nucleic acid is expressed under the control of a promoter that is directly or indirectly induced by low-oxygen or anaerobic conditions, wherein expression of the nucleic acid is activated under low-oxygen or anaerobic environments, such as the environment of the mammalian gut. Inducible promoters are described in more detail infra. Non-limiting examples of low oxygen inducible promoters are provided herein and include SEQ ID NO: 151-167. Non-limiting examples of OxyR inducible promoters are provided herein and include SEQ ID NO: 168-171. Non-limiting examples of promoters regulated by chemical inducers are provided herein and include SEQ ID NO: 173-188.

In some embodiments, the nucleic acid sequence comprises an FNR-responsive promoter linked to a gene sequence encoding an EGF fusion polypeptide. In certain embodiments, the nucleic acid comprising the FNR-responsive promoter linked to a gene sequence encoding an EGF fusion polypeptide has at least about 80%, 85%, 90%, 95%, or 99% identity with Sequence Y. In some embodiments, the nucleic acid comprising the FNR-responsive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence Y. In some specific embodiments, the nucleic acid comprising the gene sequence encoding an EGF fusion polypeptide comprises Sequence H (PelB-EGF) with an FNR-responsive element of any one of SEQ ID NO: 151-167. In other specific embodiments the nucleic acid comprising the human EGF gene sequence consists of Sequence H with an FNR-responsive element of any one of SEQ ID NO: 151-167.

Sequence Y (pFNR-PelB-EGF):
tagcggagtgtatactggcttactatgttggcactgatgagggtgtcagtgaagtgcttcatgtggcaggagaaaaaaggctgcac
cggtgcgtcagcagaatatgtgatacaggatatattccgcttcctcgctcactgactcgctacgctcggtcgttcgactgcggcgagcggaaat
ggcttacgaacggggcggagatttcctggaagatgccaggaagatacttaacagggaagtgagagggccgcggcaaagccgtttttccata
ggctccgcccccctgacaagcatcacgaaatctgacgctcaaatcagtggtggcgaaacccgacaggactataaagataccaggcgtttccc
ctggcggctccctcgtgcgctctcctgttcctgcctttcggtttaccggtgtcattccgctgttatggccgcgtttgtctcattccacgcctgac
actcagttccgggtaggcagttcgctccaagctggactgtatgcacgaaccccccgttcagtccgaccgctgcgccttatccggtaactatcgtc
ttgagtccaacccggaaagacatgcaaaagcaccactggcagcagccactggtaattgatttagaggagttagtcttgaagtcatgcgccggtta
aggctaaactgaaaggacaagttttggtgactgcgctcctccaagccagttacctcggttcaaagagttggtagctcagagaaccttcgaaaaac
cgccctgcaaggcggttttttcgttttcagagcaagagattacgcgcagaccaaaacgatctcaagaagatcatcttattaaggggtctgacgct
cagtggaacggtgcaccctgcagggctagctgataaagcgttcgcgctgcattcggcagtttaattaaAGTTGTTCTTATTGGT
GGTGTTGCTTTATGGTTGCATCGTAGTAAATGGTTGTAACAAAAGCAATTTTTCCGGCT
GTCTGTATACAAAAACGCCGCAAAGTTTGAGCGAAGTCAATAAACTCTCTACCCATTC
AGGGCAATATCTCTCTTggatccaaagtgaactctagaaataattttgtttaactttaagaaggaggtatacatatgaaatatctgtt
gcccacggctgccgcgggtctgctgctgctggcagcgcaaccggctATGGCAAATAGTGACAGCGAATGTCCGC
TGTCGCACGATGGTTATTGCCTTCATGATGGGGTGTGCATGTACATTGAGGCATTGGA
CAAATATGCCTGCAACTGTGTTGTCGGCTATATCGGCGAACGGTGTCAGTACCGTGAT
CTGAAATGGTGGGAATTACGCtaaGCATGCTAATCAGCCGTGGAATTCGGTCTCaGGAG
gAACGATTGGTAAACCCGGTGaacgcatgagAAAGCCCCCGGAAGATCACCTTCCGGGGGC
TTTtttattgcgcGGACCAAAACGAAAAAAGACGCTCGAAAGCGTCTCTTTTCTGGAATTTG
GTACCGAGGcgtaatgctctgccagtgttacaaccaattaaccaattctgattagaaaaactcatcgagcatcaaatgaaactgcaattta
ttcatatcaggattatcaataccatatttttgaaaaagccgtttctgtaatgaaggagaaaactcaccgaggcagttccataggatggcaagatc
ctggtatcggtctgcgattccgactcgtccaacatcaatacaacctattaatttcccctcgtcaaaaataaggttatcaagtgagaaatcaccat
gagtgacgactgaatccggtgagaatggcaaaagcttatgcatttctttccagacttgttcaacaggccagccattacgctcgtcatcaaaatca
ctcgcatcaaccaaaccgttattcattcgtgattgcgcctgagcgagacgaaatacgcgatcgctgttaaaaggacaattacaaacaggaatcga
atgcaaccggcgcaggaacactgccagcgcatcaacaatattttcacctgaatcaggatattcttctaatacctggaatgctgttttcccgggga
tcgcagtggtgagtaaccatgcatcatcaggagtacggataaaatgcttgatggtcggaagaggcataaattccgtcagccagtttagtctgacc
atctcatctgtaacatcattggcaacgctacctttgccatgtttcagaaacaactctggcgcatcgggcttcccatacaatcgatagattgtcgc
acctgattgcccgacattatcgcgagcccatttatacccatataaatcagcatccatgttggaatttaatcgcggcctcgagcaagacgtttccc
gttgaatatggctcataacaccccttgtattactgtttatgtaagcagacagttttattgttcatgatgatatatttttatcttgtgcaatgtaa
catcagagattttgagacacaacgtggctttgttgaataaatcgaacttttgctgagttgaaggatcagatcacgcatcttcccgacaacgcaga
ccgttccgtggcaaagcaaaagttcaaaatcaccaactggtccacctacaacaaagctctcatcaaccgtggctccctcactttctggctggatg
atggggcgattcaggcctggtatgagtcagcaacaccttcttcacgaggcagacctcagcgc

In some embodiments, the nucleic acid sequence comprises an FNR-responsive promoter linked to a gene sequence encoding an EGF fusion polypeptide. In certain embodiments, the nucleic acid comprising the FNR-responsive promoter linked to a gene sequence encoding an EGF fusion polypeptide has at least about 80%, 85%, 90%, 95%, or 99% identity with Sequence Z. In some embodiments, the nucleic acid comprising the FNR-responsive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence Z. In some specific embodiments, the nucleic acid comprising the gene sequence encoding an EGF fusion polypeptide comprises Sequence I (PhoA-EGF) with an FNR-responsive element of any one of SEQ ID NO: 151-167. In other specific embodiments the nucleic acid comprising the human EGF gene sequence consists of Sequence I with an FNR-responsive element of any one of SEQ ID NO: 151-167.

Sequence Z (pFNR-PhoA-EGF):
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaAGTTGTTCTTATTGGTGGTGTT
GCTTTATGGTTGCATCGTAGTAAATGGTTGTAACAAAAGCAATTTTTCCGGCTGTCTGT
ATACAAAAACGCCGCAAAGTTTGAGCGAAGTCAATAAACTCTCTACCCATTCAGGGC
AATATCTCTCTTggatccaaagtgaactctagaaataattttgtttaactttaagaaggaggtatacatatgaaacaaagcactattgca
ctggcactcttaccgttactgtttacccctgtgacaaaagcgAATAGTGACAGCGAATGTCCGCTGTCGCACGAT
GGTTATTGCCTTCATGATGGGGTGTGCATGTACATTGAGGCATTGGACAAATATGCCT
GCAACTGTGTTGTCGGCTATATCGGCGAACGGTGTCAGTACCGTGATCTGAAATGGTG
GGAATTACGCtaaGCATGCTAATCAGCCGTGGAATTCGGTCTCaGGAGgTACGCATGGC
ATGGATGACCCGGTGaacgcatgagAAAGCCCCCGGAAGATCACCTTCCGGGGGCTTTtttattg
cgcGGACCAAAACGAAAAAAGACGCTCGAAAGCGTCTCTTTTCTGGAATTTGGTACCG
AGGcgtaatgctctgccagtgttacaaccaattaaccaattctgattagaaaaactcatcgagcatcaaatgaaactgcaatttattcatatcag
gattatcaataccatatttttgaaaaagccgtttctgtaatgaaggagaaaactcaccgaggcagttccataggatggcaagatcctggtatcgg
tctgcgattccgactcgtccaacatcaatacaacctattaatttcccctcgtcaaaaataaggttatcaagtgagaaatcaccatgagtgacgac
tgaatccggtgagaatggcaaaagcttatgcatttctttccagacttgttcaacaggccagccattacgctcgtcatcaaaatcactcgcatcaa
ccaaaccgttattcattcgtgattgcgcctgagcgagacgaaatacgcgatcgctgttaaaaggacaattacaaacaggaatcgaatgcaaccgg
cgcaggaacactgccagcgcatcaacaatattttcacctgaatcaggatattcttctaatacctggaatgctgttttcccggggatcgcagtggt
gagtaaccatgcatcatcaggagtacggataaaatgcttgatggtcggaagaggcataaattccgtcagccagtttagtctgaccatctcatctg
taacatcattggcaacgctacctttgccatgtttcagaaacaactctggcgcatcgggcttcccatacaatcgatagattgtcgcacctgattgc
ccgacattatcgcgagcccatttatacccatataaatcagcatccatgttggaatttaatcgcggcctcgagcaagacgtttcccgttgaatatg
gctcataacaccccttgtattactgtttatgtaagcagacagttttattgttcatgatgatatatttttatcttgtgcaatgtaacatcagagat
tttgagacacaacgtggctttgttgaataaatcgaacttttgctgagttgaaggatcagatcacgcatcttcccgacaacgcagaccgttccgtg
gcaaagcaaaagttcaaaatcaccaactggtccacctacaacaaagctctcatcaaccgtggctccctcactttctggctggatgatggggcgat
tcaggcctggtatgagtcagcaacaccttcttcacgaggcagacctcagcgc

In some embodiments, the nucleic acid sequence comprises an FNR-responsive promoter linked to a gene sequence encoding an EGF fusion polypeptide. In certain embodiments, the nucleic acid comprising the FNR-responsive promoter linked to a gene sequence encoding an EGF fusion polypeptide has at least about 80%, 85%, 90%, 95%, or 99% identity with Sequence AA. In some embodiments, the nucleic acid comprising the FNR-responsive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AA. In some specific embodiments, the nucleic acid comprising the gene sequence encoding an EGF fusion polypeptide comprises Sequence J (OmpA-EGF) with an FNR-responsive element of any one of SEQ ID NO: 151-167. In other specific embodiments the nucleic acid comprising the human EGF gene sequence consists of Sequence J with an FNR-responsive element of any one of SEQ ID NO: 151-167.

Sequence AA (pFNR-OmpA-EGF):
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggcggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaAGTTGTTCTTATTGGTGGTGTT
GCTTTATGGTTGCATCGTAGTAAATGGTTGTAACAAAAGCAATTTTTCCGGCTGTCTGT
ATACAAAAACGCCGCAAAGTTTGAGCGAAGTCAATAAACTCTCTACCCATTCAGGGC
AATATCTCTCTTggatccaaagtgaactctagaaataattttgtttaactttaagaaggaggtatacatATGAAGAAAACC
GCAATTGCAATCGCCGTCGCTCTGGCGGGGTTCGCTACGGTCGCCCAAGCCAATAGTG
ACAGCGAATGTCCGCTGTCGCACGATGGTTATTGCCTTCATGATGGGGTGTGCATGTA
CATTGAGGCATTGGACAAATATGCCTGCAACTGTGTTGTCGGCTATATCGGCGAACGG
TGTCAGTACCGTGATCTGAAATGGTGGGAATTACGCtaaGCATGCTAATCAGCCGTGGA
ATTCGGTCTCaGGAGgAACGATTGGTAAACCCGGTGaacgcatgagAAAGCCCCCGGAAGA
TCACCTTCCGGGGGCTTTtttattgcgcGGACCAAAACGAAAAAAGACGCTCGAAAGCGTCT
CTTTTCTGGAATTTGGTACCGAGGcgtaatgctctgccagtgttacaaccaattaaccaattctgattagaaaaactcatcg
agcatcaaatgaaactgcaatttattcatatcaggattatcaataccatatttttgaaaaagccgtttctgtaatgaaggagaaaactcaccgag
gcagttccataggatggcaagatcctggtatcggtctgcgattccgactcgtccaacatcaatacaacctattaatttcccctcgtcaaaaataa
ggttatcaagtgagaaatcaccatgagtgacgactgaatccggtgagaatggcaaaagcttatgcatttctttccagacttgttcaacaggccag
ccattacgctcgtcatcaaaatcactcgcatcaaccaaaccgttattcattcgtgattgcgcctgagcgagacgaaatacgcgatcgctgttaaa
aggacaattacaaacaggaatcgaatgcaaccggcgcaggaacactgccagcgcatcaacaatattttcacctgaatcaggatattcttctaata
cctggaatgctgttttcccggggatcgcagtggtgagtaaccatgcatcatcaggagtacggataaaatgcttgatggtcggaagaggcataaat
tccgtcagccagtttagtctgaccatctcatctgtaacatcattggcaacgctacctttgccatgtttcagaaacaactctggcgcatcgggctt
cccatacaatcgatagattgtcgcacctgattgcccgacattatcgcgagcccatttatacccatataaatcagcatccatgttggaatttaatc
gcggcctcgagcaagacgtttcccgttgaatatggctcataacaccccttgtattactgtttatgtaagcagacagttttattgttcatgatgat
atatttttatcttgtgcaatgtaacatcagagattttgagacacaacgtggctttgttgaataaatcgaacttttgctgagttgaaggatcagat
cacgcatcttcccgacaacgcagaccgttccgtggcaaagcaaaagttcaaaatcaccaactggtccacctacaacaaagctctcatcaaccgtg
gctccctcactttctggctggatgatggggcgattcaggcctggtatgagtcagcaacaccttcttcacgaggcagacctcagcgc

In some embodiments, the nucleic acid sequence comprises a temperature sensitive promoter linked to a gene sequence encoding an EGF fusion polypeptide. In certain embodiments, the nucleic acid comprising the temperature sensitive promoter linked to a gene sequence encoding an EGF fusion polypeptide has at least about 80%, 85%, 90%, 95%, or 99% identity with Sequence AB. In some embodiments, the nucleic acid comprising the temperature sensitive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AB. In some specific embodiments, the nucleic acid comprising the gene sequence encoding an EGF fusion polypeptide comprises Sequence H (PelB-EGF) with a temperature sensitive element of any one of SEQ ID NO: 183-185. In other specific embodiments the nucleic acid comprising the human EGF gene sequence consists of Sequence H with a temperature sensitive promoter construct that further comprises a gene encoding mutant cI857 repressor that comprises any one of SEQ ID NO: 183-185.

Sequence AB (CI857-pR-PelB-EGF):
aatacaacctattaatttcccctcgtcaaaaataaggttatcaagtgagaaatcaccatgagtgacgactgaatccggtgagaatggcaaaagct
tatgcatttctttccagacttgttcaacaggccagccattacgctcgtcatcaaaatcactcgcatcaaccaaaccgttattcattcgtgattgc
gcctgagcgagacgaaatacgcgatcgctgttaaaaggacaattacaaacaggaatcgaatgcaaccggcgcaggaacactgccagcgcatcaa
caatattttcacctgaatcaggatattcttctaatacctggaatgctgttttcccggggatcgcagtggtgagtaaccatgcatcatcaggagta
cggataaaatgcttgatggtcggaagaggcataaattccgtcagccagtttagtctgaccatctcatctgtaacatcattggcaacgctaccttt
gccatgtttcagaaacaactctggcgcatcgggcttcccatacaatcgatagattgtcgcacctgattgcccgacattatcgcgagcccatttat
acccatataaatcagcatccatgttggaatttaatcgcggcctcgagcaagacgtttcccgttgaatatggctcataacaccccttgtattactg
tttatgtaagcagacagttttattgttcatgatgatatatttttatcttgtgcaatgtaacatcagagattttgagacacaacgtggctttgttg
aataaatcgaacttttgctgagttgaaggatcagatcacgcatcttcccgacaacgcagaccgttccgtggcaaagcaaaagttcaaaatcacca
actggtccacctacaacaaagctctcatcaaccgtggctccctcactttctggctggatgatggggcgattcaggcctggtatgagtcagcaaca
ccttcttcacgaggcagacctcagcgctagcggagtgtatactggcttactatgttggcactgatgagggtgtcagtgaagtgcttcatgtggca
ggagaaaaaaggctgcaccggtgcgtcagcagaatatgtgatacaggatatattccgcttcctcgctcactgactcgctacgctcggtcgttcga
ctgcggcgagcggaaatggcttacgaacggggcggagatttcctggaagatgccaggaagatacttaacagggaagtgagagggccgcggcaaag
ccgtttttccataggctccgcccccctgacaagcatcacgaaatctgacgctcaaatcagtggtggcgaaacccgacaggactataaagatacca
ggcgtttcccctggcggctccctcgtgcgctctcctgttcctgcctttcggtttaccggtgtcattccgctgttatggccgcgtttgtctcattc
cacgcctgacactcagttccgggtaggcagttcgctccaagctggactgtatgcacgaaccccccgttcagtccgaccgctgcgccttatccggt
aactatcgtcttgagtccaacccggaaagacatgcaaaagcaccactggcagcagccactggtaattgatttagaggagttagtcttgaagtcat
gcgccggttaaggctaaactgaaaggacaagttttggtgactgcgctcctccaagccagttacctcggttcaaagagttggtagctcagagaacc
ttcgaaaaaccgccctgcaaggcggttttttcgttttcagagcaagagattacgcgcagaccaaaacgatctcaagaagatcatcttattaaggg
gtctgacgctcagtggaacggtgcaccctgcagggctagctgataaagcgttcgcgctgcattcggcagtttaattaaTCAGCCAAACGT
CTCTTCAGGCCACTGACTAGCGATAACTTTCCCCACAACGGAACAACTCTCATTGCAT
GGGATCATTGGGTACTGTGGGTTTAGTGGTTGTAAAAACACCTGACCGCTATCCCTGA
TCAGTTTCTTGAAGGTAAACTCATCACCCCCAAGTCTGGCTATGCAGAAATCACCTGG
CTCAACAGCCTGCTCAGGGTCAACGAGAATTAACATTCCGTCAGGAAAGCTTGGCTTG
GAGCCTGTTGGTGCGGTCATGGAATTACCTTCAACCTCAAGCCAGAATGCAGAATCAC
TGGCTTTTTTGGTTGTGCTTACCCATCTCTCCGCATCACCTTTGGTAAAGGTTCTAAGC
TTAGGTGAGAACATCCCTGCCTGAACATGAGAAAAAACAGGGTACTCATACTCACTTC
TAAGTGACGGCTGCATACTAACCGCTTCATACATCTCGTAGATTTCTCTGGCGATTGA
AGGGCTAAATTCTTCAACGCTAACTTTGAGAATTTTTGTAAGCAATGCGGCGTTATAA
GCATTTAATGCATTGATGCCATTAAATAAAGCACCAACGCCTGACTGCCCCATCCCCA
TCTTGTCTGCGACAGATTCCTGGGATAAGCCAAGTTCATTTTTCTTTTTTTCATAAATT
GCTTTAAGGCGACGTGCGTCCTCAAGCTGCTCTTGTGTTAATGGTTTCTTTTTTGTGCT
CATACGTTAAATCTATCACCGCAAGGGATAAATATCTAACACCGTGCGTGTTGACTAT
TTTACCTCTGGCGGTGATAATGGTTGCATaataattttgtttaactttaagaaggaggtatacatatgaaatatctgttgc
ccacggctgccgcgggtctgctgctgctggcagcgcaaccggctATGGCAAATAGTGACAGCGAATGTCCGCT
GTCGCACGATGGTTATTGCCTTCATGATGGGGTGTGCATGTACATTGAGGCATTGGAC
AAATATGCCTGCAACTGTGTTGTCGGCTATATCGGCGAACGGTGTCAGTACCGTGATC
TGAAATGGTGGGAATTACGCtaaGCATGCTAATCAGCCGTGGAATTCGGTCTCaGGAGg
AACGATTGGTAAACCCGGTGaacgcatgagAAAGCCCCCGGAAGATCACCTTCCGGGGGCT
TTtttattgcgcGGACCAAAACGAAAAAAGACGCTCGAAAGCGTCTCTTTTCTGGAATTTGG
TACCGAGGcgtaatgctctgccagtgttacaaccaattaaccaattctgattagaaaaactcatcgagcatcaaatgaaactgcaatttatt
catatcaggattatcaataccatatttttgaaaaagccgtttctgtaatgaaggagaaaactcaccgaggcagttccataggatggcaagatcct
ggtatcggtctgcgattccgactcgtccaacatc

In some embodiments, the nucleic acid sequence comprises a temperature sensitive promoter linked to a gene sequence encoding an EGF fusion polypeptide. In certain embodiments, the nucleic acid comprising the temperature sensitive promoter linked to a gene sequence encoding an EGF fusion polypeptide has at least about 80%, 85%, 90%, 95%, or 99% identity with Sequence AC. In some embodiments, the nucleic acid comprising the temperature sensitive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AC. In some specific embodiments, the nucleic acid comprising the gene sequence encoding an EGF fusion polypeptide comprises Sequence I (PhoA-EGF) with a temperature sensitive element of any one of SEQ ID NO: 183-185. In other specific embodiments the nucleic acid comprising the human EGF gene sequence consists of Sequence I with a temperature sensitive promoter construct that further comprises a gene encoding mutant cI857 repressor that comprises any one of SEQ ID NO: 183-185.

Sequence AC (CI857-pR-PhoA-EGF):
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggcggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaTCAGCCAAACGTCTCTTCAGG
CCACTGACTAGCGATAACTTTCCCCACAACGGAACAACTCTCATTGCATGGGATCATT
GGGTACTGTGGGTTTAGTGGTTGTAAAAACACCTGACCGCTATCCCTGATCAGTTTCTT
GAAGGTAAACTCATCACCCCCAAGTCTGGCTATGCAGAAATCACCTGGCTCAACAGC
CTGCTCAGGGTCAACGAGAATTAACATTCCGTCAGGAAAGCTTGGCTTGGAGCCTGTT
GGTGCGGTCATGGAATTACCTTCAACCTCAAGCCAGAATGCAGAATCACTGGCTTTTT
TGGTTGTGCTTACCCATCTCTCCGCATCACCTTTGGTAAAGGTTCTAAGCTTAGGTGAG
AACATCCCTGCCTGAACATGAGAAAAAACAGGGTACTCATACTCACTTCTAAGTGACG
GCTGCATACTAACCGCTTCATACATCTCGTAGATTTCTCTGGCGATTGAAGGGCTAAA
TTCTTCAACGCTAACTTTGAGAATTTTTGTAAGCAATGCGGCGTTATAAGCATTTAATG
CATTGATGCCATTAAATAAAGCACCAACGCCTGACTGCCCCATCCCCATCTTGTCTGC
GACAGATTCCTGGGATAAGCCAAGTTCATTTTTCTTTTTTTCATAAATTGCTTTAAGGC
GACGTGCGTCCTCAAGCTGCTCTTGTGTTAATGGTTTCTTTTTTGTGCTCATACGTTAA
ATCTATCACCGCAAGGGATAAATATCTAACACCGTGCGTGTTGACTATTTTACCTCTG
GCGGTGATAATGGTTGCATaataattttgtttaactttaagaaggaggtatacatatgaaacaaagcactattgcactggcactc
ttaccgttactgtttacccctgtgacaaaagcgAATAGTGACAGCGAATGTCCGCTGTCGCACGATGGTTA
TTGCCTTCATGATGGGGTGTGCATGTACATTGAGGCATTGGACAAATATGCCTGCAAC
TGTGTTGTCGGCTATATCGGCGAACGGTGTCAGTACCGTGATCTGAAATGGTGGGAAT
TACGCtaaGCATGCTAATCAGCCGTGGAATTCGGTCTCaGGAGgAACGATTGGTAAACCC
GGTGaacgcatgagAAAGCCCCCGGAAGATCACCTTCCGGGGGCTTTtttattgcgcGGACCAAA
ACGAAAAAAGACGCTCGAAAGCGTCTCTTTTCTGGAATTTGGTACCGAGGcgtaatgctctgc
cagtgttacaaccaattaaccaattctgattagaaaaactcatcgagcatcaaatgaaactgcaatttattcatatcaggattatcaataccata
tttttgaaaaagccgtttctgtaatgaaggagaaaactcaccgaggcagttccataggatggcaagatcctggtatcggtctgcgattccgactc
gtccaacatcaatacaacctattaatttcccctcgtcaaaaataaggttatcaagtgagaaatcaccatgagtgacgactgaatccggtgagaat
ggcaaaagcttatgcatttctttccagacttgttcaacaggccagccattacgctcgtcatcaaaatcactcgcatcaaccaaaccgttattcat
tcgtgattgcgcctgagcgagacgaaatacgcgatcgctgttaaaaggacaattacaaacaggaatcgaatgcaaccggcgcaggaacactgcca
gcgcatcaacaatattttcacctgaatcaggatattcttctaatacctggaatgctgttttcccggggatcgcagtggtgagtaaccatgcatca
tcaggagtacggataaaatgcttgatggtcggaagaggcataaattccgtcagccagtttagtctgaccatctcatctgtaacatcattggcaac
gctacctttgccatgtttcagaaacaactctggcgcatcgggcttcccatacaatcgatagattgtcgcacctgattgcccgacattatcgcgag
cccatttatacccatataaatcagcatccatgttggaatttaatcgcggcctcgagcaagacgtttcccgttgaatatggctcataacacccctt
gtattactgtttatgtaagcagacagttttattgttcatgatgatatatttttatcttgtgcaatgtaacatcagagattttgagacacaacgtg
gctttgttgaataaatcgaacttttgctgagttgaaggatcagatcacgcatcttcccgacaacgcagaccgttccgtggcaaagcaaaagttca
aaatcaccaactggtccacctacaacaaagctctcatcaaccgtggctccctcactttctggctggatgatggggcgattcaggcctggtatgag
tcagcaacaccttcttcacgaggcagacctcagcgc

In some embodiments, the nucleic acid sequence comprises a temperature sensitive promoter linked to a gene sequence encoding an EGF fusion polypeptide. In certain embodiments, the nucleic acid comprising the temperature sensitive promoter linked to a gene sequence encoding an EGF fusion polypeptide has at least about 80%, 85%, 90%, 95%, or 99% identity with Sequence AD. In some embodiments, the nucleic acid comprising the temperature sensitive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AD. In some specific embodiments, the nucleic acid comprising the gene sequence encoding an EGF fusion polypeptide comprises Sequence J (OmpA-EGF) with a temperature sensitive element of any one of SEQ ID NO: 183-185. In other specific embodiments the nucleic acid comprising the human EGF gene sequence consists of Sequence J with a temperature sensitive promoter construct that further comprises a gene encoding mutant cI857 repressor that comprises any one of SEQ ID NO: 183-185.

Sequence AD (CI857-pR-OmpA-EGF):
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggcggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaTCAGCCAAACGTCTCTTCAGG
CCACTGACTAGCGATAACTTTCCCCACAACGGAACAACTCTCATTGCATGGGATCATT
GGGTACTGTGGGTTTAGTGGTTGTAAAAACACCTGACCGCTATCCCTGATCAGTTTCTT
GAAGGTAAACTCATCACCCCCAAGTCTGGCTATGCAGAAATCACCTGGCTCAACAGC
CTGCTCAGGGTCAACGAGAATTAACATTCCGTCAGGAAAGCTTGGCTTGGAGCCTGTT
GGTGCGGTCATGGAATTACCTTCAACCTCAAGCCAGAATGCAGAATCACTGGCTTTTT
TGGTTGTGCTTACCCATCTCTCCGCATCACCTTTGGTAAAGGTTCTAAGCTTAGGTGAG
AACATCCCTGCCTGAACATGAGAAAAAACAGGGTACTCATACTCACTTCTAAGTGACG
GCTGCATACTAACCGCTTCATACATCTCGTAGATTTCTCTGGCGATTGAAGGGCTAAA
TTCTTCAACGCTAACTTTGAGAATTTTTGTAAGCAATGCGGCGTTATAAGCATTTAATG
CATTGATGCCATTAAATAAAGCACCAACGCCTGACTGCCCCATCCCCATCTTGTCTGC
GACAGATTCCTGGGATAAGCCAAGTTCATTTTTCTTTTTTTCATAAATTGCTTTAAGGC
GACGTGCGTCCTCAAGCTGCTCTTGTGTTAATGGTTTCTTTTTTGTGCTCATACGTTAA
ATCTATCACCGCAAGGGATAAATATCTAACACCGTGCGTGTTGACTATTTTACCTCTG
GCGGTGATAATGGTTGCATaataattttgtttaactttaagaaggaggtatacatATGAAGAAAACCGCAATT
GCAATCGCCGTCGCTCTGGCGGGGTTCGCTACGGTCGCCCAAGCCAATAGTGACAGC
GAATGTCCGCTGTCGCACGATGGTTATTGCCTTCATGATGGGGTGTGCATGTACATTG
AGGCATTGGACAAATATGCCTGCAACTGTGTTGTCGGCTATATCGGCGAACGGTGTCA
GTACCGTGATCTGAAATGGTGGGAATTACGCtaaGCATGCTAATCAGCCGTGGAATTCG
GTCTCaGGAGgAACGATTGGTAAACCCGGTGaacgcatgagAAAGCCCCCGGAAGATCACC
TTCCGGGGGCTTTtttattgcgcGGACCAAAACGAAAAAAGACGCTCGAAAGCGTCTCTTTT
CTGGAATTTGGTACCGAGGcgtaatgctctgccagtgttacaaccaattaaccaattctgattagaaaaactcatcgagcatca
aatgaaactgcaatttattcatatcaggattatcaataccatatttttgaaaaagccgtttctgtaatgaaggagaaaactcaccgaggcagttc
cataggatggcaagatcctggtatcggtctgcgattccgactcgtccaacatcaatacaacctattaatttcccctcgtcaaaaataaggttatc
aagtgagaaatcaccatgagtgacgactgaatccggtgagaatggcaaaagcttatgcatttctttccagacttgttcaacaggccagccattac
gctcgtcatcaaaatcactcgcatcaaccaaaccgttattcattcgtgattgcgcctgagcgagacgaaatacgcgatcgctgttaaaaggacaa
ttacaaacaggaatcgaatgcaaccggcgcaggaacactgccagcgcatcaacaatattttcacctgaatcaggatattcttctaatacctggaa
tgctgttttcccggggatcgcagtggtgagtaaccatgcatcatcaggagtacggataaaatgcttgatggtcggaagaggcataaattccgtca
gccagtttagtctgaccatctcatctgtaacatcattggcaacgctacctttgccatgtttcagaaacaactctggcgcatcgggcttcccatac
aatcgatagattgtcgcacctgattgcccgacattatcgcgagcccatttatacccatataaatcagcatccatgttggaatttaatcgcggcct
cgagcaagacgtttcccgttgaatatggctcataacaccccttgtattactgtttatgtaagcagacagttttattgttcatgatgatatatttt
tatcttgtgcaatgtaacatcagagattttgagacacaacgtggctttgttgaataaatcgaacttttgctgagttgaaggatcagatcacgcat
cttcccgacaacgcagaccgttccgtggcaaagcaaaagttcaaaatcaccaactggtccacctacaacaaagctctcatcaaccgtggctccct
cactttctggctggatgatggggcgattcaggcctggtatgagtcagcaacaccttcttcacgaggcagacctcagcgc

In some embodiments, the nucleic acid sequence comprises a temperature sensitive promoter linked to a gene sequence encoding an EGF fusion polypeptide. In certain embodiments, the nucleic acid comprising the temperature sensitive promoter linked to a gene sequence encoding an EGF fusion polypeptide has at least about 80%, 85%, 90%, 95%, or 99% identity with Sequence AE. In some embodiments, the nucleic acid comprising the temperature sensitive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AE. In some specific embodiments, the nucleic acid comprising the gene sequence encoding an EGF fusion polypeptide comprises Sequence K (EGF-LARD3) with a temperature sensitive element of any one of SEQ ID NO: 183-185. In other specific embodiments the nucleic acid comprising the human EGF gene sequence consists of Sequence K with a temperature sensitive promoter construct that further comprises a gene encoding mutant cI857 repressor that comprises any one of SEQ ID NO: 183-185.

Sequence AE (CI857-pR-EGF-LARD3)
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggcggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaTCAGCCAAACGTCTCTTCAGG
CCACTGACTAGCGATAACTTTCCCCACAACGGAACAACTCTCATTGCATGGGATCATT
GGGTACTGTGGGTTTAGTGGTTGTAAAAACACCTGACCGCTATCCCTGATCAGTTTCTT
GAAGGTAAACTCATCACCCCCAAGTCTGGCTATGCAGAAATCACCTGGCTCAACAGC
CTGCTCAGGGTCAACGAGAATTAACATTCCGTCAGGAAAGCTTGGCTTGGAGCCTGTT
GGTGCGGTCATGGAATTACCTTCAACCTCAAGCCAGAATGCAGAATCACTGGCTTTTT
TGGTTGTGCTTACCCATCTCTCCGCATCACCTTTGGTAAAGGTTCTAAGCTTAGGTGAG
AACATCCCTGCCTGAACATGAGAAAAAACAGGGTACTCATACTCACTTCTAAGTGACG
GCTGCATACTAACCGCTTCATACATCTCGTAGATTTCTCTGGCGATTGAAGGGCTAAA
TTCTTCAACGCTAACTTTGAGAATTTTTGTAAGCAATGCGGCGTTATAAGCATTTAATG
CATTGATGCCATTAAATAAAGCACCAACGCCTGACTGCCCCATCCCCATCTTGTCTGC
GACAGATTCCTGGGATAAGCCAAGTTCATTTTTCTTTTTTTCATAAATTGCTTTAAGGC
GACGTGCGTCCTCAAGCTGCTCTTGTGTTAATGGTTTCTTTTTTGTGCTCATACGTTAA
ATCTATCACCGCAAGGGATAAATATCTAACACCGTGCGTGTTGACTATTTTACCTCTG
GCGGTGATAATGGTTGCATaataattttgtttaactttaagaaggaggtatacatATGAATAGTGACAGCGAA
TGTCCGCTGTCGCACGATGGTTATTGCCTTCATGATGGGGTGTGCATGTACATTGAGG
CATTGGACAAATATGCCTGCAACTGTGTTGTCGGCTATATCGGCGAACGGTGTCAGTA
CCGTGATCTGAAATGGTGGGAATTACGCATTGAGGGCCGGggttctgacggtaatgatcttatccaagg
cggtaagggcgcggacttcattgaaggcggcaaaggtaatgatacaatccgcgataactccggtcacaacacctttttgttctcagggcattttg
gtcaggatcgtattataggatatcagccgaccgatcggctggtattccagggcgctgacggcagcacggatctgcgcgaccatgcgaaagcc
gttggagcagatacggtgctgagttttggcgccgattcggttactctcgtcggggggggttaggaggcctgtggagcgagggtgtgctgatta
gttaaGCATGCTAATCAGCCGTGGAATTCGGTCTCaGGAGgAACGATTGGTAAACCCGGT
GaacgcatgagAAAGCCCCCGGAAGATCACCTTCCGGGGGCTTTtttattgcgcGGACCAAAACG
AAAAAAGACGCTCGAAAGCGTCTCTTTTCTGGAATTTGGTACCGAGGcgtaatgctctgccagtg
ttacaaccaattaaccaattctgattagaaaaactcatcgagcatcaaatgaaactgcaatttattcatatcaggattatcaataccatattttt
gaaaaagccgtttctgtaatgaaggagaaaactcaccgaggcagttccataggatggcaagatcctggtatcggtctgcgattccgactcgtcca
acatcaatacaacctattaatttcccctcgtcaaaaataaggttatcaagtgagaaatcaccatgagtgacgactgaatccggtgagaatggcaa
aagcttatgcatttctttccagacttgttcaacaggccagccattacgctcgtcatcaaaatcactcgcatcaaccaaaccgttattcattcgtg
attgcgcctgagcgagacgaaatacgcgatcgctgttaaaaggacaattacaaacaggaatcgaatgcaaccggcgcaggaacactgccagcgca
tcaacaatattttcacctgaatcaggatattcttctaatacctggaatgctgttttcccggggatcgcagtggtgagtaaccatgcatcatcagg
agtacggataaaatgcttgatggtcggaagaggcataaattccgtcagccagtttagtctgaccatctcatctgtaacatcattggcaacgctac
ctttgccatgtttcagaaacaactctggcgcatcgggcttcccatacaatcgatagattgtcgcacctgattgcccgacattatcgcgagcccat
ttatacccatataaatcagcatccatgttggaatttaatcgcggcctcgagcaagacgtttcccgttgaatatggctcataacaccccttgtatt
actgtttatgtaagcagacagttttattgttcatgatgatatatttttatcttgtgcaatgtaacatcagagattttgagacacaacgtggcttt
gttgaataaatcgaacttttgctgagttgaaggatcagatcacgcatcttcccgacaacgcagaccgttccgtggcaaagcaaaagttcaaaatc
accaactggtccacctacaacaaagctctcatcaaccgtggctccctcactttctggctggatgatggggcgattcaggcctggtatgagtcagc
aacaccttcttcacgaggcagacctcagcgc

In some embodiments, the nucleic acid sequence comprises a temperature sensitive promoter linked to a gene sequence encoding an EGF fusion polypeptide. In certain embodiments, the nucleic acid comprising the temperature sensitive promoter linked to a gene sequence encoding an EGF fusion polypeptide has at least about 80%, 85%, 90%, 95%, or 99% identity with Sequence AF. In some embodiments, the nucleic acid comprising the temperature sensitive promoter linked to a gene sequence encoding an EGF fusion polypeptide has at least about 85%, 86%, 87%, 88%, 89%, 90%, 91%, 92%, 93%, 94%, 95%, 96%, 97%, 98%, or 99% identity with Sequence AF. In some specific embodiments, the nucleic acid comprising the gene sequence encoding an EGF fusion polypeptide comprises Sequence L (EGF-HylA) with a temperature sensitive element of any one of SEQ ID NO: 183-185. In other specific embodiments the nucleic acid comprising the human EGF gene sequence consists of Sequence L with a temperature sensitive promoter construct that further comprises a gene encoding mutant cI857 repressor that comprises any one of SEQ ID NO: 183-185.

Sequence AF (CI857-pR-EGF-HylA)
tagcggagtgtatactggcttactatgttggcactgatgagggtgtcagtgaagtgcttcatgtggcaggagaaaaaaggctgcaccggtgcgt
cagcagaatatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccataggctccgc
ccccctgacaagcatcacgaaatctgacgctcaaatcagtggtggcgaaacccgacaggactataaagataccaggcgtttcccctggcggc
tccctcgtgcgctctcctgttcctgcctttcggtttaccggtgtcattccgctgttatggccgcgtttgtctcattccacgcctgacactcagtt
ccgggtaggcagttcgctccaagctggactgtatgcacgaaccccccgttcagtccgaccgctgcgccttatccggtaactatcgtcttgagtcc
aacccggaaagacatgcaaaagcaccactggcagcagccactggtaattgatttagaggagttagtcttgaagtcatgcgccggttaaggctaaa
ctgaaaggacaagttttggtgactgcgctcctccaagccagttacctcggttcaaagagttggtagctcagagaaccttcgaaaaaccgccctgc
aaggcggttttttcgttttcagagcaagagattacgcgcagaccaaaacgatctcaagaagatcatcttattaaggggtctgacgctcagtggaa
cggtgcaccctgcagggctagctgataaagcgttcgcgctgcattcggcagtttaattaaTCAGCCAAACGTCTCTTCAGG
CCACTGACTAGCGATAACTTTCCCCACAACGGAACAACTCTCATTGCATGGGATCATT
GGGTACTGTGGGTTTAGTGGTTGTAAAAACACCTGACCGCTATCCCTGATCAGTTTCTT
GAAGGTAAACTCATCACCCCCAAGTCTGGCTATGCAGAAATCACCTGGCTCAACAGC
CTGCTCAGGGTCAACGAGAATTAACATTCCGTCAGGAAAGCTTGGCTTGGAGCCTGTT
GGTGCGGTCATGGAATTACCTTCAACCTCAAGCCAGAATGCAGAATCACTGGCTTTTT
TGGTTGTGCTTACCCATCTCTCCGCATCACCTTTGGTAAAGGTTCTAAGCTTAGGTGAG
AACATCCCTGCCTGAACATGAGAAAAAACAGGGTACTCATACTCACTTCTAAGTGACG
GCTGCATACTAACCGCTTCATACATCTCGTAGATTTCTCTGGCGATTGAAGGGCTAAA
TTCTTCAACGCTAACTTTGAGAATTTTTGTAAGCAATGCGGCGTTATAAGCATTTAATG
CATTGATGCCATTAAATAAAGCACCAACGCCTGACTGCCCCATCCCCATCTTGTCTGC
GACAGATTCCTGGGATAAGCCAAGTTCATTTTTCTTTTTTTCATAAATTGCTTTAAGGC
GACGTGCGTCCTCAAGCTGCTCTTGTGTTAATGGTTTCTTTTTTGTGCTCATACGTTAA
ATCTATCACCGCAAGGGATAAATATCTAACACCGTGCGTGTTGACTATTTTACCTCTG
GCGGTGATAATGGTTGCATaataattttgtttaactttaagaaggaggtatacatATGAATAGTGACAGCGAA
TGTCCGCTGTCGCACGATGGTTATTGCCTTCATGATGGGGTGTGCATGTACATTGAGG
CATTGGACAAATATGCCTGCAACTGTGTTGTCGGCTATATCGGCGAACGGTGTCAGTA
CCGTGATCTGAAATGGTGGGAATTACGCtcaacttatgggagccaggacaatcttaatccattaattaatgaaatcag
caaaatcatttcagctgcaggtaacttcgatgttaaggaggaaagatctgccgcttctttattgcagttgtccggtaatgccagtgatttttcat
atggacggaactcaataactttgacagcatcagcataaGCATGCTAATCAGCCGTGGAATTCGGTCTCaGGAGgA
ACGATTGGTAAACCCGGTGaacgcatgagAAAGCCCCCGGAAGATCACCTTCCGGGGGCTT
TtttattgcgcGGACCAAAACGAAAAAAGACGCTCGAAAGCGTCTCTTTTCTGGAATTTGGT
ACCGAGGcgtaatgctctgccagtgttacaaccaattaaccaattctgattagaaaaactcatcgagcatcaaatgaaactgcaatttattc
atatcaggattatcaataccatatttttgaaaaagccgtttctgtaatgaaggagaaaactcaccgaggcagttccataggatggcaagatcctg
gtatcggtctgcgattccgactcgtccaacatcaatacaacctattaatttcccctcgtcaaaaataaggttatcaagtgagaaatcaccatgag
tgacgactgaatccggtgagaatggcaaaagcttatgcatttctttccagacttgttcaacaggccagccattacgctcgtcatcaaaatcactc
gcatcaaccaaaccgttattcattcgtgattgcgcctgagcgagacgaaatacgcgatcgctgttaaaaggacaattacaaacaggaatcgaatg
caaccggcgcaggaacactgccagcgcatcaacaatattttcacctgaatcaggatattcttctaatacctggaatgctgttttcccggggatcg
cagtggtgagtaaccatgcatcatcaggagtacggataaaatgcttgatggtcggaagaggcataaattccgtcagccagtttagtctgaccatc
tcatctgtaacatcattggcaacgctacctttgccatgtttcagaaacaactctggcgcatcgggcttcccatacaatcgatagattgtcgcacc
tgattgcccgacattatcgcgagcccatttatacccatataaatcagcatccatgttggaatttaatcgcggcctcgagcaagacgtttcccgtt
gaatatggctcataacaccccttgtattactgtttatgtaagcagacagttttattgttcatgatgatatatttttatcttgtgcaatgtaacat
cagagattttgagacacaacgtggctttgttgaataaatcgaacttttgctgagttgaaggatcagatcacgcatcttcccgacaacgcagaccg
ttccgtggcaaagcaaaagttcaaaatcaccaactggtccacctacaacaaagctctcatcaaccgtggctccctcactttctggctggatgatg
gggcgattcaggcctggtatgagtcagcaacaccttcttcacgaggcagacctcagcgc

In some embodiments, the nucleic acid sequence comprises a temperature sensitive promoter linked to a gene sequence encoding an ATP-binding cassette transporter polypeptide. In certain embodiments, the nucleic acid comprising the temperature sensitive promoter linked to a gene sequence encoding an ATP-binding cassette transporter polypeptide has at least about 80%, 85%, 90%, 95%, or 99% identity with Sequence AG. In some embodiments, the nucleic acid comprising the temperature sensitive promoter linked to a gene sequence encoding an ATP-binding cassette transporter polypeptide has at least about 85%, 86%, 87%, 88%, 89%, 90%, 91%, 92%, 93%, 94%, 95%, 96%, 97%, 98%, or 99% identity with Sequence AG. In some specific embodiments, the nucleic acid comprising the gene sequence encoding an ATP-binding cassette transporter polypeptide comprises Sequence M.1, M.2, and/or M.3 (ATP-binding cassette transporter) with a temperature sensitive element of any one of SEQ ID NO: 183-185. In other specific embodiments the nucleic acid comprising the human EGF gene sequence consists of Sequence M.1, M.2, and/or M.3 with a temperature sensitive promoter construct that further comprises a gene encoding mutant cI857 repressor that comprises any one of SEQ ID NO: 183-185.

Sequence AG (CI857-pR-prtDEF)
GTGTGAAATACCGCACAGATGCGTAAGGAGAAAATACCGCATCAGGCGCCAT
TCGCCATTCAGGCTGCGCAACTGTTGGGAAGGGCGATCGGTGCGGGCCTCTTCGCTAT
TACGCCAGCTTCAGCCAAACGTCTCTTCAGGCCACTGACTAGCGATAACTTTCCCCAC
AACGGAACAACTCTCATTGCATGGGATCATTGGGTACTGTGGGTTTAGTGGTTGTAAA
AACACCTGACCGCTATCCCTGATCAGTTTCTTGAAGGTAAACTCATCACCCCCAAGTC
TGGCTATGCAGAAATCACCTGGCTCAACAGCCTGCTCAGGGTCAACGAGAATTAACAT
TCCGTCAGGAAAGCTTGGCTTGGAGCCTGTTGGTGCGGTCATGGAATTACCTTCAACC
TCAAGCCAGAATGCAGAATCACTGGCTTTTTTGGTTGTGCTTACCCATCTCTCCGCATC
ACCTTTGGTAAAGGTTCTAAGCTTAGGTGAGAACATCCCTGCCTGAACATGAGAAAAA
ACAGGGTACTCATACTCACTTCTAAGTGACGGCTGCATACTAACCGCTTCATACATCT
CGTAGATTTCTCTGGCGATTGAAGGGCTAAATTCTTCAACGCTAACTTTGAGAATTTTT
GTAAGCAATGCGGCGTTATAAGCATTTAATGCATTGATGCCATTAAATAAAGCACCAA
CGCCTGACTGCCCCATCCCCATCTTGTCTGCGACAGATTCCTGGGATAAGCCAAGTTC
ATTTTTCTTTTTTTCATAAATTGCTTTAAGGCGACGTGCGTCCTCAAGCTGCTCTTGTGT
TAATGGTTTCTTTTTTGTGCTCATACGTTAAATCTATCACCGCAAGGGATAAATATCTA
ACACCGTGCGTGTTGACTATTTTACCTCTGGCGGTGATAATGGTTGCATAATCTTTGGA
TTTAAGGAGACCTCTatgaatgcttcctctgagcgagatcgttcgcttttcggcgtgctgagacagttccgccgcagtttctggag
cgtcgggattttcagcgccgtcatcaacgtgctgatgttagcgccttcggtatatatgctgcaggtgtatgaccgggtgctggcatcaggtaacg
gtatcaccctgctcatgctgactttactgatggcgggactgtgcgcatttatgggcgcgttagagtgggttcgtagtctgcttgtagtgaggctg
gtacccgtatcgatctcgcgctcaatcaggatgtctttaatgcggcatttgcccgtaatctggaggcaggtgatggccgcgccgggctggcgct
ggaccgacttaacgctgcttcgccagttcatcaccggcaacgcgcttttcgccttcttcgatgtgccatggtttccgctgtttctactggttctg
tttttgctgcacccatggctgggaatgctcgcgctgggaggcactgtggtgctagttgcgttagcctggctgaaccagcacctgaccaaccaacc
gcttgctgaagccaatcagcaatcacaacaggcgacccacttagctgacgctcagttgcgcaatgccgacgttattgaagcaatggggatgctgg
gtaacctgcgtcgacgctggcttgcgcgtcattaccgttttatttccctgcagaatcttgccagcgagcgtgccgccgcggttggcggtgcgtcc
aaatacagccgtattgcgttgcaatcattgatgctggggctgggagcactgctggcgatcgacggtaagatcacaccagggatgatgatcgccg
gtagcattttagttggtcgggtactgagtccgatcgatcagttaataggggtatggaaacagtggagctctgcgcgcattgcctggcaacgctta
acccggctgatcgccgcatatccgccgcgcccggcggcaatggctttacctgcgccggaagggcatctcagtgtggaacaggtctcacttcg
cacggtgcagggaaatacccgactgcaaaacatacatttttcgttgcaggctggcgaaacgctggtcattctcggcgcatctggttccggcaaa
tcctcgctggcgcgtttgctggtcggcgcgcagagtccgacacaaggtaaagttcgtctggatggcgctgatttgaatcaggtcgacaaaaac
acgtttggtccgaccattggttatttgccccaggacgtgcagttatttaagggctcgctcgctgaaaacatcgcacgttttggcgatgccgatcc
ggaaaaagttgtagcggcagcaaaacttgcgggggtacatgaactcattctgtctctacccaacggttatgacaccgaattgggtgatggcggc
ggcggtttgtctggcggccagagacagcgtatcggtctggcgcgcgctatgtacggcgatccttgtctgctgattctggatgaaccaaacgcc
agccttgacagcgaaggcgatcaagccctaatgcaggcgattgtcgcattgcagaaacgcggtgccactgttgtgttgattactcatcgcccg
gctttaacaacgctggctcaaaagattctgattctccacgagggccaacaacagcgtatgggactggcgcgcgatgtgctgacagaactgcaa
cagcgcagcgcagccaatcaggcacggatgaaccccacggccgcaatgcctcaataaGACCCACTTTTAAGGAGGTAT
TTAGatgacaggcatggatataaccactcaggatgaattaaacgaagcagcaatgcgcgatcgcgcttcacgtgatgaagagcgcgcatt
acggctgggctggtggctggtgctcgcgggtttcggtggttttctgctgtgggcattactggcgccgttagataaaggggtggcggtgcaagg
caacgtggtggtttcaggcaatcgtaaggtgatccaacacatgcagggcggcattgtcgatcggattcaggtgaaagacggcgaccgcgttg
ccgcaggtcaggttctcctgacgctgaatgcggtagatgcccgtaccaccagcgaagggctgggttcccaatatgatcaattaattgctcgcga
agccagactgctggcggaacagcgcaatcaatcgagtctggcggctacgccacgactggcgcaggcgcgtcagagaccggaaatggctg
ccattattgcgttgcaggaagatctacttcgtagccgtcagcaatcgctaaaactggaaatagatggagtccgcgccagcatcgatggactgga
gacttcgcttggcgcattgcaaaaggtgatgagttctaaacagtccgagcaggcaacgttgtctcagcaattacagggcctccggccgctggc
ggcggataactatgttccgcgtaacaagatgctggaaactgagcgtttgtttgctcaggtctctggcgaactggcccagacaagcggcgaggt
agggcgaacccgacgtgatattcagcagcaaaaattgaggatcgcccagcgtcagcaggaatacgacaaagaggtcaacagcgaattgtcc
gacgtacaagccaaactgaatgaggttatcagtcagcgcgagaaagcggatttcaatctggccaacgttcaggtccgtgcacctgttgccggc
accgtggtggatatgaagatcttcaccgaaggtggtgtgatcgcacccgggcaggtcatgatggacatcgttcccgaagaccaaccgctgctt
gttgatgggcgcattcctgtggaaatggtggacaaagtatggtcaggtcttccagtggaactccaattcaccgcgttttcacaaagcacaacgc
cgcgtgtaccgggaacggtcaccctgctttccgctgaccggctggtagacgaaaaagacggcactccgtactacggtctccgcattcaggtg
agcgaagaaggcaaacgcagtttgcatggtctggaaattaaaccaggtatgccggttcaggggtttgtccgcaccggagagcgttcgtttatca
attatctgtttaaacctctgatggatcgcatgcatcttgcgttaacggaagagtaaGTTAAGGTAGTTGGGGGAGGCAAT
Aatgcgaaggaaagcggtactactgacggttgttttatctctgtcatgcggcagcgcccaggcgatgggcctcctggatgcatgggaacttgc
acttcgtaatgacgcacaactgcgggcggccggtttcgaacgagacgccggtcaggaagaggtggcgatcggtcgcgctgggttgctgcca
tcgctgcaatatacctatggcgcaaattacagtcattccaaggtgacgcaacgcgatcggacgctgaataacacgactaaacgtgactacgata
actacgtatctacgcttacgctgcgtcagccgctgctggactatgcagcctgggcgcgctatcagcagggcgtcacccgcaaacttatggctg
atcagaggtttcgtgatcgtagtcaggacctgatggtgcgtttgtaccagtcctggagcgaagcattgctggcgcaggagaagcttatgctcctc
gatgcgcagcgccgtgcctatcaggagcagttagcgctgaaccgtcgcttattggctgcgggggaaggaactcaaacagacctgcgggaaa
cggaagcacgttacaccgtcactgaagcccaacgtatcgaacaggaagataccctggatgccgctatgacagatctcgaaaacatgatgggt
tcgccgctacaaattcaggatctgagtccacttgcgctggatacgttgccggataacgtaaccgaaaatcgctcgttatcacaatggcgcgaac
tgactgtgcggcacaatgcaaagctcgcggttcaacgtgaaaacgtcgattacagccgctatgaaattgagcgtaacagagccggccatctgc
ccacactggacctcgtggcgtccacccgcaacagcttgtccgaatctgaatataactacaaccagaaatacgacacccagaccgttggtttaca
ggtgagagtgccgctgtatagcggcggcgccgtttcggcatcaatgcgtcaggcagcggcggaataccagcaaagtcaggcggagctgga
taaccagacccgtcagacctttgcggaattacgccgtcagttcaacctgtgtgctaatggggccgcaaaaatccgagcctggcagatgagcgt
ggcagctgctgaggaagcgatccgcgcgacgcggcaaagcgtcgccggcggcgagcgcattaatctggacgttctgatggcggaacagg
agtggtataacgcccgccgcgagctgacagaagtcaaatatcgctggttacaggcctggctgaatttgcgttataccgccggaacactgaatg
agcaagatatgatgcaattggctgcctggtttcaatctgcgcctgtaattaataaaactggtataaataaaaccggaattaacgcagctaccggt
aataaaactaattaatacgcatggcatggatgaCCGATGGTAGTGTGGGGTCTCCCCATGCGAGAGTAGGG
AACTGCCAGGCATCAAATAAAACGAAAGGCTCAGTCGAAAGACTGGGCCTTTCGTTTT
ATCTGTTGTTTGTCGGTGAACGCTCTCCTGAGTAGGACAAATCCGCCGGGAGCGGATT
TGAACGTTGCGAAGCAACGGCCCGGAGGGTGGCGGGCAGGACGCCCGCCATAAACTG
CCAGGCATCAAATTAAGCAGAAGGCCATCCTGACGGATGGCCTTTTTGCGTGGCCAGT
GCCAAGCTTGCATGCGTGCCAGCTGCATTAATGAAGAAATCATGCTGGAAGAATAAG
CACCGACCGCAGCCACAGCGTGTAGCGCTCCCAGGCCGTCCACTCCCTGATCCGGCGC
TCATTCCACACAACATACGAGCCGGAAGCATAAAGTGTAAAGCCTGGGGTGCCTAAT
GAGTGAGCTAACTCACATTAATTGCGTTGCGCTCACTGCCCGCTTTCCAGTCGGGAAA
CCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCG
GCGAGCGGTATCAGCTCACTCAAAGGCGGTAGTACGGGTTTTGCTGCCCGCAAACGG
GCTGTTCTGGTGTTGCTAGTTTGTTATCAGAATCGCAGATCCGGCTTCAGGTTTGCCGG
CTGAAAGCGCTATTTCTTCCAGAATTGCCATGATTTTTTCCCCACGGGAGGCGTCACTG
GCTCCCGTGTTGTCGGCAGCTTTGATTCGATAAGCAGCATCGCCTGTTTCAGGCTGTCT
ATGTGTGACTGTTGAGCTGTAACAAGTTGTCTCAGGTGTTCAATTTCATGTTCTAGTTG
CTTTGTTTTACTGGTTTCACCTGTTCTATTAGGTGTTACATGCTGTTCATCTGTTACATT
GTCGATCTGTTCATGGTGAACAGCTTTAAATGCACCAAAAACTCGTAAAAGCTCTGAT
GTATCTATCTTTTTTACACCGTTTTCATCTGTGCATATGGACAGTTTTCCCTTTGATATC
TAACGGTGAACAGTTGTTCTACTTTTGTTTGTTAGTCTTGATGCTTCACTGATAGATAC
AAGAGCCATAAGAACCTCAGATCCTTCCGTATTTAGCCAGTATGTTCTCTAGTGTGGT
TCGTTGTTTTTGCGTGAGCCATGAGAACGAACCATTGAGATCATGCTTACTTTGCATGT
CACTCAAAAATTTTGCCTCAAAACTGGTGAGCTGAATTTTTGCAGTTAAAGCATCGTG
TAGTGTTTTTCTTAGTCCGTTACGTAGGTAGGAATCTGATGTAATGGTTGTTGGTATTT
TGTCACCATTCATTTTTATCTGGTTGTTCTCAAGTTCGGTTACGAGATCCATTTGTCTAT
CTAGTTCAACTTGGAAAATCAACGTATCAGTCGGGCGGCCTCGCTTATCAACCACCAA
TTTCATATTGCTGTAAGTGTTTAAATCTTTACTTATTGGTTTCAAAACCCATTGGTTAA
GCCTTTTAAACTCATGGTAGTTATTTTCAAGCATTAACATGAACTTAAATTCATCAAGG
CTAATCTCTATATTTGCCTTGTGAGTTTTCTTTTGTGTTAGTTCTTTTAATAACCACTCA
TAAATCCTCATAGAGTATTTGTTTTCAAAAGACTTAACATGTTCCAGATTATATTTTAT
GAATTTTTTTAACTGGAAAAGATAAGGCAATATCTCTTCACTAAAAACTAATTCTAAT
TTTTCGCTTGAGAACTTGGCATAGTTTGTCCACTGGAAAATCTCAAAGCCTTTAACCA
AAGGATTCCTGATTTCCACAGTTCTCGTCATCAGCTCTCTGGTTGCTTTAGCTAATACA
CCATAAGCATTTTCCCTACTGATGTTCATCATCTGAGCGTATTGGTTATAAGTGAACGA
TACCGTCCGTTCTTTCCTTGTAGGGTTTTCAATCGTGGGGTTGAGTAGTGCCACACAGC
ATAAAATTAGCTTGGTTTCATGCTCCGTTAAGTCATAGCGACTAATCGCTAGTTCATTT
GCTTTGAAAACAACTAATTCAGACATACATCTCAATTGGTCTAGGTGATTTTAATCAC
TATACCAATTGAGATGGGCTAGTCAATGATAATTACTAGTCCTTTTCCTTTGAGTTGTG
GGTATCTGTAAATTCTGCTAGACCTTTGCTGGAAAACTTGTAAATTCTGCTAGACCCTC
TGTAAATTCCGCTAGACCTTTGTGTGTTTTTTTTGTTTATATTCAAGTGGTTATAATTTA
TAGAATAAAGAAAGAATAAAAAAAGATAAAAAGAATAGATCCCAGCCCTGTGTATAA
CTCACTACTTTAGTCAGTTCCGCAGTATTACAAAAGGATGTCGCAAACGCTGTTTGCT
CCTCTACAAAACAGACCTTAAAACCCTAAAGGCTTAAGTAGCACCCTCGCAAGCTCG
ggcaaatcgctgaatattccttttgtctccgaccatc

The nucleic acid may be present on a plasmid or chromosome in the bacterial cell. In one embodiment, the nucleic acid is located on a plasmid in the bacterial cell. In another embodiment, the nucleic acid is located in the chromosome of the bacterial cell. In yet another embodiment, a native copy of the nucleic acid is located in the chromosome of the bacterial cell.

### Multiple Mechanisms of Action

In some embodiments, the bacteria are genetically engineered to include multiple mechanisms of action (MOAs), e.g., circuits producing multiple copies of the same product (e.g., to enhance copy number) or circuits performing multiple different functions. Examples of insertion sites include, but are not limited to, malE/K, insB/I, araC/BAD, lacZ, dapA, cea. For example, the recombinant bacteria may include four copies of EGF inserted at four different insertion sites, e.g., malE/K, insB/I, araC/BAD, and lacZ. Alternatively, the recombinant bacteria may include three copies of EGF inserted at three different insertion sites, e.g., malE/K, insB/I, and lacZ.

In some embodiments, the bacteria are genetically engineered to include multiple mechanisms of action (MOAs), e.g., circuits producing multiple copies of the same product (e.g., to enhance copy number) or circuits performing multiple different functions. For example, the recombinant bacteria may include four copies of the gene, gene(s), or gene cassettes for producing the payload(s) inserted at four different insertion sites. Alternatively, the recombinant bacteria may include three copies of the gene, gene(s), or gene cassettes for producing the payload(s) inserted at three different insertion sites and three copies of the gene, gene(s), or gene cassettes for producing the payload(s) inserted at three different insertion sites.

In some embodiments, under conditions where the gene, gene(s), or gene cassettes for producing the payload(s) is expressed, the recombinant bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the payload(s) as compared to unmodified bacteria of the same subtype under the same conditions.

In some embodiments, the recombinant bacteria produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of a payload under inducing conditions than unmodified bacteria of the same subtype under the same conditions. Certain unmodified bacteria will not have detectable levels of the payload. In embodiments using genetically modified forms of these bacteria, the payload will be detectable under inducing conditions.

In some embodiments, quantitative PCR (qPCR) is used to amplify, detect, and/or quantify mRNA expression levels of the gene, gene(s), or gene cassettes for producing the payload(s). Primers may be designed and used to detect mRNA in a sample according to methods known in the art. In some embodiments, a fluorophore is added to a sample reaction mixture that may contain payload RNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the payload(s).

The gene sequence(s) encoding EGF peptides for secretion may be expressed under the control of a constitutive promoter or an inducible promoter. The gene sequence(s) encoding the one or more EGF peptides for secretion are expressed under the control of a promoter that is directly or indirectly induced by exogenous environmental conditions, e.g., low-oxygen or anaerobic conditions, wherein expression of the gene sequence(s) encoding the one or more EGF peptides for secretion are activated under low-oxygen or anaerobic environments, such as the environment of the mammalian gut. The gene sequence(s) encoding the one or more EGF peptides for secretion are expressed under the control of a temperature-sensitive promoter. Alternatively, the gene sequence(s) encoding the one or more EGF peptides for secretion are expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, and a P_{TetR} promoter, each of which are described in more detail herein. Inducible promoters are described in more detail infra.

The at least one gene encoding EGF for secretion may be present on a plasmid or chromosome in the bacterial cell. In one embodiment, a native copy of the gene sequence(s) encoding EGF for secretion are located in the chromosome of the bacterial cell, and at least one gene encoding EGF for secretion from a different species of bacteria are located on a plasmid in the bacterial cell. In yet another embodiment, a native copy of the gene sequence(s) encoding EGF for secretion are located on a plasmid in the bacterial cell, and at least one gene encoding EGF for secretion from a different species of bacteria are located on a plasmid in the bacterial cell. In yet another embodiment, a native copy of the gene sequence(s) encoding EGF for secretion are located in the chromosome of the bacterial cell, and at least one gene encoding EGF for secretion from a different species of bacteria are located in the chromosome of the bacterial cell.

In some embodiments, the gene sequence(s) encoding the one or more EGF peptides for secretion are expressed on a low-copy plasmid. In some embodiments, the gene sequence(s) encoding the one or more EGF peptides for secretion are expressed on a high-copy plasmid. In some embodiments, the high-copy plasmid may be useful for increasing expression of EGF for secretion.

In some embodiments, a recombinant bacterial cell comprising at least one gene encoding EGF for secretion are expressed on a high-copy plasmid do not increase tryptophan catabolism as compared to a recombinant bacterial cell comprising the same gene expressed on a low-copy plasmid in the absence of a heterologous importer of tryptophan and/or its metabolites and additional copies of a native importer of tryptophan and/or its metabolites. In alternate embodiments, the importer of tryptophan and/or its metabolites is used in conjunction with a high-copy plasmid.

In some embodiments, the recombinant bacteria described above further comprise one or more of the modifications, mutations, and/or deletions in endogenous genes described herein.

In some embodiments, the genetically engineered microorganism further comprises a mutation and/or deletion in ldhA. In some embodiments, the genetically engineered microorganism further comprises a mutation and/or deletion in frdA. In some embodiments, the genetically engineered microorganism further comprises a mutation and/or deletion in adhE. In some embodiments, the genetically engineered microorganism further comprises a mutation and/or deletion in one or more of ldhA, frdA, and adhE.

In some embodiments, surface display could be used to display EGF on the surface of the genetically modified bacterium. In some embodiments, the recombinant bacteria and/or microorganisms encode one or more gene(s) and/or gene cassette(s) encoding EGF, which is anchored or displayed on the surface of the bacteria and/or microorganisms.

### Induction of Payloads During Strain Culture

In some embodiments, it is desirable to pre-induce payload or EGF expression and/or payload activity prior to administration. Such payload or EGF may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or EGF. In such instances, the recombinant bacteria express EGF, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, EGF and are directly or indirectly induced, while the strains is grown up for in vivo administration. Without wishing to be bound by theory, pre-induction may boost in vivo activity. This is particularly important in proximal regions of the gut which are reached first by the bacteria, e.g., the small intestine. If the bacterial residence time in this compartment is relatively short, the bacteria may pass through the small intestine without reaching full in vivo induction capacity. In contrast, if a strain is pre-induced and preloaded, the strains are already fully active, allowing for greater activity more quickly as the bacteria reach the intestine. Ergo, no transit time is “wasted”, in which the strain is not optimally active. As the bacteria continue to move through the intestine, in vivo induction occurs under environmental conditions of the gut (e.g., low oxygen, or in the presence of gut metabolites).

In one embodiment, expression of one or more payload(s), is induced during cell growth, cell expansion, fermentation, recovery, purification, formulation, and/or manufacture. In one embodiment, expression EGF is induced during cell growth, cell expansion, fermentation, recovery, purification, formulation, and/or manufacture. In one embodiment, expression of one or more payload(s), is driven from the same promoter as a multicistronic message. In one embodiment, expression of one or more payload(s) is driven from the same promoter as two or more separate messages. In one embodiment, expression of one or more payload(s) is driven from the one or more different promoters.

In some embodiments, the strains are administered without any pre-induction protocols during strain growth prior to in vivo administration.

### Anaerobic Induction

In some embodiments, cells are induced under anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then switched to anaerobic or low oxygen conditions for approximately 3 to 5 hours. In some embodiments, strains are induced under anaerobic or low oxygen conditions, e.g., to induce FNR promoter activity and drive expression of one or more payload(s) under the control of one or more FNR promoters.

In one embodiment, expression of one or more payload(s), is under the control of one or more anaerobic or low oxygen inducible promoter(s), e.g., FNR promoter(s), and is induced during cell growth, cell expansion, fermentation, recovery, purification, formulation, and/or manufacture under anaerobic or low oxygen conditions. In one embodiment, expression of EGF is under the control of one or more anaerobic or low oxygen inducible promoter(s), e.g., FNR promoter(s) and is induced during cell growth, cell expansion, fermentation, recovery, purification, formulation, and/or manufacture under anaerobic or low oxygen conditions.

In one embodiment, expression of two or more payload(s), is under the control of one or more anaerobic or low oxygen inducible promoter(s), e.g., FNR promoter(s), and is driven from the same promoter in the form of a multicistronic message under anaerobic or low oxygen conditions. In one embodiment, expression of one or more payload(s), is under the control of one or more anaerobic or low oxygen inducible promoter(s), e.g., FNR promoter(s), and is driven from the same promoter as two or more separate messages under anaerobic or low oxygen conditions. In one embodiment, expression of one or more payload(s) under the control of one or more anaerobic or low oxygen inducible promoter(s), e.g., FNR promoter(s), and is driven from the one or more different promoters under anaerobic or low oxygen conditions.

Without wishing to be bound by theory, strains that comprise one or more payload(s) under the control of an FNR promoter, may allow expression of payload(s) from these promoters in vitro, under anaerobic or low oxygen culture conditions, and in vivo, under the low oxygen conditions found in the gut.

In some embodiments, promoters inducible by arabinose, IPTG, rhamnose, tetracycline, and/or other chemical and/or nutritional inducers can be induced under anaerobic or low oxygen conditions in the presence of the chemical and/or nutritional inducer. In some embodiments, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) and/or under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more payload gene sequence(s) is under the control of one or more promoter(s) regulated by chemical and/or nutritional inducers and is induced during cell growth, cell expansion, fermentation, recovery, purification, formulation, and/or manufacture under anaerobic and/or low oxygen conditions. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, expression of one or more payload(s), is under the control of one or more promoter(s) regulated by chemical and/or nutritional inducers and is driven from the same promoter in the form of a multicistronic message under anaerobic and/or low oxygen conditions. In one embodiment, expression of one or more payload(s), is under the control of one or more promoter(s) regulated by chemical and/or nutritional inducers and is driven from the same promoter as two or more separate messages under anaerobic and/or low oxygen conditions. In one embodiment, expression of one or more payload(s), is under the control of one or more promoter(s) regulated by chemical and/or nutritional inducers and is driven from the one or more different promoters under anaerobic and/or low oxygen conditions.

In one embodiment, strains may comprise a combination of gene sequence(s), some of which are under control of a first inducible promoter and others which are under control of a second inducible promoter, both induced by chemical and/or nutritional inducers, under anaerobic or low oxygen conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g., a temperature sensitive promoter. In one embodiment, strains may comprise a combination of gene sequence(s), some of which are under control of a first inducible promoter, e.g., a FNR promoter and others which are under control of a second inducible promoter, e.g., a temperature sensitive promoter. In one embodiment, strains may comprise a combination of gene sequence(s), some of which are under control of a first inducible promoter, e.g., a chemically induced and others which are under control of a second inducible promoter, e.g., a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to a permissive temperature, for approximately 3 to 5 hours.

In some embodiments, promoters inducible by arabinose, IPTG, rhamnose, tetracycline, and/or other chemical and/or nutritional inducers described herein or known in the art can be induced under aerobic conditions in the presence of the chemical and/or nutritional inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In one embodiment, expression of one or more payload(s), is under the control of one or more promoter(s) regulated by chemical and/or nutritional inducers and is driven from the same promoter in the form of a multicistronic message under aerobic conditions. In one embodiment, expression of one or more payload(s), is under the control of one or more promoter(s) regulated by chemical and/or nutritional inducers and is driven from the same promoter as two or more separate messages under aerobic conditions. In one embodiment, expression of one or more payload(s), is under the control of one or more promoter(s) regulated by chemical and/or nutritional inducers and is driven from the one or more different promoters under aerobic conditions.

In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In some embodiments, promoters regulated by temperature are induced during cell growth, cell expansion, fermentation, recovery, purification, formulation, and/or manufacture. In one embodiment, expression of one or more payload(s) is driven directly or indirectly by one or more thermoregulated promoter(s) and is induced during cell growth, cell expansion, fermentation, recovery, purification, formulation, and/or manufacture under aerobic conditions.

In one embodiment, expression of one or more payload(s) is driven directly or indirectly by one or more thermoregulated promoter(s) and is driven from the same promoter in the form of a multicistronic message under aerobic conditions. In one embodiment, expression of one or more payload(s) is driven directly or indirectly by one or more thermoregulated promoter(s) and is driven from the same promoter as two or more separate messages under aerobic conditions. In one embodiment, expression of one or more payload(s) is driven directly or indirectly by one or more thermoregulated promoter(s) and is driven from the one or more different promoters under aerobic conditions.

In one embodiment, strains may comprise a combination of gene sequence(s), some of which are under control of a first inducible promoter and others which are under control of a second inducible promoter, both induced under aerobic conditions. In some embodiments, a strain comprises three or more different promoters which are induced under aerobic culture conditions.

In one embodiment, strains may comprise a combination of gene sequence(s), some of which are under control of a first inducible promoter and others which are under control of a second inducible promoter, both induced by chemical and/or nutritional inducers. In one embodiment, strains may comprise a combination of gene sequence(s), some of which are under control of a first inducible promoter, e.g., a chemically inducible promoter, and others which are under control of a second inducible promoter, e.g., a temperature sensitive promoter under aerobic culture conditions. In some embodiments two or more chemically induced promoter gene sequence(s) are combined with a thermoregulated construct described herein. In one embodiment, the chemical and/or nutritional inducer is arabinose and the promoter is inducible by arabinose. In one embodiment, the chemical and/or nutritional inducer is IPTG and the promoter is inducible by IPTG. In one embodiment, the chemical and/or nutritional inducer is rhamnose and the promoter is inducible by rhamnose. In one embodiment, the chemical and/or nutritional inducer is tetracycline and the promoter is inducible by tetracycline.

In one embodiment, strains may comprise a combination of gene sequence(s), some of which are under control of a first inducible promoter, e.g., a FNR promoter and others which are under control of a second inducible promoter, e.g., a temperature sensitive promoter. In one embodiment, strains may comprise a combination of gene sequence(s), some of which are under control of a first inducible promoter, e.g., a chemically induced and others which are under control of a second inducible promoter, e.g., a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequences under the control of one or more constitutive promoter(s) active under aerobic conditions.

In some embodiments, genetically engineered strains comprise gene sequence(s) which are induced under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s), which are arabinose inducible under aerobic culture conditions. In some embodiments, these strains do not further comprise FNR inducible gene sequence(s) for in vivo activation in the gut.

In some embodiments, genetically engineered strains comprise gene sequence(s), which are IPTG inducible under aerobic culture conditions. In some embodiments, these strains further comprise FNR inducible gene sequence(s) for in vivo activation in the gut. In some embodiments, these strains do not further comprise FNR inducible gene sequence(s) for in vivo activation in the gut.

In some embodiments, genetically engineered strains comprise gene sequence(s) which are arabinose inducible under aerobic culture conditions. In some embodiments, such a strain further comprises sequence(s) which are IPTG inducible under aerobic culture conditions. In some embodiments, these strains further comprise FNR inducible gene payload sequence(s) for in vivo activation in the gut. In some embodiments, these strains do not further comprise FNR inducible gene sequence(s) for in vivo activation in the gut.

As evident from the above non-limiting examples, genetically engineered strains comprise inducible gene sequence(s) which can be induced numerous combinations. For example, rhamnose or tetracycline can be used as an inducer with the appropriate promoters in addition or in lieu of arabinose and/or IPTG or with thermoregulation. Additionally, such bacterial strains can also be induced with the chemical and/or nutritional inducers under anaerobic conditions.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) are driven by an anaerobic and/or low oxygen promoter, e.g., a FNR promoter. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

In one embodiment, expression of one or more payload(s), is under the control of one or more FNR promoter(s) and is driven from the same promoter in the form of a multicistronic message under microaerobic conditions. In one embodiment, expression of one or more payload(s), is under the control of one or more FNR promoter(s) and is driven from the same promoter as two or more separate messages under microaerobic conditions. In one embodiment, expression of one or more payload(s), is under the control of one or more FNR promoter(s) and is driven from the one or more different promoters under microaerobic conditions.

Without wishing to be bound by theory, strains that comprise one or more payload(s) under the control of an FNR promoter, may allow expression of payload(s) from these promoters in vitro, under microaerobic culture conditions, and in vivo, under the low oxygen conditions found in the gut.

In some embodiments, promoters inducible by arabinose, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein. In some embodiments, strains may comprise one or more payload gene sequence(s) under the control of an FNR promoter and one or more payload gene sequence(s) under the control of a one or more thermoregulated promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

In one embodiment, expression of one or more payload(s), is under the control of one or more promoter(s) regulated by chemical and/or nutritional inducers and is driven from the same promoter in the form of a multicistronic message under microaerobic conditions. In one embodiment, expression of one or more payload(s), is under the control of one or more promoter(s) regulated by chemical and/or nutritional inducers and is driven from the same promoter as two or more separate messages under microaerobic conditions. In one embodiment, expression of one or more payload(s), is under the control of one or more promoter(s) regulated by chemical and/or nutritional inducers and is driven from the one or more different promoters under microaerobic conditions.

In one embodiment, strains may comprise a combination of gene sequence(s), some of which are under control of a first inducible promoter and others which are under control of a second inducible promoter, both induced by chemical and/or nutritional inducers, under microaerobic conditions. In one embodiment, strains may comprise a combination of gene sequence(s), some of which are under control of a first inducible promoter and others which are under control of a second inducible promoter, both induced by chemical and/or nutritional inducers. In some embodiments, the strains comprise gene sequence(s) under the control of a third inducible promoter, e.g., an anaerobic/low oxygen promoter or microaerobic promoter, e.g., FNR promoter. In one embodiment, strains may comprise a combination of gene sequence(s), some of which are under control of a first inducible promoter, e.g., a chemically induced promoter or a low oxygen or microaerobic promoter and others which are under control of a second inducible promoter, e.g., a temperature sensitive promoter. In one embodiment, strains may comprise a combination of gene sequence(s), some of which are under control of a first inducible promoter, e.g., a FNR promoter and others which are under control of a second inducible promoter, e.g., a temperature sensitive promoter. In one embodiment, strains may comprise a combination of gene sequence(s), some of which are under control of a first inducible promoter, e.g., a chemically induced and others which are under control of a second inducible promoter, e.g., a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload under the control of one or more constitutive promoter(s) active under low oxygen conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10\hat{ }8 to 1×10\hat{ }11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e, growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour, to reach an OD of 0.1 to 10, until the cells are at a density ranging from 1×10\hat{ }8 to 1×10\hat{ }11. Then the chemical inducer, e.g., arabinose or IPTG, is added to the culture. After 3 to 5 hours, an additional dose of the inducer is added to re-initiate the induction. Spiking can be repeated as needed.

In some embodiments, phasing or cycling changes in temperature in the culture. In another embodiment, adjustment of temperature may be used to improve the activity of a payload. For example, lowering the temperature during culture may improve the proper folding of the payload. In such instances, cells are first grown at a temperature optimal for growth (e.g., 37 C). In some embodiments, the cells are then induced, e.g., by a chemical inducer, to express the payload. Concurrently or after a set amount of induction time, the temperature in the media is lowered, e.g., between 25 and 35 C, to allow improved folding of the expressed payload.

In some embodiments, payload(s) are under the control of different inducible promoters, for example two different chemical inducers. In other embodiments, the payload is induced under low oxygen conditions or microaerobic conditions and a second payload is induced by a chemical inducer.

In one embodiment, expression of one or more payload(s) is under the control of one or more FNR promoter(s) and is induced during cell growth, cell expansion, fermentation, recovery, purification, formulation, and/or manufacture by using phasing or cycling or pulsing or spiking techniques.

In one embodiment, expression of one or more payload(s), is under the control of one or more FNR promoter(s) and is driven from the same promoter in the form of a multicistronic message through the employment of phasing or cycling or pulsing or spiking techniques. In one embodiment, expression of one or more payload(s), is under the control of one or more FNR promoter(s) and is driven from the same promoter as two or more separate messages through the employment of phasing or cycling or pulsing or spiking techniques. In one embodiment, expression of one or more payload(s), is under the control of one or more FNR promoter(s) and is driven from the one or more different promoters through the employment of phasing or cycling or pulsing or spiking techniques.

In some embodiments, promoters inducible by arabinose, IPTG, rhamnose, tetracycline, and/or other chemical and/or nutritional inducers can be induced through the employment of phasing or cycling or pulsing or spiking techniques in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s) and one or more payload gene sequence(s) under the control of a one or more promoter(s) which are induced by a one or more chemical and/or nutritional inducer(s), including, but not limited to, arabinose, IPTG, rhamnose, tetracycline, and/or other chemical and/or nutritional inducers described herein or known in the art. In some embodiments, strains may comprise one or more payload gene sequence(s) under the control of one or more FNR promoter(s), and one or more payload gene sequence(s) under the control of a one or more constitutive promoter(s) described herein and are induced through the employment of phasing or cycling or pulsing or spiking techniques. In some embodiments, strains may comprise one or more payload gene sequence(s) under the control of an FNR promoter and one or more payload gene sequence(s) under the control of a one or more thermoregulated promoter(s) described herein, and are induced through the employment of phasing or cycling or pulsing or spiking techniques.

Any of the strains described herein can be grown through the employment of phasing or cycling or pulsing or spiking techniques. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under anaerobic and/or low oxygen conditions.

In one embodiment, expression of one or more payload(s), is under the control of one or more promoter(s) regulated by chemical and/or nutritional inducers and is driven from the same promoter in the form of a multicistronic message and which are induced through the employment of phasing or cycling or pulsing or spiking techniques. In one embodiment, expression of one or more payload(s), is under the control of one or more promoter(s) regulated by chemical and/or nutritional inducers and is driven from the same promoter as two or more separate messages and is grown through the employment of phasing or cycling or pulsing or spiking techniques. In one embodiment, expression of one or more payload(s), is under the control of one or more promoter(s) regulated by chemical and/or nutritional inducers and is driven from the one or more different promoters, all of which are induced through the employment of phasing or cycling or pulsing or spiking techniques.

In one embodiment, strains may comprise a combination of gene sequence(s), some of which are under control of a first inducible promoter and others which are under control of a second inducible promoter, both induced by chemical and/or nutritional inducers, through the employment of phasing or cycling or pulsing or spiking techniques. In one embodiment, strains may comprise a combination of gene sequence(s), some of which are under control of a first inducible promoter and others which are under control of a second inducible promoter, both induced by chemical and/or nutritional inducers through the employment of phasing or cycling or pulsing or spiking techniques. In some embodiments, the strains comprise gene sequence(s) under the control of a a third inducible promoter, e.g., an anaerobic/low oxygen promoter, e.g., FNR promoter. In one embodiment, strains may comprise a combination of gene sequence(s), some of which are under control of a first inducible promoter, e.g., a chemically induced promoter or a low oxygen promoter and others which are under control of a second inducible promoter, e.g., a temperature sensitive promoter. In one embodiment, strains may comprise a combination of gene sequence(s), some of which are under control of a first inducible promoter, e.g., a FNR promoter and others which are under control of a second inducible promoter, e.g., a temperature sensitive promoter. In one embodiment, strains may comprise a combination of gene sequence(s), some of which are under control of a first inducible promoter, e.g., a chemically induced and others which are under control of a second inducible promoter, e.g., a temperature sensitive promoter. In some embodiments, strains may comprise one or more payload gene sequence(s) under the control of an FNR promoter and one or more payload gene sequence(s) under the control of a one or more promoter(s) which are induced by a one or more chemical and/or nutritional inducer(s), including, but not limited to, by arabinose, IPTG, rhamnose, tetracycline, and/or other chemical and/or nutritional inducers described herein or known in the art. Additionally the strains may comprise a construct which is under thermoregulatory control. In some embodiments, the bacteria strains further comprise payload sequence(s) under the control of one or more constitutive promoter(s) active under low oxygen conditions. Any of the strains described in these embodiments may be induced through the employment of phasing or cycling or pulsing or spiking techniques.

### Aerobic Induction of the FNR Promoter

FNRS24Y is a mutated form of FNR which is more resistant to inactivation by oxygen, and therefore can activate FNR promoters under aerobic conditions (see e.g., Jervis AJ The 02 sensitivity of the transcription factor FNR is controlled by Ser24 modulating the kinetics of [4Fe-4S] to [2Fe-2S] conversion, Proc Natl Acad Sci USA. 2009 Mar. 24; 106(12):4659-64, the contents of which is herein incorporated by reference in its entirety). In some embodiments, an oxygen bypass system shown and described in figures and examples is used. In this oxygen bypass system, FNRS24Y is induced by addition of arabinose and then drives the expression of EGF by binding and activating the FNR promoter under aerobic conditions. Thus, strains can be grown, produced or manufactured efficiently under aerobic conditions, while being effectively pre-induced and pre-loaded, as the system takes advantage of the strong FNR promoter resulting in of high levels of expression of EGF. This system does not interfere with or compromise in vivo activation, since the mutated FNRS24Y is no longer expressed in the absence of arabinose, and wild type FNR then binds to the FNR promoter and drives expression of EGF.

In some embodiments, FNRS24Y is expressed during aerobic culture growth and induces EGF. In other embodiments described herein, a second payload expression can also be induced aerobically, e.g., by arabinose. In a non-limiting example, EGF and FNRS24Y can in some embodiments be induced simultaneously, e.g., from an arabinose inducible promoter. In some embodiments, FNRS24Y and EGF are transcribed as a bicistronic message whose expression is driven by an arabinose promoter. In some embodiments, FNRS24Y is knocked into the arabinose operon, allowing expression to be driven from the endogenous Para promoter.

In some embodiments, a LacI promoter and IPTG induction are used in this system (in lieu of Para and arabinose induction). In some embodiments, a rhamnose inducible promoter is used in this system. In some embodiments, a temperature sensitive promoter is used to drive expression of FNRS24Y.

### Secretion

In any of the embodiments described herein, in which the genetically engineered organism, e.g., engineered bacteria, produces a protein, polypeptide, or peptide, DNA, RNA, small molecule or other molecule intended to be secreted from the microorganism, the engineered microorganism may comprise a secretion mechanism and corresponding gene sequence(s) encoding the secretion system.

In some embodiments, the recombinant bacteria further comprise a native secretion mechanism or non-native secretion mechanism that is capable of secreting the EGF molecule from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In some embodiments, the recombinant bacteria further comprise a non-native double membrane-spanning secretion system. Double membrane-spanning secretion systems include, but are not limited to, the type I secretion system (TISS), the type II secretion system (T2SS), the type III secretion system (T3SS), the type IV secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Examples of such secretion systems are shown in figures and examples. Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system or autosecreter system (T5SS), the curli secretion system, and the chaperone-usher pathway for pili assembly (Saier, 2006; Costa et al., 2015).

In some embodiments in which EGF is secreted or exported from the microorganism, the engineered microorganism comprises gene sequence(s) that includes a secretion tag. In some embodiments, the EGF protein includes a “secretion tag” of either RNA or peptide origin to direct the EGF protein to specific secretion systems. For example, a secretion tag for the Type I Hemolysin secretion system is encoded in the C-terminal 53 amino acids of the alpha hemolysin protein (HlyA).

In some embodiments, a Hemolysin-based Secretion System is used to secrete EGF. Type I Secretion systems offer the advantage of translocating their passenger peptide directly from the cytoplasm to the extracellular space, obviating the two-step process of other secretion types. The alpha-hemolysin (HlyA) of uropathogenic Escherichia coli uses HlyB, an ATP-binding cassette transporter; HlyD, a membrane fusion protein; and TolC, an outer membrane protein. The assembly of these three proteins forms a channel through both the inner and outer membranes. HlyB inserts into inner membrane to form a pore, HlyD aligns HlyB with TolC (outer membrane pore) thereby forming a channel through inner and outer membrane. Natively, this channel is used to secrete HlyA, however, to secrete EGF, the secretion signal-containing C-terminal portion of HlyA is fused to the C-terminal portion of an EGF peptide (star) to mediate secretion of this peptide. The C-terminal secretion tag can be removed by either an autocatalytic or protease-catalyzed e.g., OmpT cleavage thereby releasing the EGF protein into the extracellular milieu. In some embodiments one or more EGF proteins contain expressed as fusion protein with the 53 amino acids of the C termini of alpha-hemolysin (hlyA) of E. coli CFT073 (C terminal secretion tag).

In some embodiments, a Type V Autotransporter Secretion System is used to secrete EGF. The Type V Auto-secretion System utilizes an N-terminal Sec-dependent peptide tag (inner membrane) and C-terminal tag (outer-membrane). This system uses the Sec-system to get from the cytoplasm to the periplasm. The C-terminal tag then inserts into the outer membrane forming a pore through which the “passenger protein” threads through. Due to the simplicity of the machinery and capacity to handle relatively large protein fluxes, the Type V secretion system is attractive for the extracellular production of recombinant proteins. EGF can be fused to an N-terminal secretion signal, a linker, and the beta-domain of an autotransporter. The N-terminal, Sec-dependent signal sequence directs the protein to the SecA-YEG machinery which moves the protein across the inner membrane into the periplasm, followed by subsequent cleavage of the signal sequence. The Beta-domain is recruited to the Bam complex (‘Beta-barrel assembly machinery’) where the beta-domain is folded and inserted into the outer membrane as a beta-barrel structure. EGF is threaded through the hollow pore of the beta-barrel structure ahead of the linker sequence. Once across the outer membrane, the passenger is released from the membrane-embedded C-terminal tag by either an autocatalytic, intein-like mechanism (left side of Bam complex) or via a membrane-bound protease (black scissors; right side of Bam complex) (i.e., OmpT). For example, a membrane-associated peptidase to a complimentary protease cut site in the linker. Thus, in some embodiments, the secreted molecule, such as a heterologous protein or peptide comprises an N-terminal secretion signal, a linker, and beta-domain of an autotransporter so as to allow the molecule to be secreted from the bacteria.

The N-terminal tag is removed by the Sec system. Thus, in some embodiments, the secretion system is able to remove this tag before secreting EGF from the engineered bacteria. In the Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the molecule(s) into the extracellular milieu.

In some embodiments, the recombinant bacteria comprise a type III or a type III-like secretion system (T3SS) from Shigella, Salmonella, E. coli, Bivrio, Burkholderia, Yersinia, Chlamydia, or Pseudomonas. The traditional T3SS is capable of transporting a protein from the bacterial cytoplasm to the host cytoplasm through a needle complex. In the Type III traditional secretion system, the basal body closely resembles the flagella, however, instead of a “tail”/whip, the traditional T3SS has a syringe to inject the passenger proteins into host cells. The secretion tag is encoded by an N-terminal peptide (lengths vary and there are several different tags, see PCT/US14/020972). The N-terminal tag is not removed from the polypeptides in this secretion system.

The T3SS may be modified to secrete the molecule from the bacterial cytoplasm, but not inject the molecule into the host cytoplasm. Thus, the molecule is secreted into the gut lumen, tumor microenvironment, or other extracellular space. In some embodiments, the recombinant bacteria comprise said modified T3SS and are capable of secreting the EGF from the bacterial cytoplasm. In some embodiments, the secreted molecule, such as a heterologous protein or peptide comprises a type III secretion sequence that EGF to be secreted from the bacteria.

In the Flagellar modified Type III Secretion, the tag is encoded in 5′ untranslated region of the mRNA and thus there is no peptide tag to cleave/remove. This modified system does not contain the “syringe” portion and instead uses the basal body of the flagella structure as the pore to translocate across both membranes and out through the forming flagella. If the fliC/fliD genes (encoding the flagella “tail”/whip) are disrupted the flagella cannot fully form and this promotes overall secretion. In some embodiments, the tail portion can be removed entirely.

In some embodiments, a flagellar type III secretion pathway is used to EGF. In some embodiments, an incomplete flagellum is used to secrete EGF by recombinantly fusing the peptide to an N-terminal flagellar secretion signal of a native flagellar component. In this manner, the intracellularly expressed chimeric peptide can be mobilized across the inner and outer membranes into the surrounding host environment.

For example, a modified flagellar type III secretion apparatus in which untranslated DNA fragment upstream of the gene fliC (encoding flagellin), e.g., a 173-bp region, is fused to the gene encoding the heterologous protein or peptide can be used to secrete polypeptides of interest (See, e.g., Majander et al., Extracellular secretion of polypeptides using a modified Escherichia coli flagellar secretion apparatus. Nat Biotechnol. 2005 April; 23(4):475-81). In some cases, the untranslated region from the fliC loci may not be sufficient to mediate translocation of the passenger peptide through the flagella. Here it may be necessary to extend the N-terminal signal into the amino acid coding sequence of FliC, for example, by using the 173 bp of untranslated region along with the first 20 amino acids of FliC (see, e.g., Duan et al., Secretion of Insulinotropic Proteins by Commensal Bacteria: Rewiring the Gut To Treat Diabetes, Appl. Environ. Microbiol. December 2008 vol. 74 no. 23 7437-7438).

In alternate embodiments, the recombinant bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the recombinant bacteria comprise a TAT or a TAT-like system and are capable of secreting EGF from the bacterial cytoplasm. One of ordinary skill in the art would appreciate that the secretion systems disclosed herein may be modified to act in different species, strains, and subtypes of bacteria, and/or adapted to deliver different payloads.

In order to translocate EGF to the extracellular space, the polypeptide must first be translated intracellularly, mobilized across the inner membrane and finally mobilized across the outer membrane. EGF contains disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the reducing-environment periplasm in conjunction with a destabilizing outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the recombinant bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp is the most abundant polypeptide in the bacterial cell existing at ˜500,000 copies per cell and functions as the primary ‘staple’ of the bacterial cell wall to the peptidoglycan. Silhavy, T. J., Kahne, D. & Walker, S. The bacterial cell envelope. Cold Spring Harb Perspect Biol 2, a000414 (2010). TolA-pal and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpI can induce leaky phenotypes by promoting an excessive build-up of periplasmic protein. Mutation of the proteases can also preserve the EGF polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpl, under the control of an inducible promoter. For example, expression of lpp or other cell wall stability protein or periplasmic protease can be repressed in conditions where EGF needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, wherein peptide overexpression can be induced in conditions in which EGF needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

Table 9 and Table 10 below lists secretion systems for Gram-positive bacteria and Gram-negative bacteria.

TABLE 9
Secretion systems for gram positive bacteria

| Bacterial Strain | Relevant Secretion System |
|---|---|
| C. novyi-NT (Gram+) | Sec pathway |
| | Twin- arginine (TAT) pathway |
| C. butryicum (Gram+) | Sec pathway |
| | Twin- arginine (TAT) pathway |
| Listeria monocytogenes (Gram+) | Sec pathway |
| | Twin- arginine (TAT) pathway |

TABLE 10
Secretion Systems for Gram negative bacteria
Protein secretary pathways (SP) in gram-negative bacteria and descendants

| | | | | | | | |
|---|---|---|---|---|---|---|---|
| Type | | | | | | # Proteins/ | Energy |
| (Abbreviation) | Name | TC#² | Bacteria | Archaea | Eukarya | System | Source |

IMPS - Gram-negative bacterial inner membrane channel-forming translocases

| | | | | | | | |
|---|---|---|---|---|---|---|---|
| ABC (SIP) | ATP binding | 3.A.1 | + | + | + | 3-4 | ATP |
| | cassette translocase | | | | | | |
| SEC (IISP) | General secretory | 3.A.5 | + | + | + | ~12 | GTP |
| | translocase | | | | | | OR |
| | | | | | | | ATP + |
| | | | | | | | PMF |
| Fla/Path | Flagellum/virulence- | 3.A.6 | + | − | − | >10 | ATP |
| (IIISP) | related translocase | | | | | | |
| Conj (IVSP) | Conjugation-related | 3.A.7 | + | − | − | >10 | ATP |
| | translocase | | | | | | |
| Tat (IISP) | Twin-arginine | 2.A.64 | + | + | + | 2-4 | PMF |
| | targeting translocase | | | | (chloroplasts) | | |
| Oxal (YidC) | Cytochrome oxidase | 2.A.9 | + | + | + | 1 | None or |
| | biogenesis family | | | | (mitochondria | | PMF |
| | | | | | chloroplasts) | | |
| MscL | Large conductance | 1.A.22 | + | + | + | 1 | None |
| | mechanosensitive | | | | | | |
| | channel family | | | | | | |
| Holins | Holin functional | 1.E.1•21 | + | − | − | 1 | None |
| | superfamily | | | | | | |

Eukaryotic Organelles

| | | | | | | | |
|---|---|---|---|---|---|---|---|
| MPT | Mitochondrial | 3.A.B | − | − | + | >20 | ATP |
| | protein translocase | | | | (mitochondrial) | | |
| CEPT | Chloroplast | 3.A.9 | (+) | − | + | ≥3 | GTP |
| | envelope protein | | | | (chloroplasts) | | |
| | translocase | | | | | | |
| Bcl-2 | Eukaryotic Bcl-2 | 1.A.21 | − | − | + | 1? | None |
| | family (programmed | | | | | | |
| | cell death) | | | | | | |

Gram-negative bacterial outer membrane channel-forming translocases

| | | | | | | | |
|---|---|---|---|---|---|---|---|
| MTB (IISP) | Main terminal | 3.A.15 | +^{b} | − | − | ~14 | ATP; |
| | branch of the | | | | | | PMF |
| | general secretory | | | | | | |
| | translocase | | | | | | |
| FUP AT-1 | Fimbrial usher | 1.B.11 | +^{b} | − | − | 1 | None |
| | protein | 1.B.12 | +^{b} | | − | 1 | None |
| | Autotransporter-1 | | | | | | |
| AT-2 OMF | Autotransporter-2 | 1.B.40 | +^{b} | − | − | 1 | None |
| (ISP) | | 1.B.17 | +^{b} | | +(?) | 1 | None |
| TPS Secretin | | 1.B.20 | + | − | + | 1 | None |
| (IISP and | | 1.B.22 | +^{b} | | − | 1 | None |
| IISP) | | | | | | | |
| OmpIP | Outer membrane | 1.B.33 | + | − | + | ≥4 | None |
| | insertion porin | | | | (mitochondria; | | |
| | | | | | chloroplasts) | | |

The above tables for Gram-positive and Gram-negative bacteria list secretion systems that can be used to secrete polypeptides and other molecules from the engineered bacteria (Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety).

In some embodiments, the recombinant bacterial comprise a native or non-native secretion system described herein for the secretion of a molecule, e.g., a cytokine, antibody (e.g., scFv), metabolic enzyme, e.g., kynureninase, an others described herein.

Polypeptide Sequences of exemplary secretion tags include PhoA (SEQ ID NO: 385), PhoA (SEQ ID NO: 386), OmpF (SEQ ID NO: 387), cvaC (SEQ ID NO: 388), TorA (SEQ ID NO: 389), fdnG (SEQ ID NO: 390), dmsA (SEQ ID NO: 391), PelB (SEQ ID NO: 392), HlyA secretion signal (SEQ ID NO: 393), and HlyA secretion signal (SEQ ID NO: 394). In some embodiments, secretion tags of endogenous secreted proteins from E. coli can be used to secrete EGF. Exemplary secretion tags from secreted E coli Nissle include ECOLIN_05715 Secretion signal (SEQ ID NO: 395), ECOLIN_16495 Secretion signal (SEQ ID NO: 396), ECOLIN_19410 Secretion signal (SEQ ID NO:397), and ECOLIN_19880 Secretion signal (SEQ ID NO:398). Additional secretion tags include adhesion (SEQ ID NOS: 1091 and 1099), DsbA (SEQ ID NOS: 1092 and 1100), GltI (SEQ ID NOS: 1093 and 1101), GspD (SEQ ID NOS: 1089 and 1102), HdeB (SEQ ID NOS: 1090 and 1103), MalE (SEQ ID NOS: 1094 and 1104), OppA (SEQ ID NOS: 1095 and 1105), PelB (SEQ ID NOS: 1096 and 1106), PhoA (SEQ ID NOS: 1097 and 1107) and PpiA (SEQ ID NOS: 1098 and 1108).

In some embodiments, recombinant bacteria comprise a nucleic acid sequence that encodes a polypeptide which is at least about 80%, 85%, 90%, 95%, or 99% homologous to one or more of the sequences of SEQ ID NOS: 385-398 and 1089-1108, or a nucleic acid sequence which is at least about 80%, 85%, 90%, 95%, or 99% homologous to one or more of the sequences of SEQ ID NOS: 385-398 and 1089-1108. Any secretion tag or secretion system can be combined with any cytokine described herein, and can be used to generate a construct (plasmid based or integrated) which is driven by a directly or indirectly inducible or constitutive promoter described herein. In some embodiments, the secretion system is used in combination with one or more genomic mutations, which leads to the leaky or diffusible outer membrane phenotype (DOM), including but not limited to, lpp, nlP, tolA, pal.

In some embodiments, the secretion system is selected from the type I (e.g., hemolysin secretion system), type II, type III, type III flagellar, type IV, type V, type VI, type VII, type VIII secretion systems and modifications thereof, e.g., modified type III, modified type III flagellar secretion systems, resistance-nodulation-division (RND) multi-drug efflux pumps, a single membrane secretion system, Sec and, TAT secretion systems.

Any of the secretion systems described herein may according to the disclosure be employed to secrete EGF. In some embodiments, EGF secreted by the recombinant bacteria is modified to increase resistance to proteases, e.g., intestinal proteases.

In some embodiments, the genetically engineered microorganisms are capable of expressing any one or more of the described circuits in low-oxygen conditions, and/or in the gut, or tissue specific molecules or metabolites, and/or in the presence of molecules or metabolites associated with inflammation or immune suppression, and/or in the presence of metabolites that may be present in the gut, and/or in the presence of metabolites that may or may not be present in vivo, and may be present in vitro during strain culture, expansion, production and/or manufacture, such as arabinose and others described herein. In some embodiments, the gene sequences(s) are controlled by a promoter inducible by such conditions and/or inducers. In some embodiments, the gene sequences(s) are controlled by a constitutive promoter, as described herein. In some embodiments, the gene sequences(s) are controlled by a constitutive promoter, and are expressed in in vivo conditions and/or in vitro conditions, e.g., during expansion, production and/or manufacture, as described herein.

In some embodiments, any one or more of the described circuits are present on one or more plasmids (e.g., high copy or low copy) or are integrated into one or more sites in the microorganism's chromosome. Also, in some embodiments, the genetically engineered microorganisms are further capable of expressing any one or more of the described circuits and further comprise one or more of the following: (1) one or more auxotrophies, such as any auxotrophies known in the art and provided herein, e.g., thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art and (7) one or more circuits for the production or degradation of one or more metabolites described herein (8) combinations of one or more of such additional circuits.

Non-limiting examples of EGF are described herein. These polypeptides may be mutated to increase stability, resistance to protease digestion, and/or activity.

TABLE 11
Comparison of Secretion systems for secretion of polypeptide from engineered
bacteria

| Secretion | | | | |
|---|---|---|---|---|
| System | Tag | Cleavage | Advantages | Other features |
| Modified Type | mRNA (or | No cleavage | No peptide | May not be as suited for |
| III (flagellar) | N-terminal) | necessary | tag | larger proteins |
| | | | Endogenous | Deletion of flagellar genes |
| Type V | N- and C- | Yes | Large proteins | 2-step secretion |
| autotransport | terminal | | Endogenous | |
| | | | Cleavable | |
| Type I | C-terminal | No | | Tag; Exogenous Machinery |
| Diffusible Outer | N-terminal | Yes | Disulfide bond | May affect cell |
| Membrane | | | formation | fragility/survivability/ |
| (DOM) | | | | growth/yield |

In some embodiments, EGF secreted using components of the flagellar type III secretion system. In a non-limiting example, EGF is secreted via Type I Hemolysin Secretion, is assembled behind a fliC-5′UTR (e.g., 173-bp untranslated region from the fliC loci), and is driven by the native promoter. In other embodiments, the expression of EGF secreted using components of the flagellar type III secretion system is driven by a tet-inducible promoter. In alternate embodiments, an inducible promoter such as oxygen level-dependent promoters (e.g., FNR-inducible promoter), promoters induced by IBD specific molecules or promoters induced by inflammation or an inflammatory response (RNS, ROS promoters), and promoters induced by a metabolite that may or may not be naturally present (e.g., can be exogenously added) in the gut, e.g., arabinose is used. In some embodiments, EGF is expressed from a plasmid (e.g., a medium copy plasmid). In some embodiments, EGF is expressed from a construct which is integrated into fliC locus (thereby deleting fliC), where it is driven by the native FliC promoter. In some embodiments, an N terminal part of FliC (e.g., the first 20 amino acids of FliC) is included in the construct, to further increase secretion efficiency.

In some embodiments, EGF is secreted via Type I Hemolysin Secretion, are secreted using via a diffusible outer membrane (DOM) system. In some embodiments, EGF is fused to a N-terminal Sec-dependent secretion signal. Non-limiting examples of such N-terminal Sec-dependent secretion signals include PhoA, OmpF, OmpA, and cvaC. In alternate embodiments, EGF is fused to a Tat-dependent secretion signal. Exemplary Tat-dependent tags include TorA, FdnG, and DmsA.

In certain embodiments, the recombinant bacteria comprise deletions or mutations in one or more of the outer membrane and/or periplasmic proteins. Non-limiting examples of such proteins, one or more of which may be deleted or mutated, include lpp, pal, tolA, and/or nlpI. In some embodiments, lpp is deleted or mutated. In some embodiments, pal is deleted or mutated. In some embodiments, tolA is deleted or mutated. In other embodiments, nlpI is deleted or mutated. In yet other embodiments, certain periplasmic proteases are deleted or mutated, e.g., to increase stability of the polypeptide in the periplasm. Non-limiting examples of such proteases include degP and ompT. In some embodiments, degP is deleted or mutated. In some embodiments, ompT is deleted or mutated. In some embodiments, degP and ompT are deleted or mutated.

In some embodiments, EGF is secreted via Type I Hemolysin Secretion, are secreted via a Type V Auto-secreter (pic Protein) Secretion. In some embodiments, EGF is expressed as a fusion protein with the native Nissle auto-secreter E. coli_01635 (where the original passenger protein is replaced by EGF.

In some embodiments, EGF is secreted via Type I Hemolysin Secretion, are secreted via Type I Hemolysin Secretion. In one embodiment, EGF is expressed as fusion protein with the 53 amino acids of the C terminus of alpha-hemolysin (hlyA) of E. coli CFT073.

### Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, e.g., Zhang, 2009, Nucl. Acids Res., 37:D455-D458 and Gerdes et al., Curr. Opin. Biotechnol., 17(5):448-456, the entire contents of each reference are incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the recombinant bacteria described herein also comprise a deletion or mutation in a gene required for cell survival and/or growth. In one embodiment, the essential gene is a DNA synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil, as long as the corresponding wild-type gene product is not produced in the bacteria.

Table 15 lists exemplary bacterial genes which may be disrupted or deleted to produce an auxotrophic strain. These include, but are not limited to, genes required for oligonucleotide synthesis, amino acid synthesis, and cell wall synthesis.

TABLE 15
Non-limiting Examples of Bacterial Genes
Useful for Generation of an Auxotroph

| Amino Acid | Oligonucleotide | Cell Wall |
|---|---|---|
| cysE | thy A | dapA |
| glnA | uraA | dapB |
| ilvD | | dapD |
| leuB | | dapE |
| lysA | | dapF |
| ser A | | |
| metA | | |
| gly A | | |
| hisB | | |
| ilyA | | |
| pheA | | |
| proA | | |
| thrC | | |
| trpC | | |
| tyrA | | |

Table 16 shows the survival of various amino acid auxotrophs in the mouse gut, as detected 24 hrs and 48 hrs post-gavage. These auxotrophs were generated using BW25113, a non-Nissle strain of E. coli.

TABLE 16
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| serA | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the recombinant bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the recombinant bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the recombinant bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, me, ftsB, eno, pyrG, chpR, lgt, fbaA, pgk, yqgD, metK, yqgF, plsC, ygiT, pare, ribB, cca, ygjD, tdcF, yraL, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, ispA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, gimS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, int, ginS, fidA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the recombinant bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson, ACS Synthetic Biology (2015), the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG, and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the recombinant bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the recombinant bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the recombinant bacterium is a conditional auxotroph, wherein the expression of a heterologous gene is activated by an exogenous environmental signal. In some embodiments, the recombinant bacterium is a conditional auxotroph whose essential gene(s) is replaced using an arabinose system. In the absence of arabinose, the AraC transcription factor adopts a conformation that represses transcription of the essential gene under the control of the araBAD promoter and the bacterial cell cannot survive. In the presence of arabinose, the AraC transcription factor undergoes a conformational change that allows it to bind to and activate the araBAD promoter, which induces expression of the essential gene and maintains viability of the bacterial cell.

In some embodiments, the recombinant bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., ACS Synthetic Biology (2015) 4: 307-16, the entire contents of which are expressly incorporated herein by reference. In some embodiments, the recombinant bacterium of the disclosure is an auxotroph and also comprises kill-switch circuitry, such as any of the kill-switch components and systems described herein, as well as another biosecurity system, such a conditional origin of replication (Wright et al., 2015). In other embodiments, auxotrophic modifications may also be used to screen for mutant bacteria that produce the EGF molecule.

### Antibiotic Resistance

In some embodiments, the recombinant bacteria comprise one or more genes providing resistance to antibiotics. As used herein, “Antibiotics” are substances that kill bacteria (bactericidal), or inhibit bacterial growth (bacteriostatic). Antibiotics may be natural products, many common antibiotics used in labs today are semi-synthetic or fully synthetic compounds. An antibiotic resistance gene can be added to a bacterium of interest, either on a plasmid or integrated into the chromosome in conjunction with the EGF gene, allowing the simple detection of bacteria containing the EGF gene by growing the bacteria on selective media containing the antibiotic.

In some embodiments, the recombinant bacteria comprise one or more genes providing resistance to aminoglycosides. In some embodiments, the recombinant bacteria comprise one or more genes providing resistance to a beta-lactam. In some embodiments, the recombinant bacteria comprise one or more genes providing resistance to glycopeptides. In some embodiments, the recombinant bacteria comprise one or more genes providing resistance to macrolides. In some embodiments, the recombinant bacteria comprise one or more genes providing resistance to polypeptide antibiotics. In some embodiments, the recombinant bacteria comprise one or more genes providing resistance to a tetracycline.

In some embodiments, the recombinant bacteria comprise one or more genes providing resistance to Kanamycin. In some embodiments, the recombinant bacteria comprise one or more genes providing resistance to Spectinomycin. In some embodiments, the recombinant bacteria comprise one or more genes providing resistance to Streptomycin. In some embodiments, the recombinant bacteria comprise one or more genes providing resistance to Ampicillin. In some embodiments, the recombinant bacteria comprise one or more genes providing resistance to Carbenicillin. In some embodiments, the recombinant bacteria comprise one or more genes providing resistance to Bleomycin. In some embodiments, the recombinant bacteria comprise one or more genes providing resistance to Erythromycin. In some embodiments, the recombinant bacteria comprise one or more genes providing resistance to Polymyxin B. In some embodiments, the recombinant bacteria comprise one or more genes providing resistance to Tetracycline. In some embodiments, the recombinant bacteria comprise one or more genes providing resistance to Chloramphenicol.

### Methods of Screening

### Mutagenesis

In some embodiments, the inducible promoter is operably linked to a detectable product, e.g., GFP, and can be used to screen for mutants. In some embodiments, the inducible promoter is mutagenized, and mutants are selected based upon the level of detectable product, e.g., by flow cytometry, fluorescence-activated cell sorting (FACS) when the detectable product fluoresces. In some embodiments, one or more transcription factor binding sites is mutagenized to increase or decrease binding. In alternate embodiments, the wild-type binding sites are left intact, and the remainder of the regulatory region is subjected to mutagenesis. In some embodiments, the mutant promoter is inserted into the recombinant bacteria to increase expression of the EGF molecule under inducing conditions, as compared to unmutated bacteria of the same subtype under the same conditions. In some embodiments, the inducible promoter and/or corresponding transcription factor is a synthetic, non-naturally occurring sequence.

In some embodiments, the gene encoding EGF is mutated to increase expression and/or stability of said molecule under inducing conditions, as compared to unmutated bacteria of the same subtype under the same conditions. In some embodiments, one or more of the genes in a gene cassette for producing EGF is mutated to increase expression of said molecule under inducing conditions, as compared to unmutated bacteria of the same subtype under the same conditions. In some embodiments, the efficacy or activity of any of the importers and exporters for metabolites of interest can be improved through mutations in any of these genes. Mutations increase uptake or export of such metabolites, including but not limited to, tryptophan, e.g., under inducing conditions, as compared to unmutated bacteria of the same subtype under the same conditions. Methods for directed mutation and screening are known in the art.

### Pharmaceutical Compositions and Formulations

Pharmaceutical compositions comprising one or more recombinant bacteria, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., to produce EGF. In alternate embodiments, the pharmaceutical composition comprises two or more species, strains, and/or subtypes of bacteria that are each engineered to comprise the genetic modifications described herein, e.g., to produce EGF.

In certain embodiments, a combination of two or more different genetically engineered microorganisms can be administered at the same time. In some embodiments, the method comprises administering a subject a combination of two or more genetically engineered microorganisms, a first microorganism which expresses a first payload, and at least a second microorganism which expresses a second payload. In some embodiments, the method comprises compositions comprising a combination of two or more genetically engineered microorganisms, a first microorganisms which expresses a first payload, and at least a second microorganism which expresses a second payload.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the recombinant bacteria may range from about 10e5 to 10e12 bacteria, e.g., approximately 10e5 bacteria, approximately 10e6 bacteria, approximately 10e7 bacteria, approximately 10e8 bacteria, approximately 10e9 bacteria, approximately 10e10 bacteria, approximately 10e11 bacteria, or approximately 10e12 bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered concurrently with a meal. In one embodiment, the pharmaceutical composition is administered after the subject eats a meal.

The recombinant bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the recombinant bacteria may be formulated in a solution of sodium bicarbonate, e.g., 1 molar solution of sodium bicarbonate (to buffer an acidic cellular environment, such as the stomach, for example). The recombinant bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection. The genetically engineered microorganisms of the disclosure may be administered intrathecally. In some embodiments, the genetically engineered microorganisms may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard- or soft-shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria are well known in the art. For example, see U.S. 2015/0359894 and US 2015/0238545, the entire contents of each of which are expressly incorporated herein by reference. In yet another embodiment, the pharmaceutical composition is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

### Methods of Treatment

Another aspect provides methods of treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function. In some embodiments, the invention provides for the use of at least one genetically engineered species, strain, or subtype of bacteria described herein for the manufacture of a medicament. In some embodiments, the invention provides for the use of at least one genetically engineered species, strain, or subtype of bacteria described herein for the manufacture of a medicament for treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function. In some embodiments, the invention provides at least one genetically engineered species, strain, or subtype of bacteria described herein for use in treating autoimmune disorders, cancer, metabolic diseases, diseases relating to inborn errors of metabolism, neurological or neurodegenerative diseases, diarrheal diseases, IBD, related diseases, and other diseases that benefit from reduced gut inflammation and/or enhanced gut barrier function.

In some embodiments, the diarrheal disease is selected from the group consisting of acute watery diarrhea, e.g., cholera, acute bloody diarrhea, e.g., dysentery, and persistent diarrhea. In some embodiments, the IBD or related disease is selected from the group consisting of Crohn's disease, ulcerative colitis, collagenous colitis, lymphocytic colitis, diversion colitis, Behcet's disease, intermediate colitis, short bowel syndrome, ulcerative proctitis, proctosigmoiditis, left-sided colitis, pancolitis, gastric ulcers, duodenal ulcers, and fulminant colitis.

In some embodiments, the disease or condition is an autoimmune disorder selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis. In some embodiments, the invention provides methods for reducing, ameliorating, or eliminating one or more symptom(s) associated with these diseases, including but not limited to diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In some embodiments, the invention provides methods for reducing gut inflammation and/or enhancing gut barrier function, thereby ameliorating or preventing a systemic autoimmune disorder, e.g., asthma (Arrieta et al., 2015).

The method may comprise preparing a pharmaceutical composition with at least one genetically engineered species, strain, or subtype of bacteria described herein, and administering the pharmaceutical composition to a subject in a therapeutically effective amount.

In certain embodiments, the pharmaceutical composition described herein is administered to reduce gut inflammation, enhance gut barrier function, and/or treat or prevent an autoimmune disorder in a subject. In some embodiments, the methods of the present disclosure may reduce gut inflammation in a subject by at least about 10%, 20%, 25%, 30%, 40%, 50%, 60%, 70%, 75%, 80%, 85%, 90%, 95%, or more as compared to levels in an untreated or control subject. In some embodiments, the methods of the present disclosure may enhance gut barrier function in a subject by at least about 10%, 20%, 25%, 30%, 40%, 50%, 60%, 70%, 75%, 80%, 85%, 90%, 95%, or more as compared to levels in an untreated or control subject. In some embodiments, changes in inflammation and/or gut barrier function are measured by comparing a subject before and after administration of the pharmaceutical composition. In some embodiments, the method of treating or ameliorating the autoimmune disorder and/or the disease or condition associated with gut inflammation and/or compromised gut barrier function allows one or more symptoms of the disease or condition to improve by at least about 10%, 20%, 30%, 40%, 50%, 60%, 70%, 80%, 90%, 95%, or more.

In some embodiments, reduction is measured by comparing the levels of inflammation in a subject before and after administration of the pharmaceutical composition. In one embodiment, the levels of inflammation is reduced in the gut of the subject. In one embodiment, gut barrier function is enhanced in the gut of the subject. In another embodiment, levels of inflammation is reduced in the blood of the subject. In another embodiment, the levels of inflammation is reduced in the plasma of the subject. In another embodiment, levels of inflammation is reduced in the brain of the subject.

In one embodiment, the pharmaceutical composition described herein is administered to reduce levels of inflammation in a subject to normal levels. In another embodiment, the pharmaceutical composition described herein is administered to reduce levels of inflammation in a subject below normal.

In some embodiments, the method of treating the autoimmune disorder allows one or more symptoms of the condition or disorder to improve by at least about 10%, 20%, 30%, 40%, 50%, 60%, 70%, 80%, 90%, 95%, or more. In some embodiments, the method of treating the disorder, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, gut inflammation and/or barrier function in the subject may be measured in a biological sample, such as blood, serum, plasma, urine, fecal matter, peritoneal fluid, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to enhance gut barrier function and/or to reduce gut inflammation to baseline levels, e.g., levels comparable to those of a healthy control, in a subject. In some embodiments, the methods may include administration of the compositions to reduce gut inflammation to undetectable levels in a subject, or to less than about 1%, 2%, 5%, 10%, 20%, 25%, 30%, 40%, 50%, 60%, 70%, 75%, or 80% of the subject's levels prior to treatment. In some embodiments, the methods may include administration of the compositions to enhance gut barrier function in a subject by about 1%, 2%, 5%, 10%, 20%, 25%, 30%, 40%, 50%, 60%, 70%, 75%, 80%, 90%, 100% or more of the subject's levels prior to treatment.

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

The pharmaceutical composition may be administered alone or in combination with one or more additional therapeutic agents, e.g., corticosteroids, aminosalicylates, anti-inflammatory agents. In some embodiments, the pharmaceutical composition is administered in conjunction with an anti-inflammatory drug (e.g., mesalazine, prednisolone, methylprednisolone, butesonide), an immunosuppressive drug (e.g., azathioprine, 6-mercaptopurine, methotrexate, cyclosporine, tacrolimus), an antibiotic (e.g., metronidazole, ornidazole, clarithromycin, rifaximin, ciprofloxacin, anti-TB), other probiotics, and/or biological agents (e.g., infliximab, adalimumab, certolizumab pegol) (Triantafillidis et al., 2011). An important consideration in the selection of the one or more additional therapeutic agents is that the agent(s) should be compatible with the recombinant bacteria, e.g., the agent(s) must not kill the bacteria. In one embodiment, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal, prior to a meal, or after a meal. The dosage of the pharmaceutical composition and the frequency of administration may be selected based on the severity of the symptoms and the progression of the disease. The appropriate therapeutically effective dose and/or frequency of administration can be selected by a treating clinician.

Another aspect provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin's lymphoma, Non-Hodgkin's lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

The recombinant bacteria may be destroyed, e.g., by defense factors in tissues or blood serum (Sonnenborn et al., 2009), or by activation of a kill switch, several hours or days after administration. Thus, the pharmaceutical composition comprising the gene or gene cassette for producing the anti-cancer molecule may be re-administered at a therapeutically effective dose and frequency. In alternate embodiments, the recombinant bacteria are not destroyed within hours or days after administration and may propagate and colonize the tumor.

The immunostimulatory activity of bacterial DNA is mimicked by synthetic oligodeoxynucleotides (ODNs) expressing unmethylated CpG motifs. Bode et al., Expert Rev Vaccines. 2011 April; 10(4): 499-511. CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the genetically engineered bacteria of the invention.

In one embodiment, the genetically engineered microorganisms are administered in combination with tumor cell lysates.

The dosage of the pharmaceutical composition and the frequency of administration may be selected based on the severity of the symptoms and the progression of the cancer. The appropriate therapeutically effective dose and the frequency of administration can be selected by a treating clinician.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Example 1. EGF Strain Construction

The human epidermal growth factor (hEGF) cDNA sequence was sourced from NCBI accession number: gq214314.1 and codon optimized for expression in E. coli. Sec secretion signal-hEGF fusion protein sequences were designed by adding the codon optimized hEGF sequence to the N-terminal sec secretion signal sequence of PhoA (21 amino acids), PelB (22 amino acids), or OmpA (21 amino acids). dsDNA encoding each fusion protein open reading frame was synthesized with the addition of a strong 5′ ribosome binding site (RBS) upstream of the start codon and 20 bp flanking homology to an expression vector containing a p15a origin of replication, kanamycin resistance cassette, and the PfnrS promoter from the fumarate and nitrate reductase gene S in E. coli Nissle. Gibson assembly methods yielded expression plasmids for PhoA-hEGF (p15a_Kan_fnr_PhoA-EGF), PelB-hEGF (p15a_Kan_fnr_PelB-EGF) and OmpA-hEGF (p15a_Kan_fnr_OmpA-EGF) (Sequences O, P, Q). Each construct was confirmed via PCR amplification and sequencing.

A second set of expression constructs was created by synthesizing each fusion protein downstream of the c1857 temperature responsive promoter and corresponding repressor protein derived from lambda phage, a 5′ RBS, and 20 bp flanking homology. Synthesized expression constructs were introduced into an expression vector containing a p15a origin of replication and kanamycin resistance cassette using Gibson assembly methods to yield (p15a_Kan_c1857_PhoA-EGF, SeqNo 4), (p15a_Kan_cI857_PelB-EGF, Seq No 5), and (p15a_Kan_cI857_OmpA-EGF, Seq No 6).

An expression vector containing SC101 origin of replication and Carbenicillin resistance cassette was used to express all three units of prtDEF ABC transporter: prtD (ATP-binding cassette), prtE (membrane fusion protein), and prtF (outer membrane protein) from E. chrysanthemi. Each unit was codon optimized for E. coli, and the synthesized expression construct consisted of a temperature sensitive promoter (cI857), a ribosome binding site (RBS) upstream of each unit (prtD, prtE and prtF) respectively, and included a 20 bp flanking homology to the expression vector. Gibson assembly methods was used to assemble the expression constructs into their respective expression vector, which resulted in following expression plasmids: hEGF-LARD3 (p15a_Kan_cI857_EGF-LARD3, SeqNo AC, hEGF-HlyA (p15a_KAN_T857_EGF-HlyA, Seq No AD) and prtDEF (pSC101_Carb_c1857_PtDEF, Seq No AG). Each construct was confirmed via PCR amplification and sequencing. Expression plasmids were introduced into SYN094, a wild type E. coli Nissle strain in sets of two: hEGF-LARD3 & prtDEF or hEGF-HlyA & prtDEF, which generated strains SYN7984 & SYN7985 respectively (Table 17).

To create a Gram-negative bacterium capable of secreting bioactive proteins, a diffusible outer membrane (DOM) phenotype in the E. coli Nissle background was engineered by deleting the gene encoding the periplasmic protein pal. This alteration results in an increased rate of diffusion of periplasmic proteins to the external environment without compromising cell growth properties. The resulting “leaky membrane” chassis strain is designated SYN1557. Each expression plasmid was introduced into either SYN094, a wild type E. coli Nissle strain, or SYN1557, E. coli Nissle containing a deletion of the pal gene (DOM), generating strains SYN7881-SYN7886, SYN7838-SYN7839, and SYN9001-SYN9004 (Table 17).

TABLE 17
EcN strains expressing secretion fusion proteins.

| Strains | # |
|---|---|
| Syn094 + p15a_Kan_fnr_PhoA-EGF | SYN7881 |
| Syn094 + p15a_Kan_fnr_PelB-EGF | SYN7882 |
| Syn094 + p15a_Kan_fnr_OmpA-EGF | SYN7883 |
| Syn1557 + p15a_Kan_fnr_PhoA-EGF | SYN7884 |
| Syn1557 + p15a_Kan_fnr_PelB-EGF | SYN7885 |
| Syn1557+ p15a_Kan_fnr_OmpA-EGF | SYN7886 |
| Syn094 + p15a_Kan_cI857_PhoA-EGF | SYN7938 |
| Syn094 + p15a_Kan_cI857_PelB-EGF | SYN9001 |
| Syn094 + p15a_Kan_cI857_OmpA-EGF | SYN9002 |
| Syn1557 + p15a_Kan_cI857_PhoA-EGF | SYN7939 |
| Syn1557 + p15a_Kan_cI857_PelB-EGF | SYN9003 |
| Syn1557 + p15a_Kan_cI857_OmpA-EGF | SYN9004 |
| Syn094 + p15a_Kan_cI857_EGF-LARD3 + | SYN7984 |
| pSC101_Carb_cI857_PrtDEF | |
| Syn094 + p15a_Kan_cI857_EGF-HlyA + | SYN7985 |
| pSC101_Carb_cI857_PrtDEF | |

### Example 2. EGF Production

To assay for production of EGF from engineered strains, cells were grown overnight in 10 ml of LB media containing respective antibiotics (Kanamycin 50 for EGF plasmids in all strains, and Chloramphenicol 30 for DOM chassis) in 50 ml baffled flasks, 250 rpm. 4 ml of overnight culture was added to 100 ml of LB (containing respective antibiotics) in a 250 ml baffled flask, 37C, 250 RPM. After 4 hours, OD 600 was measured for all cells and specific volumes of the cultures were spun down and resuspended in 10 mL of fresh LB (+respective antibiotics) to yield a 10 mL culture with a final OD 600 of 2.5. Cultures were then poured in 50 ml flask and were left for 5 hours in anaerobic chamber to induce the PfnrS promoter. After induction, cultures were spun down for 8 minutes, 8000 RPM, supernatants were filtered with a 0.22 micron PES filter and used for ELISA analysis.

### Example 3. EGF in Bacterial Supernatants

EGF was measured in bacterial supernatants using a human EGF specific ELISA kit from R&D systems as per the manufacturer's instructions (Cat. number DY236). Briefly, supernatants are diluted into the working range of the assay, added to wells coated with a monoclonal mouse anti-human EGF antibody specific for human EGF and incubated for one hour. Following a wash step, a polyclonal biotinylated goat anti-human EGF antibody is added and incubated for two hours. Following a second wash step, streptavidin-horseradish peroxidase is added and incubated for 20 minutes. After an additional wash step, the HRP substrate tetramethylbenzidine and hydrogen peroxide are added and incubated for 20 minutes. A stop solution of sulfuric acid is then added and 450 nm absorbance recorded. EGF concentrations of the unknowns are calculated against a standard curve constructed from recombinant EGF processed concurrently as above. EGF was measured for the EGF-secreting strains with this method.

### Example 4. EGF Activity in Human Cell Lines

HCT116 (ATCC, cat. number CCL-247) or HT29 cells (ATCC, cat. number HTB-38) were used to measure activity of recombinant EGF secreted into the supernatant of engineered E coli strains. Briefly, cells were treated with varying concentrations of EGF for 5 minutes, after which media was removed and the cells were washed one time with PBS. Lysis buffer specific for the downstream application was used to lyse the cells in preparation for further analysis. For experiments using the EGFR inhibitor AG-1478, inhibitor was added to the wells 1 hr before treatment of EGF containing samples to a final concentration of 1 uM.

For analysis by western blot, cells in a 24 well dish were lysed with 200 uL of RIPA buffer containing the recommended amount of HALT protease and phosphatase inhibitor (ThermoFisher, cat. Number 78442). Samples were left to lyse at 4° C. for 15 minutes. After lysis, samples were transferred to a 1.5 mL Eppendorf tube and spun down at 15,000 g for 15 minutes at 4° C. 7 uL of clarified lysate was added to 6 uL of DI water, 5 uL of LDS samples buffer (4×), and 2 uL of reducing agent (10×). Samples were heated to 70° C. for 10 minutes. Samples were run on a NuPage protein gel and transferred to a pvdf membrane using the iBlot2 transfer device per the manufacturer's specifications. Membrane was blocked with 5% BSA in TBST overnight and probed for EGFR, AKT, ERK, pEGFR, pAKT, and pERK using commercially available antibodies.

For analysis by ELISA, cells in a 96-well plate were lysed with 100 uL of lysis buffer specific for target phospho-protein of interest as specified by the manufacturer's instructions. All lysis buffer was supplemented with Halt Protease and Phosphatase inhibitor (ThermoFisher, cat. Number 78442). For phospho-EGFR, DuoSet Human Phospho-EGFR ELISA kit (R&D Systems, cat. Number, DYC1095B-5) was used. For phosphor-ERK, DuoSet Phospho-ERK1/ERK2 ELISA kit (R&D Systems, cat. Number, DYC1018B-5) was used. For phosphor-AKT, DuoSet Phospho-AKT1 ELISA kit (R&D Systems, cat. Number, DYC2289C-2) was used. After lysis, cells were spun down in the 96-well plate at 500 g for 15 minutes at 4° C. Supernatant was gently removed and diluted based on manufacturer's instructions.

### Example 5: EGF Production—Temperature Sensitive Promoter

Cultures were set up for overnight growth at 30° C. Overnight cultures were subcultured, 4 mL in 100 mL of the same media used in the overnight culture. Subcultures were placed at 30° C. in a shaking incubator. After around 3 hr, cells were removed from the incubator and ODs were taken. To calculate the amount of the morning culture to spin down and then resuspend in 10 mL, X was solved using:

(OD of morning culture)(X mL of culture to spin down)=(Desired OD−OD of 2.5 is standard) (10 mL final volume)

The volume of cells calculated for each culture was spun down at 5,000 g for 5 minutes. Supernatant was removed from pelleted cells and the cells were then resuspended in 10 mL fresh media and appropriate antibiotics. ODs of these cultures were taken as well to give the starting OD at the beginning of induction, e.g., close to 2.5. Cultures were placed at 37° C. in a shaking incubator to induce protein expression. As a control, separate cultures set to the same initial OD were placed at 30° C. in a shaking incubator. After 4 hr, aliquots of the culture were spun down at 5,000 g for 5 minutes and supernatant was collected for further downstream analysis. ODs and CFUs were collected for these cultures at each timepoint. Supernatant EGF was measured in an ELISA (R&D Systems) as shown in Table 18.

TABLE 18
EGF Production

| | | | μg hEGF/5e11 |
|---|---|---|---|
| Chassis | Strain | OD | cells over 5 hours |
| WT | Syn094-CI-PhoA-EGF | 2.5 | 2.6 |
| DOM | Syn1557-CI-PhoA-EGF | 2.5 | 69.9 |

### Example 6: EGF Production—FNR Promoter

Cultures were set up for overnight growth at 37° C. In the morning, overnight cultures were subcultured, 4 mL in 100 mL of the same media used in the overnight culture. Subcultures were placed at 37° C. in a shaking incubator. After around 3 hr, cells were removed from the incubator and ODs were taken. To calculate the amount of the morning culture to spin down and then resuspend in 10 mL, X was solved using:

(OD of morning culture)(X mL of culture to spin down)=(Desired OD→OD of 2.5 for low density, 5.0 for medium density and 10.0 for high density) (10 mL final volume)

The volume of cells calculated for each culture was spun down at 5,000 g for 5 minutes. Supernatant was removed from pelleted cells and the cells were then resuspended in 10 mL fresh media and appropriate antibiotics. ODs of these cultures were taken as well to give the starting OD at the beginning of induction, e.g., close to the calculated OD. Cultures were brought into a vinyl anaerobic chamber (Coy Lab Products, 5% H₂ gas mix) and placed in a standing 37° C. incubator within the chamber to induce protein expression. After 5 hr, aliquots of the culture were spun down at 5,000 g for 5 minutes and supernatant was collected for further downstream analysis. ODs and CFUs were collected for these cultures at the 5 hr timepoint. Supernatant EGF was measured in an ELISA (R&D Systems) as shown in Table 19.

TABLE 19
EGF Production

| | | | μg hEGF/5e11 |
|---|---|---|---|
| Chassis | Strain | OD | cells over 5 hours |
| WT | Syn094-EGF-PhoA | 5.0 | 0.665 |
| WT | Syn094-EGF-PelB | 5.0 | 0.4 |
| WT | Syn094-EGF-OmpA | 10 | 0.16 |
| WT | Syn094-EGF-OmpA | 5 | 0.75 |
| WT | Syn094-EGF-OmpA | 2.5 | 1.27 |
| DOM | Syn1557-EGF-PhoA | 5.0 | 8.35 |
| DOM | Syn1557-EGF-PelB | 5.0 | 3.78 |
| DOM | Syn1557-EGF-OmpA | 10 | 0.4 |
| DOM | Syn1557-EGF-OmpA | 5 | 5.81 |
| DOM | Syn1557-EGF-OmpA | 2.5 | 8.02 |

### Example 7: EGF Production—FNR Promoter

Cultures were set up for overnight growth at 37° C. In the morning, overnight cultures were subcultured, 4 mL in 100 mL of the same media used in the overnight culture. Subcultures were placed at 37° C. in a shaking incubator. After around 3 hr, cells were removed from the incubator and ODs were taken. To calculate the amount of the morning culture to spin down and then resuspend in 10 mL, X was solved using:

(OD of morning culture)(X mL of culture to spin down)=(Desired OD→OD of 2.5 for low density, 5.0 for medium density and 10.0 for high density) (10 mL final volume)

The volume of cells we calculated for each culture was spun down at 5,000 g for 5 minutes. Supernatant was removed from pelleted cells and the cells were then resuspended in 10 mL fresh media and appropriate antibiotics. ODs of these cultures were taken as well to give the starting OD at the beginning of induction, e.g., close to the calculated OD. Cultures were placed at 37° C. in a shaking incubator to induce protein expression. After 5 hr, aliquots of the culture were spun down at 5,000 g for 5 minutes and supernatant was collected for further downstream analysis. ODs and CFUs were collected for these cultures at the 5 hr timepoint. Supernatant EGF was measured in an ELISA (R&D Systems) as shown in Table 20.

TABLE 20
EGF Production

| | | | μg hEGF/5e11 |
|---|---|---|---|
| Chassis | Strain | OD | cells for 5 hr |
| WT | Syn094-EGF-OmpA | 2.5 | 17.55 |
| DOM | Syn1557-EGF-OmpA | 2.5 | 94.65 |

### Example 8: Prototype Strains

Next, additional plasmid-based, engineered E. coli Nissle 1917 strains that secrete human EGF (EcNEGF prototypes) were constructed using EcN background strains (chassis) harboring features that are advantageous for clinical development of engineered live bacterial therapeutics. The EcN chassis used in this study are described in Table 21.

TABLE 21
Chassis modifications

Chassis Name

| | | Chassis B |
|---|---|---|
| | Chassis A | (SYN8064, |
| . Chassis modifications | (SYN8032) | SYN8248) |
| Phage 3 Knockout | Yes | Yes |
| Colibactin Knockout(Δpks island) | Yes | Yes |
| Thymidine auxotrophy (ΔthyA) | Yes | Yes |
| Diffusible Outer Membrane(Δpal) | Yes | No |

Selected prototypes for secretion of bioactive human EGF are shown in Table 22. These strains include variants on both Chassis A (diffusible outer membrane) and Chassis B (wild-type membrane) backgrounds, as well as EGF under the control of temperature-inducible (cI857) or anaerobic-inducible (FNR) promoters. All strains used in this study harbor the human EGF constructs on medium-copy plasmids.

TABLE 22
EcN-EGF Plasmid-Based Prototypes

| | Strain | | EGF | EGF secretion |
|---|---|---|---|---|
| Strain ID | Description | Chassis | promoter | peptide |
| SYN8062 | A-Temp | A | cI857 | OmpA |
| SYN8063 | A-FNR | A | FNR | OmpA |
| SYN8065 | B-Temp | B | cI857 | OmpA |
| SYN8066 | B-FNR | B | FNR | OmpA |

Biomass of EcN-EGF prototypes used in this study are described in Table 23. EcN-EGF biomass was prepared in AMBR250 bioreactors, using Fermentation Medium 3 (FM3) containing 10 mM Thymidine. The bioreactors were inoculated at 0.5 OD (600 nm) from overnight seed cultures. Cells were centrifuged at 7,800×g and resuspended in 100 mM phosphate buffer containing 15% glycerol. EcN-EGF aliquots were stored at −80° C. Total cells/mL in frozen aliquots were enumerated using by Cellometer assay (K2 Matrix software, Nexelcom, Inc.). Percent viability was determined by Sytox exclusion dye assay (ThermoFisher Scientific Catalog Num. 534862).

TABLE 23
EcN-EGF Prototype Biomass Used in This Study

| | Strain | Total Cells/mL | % Viability |
|---|---|---|---|
| Strain ID | Description | (Mean ± SEM) | (Mean ± SEM) |
| SYN8062 | A-Temp | 8.86E+10 ± 2.15E+09 | 95.27 ± 0.26 |
| SYN8063 | A-FNR | 3.40E+10 ± 5.51E+08 | 87.23 ± 1.12 |
| SYN8065 | B-Temp | 5.35E+10 ± 1.77E+09 | 92.68 ± 0.82 |
| SYN8066 | B-FNR | 6.31E+10 ± 4.93E+09 | 91.38 ± 1.23 |

### Example 9: Secretion of Bioactive Human EGF by EcN-Egf Prototypes

In Vitro hEGF Production Assay

Frozen aliquots of each EcN-EGF prototype were removed from −80° C. and thawed on ice. A 30 mL aliquot of FM3 containing 50 μg/mL kanamycin was prepared for each prototype strain. Total cells/mL in frozen aliquots were enumerated using by Cellometer assay (K2 Matrix software, Nexelcom, Inc.). In vitro hEGF production assays were performed by adjusting EcN-EGF cell density 2.5×10⁹ total cells/mL.

Thawed EcN-EGF cells were added to the 30 mL media to obtain a final concentration of 2.5×10⁹ total cells/mL. Resuspended EcN-EGF cultures were then split into three 10 mLaliquots in 50 mL flasks and incubated at 37° C. under atmospheric conditions in a shaking incubator at 250 rpm for 8 hrs.

EGF-containing supernatants were then collected by centrifuging cultures at 4,000 rpm for 10 mins in a benchtop hanging bucket centrifuge. Supernatant samples were frozen and stored at −80° C. until EGF quantitation by ELISA.

In Vitro hEGF Bioactivity Assay

HT-29 cells were seeded into a 96-well tissue culture treated plate at 90,000 cells in 90 uL and incubated overnight in a tissue culture incubator to adhere to the plate. The following morning, 10 uL of desired treatment was added to each of the wells with four replicates for each treatment group and incubated for 5 minutes. After 5 minutes stimulation, the supernatant was removed from the 96-well plate by flicking and 75 uL of Lysis buffer #4 (Cisbio Cat. number 64KL4FDF) with Halt protease/phosphatase inhibitor (Halt™ Protease and Phosphatase Inhibitor Cocktail(100×)) was added to the cells. Lysates were then stored in a −80° C. freezer until pEGFR quantitation by FRET assay.

### Bioanalytical Methods

hEGF Quantitation

EGF was measured in bacterial supernatants using a human EGF specific ELISA kit from R&D systems as per the manufacturer's instructions (Cat. number DY236). Briefly, supernatants were diluted into the working range of the assay, added to wells coated with a monoclonal mouse anti-human EGF antibody specific for human EGF and incubated for 2 hr. Following a wash step, a polyclonal biotinylated goat anti-human EGF antibody is added and incubated for two hours.

Following a second wash step, streptavidin-horseradish peroxidase is added and incubated for 20 mins. After an additional wash step, the HRP substrate tetramethylbenzidine and hydrogen peroxide were added and incubated for 20 minutes. A stop solution of sulfuric acid was then added and the absorbance of wells at both 570 nm and 450 nm was measured. After subtracting the absorbance values at 450 nm from those at 570 nm for all the samples, EGF concentrations of the unknowns were calculated against a standard curve constructed from recombinant EGF processed concurrently as above. EGF was measured for all EGF-secreting strains with this method. Dilutions of 1 in 10,000 were made in sample buffer (0.1% BSA in PBS) to bring secreted EGF in the supernatants within the range of this assay.

pEGFR Quantitation

Phospho-EGFR was measured using a pEGFR HTRF kit specific for Tyr1068 from Cisbio (Cat. 64EG1PEG) as per manufacturer's instructions. Thawed lysates were briefly mixed on a plate shaker and transferred to an opaque 384 well plate. Donor Eu³⁺ cryptate and acceptor d2 pEGFR antibodies were diluted in the provided detection buffer to the working concentration of the assay and combined in equal proportions immediately before use. The antibody mixture was added to each well and incubated for 4h at RT. The FRET signal from each sample was read at 665 nm and 615 n, and the ratio between 665 nm and 615 nm signals was taken.

### Data Analysis and Results

EcN-EGF total cell counts and viability were obtained using Cellometer K2 Matrix software (Nexelcom, Inc.). All other data were analyzed using Microsoft Excel and GraphPad Prism v9.0. EGF production values (reported in Tables 8-10) were converted from raw concentrations using Equation 1.

$$Equation 1$$

$$Formula for μ g hEGF / 5 \times {10}^{11} cells in 8 hrs conversion$$

$$\frac{μ g hEGF}{5 \times {10}^{11} cells in 8 hrs} = \frac{μ g hEGF}{mL in 8 hrs} \times \frac{5 \times {10}^{11} cells}{2.5 \times {10}^{9} total cells / mL}$$

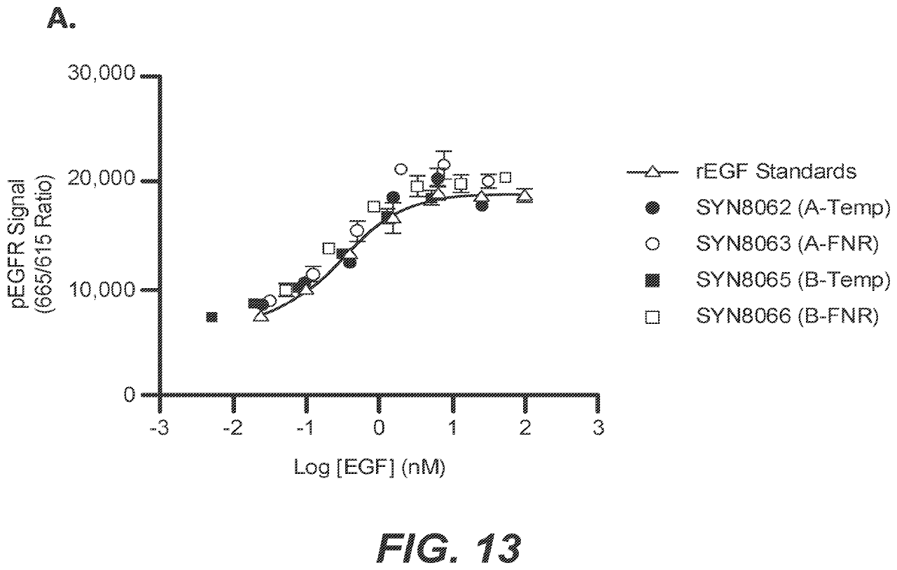

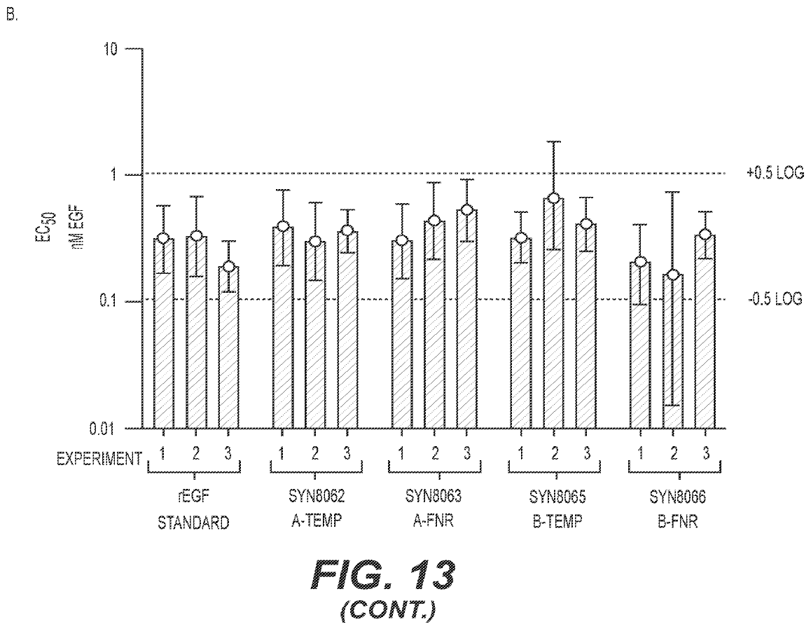

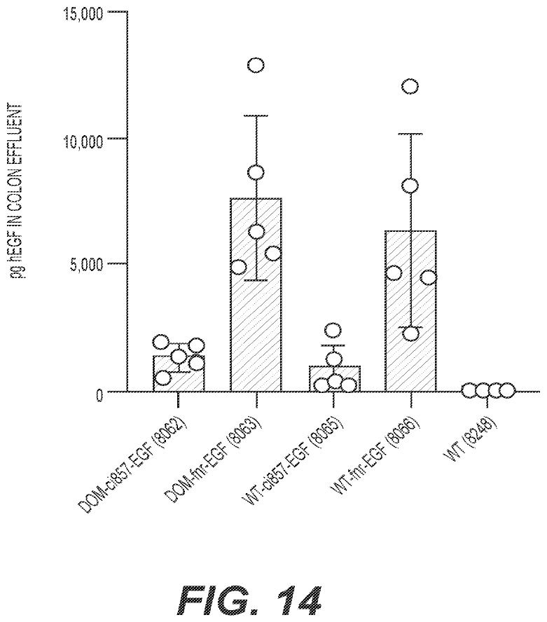

Three replicate experiments were performed demonstrating hEGF production in vitro by the EcN-EGF lead prototypes of secreted hEGF (Experiment 1, Experiment 2, and Experiment 3; see FIG. 11B, Tables 24-26). Three subsequent experiments were performed to determine the bioactivity of secreted hEGF by stimulating HT-29 cells using supernatants from the three production experiments (see FIG. 13; Tables 27-29).

TABLE 24
in vitro EGF Production Results (Experiment 1)

| | | | EGF | EGF (μg/ |
|---|---|---|---|---|
| Strain | Strain | Replicate | (μg/mL, | 5 × 10¹¹ |
| ID | Description | Culture | 8 hrs) | cells, 8 hrs) |
| SYN8062 | A-Temp | A | 0.59 | 117.85 |
| | | B | 0.77 | 154.76 |
| | | C | 1.61 | 321.22 |
| SYN8063 | A-FNR | A | 2.03 | 405.75 |
| | | B | 1.98 | 395.11 |
| | | C | 1.88 | 376.16 |
| SYN8065 | B-Temp | A | 0.33 | 65.60 |
| | | B | 0.28 | 55.40 |
| | | C | 0.36 | 71.21 |
| SYN8066 | B-FNR | A | 3.41 | 682.68 |
| | | B | 3.47 | 694.01 |
| | | C | 3.33 | 666.86 |

TABLE 25
in vitro EGF Production Results (Experiment 2)

| | | | EGF | EGF (μg/ |
|---|---|---|---|---|
| Strain | Strain | Replicate | (μg/mL, | 5 × 10¹¹ |
| ID | Description | Culture | 8 hrs) | cells, 8 hrs) |
| SYN8062 | A-Temp | A | 0.71 | 141.81 |
| | | B | 0.92 | 183.96 |
| | | C | 0.93 | 186.41 |
| SYN8063 | A-FNR | A | 2.02 | 404.61 |
| | | B | 2.07 | 414.83 |
| | | C | 1.98 | 395.84 |
| SYN8065 | B-Temp | A | 0.24 | 47.31 |
| | | B | 0.25 | 49.26 |
| | | C | 0.28 | 56.00 |
| SYN8066 | B-FNR | A | 3.04 | 607.85 |
| | | B | 3.36 | 671.32 |
| | | C | 2.86 | 666.21 |

TABLE 26
in vitro EGF Production Results (Experiment 3)

| | | | EGF | EGF (μg/ |
|---|---|---|---|---|
| Strain | Strain | Replicate | (μg/mL, | 5 × 10¹¹ |
| ID | Description | Culture | 8 hrs) | cells, 8 hrs) |
| SYN8062 | A-Temp | A | 0.96 | 192.66 |
| | | B | 1.20 | 239.20 |
| | | C | 1.22 | 244.32 |
| SYN8063 | A-FNR | A | 1.83 | 366.68 |
| | | B | 1.80 | 360.98 |
| | | C | 1.64 | 327.44 |
| SYN8065 | B-Temp | A | 0.52 | 103.79 |
| | | B | 0.49 | 97.09 |
| | | C | 0.28 | 56.92 |
| SYN8066 | B-FNR | A | 2.87 | 574.69 |
| | | B | 2.86 | 571.03 |
| | | C | 3.10 | 619.69 |

TABLE 27
in vitro EGF Bioactivity Results (Supernatants from Experiment 1)

| | EGF | | | | Signal | Plotted |
|---|---|---|---|---|---|---|
| | concen- | Log | 665 | 615 | Ratio | Value |
| | tration | [EGF] | nm | nm | (665 nm/ | (Ratio × |
| Treatment | (nM) | (nM) | Signal | Signal | 615 nm) | 10,000) |
| EGF | 100.00 | 2.00 | 588 | 313 | 1.88 | 18785.94 |
| Standards | 100.00 | 2.00 | 640 | 313 | 2.04 | 20447.28 |
| | 100.00 | 2.00 | 537 | 298 | 1.80 | 18020.13 |
| | 100.00 | 2.00 | 602 | 332 | 1.81 | 18132.53 |
| | 25.00 | 1.40 | 624 | 345 | 1.81 | 18086.96 |
| | 25.00 | 1.40 | 607 | 317 | 1.91 | 19148.26 |
| | 25.00 | 1.40 | 611 | 334 | 1.83 | 18293.41 |
| | 25.00 | 1.40 | 549 | 297 | 1.85 | 18484.85 |
| | 6.25 | 0.80 | 616 | 305 | 2.02 | 20196.72 |
| | 6.25 | 0.80 | 596 | 349 | 1.71 | 17077.36 |
| | 6.25 | 0.80 | 594 | 305 | 1.95 | 19475.41 |
| | 6.25 | 0.80 | 569 | 292 | 1.95 | 19486.30 |
| | 1.56 | 0.19 | 624 | 318 | 1.96 | 19622.64 |
| | 1.56 | 0.19 | 587 | 330 | 1.78 | 17787.88 |
| | 1.56 | 0.19 | 576 | 347 | 1.66 | 16599.42 |
| | 1.56 | 0.19 | 472 | 370 | 1.28 | 12756.76 |
| | 0.39 | −0.41 | 486 | 334 | 1.46 | 14550.90 |
| | 0.39 | −0.41 | 470 | 381 | 1.23 | 12335.96 |
| | 0.39 | −0.41 | 443 | 346 | 1.28 | 12803.47 |
| | 0.39 | −0.41 | 443 | 325 | 1.36 | 13630.77 |
| | 0.10 | −1.01 | 359 | 316 | 1.14 | 11360.76 |
| | 0.10 | −1.01 | 339 | 360 | 0.94 | 9416.67 |
| | 0.10 | −1.01 | 347 | 321 | 1.08 | 10809.97 |
| | 0.10 | −1.01 | 322 | 351 | 0.92 | 9173.79 |
| | 0.00 | — | 263 | 320 | 0.82 | 8218.75 |
| | 0.00 | — | 219 | 293 | 0.75 | 7474.40 |
| | 0.00 | — | 215 | 324 | 0.66 | 6635.80 |
| | 0.00 | — | 255 | 329 | 0.78 | 7750.76 |
| SYN8062 | 25.10 | 1.40 | 546 | 307 | 1.78 | 17785.02 |
| | 25.10 | 1.40 | 494 | 290 | 1.70 | 17034.48 |
| | 25.10 | 1.40 | 542 | 279 | 1.94 | 19426.52 |
| | 25.10 | 1.40 | 535 | 311 | 1.72 | 17202.57 |
| | 6.27 | 0.80 | 605 | 276 | 2.19 | 21920.29 |
| | 6.27 | 0.80 | 523 | 304 | 1.72 | 17203.95 |
| | 6.27 | 0.80 | 554 | 275 | 2.01 | 20145.45 |
| | 6.27 | 0.80 | 574 | 263 | 2.18 | 21825.10 |
| | 1.57 | 0.20 | 546 | 302 | 1.81 | 18079.47 |
| | 1.57 | 0.20 | 494 | 279 | 1.77 | 17706.09 |
| | 1.57 | 0.20 | 558 | 291 | 1.92 | 19175.26 |
| | 1.57 | 0.20 | 510 | 266 | 1.92 | 19172.93 |
| | 0.39 | −0.41 | 394 | 305 | 1.29 | 12918.03 |
| | 0.39 | −0.41 | 347 | 312 | 1.11 | 11121.79 |
| | 0.39 | −0.41 | 422 | 331 | 1.27 | 12749.24 |
| | 0.39 | −0.41 | 391 | 289 | 1.35 | 13529.41 |
| | 0.10 | −1.01 | 307 | 302 | 1.02 | 10165.56 |
| | 0.10 | −1.01 | 331 | 290 | 1.14 | 11413.79 |
| | 0.10 | −1.01 | 323 | 315 | 1.03 | 10253.97 |
| | 0.10 | −1.01 | 327 | 307 | 1.07 | 10651.47 |
| | 0.02 | −1.61 | 295 | 310 | 0.95 | 9516.13 |
| | 0.02 | −1.61 | 227 | 313 | 0.73 | 7252.40 |
| | 0.02 | −1.61 | 283 | 344 | 0.82 | 8226.74 |
| | 0.02 | −1.61 | 275 | 297 | 0.93 | 9259.26 |
| SYN8063 | 31.70 | 1.50 | 610 | 314 | 1.94 | 19426.75 |
| | 31.70 | 1.50 | 594 | 278 | 2.14 | 21366.91 |
| | 31.70 | 1.50 | 638 | 306 | 2.08 | 20849.67 |
| | 31.70 | 1.50 | 570 | 302 | 1.89 | 18874.17 |
| | 7.92 | 0.90 | 618 | 310 | 1.99 | 19935.48 |
| | 7.92 | 0.90 | 598 | 312 | 1.92 | 19166.67 |
| | 7.92 | 0.90 | 614 | 274 | 2.24 | 22408.76 |
| | 7.92 | 0.90 | 651 | 260 | 2.50 | 25038.46 |
| | 1.98 | 0.30 | 630 | 291 | 2.16 | 21649.48 |
| | 1.98 | 0.30 | 672 | 311 | 2.16 | 21607.72 |
| | 1.98 | 0.30 | 603 | 300 | 2.01 | 20100.00 |
| | 1.98 | 0.30 | 582 | 268 | 2.17 | 21716.42 |
| | 0.50 | −0.31 | 494 | 317 | 1.56 | 15583.60 |
| | 0.50 | −0.31 | 470 | 335 | 1.40 | 14029.85 |
| | 0.50 | −0.31 | 474 | 263 | 1.80 | 18022.81 |
| | 0.50 | −0.31 | 442 | 313 | 1.41 | 14121.41 |
| | 0.12 | −0.91 | 347 | 323 | 1.07 | 10743.03 |
| | 0.12 | −0.91 | 311 | 311 | 1.00 | 10000.00 |
| | 0.12 | −0.91 | 355 | 267 | 1.33 | 13295.88 |
| | 0.12 | −0.91 | 339 | 285 | 1.19 | 11894.74 |
| | 0.03 | −1.51 | 290 | 296 | 0.98 | 9797.30 |
| | 0.03 | −1.51 | 295 | 315 | 0.94 | 9365.08 |
| | 0.03 | −1.51 | 279 | 331 | 0.84 | 8429.00 |
| | 0.03 | −1.51 | 255 | 311 | 0.82 | 8199.36 |
| SYN8065 | 5.12 | 0.71 | 534 | 290 | 1.84 | 18413.79 |
| | 5.12 | 0.71 | 518 | 300 | 1.73 | 17266.67 |
| | 5.12 | 0.71 | 526 | 290 | 1.81 | 18137.93 |
| | 5.12 | 0.71 | 518 | 253 | 2.05 | 20474.31 |
| | 1.28 | 0.11 | 486 | 326 | 1.49 | 14907.98 |
| | 1.28 | 0.11 | 502 | 290 | 1.73 | 17310.34 |
| | 1.28 | 0.11 | 542 | 326 | 1.66 | 16625.77 |
| | 1.28 | 0.11 | 506 | 273 | 1.85 | 18534.80 |
| | 0.32 | −0.49 | 386 | 292 | 1.32 | 13219.18 |
| | 0.32 | −0.49 | 375 | 272 | 1.38 | 13786.76 |
| | 0.32 | −0.49 | 414 | 337 | 1.23 | 12284.87 |
| | 0.32 | −0.49 | 375 | 274 | 1.37 | 13686.13 |
| | 0.08 | −1.10 | 331 | 293 | 1.13 | 11296.93 |
| | 0.08 | −1.10 | 307 | 329 | 0.93 | 9331.31 |
| | 0.08 | −1.10 | 299 | 305 | 0.98 | 9803.28 |
| | 0.08 | −1.10 | 299 | 293 | 1.02 | 10204.78 |
| | 0.02 | −1.70 | 303 | 324 | 0.94 | 9351.85 |
| | 0.02 | −1.70 | 263 | 302 | 0.87 | 8708.61 |
| | 0.02 | −1.70 | 255 | 308 | 0.83 | 8279.22 |
| | 0.02 | −1.70 | 251 | 300 | 0.84 | 8366.67 |
| | 0.01 | −2.30 | 247 | 333 | 0.74 | 7417.42 |
| | 0.01 | −2.30 | 239 | 334 | 0.72 | 7155.69 |
| | 0.01 | −2.30 | 259 | 334 | 0.78 | 7754.49 |
| | 0.01 | −2.30 | 247 | 332 | 0.74 | 7439.76 |
| SYN8066 | 53.33 | 1.73 | 630 | 301 | 2.09 | 20930.23 |
| | 53.33 | 1.73 | 606 | 315 | 1.92 | 19238.10 |
| | 53.33 | 1.73 | 597 | 297 | 2.01 | 20101.01 |
| | 53.33 | 1.73 | 614 | 285 | 2.15 | 21543.86 |
| | 13.33 | 1.12 | 645 | 289 | 2.23 | 22318.34 |
| | 13.33 | 1.12 | 594 | 319 | 1.86 | 18620.69 |
| | 13.33 | 1.12 | 602 | 319 | 1.89 | 18871.47 |
| | 13.33 | 1.12 | 594 | 302 | 1.97 | 19668.87 |
| | 3.33 | 0.52 | 674 | 324 | 2.08 | 20802.47 |
| | 3.33 | 0.52 | 566 | 308 | 1.84 | 18376.62 |
| | 3.33 | 0.52 | 602 | 340 | 1.77 | 17705.88 |
| | 3.33 | 0.52 | 638 | 293 | 2.18 | 21774.74 |
| | 0.83 | −0.08 | 566 | 314 | 1.80 | 18025.48 |
| | 0.83 | −0.08 | 538 | 327 | 1.65 | 16452.60 |
| | 0.83 | −0.08 | 522 | 292 | 1.79 | 17876.71 |
| | 0.83 | −0.08 | 522 | 278 | 1.88 | 18776.98 |
| | 0.21 | −0.68 | 438 | 318 | 1.38 | 13773.58 |
| | 0.21 | −0.68 | 435 | 286 | 1.52 | 15209.79 |
| | 0.21 | −0.68 | 438 | 322 | 1.36 | 13602.48 |
| | 0.21 | −0.68 | 410 | 322 | 1.27 | 12732.92 |
| | 0.05 | −1.28 | 315 | 311 | 1.01 | 10128.62 |
| | 0.05 | −1.28 | 310 | 367 | 0.84 | 8446.87 |
| | 0.05 | −1.28 | 354 | 311 | 1.14 | 11382.64 |
| | 0.05 | −1.28 | 303 | 308 | 0.98 | 9837.66 |

TABLE 28
in vitro EGF Bioactivity Results (Supernatants from Experiment 2)

| | EGF | | | | Signal | Plotted |
|---|---|---|---|---|---|---|
| | concen- | Log | 665 | 615 | Ratio | Value |
| | tration | [EGF] | nm | nm | (665 nm/ | (Ratio × |
| Treatment | (nM) | (nM) | Signal | Signal | 615 nm) | 10,000) |
| EGF | 100.00 | 2.00 | 369 | 161 | 2.29 | 22919.25 |
| Standards | 100.00 | 2.00 | 378 | 145 | 2.61 | 26068.97 |
| | 100.00 | 2.00 | 393 | 155 | 2.54 | 25354.84 |
| | 100.00 | 2.00 | 397 | 175 | 2.27 | 22685.71 |
| | 25.00 | 1.40 | 379 | 126 | 3.01 | 30079.37 |
| | 25.00 | 1.40 | 432 | 160 | 2.70 | 27000.00 |
| | 25.00 | 1.40 | 390 | 188 | 2.07 | 20744.68 |
| | 25.00 | 1.40 | 384 | 156 | 2.46 | 24615.38 |
| | 6.25 | 0.80 | 354 | 130 | 2.72 | 27230.77 |
| | 6.25 | 0.80 | 359 | 162 | 2.22 | 22160.49 |
| | 6.25 | 0.80 | 384 | 153 | 2.51 | 25098.04 |
| | 6.25 | 0.80 | 339 | 124 | 2.73 | 27338.71 |
| | 1.56 | 0.19 | 299 | 133 | 2.25 | 22481.20 |
| | 1.56 | 0.19 | 372 | 138 | 2.70 | 26956.52 |
| | 1.56 | 0.19 | 360 | 137 | 2.63 | 26277.37 |
| | 1.56 | 0.19 | 343 | 157 | 2.18 | 21847.13 |
| | 0.39 | −0.41 | 265 | 154 | 1.72 | 17207.79 |
| | 0.39 | −0.41 | 256 | 145 | 1.77 | 17655.17 |
| | 0.39 | −0.41 | 258 | 135 | 1.91 | 19111.11 |
| | 0.39 | −0.41 | 306 | 152 | 2.01 | 20131.58 |
| | 0.10 | −1.01 | 190 | 141 | 1.35 | 13475.18 |
| | 0.10 | −1.01 | 241 | 162 | 1.49 | 14876.54 |
| | 0.10 | −1.01 | 226 | 169 | 1.34 | 13372.78 |
| | 0.10 | −1.01 | 207 | 144 | 1.44 | 14375.00 |
| | 0.02 | −1.61 | 170 | 139 | 1.22 | 12230.22 |
| | 0.02 | −1.61 | 215 | 152 | 1.41 | 14144.74 |
| | 0.02 | −1.61 | 203 | 154 | 1.32 | 13181.82 |
| | 0.02 | −1.61 | 176 | 148 | 1.19 | 11891.89 |
| | 0.00 | — | 165 | 176 | 0.94 | 9375.00 |
| | 0.00 | — | 154 | 145 | 1.06 | 10620.69 |
| | 0.00 | — | 180 | 168 | 1.07 | 10714.29 |
| | 0.00 | — | 165 | 156 | 1.06 | 10576.92 |
| SYN8062 | 11.08 | 1.04 | 341 | 138 | 2.47 | 24710.14 |
| | 11.08 | 1.04 | 340 | 169 | 2.01 | 20118.34 |
| | 11.08 | 1.04 | 356 | 154 | 2.31 | 23116.88 |
| | 11.08 | 1.04 | 359 | 140 | 2.56 | 25642.86 |
| | 2.77 | 0.44 | 340 | 130 | 2.62 | 26153.85 |
| | 2.77 | 0.44 | 348 | 146 | 2.38 | 23835.62 |
| | 2.77 | 0.44 | 351 | 156 | 2.25 | 22500.00 |
| | 2.77 | 0.44 | 343 | 128 | 2.68 | 26796.88 |
| | 0.69 | −0.16 | 276 | 155 | 1.78 | 17806.45 |
| | 0.69 | −0.16 | 288 | 143 | 2.01 | 20139.86 |
| | 0.69 | −0.16 | 291 | 145 | 2.01 | 20068.97 |
| | 0.69 | −0.16 | 311 | 134 | 2.32 | 23208.96 |
| | 0.17 | −0.76 | 224 | 160 | 1.40 | 14000.00 |
| | 0.17 | −0.76 | 218 | 133 | 1.64 | 16390.98 |
| | 0.17 | −0.76 | 213 | 169 | 1.26 | 12603.55 |
| | 0.17 | −0.76 | 233 | 121 | 1.93 | 19256.20 |
| | 0.04 | −1.36 | 175 | 175 | 1.00 | 10000.00 |
| | 0.04 | −1.36 | 182 | 144 | 1.26 | 12638.89 |
| | 0.04 | −1.36 | 188 | 156 | 1.21 | 12051.28 |
| | 0.04 | −1.36 | 166 | 154 | 1.08 | 10779.22 |
| | 0.01 | −1.97 | 159 | 145 | 1.10 | 10965.52 |
| | 0.01 | −1.97 | 194 | 171 | 1.13 | 11345.03 |
| | 0.01 | −1.97 | 163 | 147 | 1.11 | 11088.44 |
| | 0.01 | −1.97 | 177 | 148 | 1.20 | 11959.46 |
| SYN8063 | 31.61 | 1.50 | 356 | 140 | 2.54 | 25428.57 |
| | 31.61 | 1.50 | 353 | 152 | 2.32 | 23223.68 |
| | 31.61 | 1.50 | 359 | 174 | 2.06 | 20632.18 |
| | 31.61 | 1.50 | 332 | 141 | 2.35 | 23546.10 |
| | 7.90 | 0.90 | 345 | 156 | 2.21 | 22115.38 |
| | 7.90 | 0.90 | 355 | 151 | 2.35 | 23509.93 |
| | 7.90 | 0.90 | 373 | 180 | 2.07 | 20722.22 |
| | 7.90 | 0.90 | 345 | 160 | 2.16 | 21562.50 |
| | 1.98 | 0.30 | 375 | 159 | 2.36 | 23584.91 |
| | 1.98 | 0.30 | 345 | 164 | 2.10 | 21036.59 |
| | 1.98 | 0.30 | 368 | 161 | 2.29 | 22857.14 |
| | 1.98 | 0.30 | 326 | 168 | 1.94 | 19404.76 |
| | 0.49 | −0.31 | 304 | 180 | 1.69 | 16888.89 |
| | 0.49 | −0.31 | 288 | 160 | 1.80 | 18000.00 |
| | 0.49 | −0.31 | 329 | 183 | 1.80 | 17978.14 |
| | 0.49 | −0.31 | 301 | 168 | 1.79 | 17916.67 |
| | 0.12 | −0.91 | 220 | 168 | 1.31 | 13095.24 |
| | 0.12 | −0.91 | 226 | 152 | 1.49 | 14868.42 |
| | 0.12 | −0.91 | 208 | 159 | 1.31 | 13081.76 |
| | 0.12 | −0.91 | 246 | 145 | 1.70 | 16965.52 |
| | 0.03 | −1.51 | 213 | 155 | 1.37 | 13741.94 |
| | 0.03 | −1.51 | 206 | 156 | 1.32 | 13205.13 |
| | 0.03 | −1.51 | 210 | 176 | 1.19 | 11931.82 |
| | 0.03 | −1.51 | 218 | 173 | 1.26 | 12601.16 |
| SYN8065 | 3.70 | 0.57 | 301 | 153 | 1.97 | 19673.20 |
| | 3.70 | 0.57 | 382 | 146 | 2.62 | 26164.38 |
| | 3.70 | 0.57 | 361 | 121 | 2.98 | 29834.71 |
| | 3.70 | 0.57 | 386 | 156 | 2.47 | 24743.59 |
| | 0.92 | −0.03 | 342 | 176 | 1.94 | 19431.82 |
| | 0.92 | −0.03 | 329 | 143 | 2.30 | 23006.99 |
| | 0.92 | −0.03 | 325 | 174 | 1.87 | 18678.16 |
| | 0.92 | −0.03 | 321 | 137 | 2.34 | 23430.66 |
| | 0.23 | −0.64 | 262 | 177 | 1.48 | 14802.26 |
| | 0.23 | −0.64 | 207 | 134 | 1.54 | 15447.76 |
| | 0.23 | −0.64 | 221 | 154 | 1.44 | 14350.65 |
| | 0.23 | −0.64 | 238 | 133 | 1.79 | 17894.74 |
| | 0.06 | −1.24 | 243 | 173 | 1.40 | 14046.24 |
| | 0.06 | −1.24 | 175 | 189 | 0.93 | 9259.26 |
| | 0.06 | −1.24 | 230 | 148 | 1.55 | 15540.54 |
| | 0.06 | −1.24 | 207 | 150 | 1.38 | 13800.00 |
| | 0.01 | −1.84 | 201 | 161 | 1.25 | 12484.47 |
| | 0.01 | −1.84 | 195 | 161 | 1.21 | 12111.80 |
| | 0.01 | −1.84 | 191 | 158 | 1.21 | 12088.61 |
| | 0.01 | −1.84 | 207 | 145 | 1.43 | 14275.86 |
| | 0.00 | −2.44 | 162 | 161 | 1.01 | 10062.11 |
| | 0.00 | −2.44 | 179 | 153 | 1.17 | 11699.35 |
| | 0.00 | −2.44 | 211 | 165 | 1.28 | 12787.88 |
| | 0.00 | −2.44 | 158 | 149 | 1.06 | 10604.03 |
| SYN8066 | 47.49 | 1.68 | 366 | 162 | 2.26 | 22592.59 |
| | 47.49 | 1.68 | 374 | 157 | 2.38 | 23821.66 |
| | 47.49 | 1.68 | 391 | 161 | 2.43 | 24285.71 |
| | 47.49 | 1.68 | 373 | 157 | 2.38 | 23757.96 |
| | 11.87 | 1.07 | 343 | 157 | 2.18 | 21847.13 |
| | 11.87 | 1.07 | 366 | 196 | 1.87 | 18673.47 |
| | 11.87 | 1.07 | 382 | 133 | 2.87 | 28721.80 |
| | 11.87 | 1.07 | 350 | 153 | 2.29 | 22875.82 |
| | 2.97 | 0.47 | 345 | 190 | 1.82 | 18157.89 |
| | 2.97 | 0.47 | 358 | 185 | 1.94 | 19351.35 |
| | 2.97 | 0.47 | 439 | 177 | 2.48 | 24802.26 |
| | 2.97 | 0.47 | 366 | 153 | 2.39 | 23921.57 |
| | 0.74 | −0.13 | 323 | 146 | 2.21 | 22123.29 |
| | 0.74 | −0.13 | 325 | 158 | 2.06 | 20569.62 |
| | 0.74 | −0.13 | 350 | 170 | 2.06 | 20588.24 |
| | 0.74 | −0.13 | 358 | 169 | 2.12 | 21183.43 |
| | 0.19 | −0.73 | 239 | 153 | 1.56 | 15620.92 |
| | 0.19 | −0.73 | 258 | 170 | 1.52 | 15176.47 |
| | 0.19 | −0.73 | 247 | 117 | 2.11 | 21111.11 |
| | 0.19 | −0.73 | 253 | 146 | 1.73 | 17328.77 |
| | 0.05 | −1.33 | 230 | 161 | 1.43 | 14285.71 |
| | 0.05 | −1.33 | 243 | 161 | 1.51 | 15093.17 |
| | 0.05 | −1.33 | 215 | 169 | 1.27 | 12721.89 |
| | 0.05 | −1.33 | 195 | 169 | 1.15 | 11538.46 |

TABLE 29
in vitro EGF Bioactivity Results (Supernatants from Experiment 3)

| | EGF | | | | Signal | Plotted |
|---|---|---|---|---|---|---|
| | concen- | Log | 665 | 615 | Ratio | Value |
| | tration | [EGF] | nm | nm | (665 nm/ | (Ratio × |
| Treatment | (nM) | (nM) | Signal | Signal | 615 nm) | 10,000) |
| EGF | 100.00 | 2.00 | 773 | 402 | 1.92 | 19228.86 |
| Standards | 100.00 | 2.00 | 905 | 378 | 2.39 | 23941.80 |
| | 100.00 | 2.00 | 834 | 380 | 2.19 | 21947.37 |
| | 100.00 | 2.00 | 914 | 394 | 2.32 | 23197.97 |
| | 25.00 | 1.40 | 862 | 354 | 2.44 | 24350.28 |
| | 25.00 | 1.40 | 905 | 414 | 2.19 | 21859.90 |
| | 25.00 | 1.40 | 931 | 425 | 2.19 | 21905.88 |
| | 25.00 | 1.40 | 875 | 373 | 2.35 | 23458.45 |
| | 6.25 | 0.80 | 835 | 389 | 2.15 | 21465.30 |
| | 6.25 | 0.80 | 836 | 374 | 2.24 | 22352.94 |
| | 6.25 | 0.80 | 927 | 410 | 2.26 | 22609.76 |
| | 6.25 | 0.80 | 907 | 386 | 2.35 | 23497.41 |
| | 1.56 | 0.19 | 781 | 365 | 2.14 | 21397.26 |
| | 1.56 | 0.19 | 856 | 370 | 2.31 | 23135.14 |
| | 1.56 | 0.19 | 874 | 418 | 2.09 | 20909.09 |
| | 1.56 | 0.19 | 854 | 354 | 2.41 | 24124.29 |
| | 0.39 | −0.41 | 610 | 368 | 1.66 | 16576.09 |
| | 0.39 | −0.41 | 713 | 410 | 1.74 | 17390.24 |
| | 0.39 | −0.41 | 713 | 381 | 1.87 | 18713.91 |
| | 0.39 | −0.41 | 717 | 358 | 2.00 | 20027.93 |
| | 0.10 | −1.01 | 442 | 377 | 1.17 | 11724.14 |
| | 0.10 | −1.01 | 506 | 413 | 1.23 | 12251.82 |
| | 0.10 | −1.01 | 439 | 397 | 1.11 | 11057.93 |
| | 0.10 | −1.01 | 510 | 393 | 1.30 | 12977.10 |
| | 0.02 | −1.61 | 347 | 362 | 0.96 | 9585.64 |
| | 0.02 | −1.61 | 406 | 386 | 1.05 | 10518.13 |
| | 0.02 | −1.61 | 394 | 390 | 1.01 | 10102.56 |
| | 0.02 | −1.61 | 370 | 393 | 0.94 | 9414.76 |
| SYN8062 | 15.05 | 1.18 | 796 | 395 | 2.02 | 20151.90 |
| | 15.05 | 1.18 | 730 | 397 | 1.84 | 18387.91 |
| | 15.05 | 1.18 | 757 | 365 | 2.07 | 20739.73 |
| | 15.05 | 1.18 | 745 | 360 | 2.07 | 20694.44 |
| | 3.76 | 0.58 | 793 | 419 | 1.89 | 18926.01 |
| | 3.76 | 0.58 | 802 | 412 | 1.95 | 19466.02 |
| | 3.76 | 0.58 | 869 | 384 | 2.26 | 22630.21 |
| | 3.76 | 0.58 | 794 | 376 | 2.11 | 21117.02 |
| | 0.94 | −0.03 | 785 | 451 | 1.74 | 17405.76 |
| | 0.94 | −0.03 | 733 | 402 | 1.82 | 18233.83 |
| | 0.94 | −0.03 | 701 | 413 | 1.70 | 16973.37 |
| | 0.94 | −0.03 | 709 | 386 | 1.84 | 18367.88 |
| | 0.24 | −0.63 | 474 | 408 | 1.16 | 11617.65 |
| | 0.24 | −0.63 | 522 | 403 | 1.30 | 12952.85 |
| | 0.24 | −0.63 | 482 | 403 | 1.20 | 11960.30 |
| | 0.24 | −0.63 | 522 | 414 | 1.26 | 12608.70 |
| | 0.06 | −1.23 | 390 | 384 | 1.02 | 10156.25 |
| | 0.06 | −1.23 | 346 | 404 | 0.86 | 8564.36 |
| | 0.06 | −1.23 | 346 | 368 | 0.94 | 9402.17 |
| | 0.06 | −1.23 | 371 | 405 | 0.92 | 9160.49 |
| | 0.01 | −1.83 | 351 | 393 | 0.89 | 8931.30 |
| | 0.01 | −1.83 | 326 | 438 | 0.74 | 7442.92 |
| | 0.01 | −1.83 | 331 | 427 | 0.78 | 7751.76 |
| | 0.01 | −1.83 | 339 | 380 | 0.89 | 8921.05 |
| SYN8063 | 28.65 | 1.46 | 781 | 395 | 1.98 | 19772.15 |
| | 28.65 | 1.46 | 873 | 344 | 2.54 | 25377.91 |
| | 28.65 | 1.46 | 825 | 400 | 2.06 | 20625.00 |
| | 28.65 | 1.46 | 885 | 385 | 2.30 | 22987.01 |
| | 7.16 | 0.86 | 785 | 402 | 1.95 | 19527.36 |
| | 7.16 | 0.86 | 834 | 352 | 2.37 | 23693.18 |
| | 7.16 | 0.86 | 793 | 374 | 2.12 | 21203.21 |
| | 7.16 | 0.86 | 789 | 363 | 2.17 | 21735.54 |
| | 1.79 | 0.25 | 730 | 385 | 1.90 | 18961.04 |
| | 1.79 | 0.25 | 817 | 417 | 1.96 | 19592.33 |
| | 1.79 | 0.25 | 830 | 429 | 1.93 | 19347.32 |
| | 1.79 | 0.25 | 821 | 400 | 2.05 | 20525.00 |
| | 0.45 | −0.35 | 538 | 397 | 1.36 | 13551.64 |
| | 0.45 | −0.35 | 610 | 390 | 1.56 | 15641.03 |
| | 0.45 | −0.35 | 574 | 408 | 1.41 | 14068.63 |
| | 0.45 | −0.35 | 597 | 399 | 1.50 | 14962.41 |
| | 0.11 | −0.95 | 402 | 371 | 1.08 | 10835.58 |
| | 0.11 | −0.95 | 462 | 343 | 1.35 | 13469.39 |
| | 0.11 | −0.95 | 434 | 418 | 1.04 | 10382.78 |
| | 0.11 | −0.95 | 474 | 389 | 1.22 | 12185.09 |
| | 0.03 | −1.55 | 330 | 353 | 0.93 | 9348.44 |
| | 0.03 | −1.55 | 355 | 384 | 0.92 | 9244.79 |
| | 0.03 | −1.55 | 335 | 393 | 0.85 | 8524.17 |
| | 0.03 | −1.55 | 338 | 366 | 0.92 | 9234.97 |
| SYN8065 | 8.11 | 0.91 | 774 | 414 | 1.87 | 18695.65 |
| | 8.11 | 0.91 | 913 | 406 | 2.25 | 22487.68 |
| | 8.11 | 0.91 | 761 | 375 | 2.03 | 20293.33 |
| | 8.11 | 0.91 | 821 | 373 | 2.20 | 22010.72 |
| | 2.03 | 0.31 | 764 | 400 | 1.91 | 19100.00 |
| | 2.03 | 0.31 | 894 | 386 | 2.32 | 23160.62 |
| | 2.03 | 0.31 | 757 | 381 | 1.99 | 19868.77 |
| | 2.03 | 0.31 | 871 | 394 | 2.21 | 22106.60 |
| | 0.51 | −0.30 | 594 | 424 | 1.40 | 14009.43 |
| | 0.51 | −0.30 | 637 | 368 | 1.73 | 17309.78 |
| | 0.51 | −0.30 | 610 | 418 | 1.46 | 14593.30 |
| | 0.51 | −0.30 | 630 | 392 | 1.61 | 16071.43 |
| | 0.13 | −0.90 | 422 | 385 | 1.10 | 10961.04 |
| | 0.13 | −0.90 | 394 | 428 | 0.92 | 9205.61 |
| | 0.13 | −0.90 | 418 | 382 | 1.09 | 10942.41 |
| | 0.13 | −0.90 | 495 | 396 | 1.25 | 12500.00 |
| | 0.03 | −1.50 | 367 | 422 | 0.87 | 8696.68 |
| | 0.03 | −1.50 | 366 | 426 | 0.86 | 8591.55 |
| | 0.03 | −1.50 | 323 | 421 | 0.77 | 7672.21 |
| | 0.03 | −1.50 | 371 | 393 | 0.94 | 9440.20 |
| | 0.01 | −2.10 | 319 | 409 | 0.78 | 7799.51 |
| | 0.01 | −2.10 | 326 | 377 | 0.86 | 8647.21 |
| | 0.01 | −2.10 | 335 | 380 | 0.88 | 8815.79 |
| | 0.01 | −2.10 | 322 | 395 | 0.82 | 8151.90 |
| SYN8066 | 44.90 | 1.65 | 898 | 365 | 2.46 | 24602.74 |
| | 44.90 | 1.65 | 894 | 370 | 2.42 | 24162.16 |
| | 44.90 | 1.65 | 849 | 371 | 2.29 | 22884.10 |
| | 44.90 | 1.65 | 802 | 345 | 2.32 | 23246.38 |
| | 11.22 | 1.05 | 849 | 337 | 2.52 | 25192.88 |
| | 11.22 | 1.05 | 890 | 401 | 2.22 | 22194.51 |
| | 11.22 | 1.05 | 876 | 393 | 2.23 | 22290.08 |
| | 11.22 | 1.05 | 857 | 378 | 2.27 | 22671.96 |
| | 2.81 | 0.45 | 860 | 381 | 2.26 | 22572.18 |
| | 2.81 | 0.45 | 858 | 416 | 2.06 | 20625.00 |
| | 2.81 | 0.45 | 940 | 420 | 2.24 | 22380.95 |
| | 2.81 | 0.45 | 856 | 401 | 2.13 | 21346.63 |
| | 0.70 | −0.15 | 730 | 397 | 1.84 | 18387.91 |
| | 0.70 | −0.15 | 746 | 398 | 1.87 | 18743.72 |
| | 0.70 | −0.15 | 734 | 426 | 1.72 | 17230.05 |
| | 0.70 | −0.15 | 782 | 394 | 1.98 | 19847.72 |
| | 0.18 | −0.76 | 562 | 362 | 1.55 | 15524.86 |
| | 0.18 | −0.76 | 474 | 405 | 1.17 | 11703.70 |
| | 0.18 | −0.76 | 510 | 388 | 1.31 | 13144.33 |
| | 0.18 | −0.76 | 482 | 356 | 1.35 | 13539.33 |
| | 0.04 | −1.36 | 382 | 368 | 1.04 | 10380.43 |
| | 0.04 | −1.36 | 355 | 383 | 0.93 | 9268.93 |
| | 0.04 | −1.36 | 402 | 400 | 1.01 | 10050.00 |
| | 0.04 | −1.36 | 406 | 428 | 0.95 | 9485.98 |

These data demonstrate that each of the EcN-EGF prototypes (SYN8062, SYN8063, SYN8065, and SYN8066) display in vitro EGF production of ≥1 μg hEGF/5×10\hat{ }11 cells in vitro across three replicate experiments.

The results of experiments demonstrating the in vitro bioactivity of secreted hEGF in stimulated HT-29 cells are shown in FIG. 13 and Tables 27-29. Overall, these data demonstrate that each of the EcN-EGF prototypes (SYN8062, SYN8063, SYN8065, and SYN8066) are capable of inducing Phospho-EGFR levels comparable to signals observed with recombinant EGF in vitro across three replicate experiments. In all cases, the 95% confidence interval for the EC50 (calculated using GraphPad Prism v9.0 software) intersects this range, indicating that the EC50 estimate is consistent with the expected bioactivity of human EGF.

In conclusion, the results presented in this study demonstrate that the four EcN-EGF prototypes investigated (SYN8062, SYN8063, SYN8065, and SYN8066) all secretes ≥1 μg hEGF/5e11 cells in vitro across three replicate experiments. Moreover, phospho-EGFR levels measured by ELISA in human epithelial cells induced by secreted hEGF are comparable to signals observed with recombinant EGF (rEGF). Comparability is defined as signal at EC50 values of rEGF (+0.5 log) across three replicate experiments.

### Example 10. In Vivo Biodistribution of Engineered EcN

The objective of this study was to determine the biodistribution in the gastrointestinal (GI) tract of prototype EGF-secreting strains and to assess the effect of bacterial engineering on GI distribution and fecal excretion of SYN8062 and SYN8063 in C57BL/6J mice using CFU measurements. EcN prototypes used in this study are described in Table 30.

TABLE 30
EcN-EGF Plasmid-Based Prototypes Used in This Study

| | Strain | | EGF | EGF secretion |
|---|---|---|---|---|
| Strain ID | Description | Chassis | promoter | peptide |
| SYN094 | EcN Control | EcN | N/A | N/A |
| SYN8062 | A-Temp | A | cI857 | OmpA |
| SYN8063 | A-FNR | A | FNR | OmpA |

EcN biomass was prepared in AMBR250 bioreactors and cells were centrifuged at 7,800×g and resuspended in 100 mM phosphate buffer containing 15% glycerol. EcN-EGF aliquots were stored at −80° C. EcN strain biomass used in this study is described in Table 31

TABLE 31
Engineered EcN Strain Biomass Used in this Study

| | | CFU/mL |
|---|---|---|
| Strain ID | Strain Description | (Mean ± SD) |
| SYN094 | Control strain | 6.27E+10 ± 4.04E+09 |
| SYN8062 | A-Temp-OmpA-EGF | 6.80E+10 ± 3.46E+09 |
| SYN8063 | A-FNR-OmpA-EGF | 6.53E+10 ± 4.16E+09 |

### In Vivo Study Design

### In Vivo EcN Fitness and Viability Experimental Design

A description of the experimental design and treatment groups used to determine in vivo fitness and viability of EcN is provided in Table 32.

TABLE 32
In Vivo EcN Fitness and Viability Experimental Design

| | | | Dosing | StrainDose | | Collection Time |
|---|---|---|---|---|---|---|
| Group | N | Treatment | route | (CFU) | Collection | Points |
| 1 | 25 | Vehicle | PO | 1E+10 | Gut effluents | Gut effluents: 1, 3, 6, |
| 2 | 25 | SYN094 | PO | 1E+10 | (Stomach, SI, | and 24 hrs post-dose |
| 3 | 25 | SYN8062 | PO | 1E+10 | Cecum, Colon | Feces: 1, 3, 6, 24 hrs, |
| 4 | 25 | SYN8063 | PO | 1E+10 | andFeces) | and 24 hrs thereafter |
| | | | | | | until strain is |
| | | | | | | undetectable. |
| | | | | | | N = 5 mice/group/ |
| | | | | | | time point |

### Bioanalytical Methods

### In Vivo Experimental Design for CFU Measurements

C57BL/6J mice were group housed and assigned to treatment groups. Mice received a single oral dose of the treatment and were sacrificed by CO2 asphyxiation at their assigned time. Feces were collected fresh by free catch, and gut effluents were collected from stomach, small intestine, cecum, and colon by flushing with 500 uL of PBS. Feces and gut effluents were placed into pre-weighed bead-bug tubes containing 500 uL of PBS, weighed, and then processed for serial dilution plating to determine viable colony-forming units (CFUs) immediately after collection.

### Quantitation of Bacterial CFUs in Feces.

Feces and gut effluents were processed for quantification of bacteria by serial dilution and plating on streptomycin- or thymidine- and kanamycin-containing plates. For serial dilution, 1 mL of PBS was added to each bead-bug tube. The tubes were then homogenized and 150 uL was removed from each tube and plated into Row A of a 96-well conical-bottom plate (12 samples per plate). From Row A, 10 uL of homogenate was removed and placed into each consecutive well in that sample column (eg, taken from Row A1; 10 uL into Row B1, Row C1, Row D1 and so on). A 10-point dilution series was performed, making a 1:10 serial dilution with PBS from the initial 10 uL fecal sample (90 uL of PBS was added). The dilutions were then plated on LB agar plates supplemented with streptomycin (300 ug/mL) or kanamycin (3 mM) and thymidine (3 mM) and incubated overnight at 37° C. Twenty-four hours after the incubation began, plates were removed from the incubator, and colonies were manually counted.

### Data Analysis and Results

EcN total cell counts and viability were obtained using Cellometer K2 Matrix software (Nexelcom, Inc.). Raw data were entered in Microsoft excel (Microsoft Excel, Seattle, WA) spreadsheet and transferred to GraphPad Prism v9.0 (GraphPad Software, San Diego, CA). Statistical analysis was performed using GraphPad Prism, and the specific tests used are indicated in the legend to each respective figure. Significance was set as p<0.05.

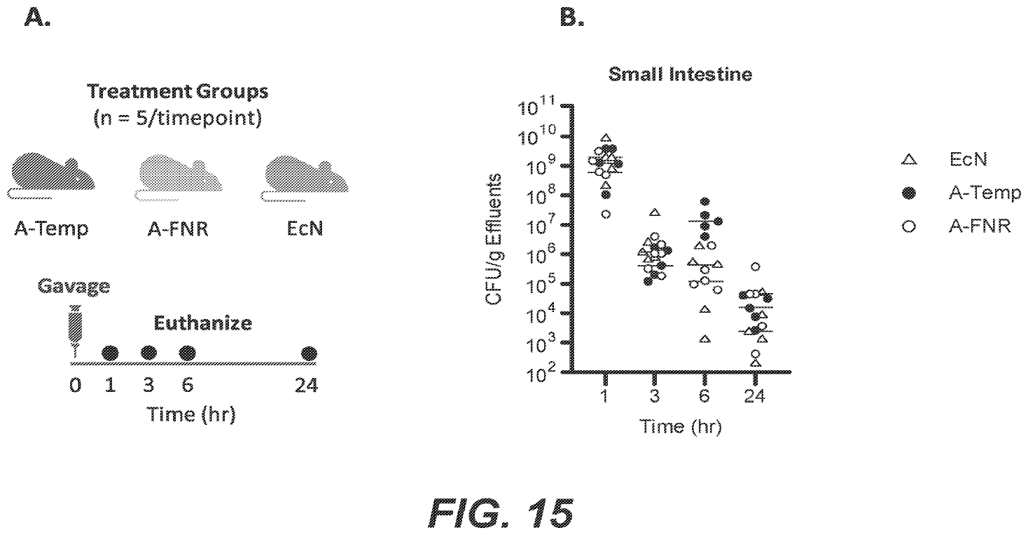

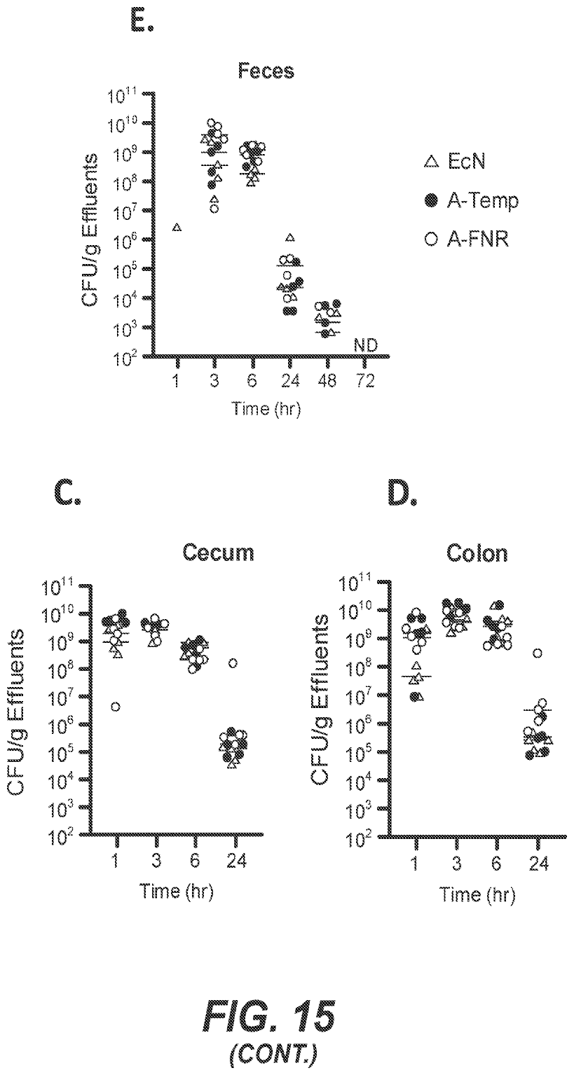

To determine in vivo viability and gut transit of chassis A EcN expressing EGF, two strains were and compared to wildtype EcN. Mice received a single oral bolus of EcN WT (SYN094), A-PcI857-EGF (SYN8062), A-PfnrS-EGF (SYN8063) at 1×10¹⁰ CFU. FIG. 15 shows abundance in effluents that were collected and counted at indicated times from the small intestine, cecum, colon, and feces. The tabular CFU counts used for construction of these charts are included in Table 33 and Table 34. In the effluents obtained from the small intestine, cecum, and colon of mice dosed with either SYN094 (control), SYN8062 or SYN8063, no substantial difference was observed in the survival or GI distribution over the duration of the study. No significant difference in fecal recovery was observed between the three strains at all time points.

TABLE 33
Tabular Results Supporting Bacterial Strain Effluent Recovery

| Gut | | Time of collection (hours) | | | |
|---|---|---|---|---|---|
| segment | Strain | 1 | 3 | 6 | 24 |
| Small | SYN094 | 3.0 × 10⁹ ± 1.7 × 10⁹ | 6.5 × 10⁶ ± 5.1 × 10⁶ | 6.0 × 10⁵ ± 3.5 × 10⁵ | 1.4 × 10⁴ ± 1.0 × 10⁴ |
| intestine | SYN8062 | 2.3 × 10⁹ ± 8.5 × 10⁸ | 7.7 × 10⁵ ± 3.3 × 10⁵ | 2.3 × 10⁷ ± 1.1 × 10⁷ | 2.1 × 10⁴ ± 7.9 × 10³ |
| | SYN8063 | 1.2 × 10⁹ ± 6.0 × 10⁸ | 1.6 × 10⁶ ± 7.1 × 10⁵ | 5.3 × 10⁵ ± 3.8 × 10⁵ | 9.3 × 10⁴ ± 6.9 × 10⁴ |
| Cecum | SYN094 | 1.5 × 10⁹ ± 6.1 × 10⁸ | 2.3 × 10⁹ ± 4.4 × 10⁸ | 6.1 × 10⁸ ± 1.2 × 10⁸ | 1.1 × 10⁵ ± 3.3 × 10⁴ |
| | SYN8062 | 6.0 × 10⁹ ± 7.4 × 10⁸ | 3.6 × 10⁹ ± 1.6 × 10⁸ | 5.5 × 10⁸ ± 1.6 × 10⁸ | 2.1 × 10⁵ ± 8.2 × 10⁴ |
| | SYN8063 | 2.2 × 10⁹ ± 1.0 × 10⁹ | 3.0 × 10⁹ ± 9.3 × 10⁸ | 2.4 × 10⁸ ± 5.6 × 10⁷ | 3.1 × 10⁷ ± 3.0 × 10⁷ |
| Colon | SYN094 | 4.5 × 10⁸ ± 4.0 × 10⁸ | 5.5 × 10⁹ ± 1.8 × 10⁹ | 5.0 × 10⁹ ± 2.6 × 10⁹ | 2.4 × 10⁵ ± 6.7 × 10⁴ |
| | SYN8062 | 2.7 × 10⁹ ± 1.1 × 10⁹ | 1.2 × 10¹⁰ ± 2.1 × 10⁹ | 5.0 × 10⁹ ± 2.4 × 10⁹ | 5.2 × 10⁵ ± 3.1 × 10⁵ |
| | SYN8063 | 2.5 × 10⁹ ± 1.4 × 10⁹ | 5.0 × 10⁹ ± 1.3 × 10⁹ | 1.0 × 10⁹ ± 3.7 × 10⁸ | 6.3 × 10⁷ ± 6.0 × 10⁷ |

N = 5 mice per group per time point.

TABLE 34
Tabular Results Supporting Bacterial Strain Fecal Recovery

| Fecal | | | |
|---|---|---|---|
| collection | | | |
| time (hours) | SYN094 | SYN8062 | SYN8063 |
| 1 | 5.2 × 10⁵ ± | N/D | N/D |
| | 5.2 × 10⁵ | | |
| 3 | 1.1 × 10⁹ ± | 1.5 × 10⁹ ± | 5.0 × 10⁹ ± |
| | 5.9 × 10⁸ | 7.8 × 10⁸ | 1.8 × 10⁹ |
| 6 | 4.8 × 10⁸ ± | 8.7 × 10⁸ ± | 1.1 × 10⁹ ± |
| | 3.2 × 10⁸ | 2.3 × 10⁸ | 2.3 × 10⁸ |
| 24 | 3.1 × 10⁵ ± | 4.6 × 10⁴ ± | 1.2 × 10⁵ ± |
| | 2.9 × 10⁵ | 3.1 × 10⁴ | 5.1 × 10⁴ |
| 48 | 1.1 × 10³ ± | 2.7 × 10³ ± | 1.6 × 10³ ± |
| | 6.1 × 10² | 1.3 × 10³ | 1.0 × 10³ |
| 72 | N/D | N/D | N/D |

N = 5 mice per group per time point, except for 24 h time point SYN094, SYN8063 n = 4 per group

The results in this study demonstrate the biodistribution of engineered EcN. EcN-EGF prototypes (SYN8062, SYN8063) in chassis A follow the same CFU kinetics as EcN WT strain (SYN094). EcN rapidly clears SI and is abundant in distal gut up to 6 hrs post dose. Data suggests no significant effect of modifications on transit.

### Example 11. In Vivo EGF Secretion by Prototype Strains in Naïve Mice

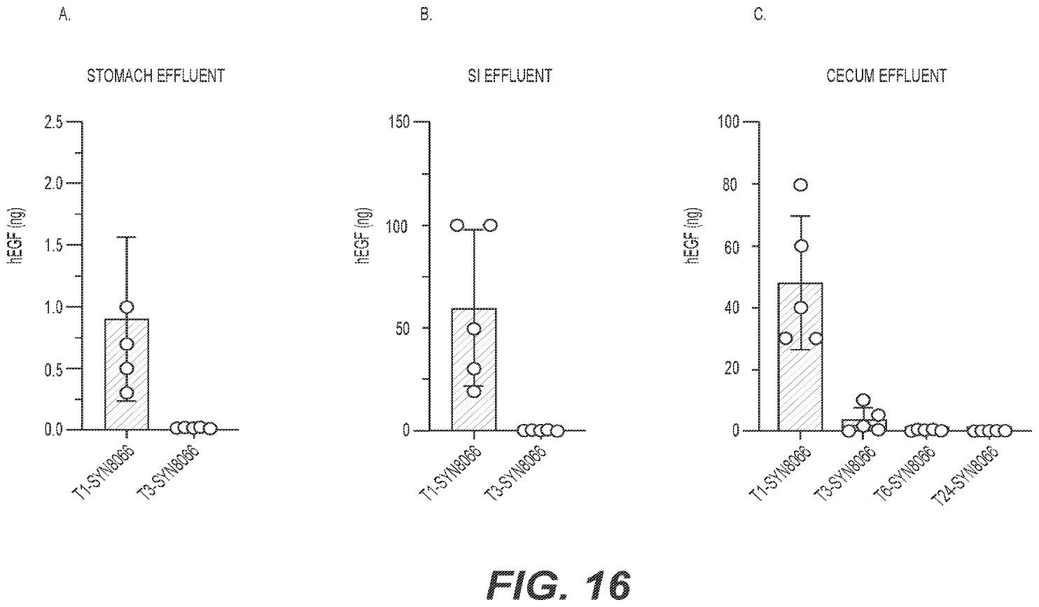

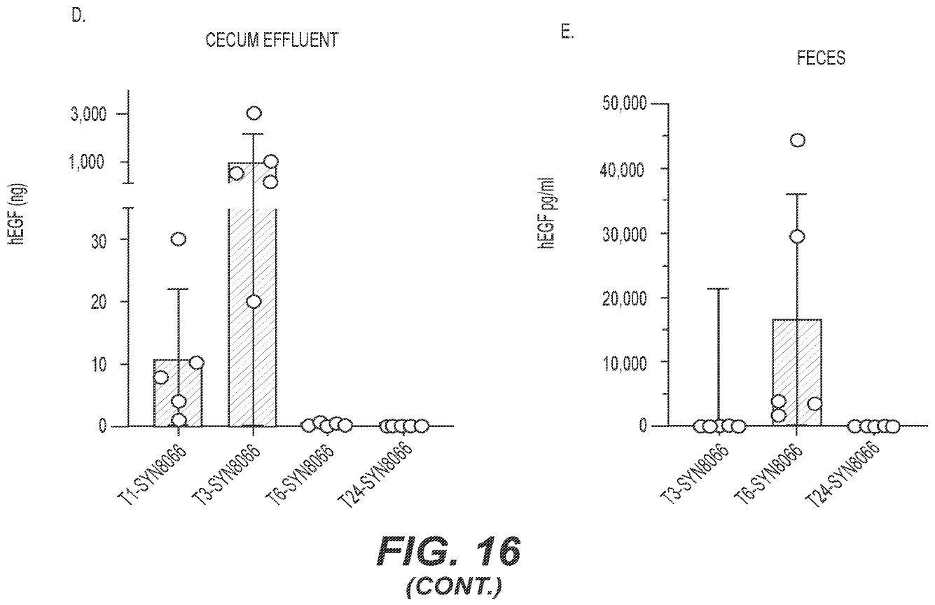

The objective of this study was to determine the ability of prototype EGF-secreting strains to produce EGF in the GI tract. C57BL/6J mice (female, n=5) were group housed and assigned to treatment groups. Mice received a single oral dose of bacterial cells (1e10) and were sacrificed by CO2 asphyxiation at 1, 3, 6 or 24 hours. Effluents (small intestine, cecum, colon) were collected as described above in Example 10. ELISA for EGF was performed per manufacturer's instructions (Invitrogen). Preliminary results are shown in FIG. 16. High levels of hEGF were detected in small intestine (SI) and cecum at 1 hr post-dose and in colon 3 hr post-dose. At 6 hours post dose, these quantities dropped to nanogram levels.

### Example 12. In Vivo Biodistribution and EGF Secretion of Engineered EcN in a DSS Model

Two studies were conducted to determine (1) the biodistribution in the gastrointestinal (GI) tract of prototype EGF-secreting strains and (2) the ability of the strains to produce and secrete EGF in the various GI compartments over time in a DDS model of IBD. EcN prototypes used in these studies are SYN8066 (Chassis B, FNR) and SYN8248 (Chassis B).

In both studies, female mice were randomized into different treatment groups (n=5) by weight and put on 3% DSS drinking water for 7 days. After day 6, DSS water was removed and substituted with normal water. On day 7, mice (female, N=5) received a single oral dose of the treatment (1e10 cells) and were sacrificed by CO2 asphyxiation at 1, 3, 6 or 24 hours and effluents were collected as described in Example 10 above.

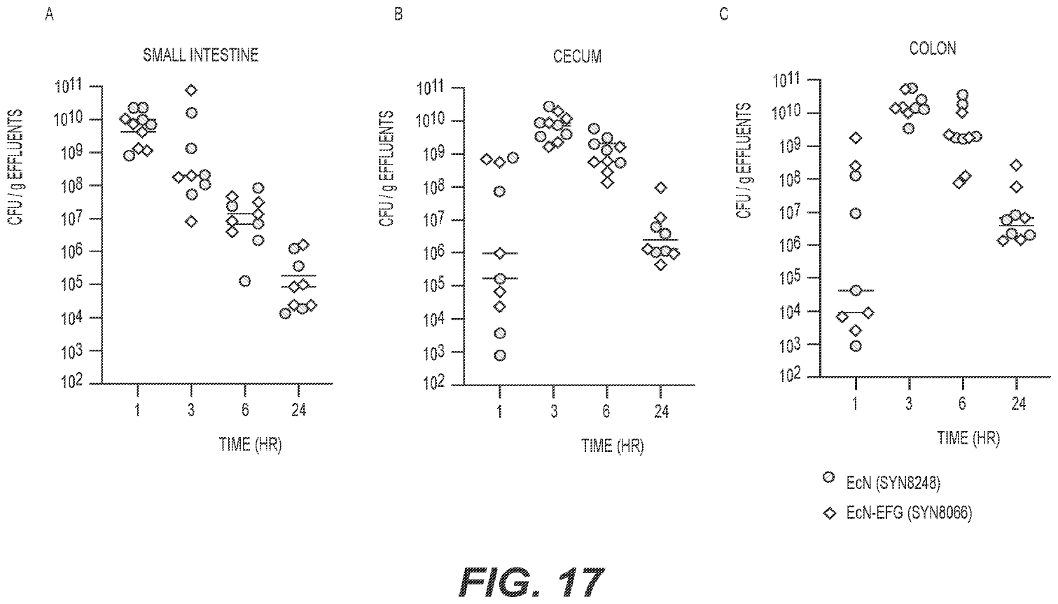

For the biodistribution study, bacteria were quantitated as described above in Example 10. Results are shown in FIG. 17. No substantial difference was observed in the survival or GI distribution over the duration of the study between the EGF secreting strain SYN8066 and the chassis B only strain SYN8248. Data suggests no significant effect of modifications on transit. In the DSS model, EcN cleared the small intestine and was abundant in distal gut up to 24 hrs post dose, which is substantially longer than in naïve mice, where much lower bacterial levels were observed at the 24-hour timepoint. These results demonstrate that the biodistribution of engineered EcN in the gastrointestinal tract in the DSS model differs from the biodistribution in naïve mice, in that transit through the lower intestine is delayed in DSS mice as compared to naïve mice.

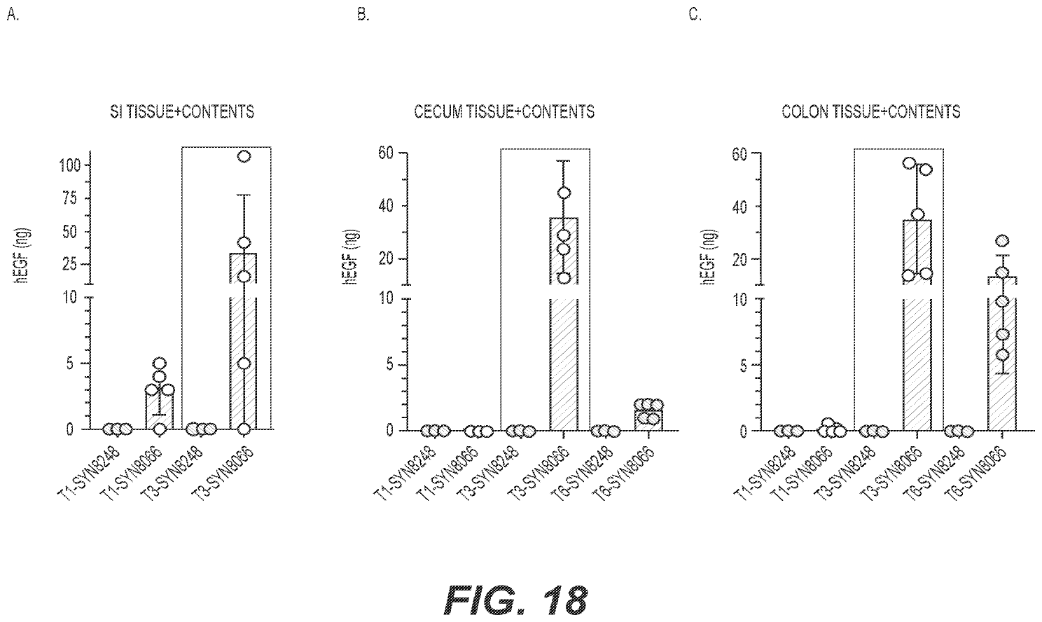

To assess secretion levels in the gastrointestinal tract, mice (female, n=5) were treated with DSS as described above and on day 8 received a single oral dose of bacteria (leO cells). Mice were sacrificed at the 1-, 3-, or 6-hour time points, and effluents were collected for measurement of EFG secretion by ELISA. Results are shown in FIG. 18. In this initial study, similar levels of hEGF were produced in DSS colitis mice as observed previously in healthy mice (˜30 ng in colon at 3h post-dose and ˜10 ng at 6h post-dose).

### Example 13. Construction of Integrated Strains

Strains having 1-, 2-, and 3-copies of integrated FNR-EGF in a chassis B background were generated. Integrated strains are listed in Table 35.

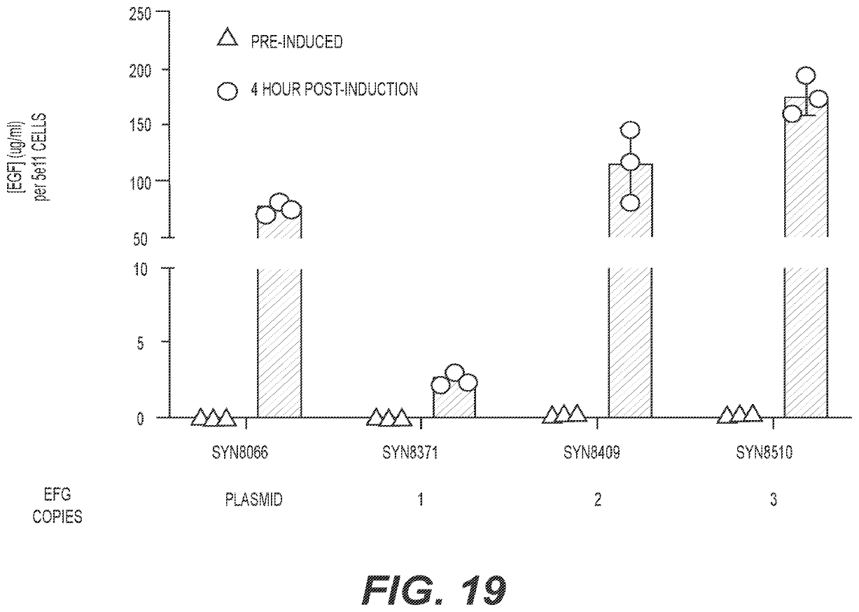

To determine the EGF production capability of the integrated strains EcN-EGF cells were added to 30 mL media to obtain a final concentration of 2.5×10\hat{ }9 total cells/mL. Resuspended EcN-EGF cultures were then split into three 10 mL aliquots in 50 mL flasks and incubated at 37° C. under atmospheric conditions in a shaking incubator at 250 rpm for 8 hrs. EGF-containing supernatants were then collected by centrifuging cultures at 4,000 rpm for 10 mins in a benchtop hanging bucket centrifuge. Supernatant samples were frozen and stored at −80° C. until EGF quantitation by ELISA. Results show that 2-3 copy integrated strains show favorable in vitro activity compared to plasmid-based B-FNR (SYN8066) (FIG. 19).

TABLE 35
Integrated strains

| Strain | |
|---|---|
| number | Description |
| SYN8371 | Single copy integration of fnr_ompA_EGF into |
| | SYN8064 (Chassis B (no DOM mutation)). Strain |
| | has: ΔthyA, ΔclbKO, ΔΦ. |
| SYN8409 | Double copy integration of fnr_ompA_EGF |
| | attB7 and attB5 sites of SYN8064 (Chassis B). |
| | (Chassis (no DOM mutation)). Strain has: |
| | ΔthyA, ΔclbKO, ΔΦ |
| SYN8510 | Three copy integration of fnr_ompA_EGF attB2, |
| | attB7 and attB5 sites of SYN8064 (Chassis B). |
| | (Chassis B (no DOM mutation)). Strain has: |
| | ΔthyA, ΔclbKO, ΔΦ |

1. A recombinant bacterium comprising a polynucleotide sequence encoding a EGF polypeptide fused to a secretion tag, wherein the polynucleotide sequence is operably linked to an FNR-inducible promoter or temperature-sensitive promoter.

2. The recombinant bacterium of claim 1, wherein the secretion tag is any of PhoA, PelB, OmpA, LARD3, or HylA.

3. The recombinant bacterium of claim 1 or 2, wherein the EGF polypeptide comprises at least 80%, 85%, 90%, 95%, 96%, 97%, 98%, or 99% identity to Sequence A or a functional fragment thereof.

4. The recombinant bacterium of any one of claims 1-3, wherein the EGF polypeptide comprises Sequence A or a functional fragment thereof.

5. The recombinant bacterium of any one of claims 2-4, wherein the PhoA secretion tag polypeptide comprises at least 80%, 85%, 90%, 95%, 96%, 97%, 98%, or 99% identity to Sequence D.

6. The recombinant bacterium of claim 5, wherein the PhoA secretion tag polypeptide comprises Sequence D.

7. The recombinant bacterium of any one of claims 2-4, wherein the PelB secretion tag polypeptide comprises at least 80%, 85%, 90%, 95%, 96%, 97%, 98%, or 99% identity to Sequence C.

8. The recombinant bacterium of claim 7, wherein the PelB secretion tag polypeptide comprises Sequence C.

9. The recombinant bacterium of any one of claims 2-4, wherein the OmpA secretion tag polypeptide comprises at least 80%, 85%, 90%, 95%, 96%, 97%, 98%, or 99% identity to Sequence E.

10. The recombinant bacterium of claim 9, wherein the OmpA secretion tag polypeptide comprises Sequence E.

11. The recombinant bacterium of any one of claims 2-4, wherein the LARD3 secretion tag polypeptide comprises at least 80%, 85%, 90%, 95%, 96%, 97%, 98%, or 99% identity to Sequence F.

12. The recombinant bacterium of claim 11, wherein the LARD3 secretion tag polypeptide comprises Sequence F.

13. The recombinant bacterium of any one of claims 2-4, wherein the HylA secretion tag polypeptide comprises at least 80%, 85%, 90%, 95%, 96%, 97%, 98%, or 99% identity to Sequence G.

14. The recombinant bacterium of claim 13, wherein the HylA secretion tag polypeptide comprises Sequence G.

15. The recombinant bacterium of any one of claims 1-14, comprising a low oxygen-inducible promoter.

16. The recombinant bacterium of any one of claims 1-14, comprising a temperature-sensitive inducible promoter.

17. The recombinant bacterium of claim 15, wherein the low oxygen-inducible promoter comprises at least 80%, 85%, 90%, 95%, 96%, 97%, 98%, or 99% identity to any one of SEQ ID NOs: 151-167.

18. The recombinant bacterium of claim 17, wherein the low oxygen-inducible promoter comprises any one of SEQ ID NOs: 151-167.

19. The recombinant bacterium of claim 16, wherein the temperature-sensitive promoter is a pR promoter and further comprises a CI857 repressor.

20. The recombinant bacterium of claim 19, wherein the CI857 repressor-pR promoter comprises at least 80%, 85%, 90%, 95%, 96%, 97%, 98%, or 99% identity to any one of SEQ ID NOs: 183-185.

21. The recombinant bacterium of claim 20, wherein the CI857 repressor-pR promoter comprises any one of SEQ ID NOs: 183-185.

22. The recombinant bacterium of any one of claims 1-21, wherein the EGF fusion polypeptide comprises at least 80%, 85%, 90%, 95%, 96%, 97%, 98%, or 99% identity to Sequences H, I, J, K, or L, or the EGF fusion polynucleotide comprises at least 80%, 85%, 90%, 95%, 96%, 97%, 98%, or 99% identity to Sequences N, O, P, Q, R, Y, Z, AA, AB, AC, AD, AE, or AF.

23. The recombinant bacterium of any one of claims 1-21, wherein the EGF fusion polypeptide comprises Sequences H, I, J, K, or L, or the EGF fusion polynucleotide comprises Sequences N, O, P, Q, R, Y, Z, AA, AB, AC, AD, AE, or AF.

24. The recombinant bacterium of any one of claims 1-23, comprising a gene sequence encoding a polypeptide linker and/or a stabilizing polypeptide.

25. The recombinant bacterium of claim 24, wherein the secretion tag is linked to the N terminus of EGF via a peptide bond or a polypeptide linker.

26. The recombinant bacterium of claim 24, wherein the secretion tag is linked to the C terminus of EGF via a peptide bond or a polypeptide linker.

27. The recombinant bacterium of any one of claims 1-26, wherein the secretion tag is cleaved after secretion of EGF into the extracellular environment.

28. The recombinant bacterium of claims 1-27, wherein the bacterium is selected from the group consisting of Bacteroides, Bifidobacterium, Clostridium, Escherichia, Lactobacillus, and Lactococcus.

29. The recombinant bacterium of claims 1-28, wherein the bacterium is selected from Clostridium novyi NT, Clostridium butyricum, and Bifidobacterium longum.

30. The recombinant bacterium of claim any of claims 1-29, wherein the bacterium is Escherichia coli strain Nissle.

31. The recombinant bacterium of claims 1-30, wherein the bacterium has a mutated gene encoding a periplasmic protein pal and expresses the EGF fusion polypeptide.

32. The recombinant bacterium of claims 1-30, wherein the bacterium has a Type 1 secretion system and expresses the EGF fusion polypeptide.

33. The recombinant bacterium of any one of claims 1-32, wherein the bacterium is capable of secreting about 0.1, 0.2, 0.3, 0.4, 0.5, 0.6, 0.7, 0.8, 0.9, or 1.0 ug EGF/5e11 cells over 4 hours under inducing conditions.

34. The recombinant bacterium of any one of claims 1-32, wherein the bacterium is capable of secreting about 1, 1.5, 2, 2.5, 3, 3.5, 4, 4.5, 5, 5.5, 6, 6.5, 7, 7.5, 8, 8.5, 9, 9.5, or 10 ug EGF/5e11 cells over 4 hours under inducing conditions.

35. The recombinant bacterium of any one of claims 1-32, wherein the bacterium is capable of secreting about 10, 20, 30, 40, 50, 60, 70, 80, 90, 100, 110, or 120 ug EGF/5e11 cells over 4 hours under inducing conditions.

36. The recombinant bacterium of any one of claims 1-32, wherein the bacterium is capable of secreting at least about 110, 120, 130, 140, 150, 160, 170, 180, 190, or 200 μg EGF/5e11 cells over 4 hours under inducing conditions.

37. The recombinant bacterium of any one of claims 1-32, wherein the bacterium is capable of secreting at least about 210, 220, 230, 240, 250, 260, 270, 280, 290, or 300 μg EGF/5e11 cells over 4 hours under inducing conditions.

38. The recombinant bacterium of any one of claims 1-32, wherein the bacterium is capable of secreting at least about 350, 400, 450, 500, 550, 600, 650, 700, 750, or 800 μg EGF/5e11 cells over 4 hours under inducing conditions.

39. The recombinant bacterium of any one of claims 1-32, wherein the bacterium is capable of secreting about 0.1, 0.2, 0.3, 0.4, 0.5, 0.6, 0.7, 0.8, 0.9, or 1.0 ug EGF/5e11 cells over 8 hours under inducing conditions.

40. The recombinant bacterium of any one of claims 1-32, wherein the bacterium is capable of secreting about 1, 1.5, 2, 2.5, 3, 3.5, 4, 4.5, 5, 5.5, 6, 6.5, 7, 7.5, 8, 8.5, 9, 9.5, or 10 ug EGF/5e11 cells over 8 hours under inducing conditions.

41. The recombinant bacterium of any one of claims 1-32, wherein the bacterium is capable of secreting about 10, 20, 30, 40, 50, 60, 70, 80, 90, 100, 110, or 120 ug/mL EGF/5e11 cells over 8 hours under inducing conditions.

42. The recombinant bacterium of any one of claims 1-32, wherein the bacterium is capable of secreting at least about 40, 50, 60, 70, 80, 90, or 100 μg EGF/5e11 cells over 8 hours under inducing conditions.

43. The recombinant bacterium of any one of claims 1-32, wherein the bacterium is capable of secreting at least about 110, 120, 130, 140, 150, 160, 170, 180, 190, or 200 μg EGF/5e11 cells over 8 hours under inducing conditions.

44. The recombinant bacterium of any one of claims 1-32, wherein the bacterium is capable of secreting at least about 210, 220, 230, 240, 250, 260, 270, 280, 290, or 300 μg EGF/5e11 cells over 8 hours under inducing conditions.

45. The recombinant bacterium of any one of claims 1-32, wherein the bacterium is capable of secreting at least about 350, 400, 450, 500, 550, 600, 650, or 700, 750, or 800 μg EGF/5e11 cells over 8 hours under inducing conditions.

46. The bacterium of any one of claims 33-45, wherein the inducing conditions are low oxygen or anaerobic conditions.

47. The bacterium of any one of claims 33-45, wherein the inducing conditions are a temperature between about 37° C. and about 42° C.

48. The recombinant bacterium of any one of claims 1-47, wherein the recombinant bacterium comprises a deletion in the pks island.

49. A pharmaceutical composition comprising the bacterium of any one of claims 1-48.

50. A method of treating a disorder comprising the step of administering to a patient in need thereof the bacterium of any one of claims 1-48 or the composition of claim 49.

51. The method of claim 50, wherein the disorder is selected from a group consisting of autoimmune disorders, cancer, metabolic diseases, diseases relating to inborn errors of metabolism, and neurological or neurodegenerative diseases.

52. The method of claim 51, wherein the autoimmune disorder is selected from the group consisting of acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, antiphospholipid syndrome (APS), autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogan syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibrosing alveolitis, Giant cell arteritis (temporal arteritis), Giant cell myocarditis, Glomerulonephritis, Goodpasture's syndrome, Granulomatosis with Polyangiitis (GPA), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile idiopathic arthritis, Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (Systemic Lupus Erythematosus), chronic Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/giant cell arteritis, thrombocytopenic purpura (TTP), Tolosa-Hunt syndrome, transverse myelitis, type 1 diabetes, asthma, ulcerative colitis, undifferentiated connective tissue disease (UCTD), uveitis, vasculitis, vesiculobullous dermatosis, vitiligo, and Wegener's granulomatosis.

53. The method of claim 51, wherein the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor.

54. The method of claim 51, wherein the metabolic disorder or condition is selected from the group consisting of: type 1 diabetes; type 2 diabetes; metabolic syndrome; Bardet-Biedel syndrome; Prader-Willi syndrome; non-alcoholic fatty liver disease; tuberous sclerosis; Albright hereditary osteodystrophy; brain-derived neurotrophic factor (BDNF) deficiency; Single-minded 1 (SIM1) deficiency; leptin deficiency; leptin receptor deficiency; pro-opiomelanocortin (POMC) defects; proprotein convertase subtilisin/kexin type 1 (PCSK1) deficiency; Src homology 2B1 (SH2B1) deficiency; pro-hormone convertase 1/3 deficiency; melanocortin-4-receptor (MC4R) deficiency; Wilms tumor, aniridia, genitourinary anomalies, and mental retardation (WAGR) syndrome; pseudohypoparathyroidism type 1A; Fragile X syndrome; Borjeson-Forsmann-Lehmann syndrome; Alstrom syndrome; Cohen syndrome; and ulnar-mammary syndrome.
